SEQUENCE LISTING (161 sequences)

SEQ ID NO: 1 - protein, 856 aa, Human Immunodeficiency Virus
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 2 - protein, 633 aa, synthetic construct
  REGION 1..633: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKNVSTVQCTHGIKPVVSTQLLLNGSLAEEEVMIRSENITNNAKNIIVQFNTPVQINCTRPNNMTRKSIRGPGQAFYALGDIIGDIRQPHCTVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKRRVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDLLALD

SEQ ID NO: 3 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQACPKVSFEPIPIHYCAPAGFAILKCKDKKFNGTGPCKNVSTVQCTHGIKPVVSTQLLLNGSLAEEEVMIRSENITNNAKNIIVQFNTPVQINCTRPNNMTRKSIRIGPGQAFYALGDIIGDIRQPHCTVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGIKQLQARVLAVERYLEVQQLLGIWGCSGKLICCTNVPWNSSWSNRNLSEIWDNMTWLQWDKEIGNYTQIIYGLLEESQFQQEINEQDNLTLD

SEQ ID NO: 4 - protein, 660 aa, synthetic construct
  REGION 1..660: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGYNVTYNVTGGYNVTYNVTIGGSGG

SEQ ID NO: 5 - protein, 629 aa, synthetic construct
  REGION 1..629: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINETD

SEQ ID NO: 6 - protein, 692 aa, synthetic construct
  REGION 1..692: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGGSSGVRLWNTTKIMLGQVHNVTKGWLIFVAEQEELYVRVQNGFRKVQLEARTPLPR

SEQ ID NO: 7 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTTTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESNFTQEINEQDNLTLD

SEQ ID NO: 8 - protein, 635 aa, synthetic construct
  REGION 1..635: note = Recombinant HIV-1 Env protein
NSTAENLWVTVYYGVPVWKEAKTTLFCASDAKAYEKEVHNVWATHACVPTDPNPQEMVLENVTENFNMWKNDMVDQMHEDIISLWDQSLKPCVKLTPLCVTLNCTNVNVTNTNNNNMKEEMKNCSFNTTTEIRDKKQKEYALFYRLDIVPLNENSSEYRLINCNTSTCTQICPKVSFDPIPIHYCAPAGYAILKCNNKTFNGTGPCNNVSTVQCTHGIKPVVSTQLLLNGSLAEEEIIIRSENLTDNAKTIIVHLNESVEINCTRPNNMTRKSIRIGPGQTFYALGDIIGDIRQPHCNISEAKWNKTLQRVKKKLKEHFPNKTIKFAPSSGGDLEITTHSFNCRGEFFYCNTSKLFNSTYNNTTSNSTITLPCRIKQIINMWQEVGRCMYAPPIAGNITCKSNITGLLLTRDGGNNNNNTETFRPGGGDMRDNWRSELYKYKVVEIKPLGIAPTKCNRTVVENSTHKNLTHHMRRRRRRAVGLGAVFLGFLGAAGSTMGAASNTLTVQARQLLSGIVQQQSNLLRAPEAQQHMLQLGVWGFKQLQARVLAIERYLEVQQLLGIWGCSGKLICCTAVPWNSTWSNKTQEDIWDNMTWMQWDREIGNYTDTIYRLLEESQFQQEINEKDNLTLPNST

SEQ ID NO: 9 - protein, 651 aa, synthetic construct
  REGION 1..651: note = Recombinant HIV-1 Env protein
NSTAENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGNSTHKNLTHHMRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLPNST

SEQ ID NO: 10 - protein, 651 aa, synthetic construct
  REGION 1..651: note = Recombinant HIV-1 Env protein
NSTAENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGNSTHKNLTHHMRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSTWSNRNLTEIWDNMTWLQWDKEIGNYTQIIYGLLEESQFQQEINEQDNLTLPNST

SEQ ID NO: 11 - protein, 11 aa, synthetic construct
  REGION 1..11: note = Glycosylation sequence
NSTHKNLTHHM

SEQ ID NO: 12 - protein, 26 aa, synthetic construct
  REGION 1..26: note = Glycosylation sequence
GGYNVTYNVTGGYNVTYNVTIGGSGG

SEQ ID NO: 13 - protein, 58 aa, synthetic construct
  REGION 1..58: note = Glycosylation sequence
GGGSSGVRLWNTTKIMLGQVHNVTKGWLIFVAEQEELYVRVQNGFRKVQLEARTPLPR

SEQ ID NO: 14 - protein, 7 aa, synthetic construct
  REGION 1..7: note = Recombinant peptide sequence
GRRRRRR

SEQ ID NO: 15 - protein, 10 aa, synthetic construct
  REGION 1..10: note = Recombinant peptide sequence
GGSGRRRRRR

SEQ ID NO: 16 - protein, 10 aa, synthetic construct
  REGION 1..10: note = Recombinant peptide sequence
GRRRRRRRRR

SEQ ID NO: 17 - protein, 18 aa, synthetic construct
  REGION 1..18: note = Recombinant peptide sequence
GNSTHKQLTHHMRRRRRR

SEQ ID NO: 18 - protein, 10 aa, synthetic construct
  REGION 1..10: note = Recombinant peptide sequence
GRRRSRRRRR

SEQ ID NO: 19 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKNVSTVQCTHGIKPVVSTQLLLNGSLAEEEVMIRSENITNNAKNIIVQFNTPVQINCTRPNNMTRKSIRIGPGQAFYALGDIIGDIRQPHCTVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKRRVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDLLALD

SEQ ID NO: 20 - protein, 616 aa, synthetic construct
  REGION 1..616: note = Recombinant HIV-1 Env protein
NLWVTVYYGVPVWKEAKTTLFCASDAKAYEKEVHNVWATHACVPTDPNPQEMVLENVTENFNMWKNDMVDQMHEDIISLWDQSLKPCVKLTPLCVTLNCTNVNVTNTNNNNMKEEMKNCSFNTTTEIRDKKQKEYALFYRLDIVPLNENSSEYRLINCNTSTITQICPKVSFDPIPIHYCAPAGYAILKCNNKTFNGTGPCNNVSTVQCTHGIKPVVSTQLLLNGSLAEEEIIIRSENLTDNAKTIIVHLNESVEINCTRPNNMTRKSIRIGPGQTFYALGDIIGDIRQPHCNISEAKWNKTLQRVKKKLKEHFPNKTIKFAPSSGGDLEITTHSFNCRGEFFYCNTSKLFNSTYNNTTSNSTITLPCRIKQIINMWQEVGRAMYAPPIAGNITCKSNITGLLLTRDGGNNNNNTETFRPGGGDMRDNWRSELYKYKVVEIKPLGIAPTKCKRRVVERRRRRRAVGLGAVFLGFLGAAGSTMGAASNTLTVQARQLLSGIVQQQSNLLRAPEAQQHMLQLGVWGFKQLQARVLAIERYLEVQQLLGIWGCSGKLICCTAVPWNSSWSNKSQEDIWDNMTWMQWDREIGNYTDTIYRLLEESQFQQEINEKDLLALD

SEQ ID NO: 21 - protein, 6 aa, synthetic construct
  REGION 1..6: note = Recombinant peptide sequence
RRRRRR

SEQ ID NO: 22 - protein, 845 aa, Human Immunodeficiency Virus
MRVMGIQRNCQHLLRWGTMILGMIIICSAAENLWVTVYYGVPVWKDAETTLFCASDAKAYETEMHNVWATHACVPTDPNPQEIHLENVTEEFNMWKNNMVEQMHTDIISLWDQSLKPCVKLTPLCVTLNCSNVNVTNNTTNTHEEEIKNCSFNMTTELRDKKQKVYSLFYRLDVVQINENNSNSSYRLINCNTSAITQACPKVSFEPIPIHYCAPAGFAILKCKDKEFNGTGPCKNVSTVQCTHGIKPVVSTQLLLNGSLAEEEVIIRSENITNNAKTIIVQLTKPVKINCTRPNNNTRKSIRIGPGQAFYATGDIIGDIRQAHCNVSRSEWNKTLQKVAKQLRKYFKNKTIIFTNSSGGDLEITTHSFNCGGEFFYCNTSGLFNSTWNNGTMKNTITLPCRIKQIINMWQRAGQAMYAPPIQGVIRCESNITGLLLTRDGGNNNTNETFRPGGGDMRDNWRSELYKYKVVKIEPLGVAPTRAKRRVVEREKRAVGIGAVFLGFLGAAGSTMGAASITLTVQARQLLSGIVQQQSNLLRAIEAQQHLLKLTVWGIKQLQARVLAVERYLKDQQLLGIWGCSGKLICTTNVPWNSSWSNKSQNEIWDNMTWLQWDKEISNYTHIIYNLIEESQNQQEKNEQDLLALDKWANLWNWFDISNWLWYIKIFIMIVGGLIGLRIVFAVLSVINRVRQGYSPLSFQTHTPNPRGLDRPGRIEEEGGEQGRDRSIRLVSGFLALAWDDLRSLCLFSYHRLRDFILIAARTVELLGHSSLKGLRLGWEGLKYLWNLLLYWGRELKISAINLVDTIAIAVAGWTDRVIEIGQRIGRAILHIPRRIRQGLERALL

SEQ ID NO: 23 - protein, 843 aa, Human Immunodeficiency Virus
MRVMGTQRNYQHLWRWGILILGMLIMCKATDLWVTVYYGVPVWKDADTTLFCASDAKAYDTEVHNVWATHACVPTDPNPQEVNLENVTEDFNMWKNNMVEQMHEDIISLWDQSLKPCVKLTPLCVTLNCSNANTTNNSTMEEIKNCSYNITTELRDKTQKVYSLFYKLDVVQLDESNKSEYYYRLINCNTSAITQACPKVSFEPIPIHYCAPAGFAILKCKDPRFNGTGSCNNVSSVQCTHGIKPVASTQLLLNGSLAEGKVMIRSENITNNAKNIIVQFNKPVPITCIRPNNNTRKSIRFGPGQAFYTNDIIGDIRQAHCNINKTKWNATLQKVAEQLREHFPNKTIIFTNSSGGDLEITTHSFNCGGEFFYCNTTGLFNSTWKNGTTNNTEQMITLPCRIKQIINMWQRVGRAMYAPPIAGVIKCTSNITGIILTRDGGNNETETFRPGGGDMRDNWRSELYKYKVVKIEPLGVAPTRAKRRVVEREKRAVGMGAVFLGFLGAAGSTMGAASITLTVQARQLLSGIVQQQSNLLKAIEAQQHLLKLTVWGIKQLQARVLALERYLQDQQLLGIWGCSGKLICATTVPWNSSWSNKTQEEIWNNMTWLQWDKEISNYTNIIYKLLEESQNQQEKNEQDLLALDKWANLWNWFNITNWLWYIRIFIMIVGGLIGLRIVIAIISVVNRVRQGYSPLSFQIPTPNPEGLDRPGRIEEGGGEQGRDRSIRLVSGFLALAWDDLRSLCLFSYHRLRDCILIAARTVELLGHSSLKGLRLGWEGLKYLWNLLLYWGRELKNSAISLLDTIAVAVAEWTDRVIEIGQRACRAILNIPRRIRQGFERALL

SEQ ID NO: 24 - protein, 841 aa, Human Immunodeficiency Virus
MRVKGIRKNYQHLWRWGTMLLGMLMICSAAEKLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDLMNATNTNTTIIYRWRGEIKNCSFNITTSIRDKVQKEYALFYKLDVVPIDNDNTSYRLISCNTSVITQACPKVSFEPIPIHYCAPAGFAILKCNDKKFNGTGPCTNVSTVQCTHGIRPVVSTQLLLNGSLAEEEVVIRSENFTDNAKTIIVQLNESVEINCTRPNNNTRKSIHIGPGRAFYTTGEIIGDIRQAHCNISRAKWNNTLKQIVKKLREQFGNKTIVFNQSSGGDPEIVMHSFNCGGEFFYCNTTQLFNSTWNGTWNNTEGNITLPCRIKQIINMWQEVGKAMYAPPIRGQIRCSSNITGLLLTRDGGNNETEIFRPGGGDMRDNWRSELYKYKVVKIEPLGVAPTKAKRRVVQREKRAVGIGAMFLGFLGAAGSTMGAASMTLTVQARQLLSGIVQQQNNLLRAIEAQQHLLQLTVWGIKQLQARVLAVERYLKDQQLLGIWGCSGKLICTTAVPWNASWSNKSLDEIWDNMTWMEWEREIDNYTSLIYTLIEESQNQQEKNEQELLELDKWASLWNWFDITNWLWYIKIFIMIVGGLVGLRIVFAVLSIVNRVRQGYSPLSFQTRLPAPRGPDRPEGIEEEGGERDRDRSGRLVDGFLALIWDDLRSLCLFSYHRLRDLLLIVTRIVELLGRRGWEVLKYWWNLLQYWSQELKNSAVSLLNATAIAVAEGTDRVIEVVQRACRAILHIPRRIRQGLERALL

SEQ ID NO: 25 - protein, 835 aa, Human Immunodeficiency Virus
MRVRGILRNCQQWWIWGILGFWMLMICNVVGNLWVTVYYGVPVWKEAKTTLFCASDAKAYEKEVHNVWATHACVPTDPNPQEIVLENVTENFNMWKNDMVDQMHEDIISLWDQSLKPCVKLTPLCVTLNCTNATNATNTMGEIKNCSFNITTELRDKKQKVYALFYRLDIVPLNENNSYRLINCNTSAITQACPKVSFDPIPIHYCAPAGYAILKCNNKTFNGTGPCNNVSTVQCTHGIKPVVSTQLLLNGSLAEEEIIIRSENLTNNAKTIIVHLNESVEIVCTRPNNNTRKSIRIGPGQTFYATGDIIGDIRQAHCNISEDKWNKTLQKVSKKLKEHFPNKTIKFEPSSGGDLEITTHSFNCRGEFFYCNTSKLFNSTYNSTNSTITLPCRIKQIINMWQEVGRAMYAPPIAGNITCKSNITGLLLTRDGGKNNTETFRPGGGDMRDNWRSELYKYKVVEIKPLGIAPTKAKRRVVEREKRAVGIGAVFLGFLGAAGSTMGAASITLTVQARQLLSGIVQQQSNLLRAIEAQQHMLQLTVWGIKQLQTRVLAIERYLKDQQLLGIWGCSGKLICTTAVPWNSSWSNKSQEDIWDNMTWMQWDREISNYTDTIYRLLEDSQNQQEKNEKDLLALDSWKNLWNWFDITNWLWYIKIFIMIVGGLIGLRIIFAVLSIVNRVRQGYSPLSFQTLTPNPRGPDRLGRIEEEGGEQDRDRSIRLVSGFLALAWDDLRSLCLFSYHRLRDFILIAARAVELLGRSSLRGLQRGWEALKYLGSLVQYWGLELKKSAISLLDTIAIAVAEGTDRIIELIQRICRAIRNIPRRIRQGFEAALQ

SEQ ID NO: 26 - protein, 840 aa, Human Immunodeficiency Virus
MRVRGIQRNYQHLWRWGIMLLGMLMICSVAENLWVTVYYGVPVWKEATTTLFCASDAKSYKTEAHNIWATHACVPTDPNPQEIELENVTENFNMWKNNMVEQMHEDIISLWDQSLKPCVKLTPLCVTLNCTDVKRNNTSNDTNEGEMKNCSFNITTEIRDKKKQVHALFYKLDVVPIDDNNSNTSYRLINCNTSAITQACPKVTFEPIPIHYCAPAGFAILKCKDKKFNGTGPCKNVSTVQCTHGIRPVVSTQLLLNGSLAEEEIIIRSENLTNNAKIIIVQLNESVTINCTRPYNNTRQRTPIGPGQALYTTRIKGDIRQAHCNISRAEWNKTLQQVAKKLGDLLNKTTIIFKPSSGGDPEITTHSFNCGGEFFYCNTSRLFNSTWNNTKWNSTGKITLPCRIKQIINMWQGVGKAMYAPPIEGLIKCSSNITGLLLTRDGGANNSHNETFRPGGGDMRDNWRSELYKYKVVKIEPLGVAPTRAKRRVVEREKRAIGLGAMFLGFLGAAGSTMGAASMTLTVQARQLLSGIVQQQNNLLRAIEAQQHLLQLTVWGIKQLQARILAVERYLKDQQLLGIWGCSGKHICTTTVPWNSSWSNKSLDEIWNNMTWMEWEREIDNYTGLIYSLIEESQNQQEKNEQELLELDKWASLWNWFSITQWLWYIKIFIMIVGGLIGLRIVFAVLSLVNRVRQGYSPLSFQTLLPAPRGPDRPEGIEEEGGEQGRGRSIRLVNGFSALIWDDLRNLCLFSYHRLRDLILIAARIVELLGRRGWEALKYLWNLLQYWIQELKNSAISLFDTTAIAVAEGTDRVIEIVQRACRAILNIPTRIRQGLERALL

SEQ ID NO: 27 - protein, 832 aa, Human Immunodeficiency Virus
MRVRGMQRNWQHLGKWGLLFLGILIICNAAENLWVTVYYGVPVWKEATTTLFCASDAKSYEKEVHNVWATHACVPTDPNPQEVVLENVTENFDMWKNNMVEQMHTDIISLWDQSLKPCVKLTPLCVTLNCTDVNATNNDTNDNKTGAIQNCSFNMTTEVRDKKLKVHALFYKLDIVPISNNNSKYRLINCNTSTITQACPKVSWDPIPIHYCAPAGYAILKCNDKRFNGTGPCKNVSTVQCTHGIKPVVSTQLLLNGSLAEEDIIIRSQNISDNAKTIIVHLNESVQINCTRPNNNTRKSIHLGPGQAFYATGEIIGDIRKAHCNISGTQWNKTLEQVKAKLKSHFPNKTIKFNSSSGGDLEITMHSFNCRGEFFYCNTSGLFNDTGSNGTITLPCRIKQIVNMWQEVGRAMYAAPIAGNITCNSNITGLLLTRDGGQNNTETFRPGGGNMKDNWRSELYKYKVVEIEPLGVAPTKAKRQVVKRERRAVGIGAVFLGFLGAAGSTMGAASITLTVQARQLLSGIVQQQNNLLRAIEAQQHLLQLTVWGIKQLQARVLAVERYLKDQQLLGLWGCSGKLICTTNVPWNSSWSNKSQDEIWNNMTWMEWEKEISNYSNIIYRLIEESQNQQEKNEQELLALDKWASLWNWFDISNWLWYIKIFIMIVGGLIGLRIVFAVLSIVNRVRKGYSPLSLQTLIPSPREPDRPEGIEEGGGEQGKDRSVRLVNGFLALVWDDLRNLCLFSYRHLRDFILIAARIVDRGLRRGWEALKYLGNLTQYWSQELKNSAISLLNTTAIVVAEGTDRVIEALQRAGRAVLNIPRRIRQGLERALL

SEQ ID NO: 28 - protein, 831 aa, Human Immunodeficiency Virus
MRVREMQRNWQHLGKWGLLFLGILIICNAADNLWVTVYYGVPVWKEATTTLFCASDAKAYEREVHNVWATYACVPTDPSPQELVLGNVTENFNMWKNNMVDQMHEDIISLWDQSLKPCVKLTPLCVTLNCTDVNVTINTTNVTLGEIKNCSFNITTEIKDKKKKEYALFYRLDVVPINNSIVYRLISCNTSTVTQACPKVSFEPIPIHYCAPAGFAILKCNDKKFNGTGLCRNVSTVQCTHGIRPVVSTQLLLNGSLAEEDIIIRSENISDNTKTIIVQFNRSVEINCTRPNNNTRKSIRIGPGRAFYATGDIIGDIRKAYCNINRTLWNETLKKVAEEFKNHFNITVTFNPSSGGDLEITTHSFNCRGEFFYCNTSDLFNNTEVNNTKTITLPCRIRQFVNMWQRVGRAMYAPPIAGQIQCNSNITGLLLTRDGGKNGSETLRPGGGDMRDNWRSELYKYKVVKIEPLGVAPTKAKRQVVQREKRAVGIGAVLLGFLGAAGSTMGAASITLTVQARQLLSGIVQQQSNLLKAIEAQQHLLQLTVWGIKQLQARILAVERYLKDQQLLGIWGCSGKLICTTNVPWNSSWSNKSQDEIWDNMTWMQWEKEISNYTDTIYRLIEDAQNQQEKNEQDLLALDKWDNLWSWFTITNWLWYIKIFIMIVGGLIGLRIVFAVLSVVNRVRQGYSPLSLQTLIPNPRGPERPGGIEEEGGEQDRDRSIRLVSGFLALAWDDLRSLCLFSYRHLRDFILIAARTVDMGLKRGWEALKYLWNLPQYWGQELKNSAISLLDTTAIAVAEGTDRIIEVLQRAGRAVLHIPRRIRQGFERALL

SEQ ID NO: 29 - protein, 847 aa, Human Immunodeficiency Virus
MRVKGIRKSYQYLWKGGTLLLGILMICSAVEKLWVTVYYGVPVWKEATTTLFCASDAKAYDTEVHNVWATHACVPTDPNPQEVVLENVTEHFNMWKNNMVEQMQEDIISLWDQSLKPCVKLTPLCVTLNCKDVNATNTTNDSEGTMERGEIKNCSFNITTSIRDEVQKEYALFYKLDVVPIDNNNTSYRLISCDTSVITQACPKISFEPIPIHYCAPAGFAILKCNDKTFNGKGPCKNVSTVQCTHGIRPVVSTQLLLNGSLAEEEVVIRSDNFTNNAKTIIVQLKESVEINCTRPNNNTRKSIHIGPGRAFYTTGEIIGDIRQAHCNISRAKWNDTLKQIVIKLREQFENKTIVFNHSSGGDPEIVMHSFNCGGEFFYCNSTQLFNSTWNNNTEGSNNTEGNTITLPCRIKQIINMWQEVGKAMYAPPIRGQIRCSSNITGLLLTRDGGINENGTEIFRPGGGDMRDNWRSELYKYKVVKIEPLGVAPTKAKRRVVQREKRAVGIGAVFLGFLGAAGSTMGAASMTLTVQARLLLSGIVQQQNNLLRAIEAQQRMLQLTVWGIKQLQARVLAVERYLGDQQLLGIWGCSGKLICTTAVPWNASWSNKSLDRIWNNMTWMEWEREIDNYTSEIYTLIEESQNQQEKNEQELLELDKWASLWNWFDITKWLWYIKIFIMIVGGLVGLRLVFTVLSIVNRVRQGYSPLSFQTLLPAPRGPDRPEGIEEEGGERDRDRSGRLVNGFLALIWVDLRSLCLFSYHRLRDLLLTVTRIVELLGRRGWEVLKYWWNLLQYWSQELKNSAVSLLNATAIAVAEGTDRIIEALQRTYRAILHIPTRIRQGLERALL

SEQ ID NO: 30 - protein, 855 aa, Human Immunodeficiency Virus
MRVKEKYQHLWRWGWRWGTMLLGMLMICSATEKLWVTVYYGVPVWKEATTTLFCASDAKAYDTEVHNVWATHACVPTDPNPQEVELENVTENFNMWKNNMVEQMHEDIISLWDQSLKPCVKLTPLCVTLNCTDLRNATNGNDTNTTSSSREMMGGGEMKNCSFKITTNIRGKVQKEYALFYKLDIVPIDNNSNNRYRLISCNTSVITQACPKISFEPIPIHYCAPAGFAILKCKDKKFNGKGPCSNVSTVQCTHGIRPVVSTQLLLNGSLAEEEVVIRSENFADNAKTIIVQLNESVEINCTRPNNNTRKSIHIGPGRALYTTGKIIGDIRQAHCNLSRAKWNDTLNKIVIKLREQFGNKTIVFKHSSGGDPEIVTHSFNCGGEFFYCNSTQLFNSTWNVTEESNNTVENNTITLPCRIKQIINMWQKVGRAMYAPPIRGQIRCSSNITGLLLTRDGGPEDNKTEVFRPGGGDMRDNWRSELYKYKVVKIEPLGVAPTKAKRRVVQREKRAVGIGAVFLGFLGAAGSTMGAASMTLTVQARLLLSGIVQQQNNLLRAIEAQQHLLQLTVWGIKQLQARVLAVERYLRDQQLLGIWGCSGKLICTTAVPWNASWSNKSLNKIWDNMTWMEWDREINNYTSIIYSLIEESQNQQEKNEQELLELDKWASLWNWFEITEWLWYIKIFIMIIGGLIGLRIVFSVLSIMNRVRQGYSPLSFQTHLPASRGPDRPGGIEEEGGERDRDRSGRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 31 - protein, 860 aa, Human Immunodeficiency Virus
MRVMGIQRNCQHLFRWGTMILGMIIICSAAENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVMIRSENITNNAKNILVQFNTPVQINCTRPNNNTRKSIRIGPGQAFYATGDIIGDIRQAHCTVSKATWNETLGKVVKQLRKHFGNNTIIRFANSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRAKRRVVGREKRAVGIGAVFLGFLGAAGSTMGAASMTLTVQARNLLSGIVQQQSNLLRAIEAQQHLLKLTVWGIKQLQARVLAVERYLRDQQLLGIWGCSGKLICTTNVPWNSSWSNRNLSEIWDNMTWLQWDKEISNYTQIIYGLLEESQNQQEKNEQDLLALDKWASLWNWFDISNWLWYIKIFIMIVGGLIGLRIVFAVLSVIHRVRQGYSPLSFQTHTPNPRGLDRPERIEEEDGEQDRGRSTRLVSGFLALAWDDLRSLCLFCYHRLRDFILIAARIVELLGHSSLKGLRLGWEGLKYLWNLLAYWGRELKISAINLFDTIAIAVAEWTDRVIEIGQRLCRAFLHIPRRIRQGLERALL

SEQ ID NO: 32 - protein, 849 aa, Human Immunodeficiency Virus
MKVMGTKKNYQHLWRWGIMLLGMLMMSSAAEQLWVTVYYGVPVWREANTTLFCASDAKAYDTEVHNVWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTGYRLINCNTSVITQACPKVSFEPIPIHYCAPAGFAILKCNNKTFNGKGPCRNVSTVQCTHGIKPVVSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRAKRRVVQREKRAVTLGAVFLGFLGAAGSTMGAASLTLTVQARLLLSGIVQQQSNLLRAIEAQQHMLQLTVWGIKQLQARVLAIERYLKDQQLLGIWGCSGKLICTTTVPWNTSWSNKSYDYIWNNMTWMQWEREIDNYTGFIYTLIEESQNQQEKNELELLELDKWASLWNWFNITNWLWYIKLFIMIIGGLVGLRIVCAVLSIVNRVRQGYSPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERALL

SEQ ID NO: 33 - protein, 858 aa, Human Immunodeficiency Virus
MRVRGIPRNWPQWWIWGILGFWMIIICRVVGNLDLWVTVYYGVPVWKEAKTTLFCASDAKAYDKEVHNVWATHACVPTDPNPQEIVLENVTENFNMWKNDMVDQMHEDIISLWDQSLKPCVKLTPLCVTLNCKNVNISANANATATLNSSMNGEIKNCSFNTTTELRDKKQKVYALFYKPDVVPLNGGEHNETGEYILINCNSSTITQACPKVSFDPIPIHYCAPAGYAILKCNNKTFNGTGPCNNVSTVQCTHGIKPVVSTQLLLNGSLAEEEIIVRSENLTNNIKTIIVHLNKSVEIKCTRPNNNTRKSVRIGPGQTFYATGEIIGDIREAHCNISRETWNSTLIQVKEKLREHYNKTIKFEPSSGGDLEVTTHSFNCRGEFFYCDTTKLFNETKLFNESEYVDNKTIILPCRIKQIINMWQEVGRAMYAPPIEGNITCKSNITGLLLTWDGGENSTEGVFRPGGGNMKDNWRSELYKYKVVEIKPLGVAPTKSKRKVVGREKRAVGLGAVLLGFLGAAGSTMGAASITLTVQARQLLSGIVQQQSNLLRAIEAQQHLLQLTVWGIKQLQTRVLAIERYLKDQQLLGLWGCSGKLICATAVPWNSSWSNKSLGDIWDNMTWMQWDREISNYTNTIFRLLEDSQNQQEKNEKDLLALDSWKNLWNWFDITNWLWYIKIFIMIVGGLIGLRIIFGVLAIVKRVRQGYSPLSFQTLIPNPRGPDRLGRIEEEGGEQDKDRSIRLVSGFLALAWDDLRSLCLFSYHQLRDFILTAARAAELLGRSSLRGLQRGWEVLKYLGNLVQYWGLELKRSAINLFDTIAIAVAEGTDRIIEVIQRICRAIRYIPTRIRQGFEAALL

SEQ ID NO: 34 - protein, 852 aa, Human Immunodeficiency Virus
MTVTGTWRNYQQWWIWGILGFWMLMICNGLWVTVYYGVPVWREAKTTLFCASDAKSYEKEVHNVWATHACVPTDPNPQELVLKNVTENFNMWKNDMVDQMHEDIISLWDQSLKPCVKLTPLCVTLNCSDAKVNINATYNGTREEIKNCSFNATTELRDKKKKEYALFYRLDIVPLNKEGNNNSEYRLINCNTSVITQACPKVTFDPIPIHYCAPAGYAILKCNNKTFNGTGPCNNVSTVQCTHGIKPVVSTQLLLNGSLAEEEIIIRSENLTDNVKTIIVHLNESVEINCTRPNNNTRKSIRIGPGQTFYATGDIIGDIRQAHCNISEIKWEKTLQRVSEKLREHFNKTIIFNQSSGGDLEITTHSFNCGGEFFYCNTSDLFFNKTFDETYSTGSNSTNSTITLPCRIKQIINMWQEVGRAMYASPIAGEITCKSNITGLLLTRDGGGNNSTEETFRPGGGNMRDNWRSELYKYKVVEVKPLGIAPTEARRRVVQKEKRAVVGLGAVFLGFLGTAGSTMGAASITLTVQARQLLSGIVQQQSNLLRAIEAQQHMLQLTVWGIKQLQARVLAIERYLKDQQLLGMWGCSGKLICTTNVYWNSSWSNKTYNEIWDNMTWMQWDREIDNYTDTIYKLLEVSQKQQESNEKDLLALDSWNNLWNWFDISKWLWYIKIFIMIVGGLIGLRIIFAVLSLVNRVRQGYSPLSFQTLTPNPRELDRLGGIEEEGGEQDRDRSIRLVSGFFSLAWNDLRSLCLFCYHRLRDFILIAGRAVELLGRSSLQGLQRGWEILKYLGSLVQYWGLELKKSAINLFDTIAIAVAEGTDRIIEFLQRIVRAILHIPRRIRQGFEAALQ

SEQ ID NO: 35 - protein, 659 aa, synthetic construct
  REGION 1..659: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGYNVTYNVTGYNVTYNVTIGGSGG

SEQ ID NO: 36 - protein, 661 aa, synthetic construct
  REGION 1..661: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGYNVTYNVTGSGYNVTYNVTIGGSGG

SEQ ID NO: 37 - protein, 658 aa, synthetic construct
  REGION 1..658: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGYNVTYNVTGSGYNVTYIGGSGG

SEQ ID NO: 38 - protein, 663 aa, synthetic construct
  REGION 1..663: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGYNVTYNVTGGSGGYNVTYNVTIGGSGG

SEQ ID NO: 39 - protein, 637 aa, synthetic construct
  REGION 1..637: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTNVTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLD

SEQ ID NO: 40 - protein, 638 aa, synthetic construct
  REGION 1..638: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTYNVTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLD

SEQ ID NO: 41 - protein, 641 aa, synthetic construct
  REGION 1..641: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTNVTYNVTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLD

SEQ ID NO: 42 - protein, 643 aa, synthetic construct
  REGION 1..643: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTNVTGGYNVTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLD

SEQ ID NO: 43 - protein, 643 aa, synthetic construct
  REGION 1..643: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTNVTGGYNVTDDGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLD

SEQ ID NO: 44 - protein, 634 aa, synthetic construct
  REGION 1..634: note = Recombinant HIV-1 Env protein
AENLTVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLD

SEQ ID NO: 45 - protein, 634 aa, synthetic construct
  REGION 1..634: note = Recombinant HIV-1 Env protein
ANNTWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLD

SEQ ID NO: 46 - protein, 635 aa, synthetic construct
  REGION 1..635: note = Recombinant HIV-1 Env protein
NVTNLTVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLD

SEQ ID NO: 47 - protein, 636 aa, synthetic construct
  REGION 1..636: note = Recombinant HIV-1 Env protein
NVTNNTWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLD

SEQ ID NO: 48 - protein, 637 aa, synthetic construct
  REGION 1..637: note = Recombinant HIV-1 Env protein
NVTYNNTWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLD

SEQ ID NO: 49 - protein, 664 aa, synthetic construct
  REGION 1..664: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTNVTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGYNVTYNVTGSGYNVTYNVTIGGSGG

SEQ ID NO: 50 - protein, 670 aa, synthetic construct
  REGION 1..670: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTNVTGGYNVTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGYNVTYNVTGSGYNVTYNVTIGGSGG

SEQ ID NO: 51 - protein, 657 aa, synthetic construct
  REGION 1..657: note = Recombinant HIV-1 Env protein
AENLTVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGYNVTYNVTGSGYNVTIGGSGG

SEQ ID NO: 52 - protein, 661 aa, synthetic construct
  REGION 1..661: note = Recombinant HIV-1 Env protein
AENLTVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGYNVTYNVTGSGYNVTYNVTIGGSGG

SEQ ID NO: 53 - protein, 657 aa, synthetic construct
  REGION 1..657: note = Recombinant HIV-1 Env protein
ANNTWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGYNVTYNVTGSGYNVTIGGSGG

SEQ ID NO: 54 - protein, 661 aa, synthetic construct
  REGION 1..661: note = Recombinant HIV-1 Env protein
ANNTWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGYNVTYNVTGSGYNVTYNVTIGGSGG

SEQ ID NO: 55 - protein, 664 aa, synthetic construct
  REGION 1..664: note = Recombinant HIV-1 Env protein
NVTYNNTWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGYNVTYNVTGSGYNVTYNVTIGGSGG

SEQ ID NO: 56 - protein, 662 aa, synthetic construct
  REGION 1..662: note = Recombinant HIV-1 Env protein
NVTNLTVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGYNVTYNVTGSGYNVTYNVTIGGSGG

SEQ ID NO: 57 - protein, 637 aa, synthetic construct
  REGION 1..637: note = Recombinant HIV-1 Env protein
ANNTWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTNVTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLD

SEQ ID NO: 58 - protein, 638 aa, synthetic construct
  REGION 1..638: note = Recombinant HIV-1 Env protein
NVTNLTVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTNVTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLD

SEQ ID NO: 59 - protein, 640 aa, synthetic construct
  REGION 1..640: note = Recombinant HIV-1 Env protein
NVTYNNTWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTNVTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLD

SEQ ID NO: 60 - protein, 665 aa, synthetic construct
  REGION 1..665: note = Recombinant HIV-1 Env protein
NVTNLTVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTNVTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGYNVTYNVTGSGYNVTYNVTIGGSGG

SEQ ID NO: 61 - protein, 667 aa, synthetic construct
  REGION 1..667: note = Recombinant HIV-1 Env protein
NVTYNNTWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTNVTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGYNVTYNVTGSGYNVTYNVTIGGSGG

SEQ ID NO: 62 - protein, 673 aa, synthetic construct
  REGION 1..673: note = Recombinant HIV-1 Env protein
NVTYNNTWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTNVTGGYNVTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGYNVTYNVTGSGYNVTYNVTIGGSGG

SEQ ID NO: 63 - protein, 669 aa, synthetic construct
  REGION 1..669: note = Recombinant HIV-1 Env protein
NVTYNNTWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTNVTGGYNVTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGYNVTYNVTGSGYNVTIGGSGG

SEQ ID NO: 64 - protein, 662 aa, synthetic construct
  REGION 1..662: note = Recombinant HIV-1 Env protein
NVTYNNTWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTNVTGGYNVTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGYNVTYNVTIGGSGG

SEQ ID NO: 65 - protein, 654 aa, synthetic construct
  REGION 1..654: note = Recombinant HIV-1 Env protein
NVTNLTVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTNVTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGYNVTYNVTIGGSGG

SEQ ID NO: 66 - protein, 661 aa, synthetic construct
  REGION 1..661: note = Recombinant HIV-1 Env protein
NVTNLTVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTNVTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGYNVTYNVTGSGYNVTIGGSGG

SEQ ID NO: 67 - protein, 653 aa, synthetic construct
  REGION 1..653: note = Recombinant HIV-1 Env protein
AENLTVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTNVTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGYNVTYNVTIGGSGG

SEQ ID NO: 68 - protein, 653 aa, synthetic construct
  REGION 1..653: note = Recombinant HIV-1 Env protein
ANNTWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTNVTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGYNVTYNVTIGGSGG

SEQ ID NO: 69 - protein, 660 aa, synthetic construct
  REGION 1..660: note = Recombinant HIV-1 Env protein
ANNTWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTNVTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGYNVTYNVTGSGYNVTIGGSGG

SEQ ID NO: 70 - protein, 692 aa, synthetic construct
  REGION 1..692: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGGSSGVRLWATRQAMLGQVHEVPEGWLIFVAEQEELYVRVQNGFRKVQLEARTPLPR

SEQ ID NO: 71 - protein, 694 aa, synthetic construct
  REGION 1..694: note = Recombinant HIV-1 Env protein
NVTNNTWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGGSSGVRLWATRQAMLGQVHEVPEGWLIFVAEQEELYVRVQNGFRKVQLEARTPLPR

SEQ ID NO: 72 - protein, 695 aa, synthetic construct
  REGION 1..695: note = Recombinant HIV-1 Env protein
NVTYNNTWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGGSSGVRLWATRQAMLGQVHEVPEGWLIFVAEQEELYVRVQNGFRKVQLEARTPLPR

SEQ ID NO: 73 - protein, 698 aa, synthetic construct
  REGION 1..698: note = Recombinant HIV-1 Env protein
NVTYNNTWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTNVTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGGSSGVRLWATRQAMLGQVHEVPEGWLIFVAEQEELYVRVQNGFRKVQLEARTPLPR

SEQ ID NO: 74 - protein, 692 aa, synthetic construct
  REGION 1..692: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGNVTGVRLWATRQAMLGQVHEVPEGWLIFVAEQEELYVRVQNGFRKVQLEARTPLPR

SEQ ID NO: 75 - protein, 692 aa, synthetic construct
  REGION 1..692: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGGSSGVRLWATRQAMLGQVHNVTKGWLIFVAEQEELYVRVQNGFRKVQLEARTPLPR

SEQ ID NO: 76 - protein, 692 aa, synthetic construct
  REGION 1..692: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGGSSGVRLWNTTKIMLGQVHEVPEGWLIFVAEQEELYVRVQNGFRKVQLEARTPLPR

SEQ ID NO: 77 - protein, 692 aa, synthetic construct
  REGION 1..692: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGGSSGVRLWNTTKIMLGQVHNVTKGWLIFVAENETLYVRVQNGFRKVQLNVTTPLPR

SEQ ID NO: 78 - protein, 694 aa, synthetic construct
  REGION 1..694: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGSGGSSGVRLWATRQAMLGQVHEVPEGWLIFVAEQEELYVRVQNGFRKVQLEARTPLPR

SEQ ID NO: 79 - protein, 696 aa, synthetic construct
  REGION 1..696: note = Recombinant HIV-1 Env protein
NVTNNTWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGSGGSSGVRLWATRQAMLGQVHEVPEGWLIFVAEQEELYVRVQNGFRKVQLEARTPLPR

SEQ ID NO: 80 - protein, 697 aa, synthetic construct
  REGION 1..697: note = Recombinant HIV-1 Env protein
NVTYNNTWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGSGGSSGVRLWATRQAMLGQVHEVPEGWLIFVAEQEELYVRVQNGFRKVQLEARTPLPR

SEQ ID NO: 81 - protein, 700 aa, synthetic construct
  REGION 1..700: note = Recombinant HIV-1 Env protein
NVTYNNTWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTNVTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGSGGSSGVRLWATRQAMLGQVHEVPEGWLIFVAEQEELYVRVQNGFRKVQLEARTPLPR

SEQ ID NO: 82 - protein, 706 aa, synthetic construct
  REGION 1..706: note = Recombinant HIV-1 Env protein
NVTYNNTWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTNVTGGYNVTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGSGGSSGVRLWATRQAMLGQVHEVPEGWLIFVAEQEELYVRVQNGFRKVQLEARTPLPR

SEQ ID NO: 83 - protein, 694 aa, synthetic construct
  REGION 1..694: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGSGNVTGVRLWATRQAMLGQVHEVPEGWLIFVAEQEELYVRVQNGFRKVQLEARTPLPR

SEQ ID NO: 84 - protein, 694 aa, synthetic construct
  REGION 1..694: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGSGGSSGVRLWNTTKIMLGQVHNVTKGWLIFVAEQEELYVRVQNGFRKVQLEARTPLPR

SEQ ID NO: 85 - protein, 697 aa, synthetic construct
  REGION 1..697: note = Recombinant HIV-1 Env protein
NVTYNNTWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGSGGSSGVRLWNTTKIMLGQVHNVTKGWLIFVAEQEELYVRVQNGFRKVQLEARTPLPR

SEQ ID NO: 86 - protein, 694 aa, synthetic construct
  REGION 1..694: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGSGGSSGVRLWNTTKIMLGQVHNVTKGWLIFVAENETLYVRVQNGFRKVQLNVTTPLPR

SEQ ID NO: 87 - protein, 694 aa, synthetic construct
  REGION 1..694: note = Recombinant HIV-1 Env protein
SSGVRLWATRQAMLGQVHEVPEGWLIFVAEQEELYVRVQNGFRKVQLEARTPLPRGSGGGAENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLD

SEQ ID NO: 88 - protein, 710 aa, synthetic construct
  REGION 1..710: note = Recombinant HIV-1 Env protein
SSGVRLWATRQAMLGQVHEVPEGWLIFVAEQEELYVRVQNGFRKVQLEARTPLPRGSGGGAENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLDGGYNVTYNVTIGGSGG

SEQ ID NO: 89 - protein, 697 aa, synthetic construct
  REGION 1..697: note = Recombinant HIV-1 Env protein
SSGVRLWATRQAMLGQVHEVPEGWLIFVAEQEELYVRVQNGFRKVQLEARTPLPRGSGGGNVTYNNTWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLD

SEQ ID NO: 90 - protein, 685 aa, synthetic construct
  REGION 1..685: note = Recombinant HIV-1 Env protein
SSGVRLWATRQAMLGQVHEVPEGWLIFVAEQEELYVRVQNGFRKVQLEGSGAENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLD

SEQ ID NO: 91 - protein, 688 aa, synthetic construct
  REGION 1..688: note = Recombinant HIV-1 Env protein
SSGVRLWATRQAMLGQVHEVPEGWLIFVAEQEELYVRVQNGFRKVQLEGSGNVTYNNTWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLD

SEQ ID NO: 92 - protein, 687 aa, synthetic construct
  REGION 1..687: note = Recombinant HIV-1 Env protein
SSGVRLWATRQAMLGQVHEVPEGWLIFVAEQEELYVRVQNGFRKVQLEGSGGGAENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLD

SEQ ID NO: 93 - protein, 690 aa, synthetic construct
  REGION 1..690: note = Recombinant HIV-1 Env protein
SSGVRLWATRQAMLGQVHEVPEGWLIFVAEQEELYVRVQNGFRKVQLEGSGGGNVTYNNTWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLD

SEQ ID NO: 94 - protein, 691 aa, synthetic construct
  REGION 1..691: note = Recombinant HIV-1 Env protein
NVTGVRLWNTTKIMLGQVHNVTKGWLIFVAENETLYVRVQNGFRKVQLEGSGGGNVTYNNTWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLD

SEQ ID NO: 95 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINETDNLTLD

SEQ ID NO: 96 - protein, 630 aa, synthetic construct
  REGION 1..630: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINETDG

SEQ ID NO: 97 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFNQTINEQDNLTLD

SEQ ID NO: 98 - protein, 627 aa, synthetic construct
  REGION 1..627: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFNQTINE

SEQ ID NO: 99 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEENQTQQEINEQDNLTLD

SEQ ID NO: 100 - protein, 627 aa, synthetic construct
  REGION 1..627: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEENQTQQEINE

SEQ ID NO: 101 - protein, 623 aa, synthetic construct
  REGION 1..623: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEENQTQQ

SEQ ID NO: 102 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGNLTESQFQQEINEQDNLTLD

SEQ ID NO: 103 - protein, 627 aa, synthetic construct
  REGION 1..627: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGNLTESQFQQEINE

SEQ ID NO: 104 - protein, 623 aa, synthetic construct
  REGION 1..623: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGNLTESQFQQ

SEQ ID NO: 105 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTTTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLD

SEQ ID NO: 106 - protein, 623 aa, synthetic construct
  REGION 1..623: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTTTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQ

SEQ ID NO: 107 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLNVTARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLD

SEQ ID NO: 108 - protein, 623 aa, synthetic construct
  REGION 1..623: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLNVTARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQ

SEQ ID NO: 109 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQANQTLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLD

SEQ ID NO: 110 - protein, 623 aa, synthetic construct
  REGION 1..623: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQANQTLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQ

SEQ ID NO: 111 - protein, 627 aa, synthetic construct
  REGION 1..627: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTTTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFNQTINE

SEQ ID NO: 112 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQACPKVSFEPIPIHYCAPAGFAILKCKDKKFNGTGPCKNVSTVQCTHGIKPVVSTQLLLNGSLAEEEVMIRSENITNNAKNIIVQFNTPVQINCTRPNNMTRKSIRIGPGQAFYALGDIIGDIRQPHCTVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKNRTVGRRRRRRAVGIGAVFLGFLGAAGSTMGAASNTLTVQARQLLSGIVQQQSNLLRAPEAQQHLLKLGVWGIKQLQARVLAVERYLEVQQLLGIWGCSGKLICCTNVPWNSSWSNRNLSEIWDNMTWLQWDKEIGNYTQIIYGLLEESQFQQEINEQDLLALD

SEQ ID NO: 113 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQACPKVSFEPIPIHYCAPAGFAILKCKDKKFNGTGPCKNVSTVQCTHGIKPVVSTQLLLNGSLAEEEVMIRSENITNNAKNIIVQFNTPVQINCTRPNNMTRKSIRIGPGQAFYALGDIIGDIRQPHCTVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKNRTVGRRRRRRAVGINATFLGFLGAAGSTMGAASNTLTVQARQLLSGIVQQQSNLLRAPEAQQHLLKLGVWGIKQLQARVLAVERYLEVQQLLGIWGCSGKLICCTNVPWNSSWSNRNLSEIWDNMTWLQWDKEIGNYTQIIYGLLEESNFTQEINEQDLLALD

SEQ ID NO: 114 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQACPKVSFEPIPIHYCAPAGFAILKCKDKKFNGTGPCKNVSTVQCTHGIKPVVSTQLLLNGSLAEEEVMIRSENITNNAKNIIVQFNTPVQINCTRPNNMTRKSIRIGPGQAFYALGDIIGDIRQPHCTVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKRNVTGRRRRRRAVGIGAVFLGFLGAAGSTMGAASNTLTVQARQLLSGIVQQQSNLLRAPEAQQHLLKLGVWGIKQLQARVLAVERYLEVQQLLGIWGCSGKLICCTNVPWNSSWSNRNLSEIWDNMTWLQWDKEIGNYTQIIYGLLEESQFQQEINEQDLLALD

SEQ ID NO: 115 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQACPKVSFEPIPIHYCAPAGFAILKCKDKKFNGTGPCKNVSTVQCTHGIKPVVSTQLLLNGSLAEEEVMIRSENITNNAKNIIVQFNTPVQINCTRPNNMTRKSIRIGPGQAFYALGDIIGDIRQPHCTVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKRNVTGRRRRRRAVGINATFLGFLGAAGSTMGAASNTLTVQARQLLSGIVQQQSNLLRAPEAQQHLLKLGVWGIKQLQARVLAVERYLEVQQLLGIWGCSGKLICCTNVPWNSSWSNRNLSEIWDNMTWLQWDKEIGNYTQIIYGLLEESNFTQEINEQDLLALD

SEQ ID NO: 116 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQACPKVSFEPIPIHYCAPAGFAILKCKDKKFNGTGPCKNVSTVQCTHGIKPVVSTQLLLNGSLAEEEVMIRSENITNNAKNIIVQFNTPVQINCTRPNNMTRKSIRIGPGQAFYALGDIIGDIRQPHCTVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKRRVVGRRRRRRAVGIGAVFLGFLGAAGSTMGAASNTLTVQARQLLSGIVQQQSNLLRAPEAQQHLLKLGVWGIKQLQARVLAVERYLEVQQLLGIWGCSGKLICCTNVPWNSSWSNRNLSEIWDNMTWLQWDKEIGNYTQIIYGLLEESQFQQEINEQDLNATD

SEQ ID NO: 117 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQACPKVSFEPIPIHYCAPAGFAILKCKDKKFNGTGPCKNVSTVQCTHGIKPVVSTQLLLNGSLAEEEVMIRSENITNNAKNIIVQFNTPVQINCTRPNNMTRKSIRIGPGQAFYALGDIIGDIRQPHCTVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKRRVVGRRRRRRAVGINATFLGFLGAAGSTMGAASNTLTVQARQLLSGIVQQQSNLLRAPEAQQHLLKLGVWGIKQLQARVLAVERYLEVQQLLGIWGCSGKLICCTNVPWNSSWSNRNLSEIWDNMTWLQWDKEIGNYTQIIYGLLEESNFTQEINEQDLNATD

SEQ ID NO: 118 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQACPKVSFEPIPIHYCAPAGFAILKCKDKKFNGTGPCKNVSTVQCTHGIKPVVSTQLLLNGSLAEEEVMIRSENITNNAKNIIVQFNTPVQINCTRPNNMTRKSIRIGPGQAFYALGDIIGDIRQPHCTVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKNRTVGRRRRRRAVGIGAVFLGFLGAAGSTMGAASNTLTVQARQLLSGIVQQQSNLLRAPEAQQHLLKLGVWGIKQLQARVLAVERYLEVQQLLGIWGCSGKLICCTNVPWNSSWSNRNLSEIWDNMTWLQWDKEIGNYTQIIYGLLEESQFQQEINEQDLLNLT

SEQ ID NO: 119 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQACPKVSFEPIPIHYCAPAGFAILKCKDKKFNGTGPCKNVSTVQCTHGIKPVVSTQLLLNGSLAEEEVMIRSENITNNAKNIIVQFNTPVQINCTRPNNMTRKSIRIGPGQAFYALGDIIGDIRQPHCTVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKNRTVGRRRRRRAVGINATFLGFLGAAGSTMGAASNTLTVQARQLLSGIVQQQSNLLRAPEAQQHLLKLGVWGIKQLQARVLAVERYLEVQQLLGIWGCSGKLICCTNVPWNSSWSNRNLSEIWDNMTWLQWDKEIGNYTQIIYGLLEESNFTQEINEQDLLNLT

SEQ ID NO: 120 - protein, 637 aa, synthetic construct
  REGION 1..637: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQACPKVSFEPIPIHYCAPAGFAILKCKDKKFNGTGPCKNVSTVQCTHGIKPVVSTQLLLNGSLAEEEVMIRSENITNNAKNIIVQFNTPVQINCTRPNNMTRKSIRIGPGQAFYALGDIIGDIRQPHCTVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKNRTVGRRRRRRAVGIGAVFLGFLGAAGSTMGAASNTLTVQARQLLSGIVQQQSNLLRAPEAQQHLLKLGVWGIKQLQARVLAVERYLEVQQLLGIWGCSGKLICCTNVPWNSSWSNRNLSEIWDNMTWLQWDKEIGNYTQIIYGLLEESQFQQEINEQDLLNLTALD

SEQ ID NO: 121 - protein, 637 aa, synthetic construct
  REGION 1..637: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQACPKVSFEPIPIHYCAPAGFAILKCKDKKFNGTGPCKNVSTVQCTHGIKPVVSTQLLLNGSLAEEEVMIRSENITNNAKNIIVQFNTPVQINCTRPNNMTRKSIRIGPGQAFYALGDIIGDIRQPHCTVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKNRTVGRRRRRRAVGINATFLGFLGAAGSTMGAASNTLTVQARQLLSGIVQQQSNLLRAPEAQQHLLKLGVWGIKQLQARVLAVERYLEVQQLLGIWGCSGKLICCTNVPWNSSWSNRNLSEIWDNMTWLQWDKEIGNYTQIIYGLLEESNFTQEINEQDLLNLTALD

SEQ ID NO: 122 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQACPKVSFEPIPIHYCAPAGFAILKCKDKKFNGTGPCKNVSTVQCTHGIKPVVSTQLLLNGSLAEEEVMIRSENITNNAKNIIVQFNTPVQINCTRPNNMTRKSIRIGPGQAFYALGDIIGDIRQPHCTVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGINATFLGFLGAAGSTMGAASNTLTVQARQLLSGIVQQQSNLLRAPEAQQHLLKLGVWGIKQLQARVLAVERYLEVQQLLGIWGCSGKLICCTNVPWNSSWSNRNLSEIWDNMTWLQWDKEIGNYTQIIYGLLEESNFTQEINEQDNLTLD

SEQ ID NO: 123 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQACPKVSFEPIPIHYCAPAGFAILKCKDKKFNGTGPCKNVSTVQCTHGIKPVVSTQLLLNGSLAEEEVMIRSENITNNAKNIIVQFNTPVQINCTRPNNMTRKSIRIGPGQAFYALGDIIGDIRQPHCTVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGINATFLGFLGAAGSTMGAASNTTTVQARQLLSGIVQQQSNLLRAPEAQQHLLKLGVWGIKQLQARVLAVERYLEVQQLLGIWGCSGKLICCTNVPWNSSWSNRNLSEIWDNMTWLQWDKEIGNYTQIIYGLLEESNFTQEINEQDNLTLD

SEQ ID NO: 124 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQACPKVSFEPIPIHYCAPAGFAILKCKDKKFNGTGPCKNVSTVQCTHGIKPVVSTQLLLNGSLAEEEVMIRSENITNNAKNIIVQFNTPVQINCTRPNNMTRKSIRIGPGQAFYALGDIIGDIRQPHCTVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGINATFLGFLGAAGSTMGAASNTTTVQARQLLSGIVQQQSNLLRAPEAQQHLLKLGVWGIKQLQARVLAVERYLEVQQLLGIWGCSGKLICCTNVPWNSSWSNRNLSEIWDNMTWLQWDKEIGNYTQIIYGLLEESQFQQEINEQDNLTLD

SEQ ID NO: 125 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQACPKVSFEPIPIHYCAPAGFAILKCKDKKFNGTGPCKNVSTVQCTHGIKPVVSTQLLLNGSLAEEEVMIRSENITNNAKNIIVQFNTPVQINCTRPNNMTRKSIRIGPGQAFYALGDIIGDIRQPHCTVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTTTVQARQLLSGIVQQQSNLLRAPEAQQHLLKLGVWGIKQLQARVLAVERYLEVQQLLGIWGCSGKLICCTNVPWNSSWSNRNLSEIWDNMTWLQWDKEIGNYTQIIYGLLEESNFTQEINEQDNLTLD

SEQ ID NO: 126 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKNVSTVQCTHGIKPVVSTQLLLNGSLAEEEVMIRSENITNNAKNIIVQFNTPVQINCTRPNNMTRKSIRIGPGQAFYALGDIIGDIRQPHCTVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKNRTVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDLLALD

SEQ ID NO: 127 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKNVSTVQCTHGIKPVVSTQLLLNGSLAEEEVMIRSENITNNAKNIIVQFNTPVQINCTRPNNMTRKSIRIGPGQAFYALGDIIGDIRQPHCTVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKNRTVGRRRRRRAVGINATFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESNFTQEINEQDLLALD

SEQ ID NO: 128 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKNVSTVQCTHGIKPVVSTQLLLNGSLAEEEVMIRSENITNNAKNIIVQFNTPVQINCTRPNNMTRKSIRIGPGQAFYALGDIIGDIRQPHCTVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKRNVTGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDLLALD

SEQ ID NO: 129 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKNVSTVQCTHGIKPVVSTQLLLNGSLAEEEVMIRSENITNNAKNIIVQFNTPVQINCTRPNNMTRKSIRIGPGQAFYALGDIIGDIRQPHCTVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKRNVTGRRRRRRAVGINATFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESNFTQEINEQDLLALD

SEQ ID NO: 130 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKNVSTVQCTHGIKPVVSTQLLLNGSLAEEEVMIRSENITNNAKNIIVQFNTPVQINCTRPNNMTRKSIRIGPGQAFYALGDIIGDIRQPHCTVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKRRVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDLNATD

SEQ ID NO: 131 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKNVSTVQCTHGIKPVVSTQLLLNGSLAEEEVMIRSENITNNAKNIIVQFNTPVQINCTRPNNMTRKSIRIGPGQAFYALGDIIGDIRQPHCTVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKRNVTGRRRRRRAVGINATFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESNFTQEINEQDLNATD

SEQ ID NO: 132 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKNVSTVQCTHGIKPVVSTQLLLNGSLAEEEVMIRSENITNNAKNIIVQFNTPVQINCTRPNNMTRKSIRIGPGQAFYALGDIIGDIRQPHCTVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKNRTVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDLLNLT

SEQ ID NO: 133 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKNVSTVQCTHGIKPVVSTQLLLNGSLAEEEVMIRSENITNNAKNIIVQFNTPVQINCTRPNNMTRKSIRIGPGQAFYALGDIIGDIRQPHCTVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKNRTVGRRRRRRAVGINATFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESNFTQEINEQDLLNLT

SEQ ID NO: 134 - protein, 637 aa, synthetic construct
  REGION 1..637: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKNVSTVQCTHGIKPVVSTQLLLNGSLAEEEVMIRSENITNNAKNIIVQFNTPVQINCTRPNNMTRKSIRIGPGQAFYALGDIIGDIRQPHCTVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKNRTVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDLLNLTALD

SEQ ID NO: 135 - protein, 637 aa, synthetic construct
  REGION 1..637: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKNVSTVQCTHGIKPVVSTQLLLNGSLAEEEVMIRSENITNNAKNIIVQFNTPVQINCTRPNNMTRKSIRIGPGQAFYALGDIIGDIRQPHCTVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKNRTVGRRRRRRAVGINATFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESNFTQEINEQDLLNLTALD

SEQ ID NO: 136 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKNRTVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDLLALD

SEQ ID NO: 137 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKNRTVGRRRRRRAVGINATFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESNFTQEINEQDLLALD

SEQ ID NO: 138 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKRNVTGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDLLALD

SEQ ID NO: 139 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKRNVTGRRRRRRAVGINATFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESNFTQEINEQDLLALD

SEQ ID NO: 140 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKRRVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDLNATD

SEQ ID NO: 141 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKRRVVGRRRRRRAVGINATFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESNFTQEINEQDLNATD

SEQ ID NO: 142 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKNRTVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDLLNLT

SEQ ID NO: 143 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKNRTVGRRRRRRAVGINATFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESNFTQEINEQDLLNLT

SEQ ID NO: 144 - protein, 637 aa, synthetic construct
  REGION 1..637: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKNRTVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDLLNLTALD

SEQ ID NO: 145 - protein, 637 aa, synthetic construct
  REGION 1..637: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCKNRTVGRRRRRRAVGINATFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESNFTQEINEQDLLNLTALD

SEQ ID NO: 146 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGINATFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESNFTQEINEQDNLTLD

SEQ ID NO: 147 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGINATFLGFLGAAGSTMGAASNTTTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESNFTQEINEQDNLTLD

SEQ ID NO: 148 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGIGAVFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESNFTQEINEQDNLTLD

SEQ ID NO: 149 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGINATFLGFLGAAGSTMGAASNTLTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLD

SEQ ID NO: 150 - protein, 634 aa, synthetic construct
  REGION 1..634: note = Recombinant HIV-1 Env protein
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQICPKVSFEPIPIHYCAPAGFAILKCKDKKFNGTGPCKSVSTVQCTHGIKPVVSTQLLLNGSLAEEEVMIRSENITNNAKNIIVQFNTPVQINCTRPNNMTRKSIRIGPGQAFYALGDIIGDIRQPHCNVSKATWNETLGKVVKQLRKHFGNNTIIFFANSSGGDLEVTTHSFNCGGEFFYCNTSGLFNSTWISNTSVQGSNSTGSNDSITLPCRIKQIINMWQRVGQCMYAPPIQGVIRCVSNITGLILTRDGGSTNSTTETFRPGGGDMRDNWRSELYKYKVVKIEPLGVAPTRCNRTVVGRRRRRRAVGINATFLGFLGAAGSTMGAASNTTTVQARQLLSGIVQQQSNLLRAPEAQQHLLKLGVWGFKQLQARVLAVERYLEVQQLLGIWGCSGKLICCTNVPWNSSWSNRNLSEIWDNMTWLQWDKEIGNYTQIIYGLLEESQFQQEINEQDNLTLD

SEQ ID NO: 151 - protein, 10 aa, synthetic construct
  REGION 1..10: note = Glycosylation sequence
AENLWVTYYY

SEQ ID NO: 152 - protein, 16 aa, synthetic construct
  REGION 1..16: note = Glycosylation sequence
GPGNSTAENLWVTYYY

SEQ ID NO: 153 - protein, 19 aa, synthetic construct
  REGION 1..19: note = Glycosylation sequence
GVAPTRCKRRVVGRRRRRR

SEQ ID NO: 154 - protein, 30 aa, synthetic construct
  REGION 1..30: note = Glycosylation sequence
GVAPTRCKRRVVGNSTHKQLTHHMRRRRRR

SEQ ID NO: 155 - protein, 30 aa, synthetic construct
  REGION 1..30: note = Glycosylation sequencev
GVAPTRCNRTVVGNSTHKQLTHHMRRRRRR

SEQ ID NO: 156 - protein, 30 aa, synthetic construct
  REGION 1..30: note = Glycosylation sequence
GVAPTRCNRTVVGNSTHKNLTHHMRRRRRR

SEQ ID NO: 157 - protein, 9 aa, synthetic construct
  REGION 1..9: note = Glycosylation sequence
NEQDLLALD

SEQ ID NO: 158 - protein, 12 aa, synthetic construct
  REGION 1..12: note = Glycosylation sequence
NEQDNLTLPNST

SEQ ID NO: 159 - protein, 10 aa, synthetic construct
  REGION 1..10: note = Glycosylation sequence
GPGNSTAENL

SEQ ID NO: 160 - protein, 26 aa, synthetic construct
  REGION 1..26: note = Glycosylation sequence
APTRCNRTVVGVSTHKNLTHHMRRRR

SEQ ID NO: 161 - protein, 10 aa, synthetic construct
  REGION 1..10: note = Glycosylation sequence
QDNLTPLNST

(19) United States
(12) Patent Application Publication
(10) Pub. No.: US 2025/0197455 A1
(43) Pub. Date: Jun. 19, 2025

(54) BASE-COVERED HIV-1 ENVELOPE ECTODOMAINS AND THEIR USE

(71) Applicant: TThe United States of America, as represented by the Secretary, Department of Health and Human Servi, Bethesda, MD (US)

(72) Inventors: Peter Kwong, Washington, DC (US); Tongqing Zhou, Boyds, MD (US); Adam Olia, Silver Spring, MD (US); Reda Rawi, North Bethesda, MD (US); Anita Shah, Fairfax, VA (US); Darcy Harris, Bethesda, MD (US); Ridhi Chaudhary, Bethesda, MD (US); Cheng Cheng, Bethesda, MD (US); Yongping Yang, Potomac, MD (US)

(73) Assignee: TThe United States of America, as represented by the Secretary, Department of Health and Human Servi, Bethesda, MD (US)

(21) Appl. No.: 18/852,276

(22) PCT Filed: Mar. 27, 2023
(86) PCT No.: PCT/US2023/065009

§ 371 Date: Sep. 27, 2024

Related U.S. Application Data
(60) Provisional application No. 63/324,121, filed on Mar. 27, 2022.

(51) Int. Cl. C07K 14/005; A61P 31/18

(52) U.S. Cl. CPC C07K 14/005; A61P 31/18; C12N 2740/16122; C12N 2740/16134

(57) ABSTRACT

Immunogens comprising a soluble HIV-1 Env ectodomain trimer stabilized in a prefusion closed conformation and comprising modifications to introduce N-linked glycan sequons at the membrane-proximal base of the trimer, as well as methods of their use and production are disclosed. In several implementations, the immunogen can be used to elicit an immune response to HIV-1 in a subject.

### CROSS REFERENCE TO RELATED APPLICATIONS

This application claims priority to U.S. Provisional Application No. 63/324,121, filed Mar. 27, 2022, which is incorporated by reference in its entirety.

### FIELD

This disclosure relates to soluble Human immunodeficiency virus type 1 (HIV-1) Envelope (Env) ectodomain trimers for treatment and inhibition of HIV-1 infection and disease.

### BACKGROUND

Millions of people are infected with HIV-1 worldwide, and 2.5 to 3 million new infections have been estimated to occur yearly. Although effective antiretroviral therapies are available, over a million succumb to AIDS every year, especially in sub-Saharan Africa, underscoring the need to develop measures to prevent the spread of this disease.

An enveloped virus, HIV-1 hides from humoral recognition behind a wide array of protective mechanisms. The major envelope protein of HIV-1 is a glycoprotein of approximately 160 kD (gp160). During infection, proteases of the host cell cleave gp160 into gp120 and gp41. Gp41 is an integral membrane protein, while gp120 protrudes from the mature virus. Together gp120 and gp41 make up the HIV-1 Env spike, which is a target for neutralizing antibodies.

It is believed that immunization with an effective immunogen based on the HIV-1 Env glycoprotein can elicit a neutralizing response, which may be protective against HIV-1 infection. However, despite extensive effort, a need remains for agents capable of such action.

### SUMMARY

This disclosure provides novel soluble HIV-1 Env ectodomain trimers that include one or more amino acid substitutions that “lock” the soluble HIV-1 Env trimer in a prefusion closed conformation with a reduced binding affinity for CD4 relative to non-modified trimers, but that retain binding affinity for broadly neutralizing HIV-1 antibodies, and which further comprise modifications to reduce the immunodominance of the membrane-proximal “base” of the trimer. The base of the trimer is exposed in soluble forms of the HIV-1 Env ectodomain trimer but not in membrane-anchored forms of the trimer. N-linked glycan sequons are introduced into the HIV-1 Env ectodomain sequence to cover the base of the soluble trimer. The disclosed soluble HIV-1 Env ectodomain trimers can be used to elicit a neutralizing immune response to HIV-1 in a subject.

The soluble HIV-1 envelope (Env) ectodomain trimer comprises protomers comprising or consisting of HIV-1 Env positions 31 to one of 653-664 that are modified with amino acid substitutions, additions, and/or insertions. The modifications include amino acid substitutions for stabilization of the trimer in a prefusion closed conformation, comprising: (i) cysteine substitutions at HIV-1 Env positions 501 and 605 that form a non-natural intra-protomer disulfide bond; (ii) cysteine substitutions at HIV-1 Env positions 201 and 433 that form a non-natural intra-protomer disulfide bond; (iii) a proline substitution at HIV-1 Env position 559; and (iv) methionine, leucine, and proline substitutions at HIV-1 Env positions 302, 320, and 329, respectively. The modifications also include amino acid substitutions, additions, and/or insertions to introduce N-linked glycan sequons as follows: (i) optionally an addition of three amino acids immediately N-terminal to HIV-1 Env position 31 to introduce an N-linked glycan sequon; (ii) amino acid substitutions and/or insertions to introduce one, two, or three N-linked glycan sequons between HIV-1 Env positions 502 and 509, wherein no more than 15 amino acids are inserted; and (iii) amino acid substitutions between HIV-1 Env positions 650-664 to introduce one or two N-linked glycan sequons, and/or addition of up to 60 amino acids at the C-terminus of the protomers to introduce one to five N-linked glycan sequons. Additionally, the protomers of the soluble HIV-1 Env ectodomain trimer optionally comprise a substitution of RRRRRR (SEQ ID NO: 21) for the amino acids of a gp120/gp41 furin cleavage site. The HIV-1 Env positions are according to HXB2 numbering. The soluble HIV-1 Env ectodomain trimer elicits an immune response to HIV-1.

In some implementations, the protomers of the trimer comprise or consist of an amino acid sequence set forth as any one of SEQ ID NOs: 2-10.

Nucleic acid molecules encoding the disclosed soluble HIV-1 Env ectodomain trimers are also provided. In some implementations, the nucleic acid molecule can encode a precursor protein of a gp120-gp41 protomer of a disclosed soluble HIV-1 Env trimer. Expression vectors (such as an inactivated or attenuated viral vector) including the nucleic acid molecules are also provided.

Immunogenic compositions including one or more of the disclosed soluble HIV-1 Env ectodomain trimers are also provided. The composition may be contained in a unit dosage form. The composition can further include an adjuvant.

Methods of eliciting an immune response to HIV-1 envelope protein in a subject are disclosed, as are methods of treating, inhibiting or preventing an HIV-1 infection in a subject. In such methods a subject, such as a human subject, is administered an effective amount of a disclosed soluble HIV-1 Env ectodomain trimer to elicit the immune response. The subject can be, for example, a human subject at risk of or having an HIV-1 infection.

The foregoing and other features and advantages of this disclosure will become more apparent from the following detailed description of several implementations which proceeds with reference to the accompanying figures.

### BRIEF DESCRIPTION OF THE FIGURES

FIGS. 1A-1D. Design and selection of glycan base-covered Env trimers. (A) BG505 DS-SOSIP Env trimer model highlighting sequence segments that comprise the exposed protein base. (B) Close-up view and sequence information showing location of N-linked glycans introduced in each sequence segment. Italicized numbers denote sequence insertions, and “BG505” refers to protein-base BG505 trimer. (C) Matrix of 16 Env variants and antigenic criteria used for selection.

The first half of the table describes the glycan sequons that were added to each construct with + or − notation. The second half of the table summarizes the antibody binding of each construct, denoted with Yes or No binding, or in light grey with the number of base binding antibodies blocked from binding. The N28-502-660-665, N28-JCBv3-660, N28-JCBv2-660-665, and N28-JCBv3-660-665 constructs were selected for further characterization. (D) Glycan-base BG505 trimer, modeled with six-introduced N-linked glycans. Sequences shown are: AENLWVTYYY (SEQ ID NO: 151), GPGNSTAENLWVTYYY (SEQ ID NO: 152), GVAPTRCKRRVVGRRRRRR (SEQ ID NO: 153), GVAPTRCKRRVVGNSTHKQLTHHMRRRRRR (SEQ ID NO: 154), GVAPTRCNRTVVGNSTHKQLTHHMRRRRRR (SEQ ID NO: 155), GVAPTRCNRTVVGNSTHKNLTHHMRRRRRR(SEQ ID NO: 156), NEQDLLALD (SEQ ID NO: 157), NEQDNLTLPNST (SEQ ID NO: 158)

FIGS. 2A-2F. Physical properties of glycan base-covered Env trimers. (A) SDS-PAGE of affinity purified glycan-base constructs from BG505, left side, and ConC, right side. (C) Table showing the number of additional protein N-linked glycosylation (PNG) sites for each construct along with the percentage of furin cleavage, as determined by densitometric of the Coomassie gel in panel (A). The BG505-N28-JCBv3-660-665 and ConcC-N28-JCBv3-660-665 constructs were selected as the top candidates to move on for further characterization. (D) Size exclusion chromatography profiles of the two selected constructs. (E) Differential scanning calorimetry scans of the two constructs. (F) Negative stain EM 2D class averages of BG505 and ConC glycan-base constructs.

FIGS. 3A-3D. Glycosylation and antigenic profiles of BG505 and ConC base-covered trimers. (A) Mass spectrometry glycosylation analysis of protein-base and glycan-base constructs of BG505 and ConC (B) Surface plasmon resonance analysis of soluble CD4 binding to glycan-base BG505 and ConC, as compared to protein-base BG505. (C-D) MSD antibody binding analysis of glycan-base BG505 and ConC as compared to their protein-base counterparts, all stabilized by DS-SOSIP-2G-3Mut-Rns7Mut.

FIGS. 4A-4C. CryoEM of ConC Glycan-Base Trimer. (A) CryoEM 2D class averages for ConC glycan-base construct. The classification ConC showed a nearly fully trimeric appearance. (B) CryoEM reconstruction of the ConC glycan-base trimer at 4.1 Å resolution. (C) Representation of RMSD of glycan-base ConC from protein-base ConC (PDB ID: 6CK9). Increasing RMSD are shown, and residues that could not be resolved in the glycan-base trimer reconstruction have been modeled.

FIGS. 5A-5F. Trimer immunogenicity in mice reveals reciprocally symmetric responses. (A) Immunization scheme and groups are shown for mice immunized with protein-base or glycan-base variants of BG505 and ConC Env trimers. (B) Anti-trimer ELISA responses at week 8 for BG505 trimer-immunized groups and (C) ConC trimer-immunized groups. (D) Immunization schema and groups are shown for mice immunized with protein-base or glycan-base trimers using FP-primed, sequential or FP+trimer cocktail-primed vaccine regimens (E) Longitudinal trimer-specific serum immunogenicity is shown for each group, as assessed by ELISA against protein-base BG505 trimer (top) or glycan-base BG505 trimer (bottom). Immunization timepoints are indicated by vertical dotted lines. (F) Percent neutralization against BG505.N611Q virus is shown after the final immunization using a 1:50 serum dilution for FP-primed, sequential groups (left) and cocktail-primed groups (right). Mean±SD are shown.

FIGS. 6A-6D. Glycan-base BG505 trimer elicit responses capable of dissembling trimer. (A) Immunization scheme and groups are shown for guinea pigs immunized with protein-base or glycan-base BG505 trimer variants. (B) Anti-trimer ELISA responses at weeks 0, 2 and 6 are shown for each group against protein-base BG505 Env (left) and glycan-base BG505 Env (right) (C) Week 6 ELISA endpoint titers using a starting dilution of 1:1000 and cross-reactivity (%) of responses are listed for each animal (left) and group comparison shown for week 6 titers against each trimer (right) (D) Electron microscopy of polyclonal epitope mapping (EMPEM) of antibodies elicited by immunization. Fabs were generate from whole plasma IgG and incubated with indicated HIV-1 Env. Negative stain EM were carried out using Env-Fab complexes purified by size exclusion chromatography, 2D averages were shown to the left of each panel. Representative 3D reconstructions were shown to the right with HIV-1 Env or Env fragments, and Fabs.

FIGS. 7A-7D. ConC trimer immunogenicity in guinea pigs reveals reciprocally symmetric responses. (A) Immunization scheme and groups shown for guinea pigs immunized with protein-base or glycan-base ConC Env trimers (B) Anti-trimer ELISA responses at weeks 0, 2 and 6 are shown for each group against protein-base ConC Env (left) and glycan-base ConC Env (right) (C) Week 6 ELISA endpoint titers using a starting dilution of 1:1000 and cross-reactivity (%) of responses are listed for each animal (left) and group comparison shown for week 6 titers against each ConC trimer (right) (D) Electron microscopy of polyclonal epitope mapping (EMPEM) of antibodies elicited by immunization. Fabs were generate from whole plasma IgG and incubated with indicated HIV-1 Env. Negative stain EM were carried out using Env-Fab complexes purified by size exclusion chromatography, 2D averages were shown to the left of each panel. Representative 3D reconstructions were shown to the right.

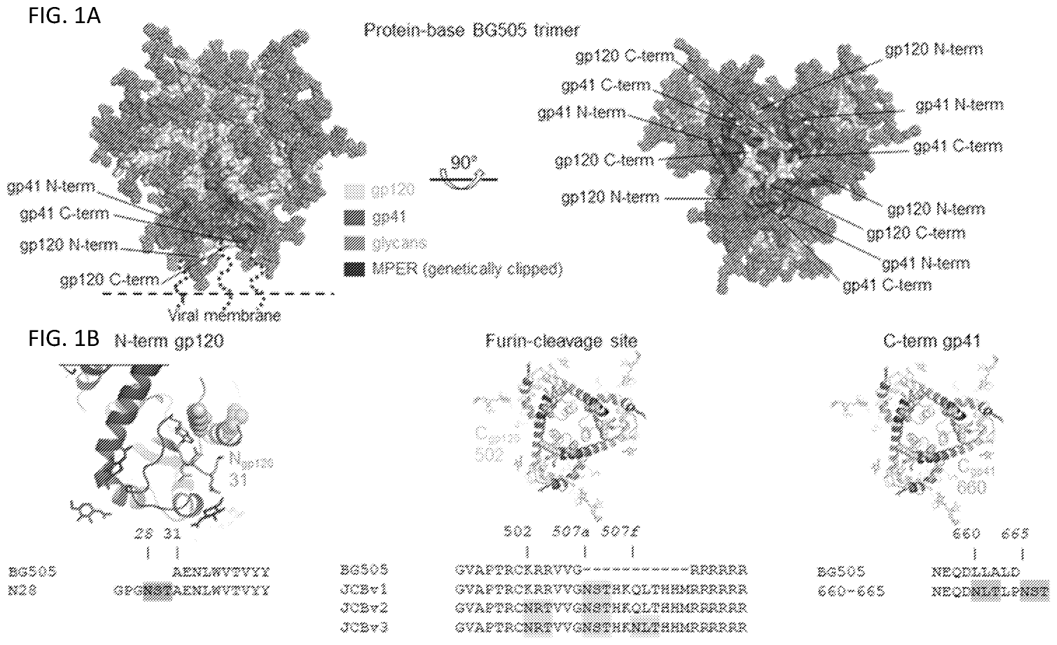

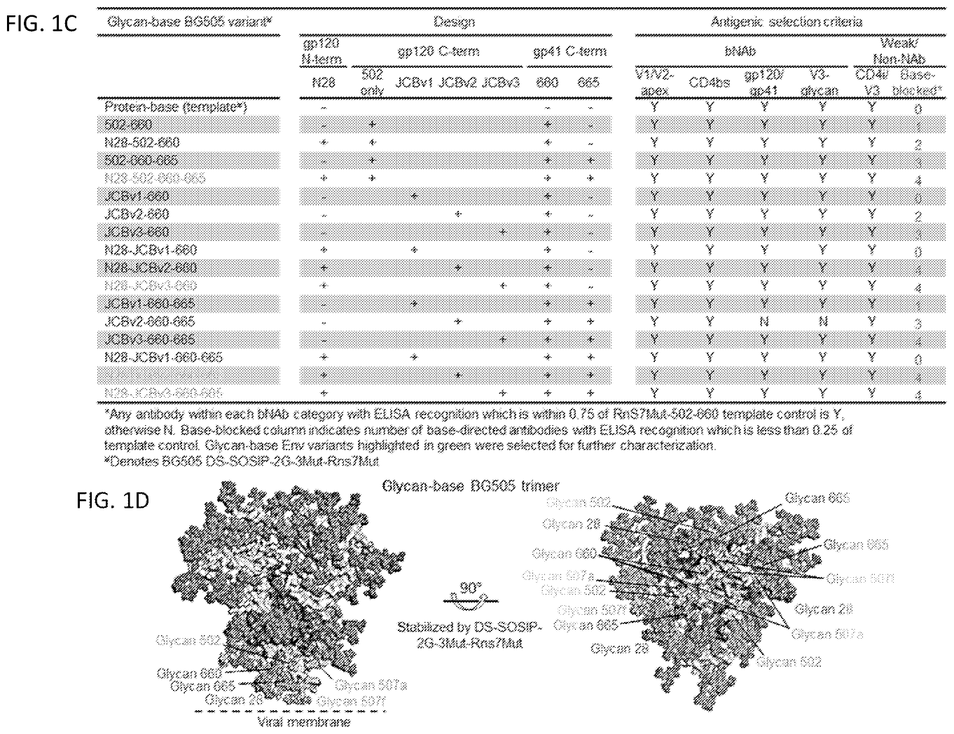

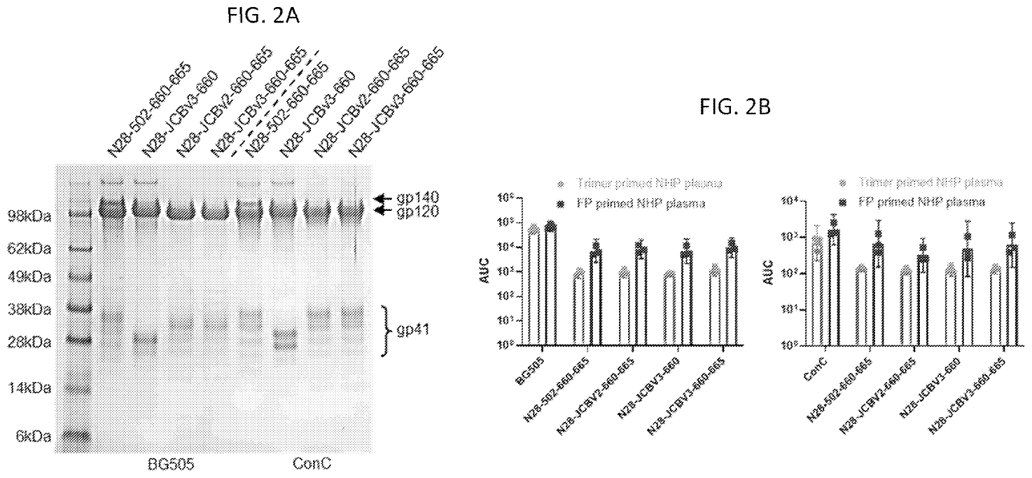

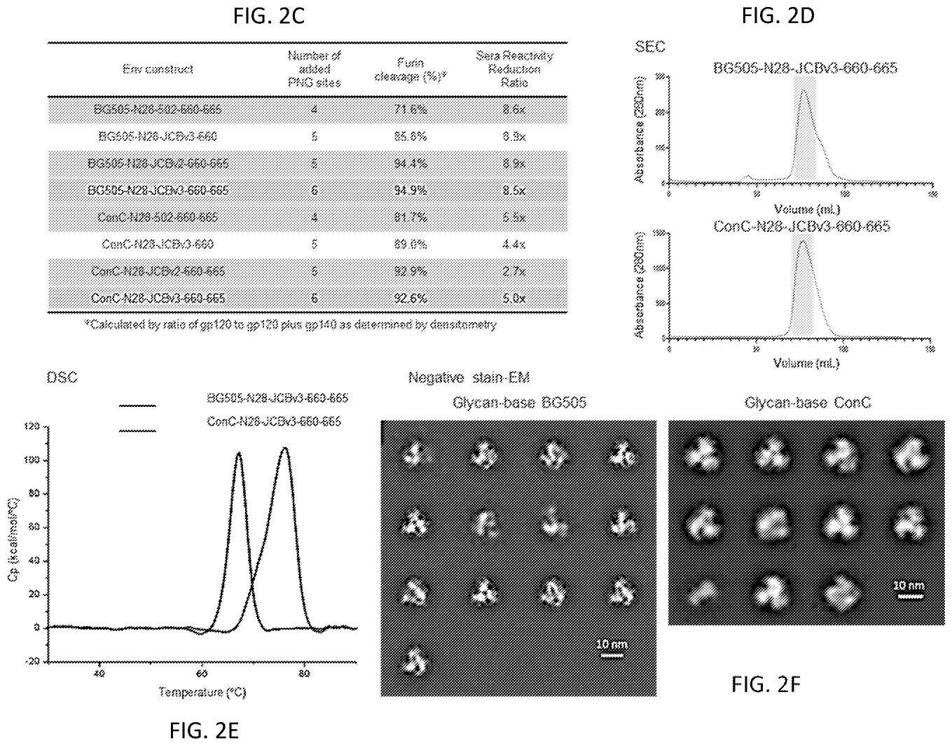

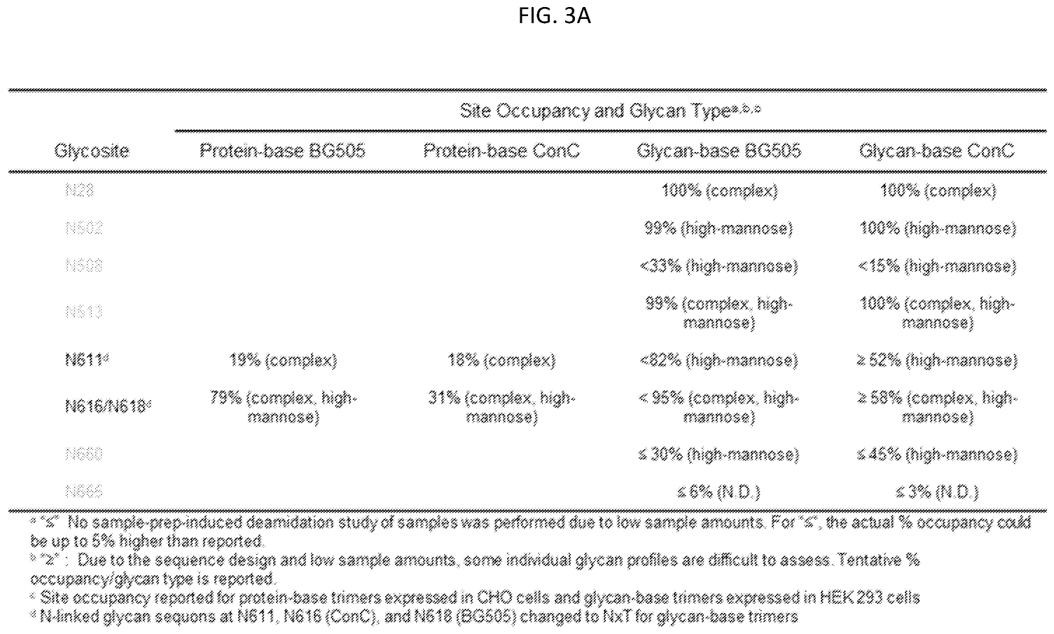

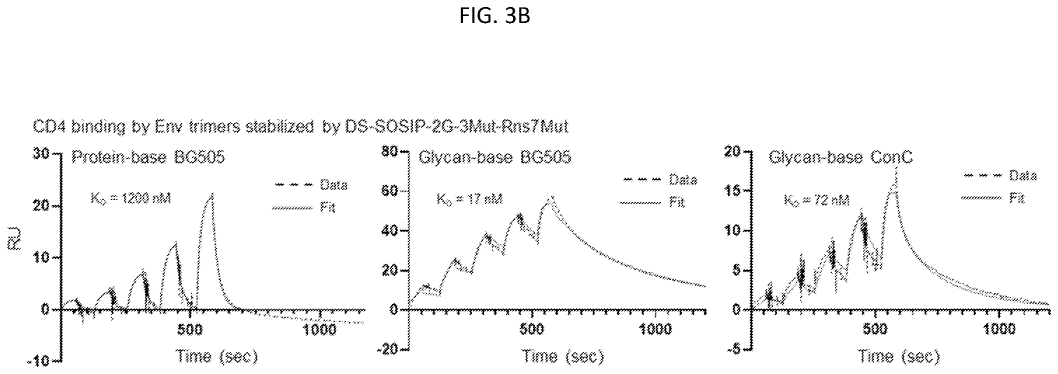

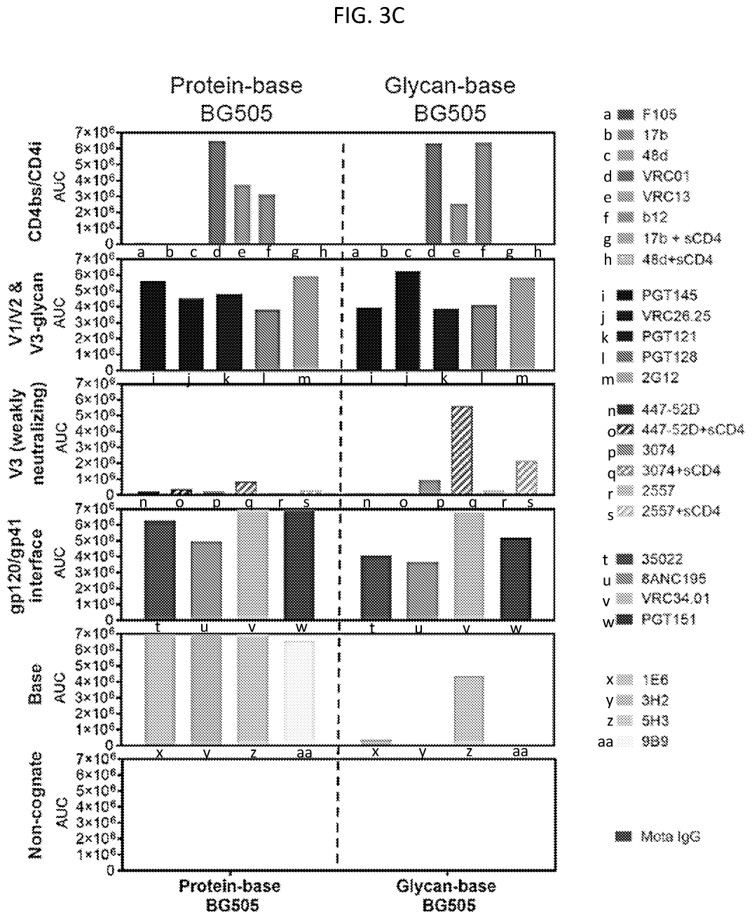

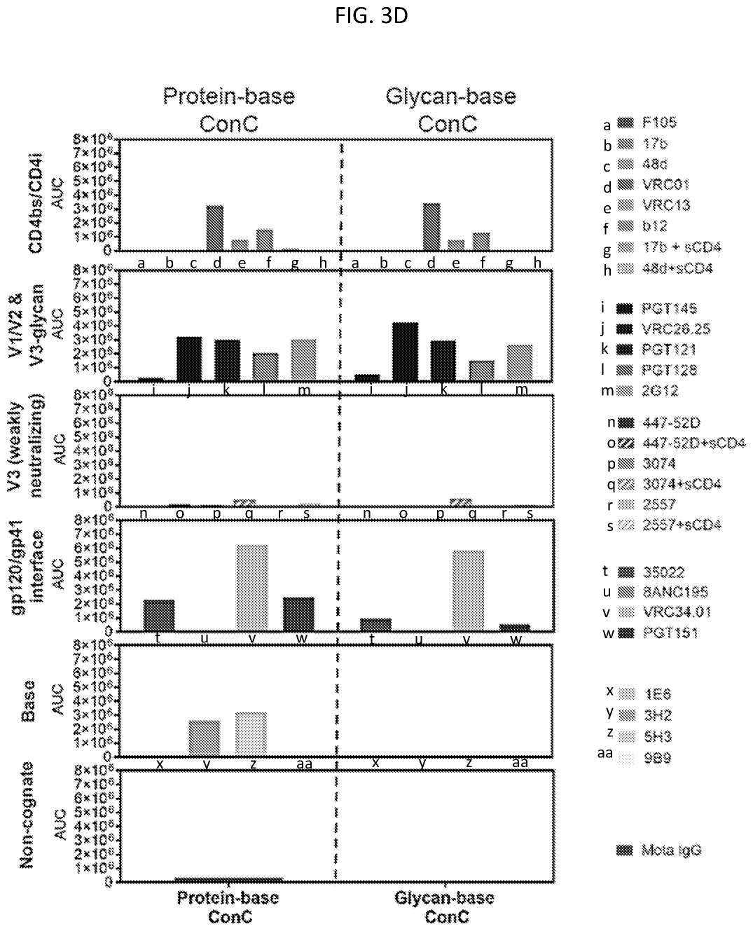

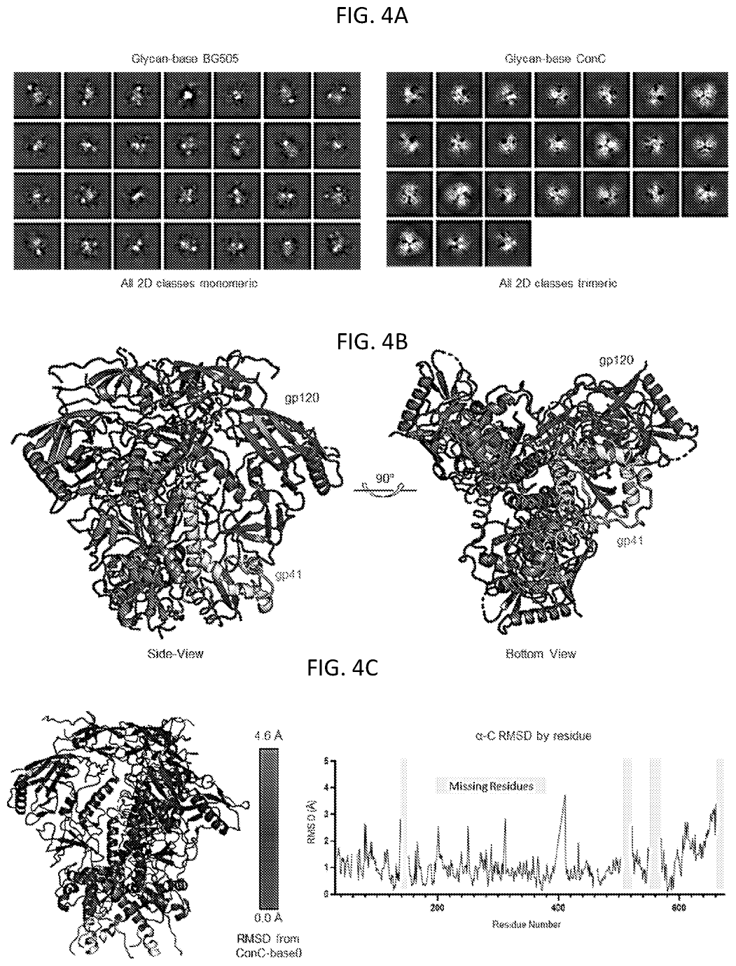

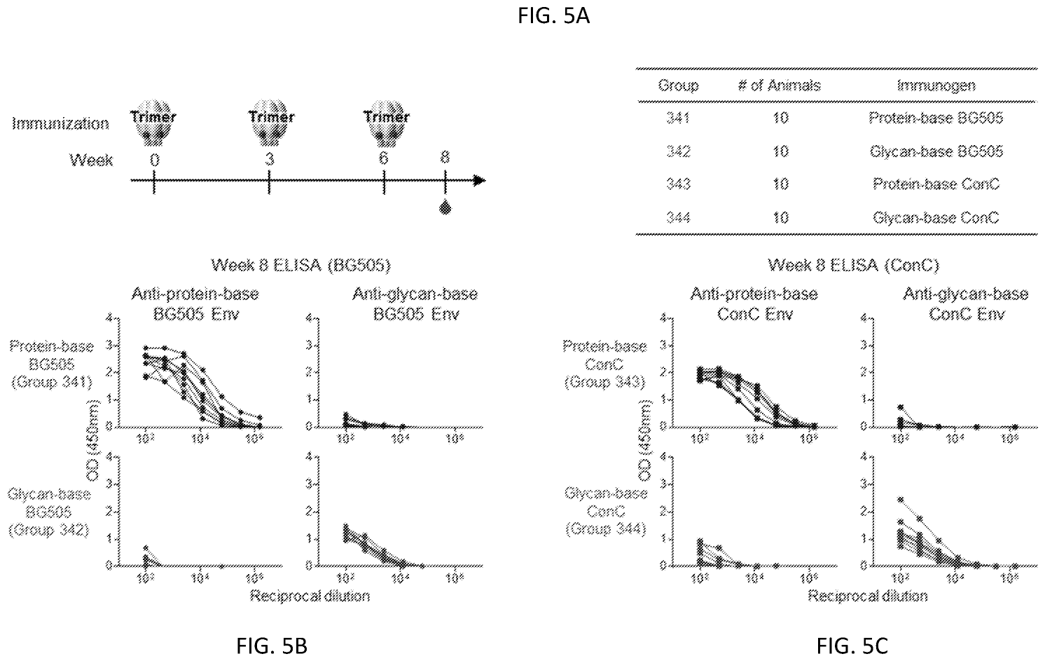

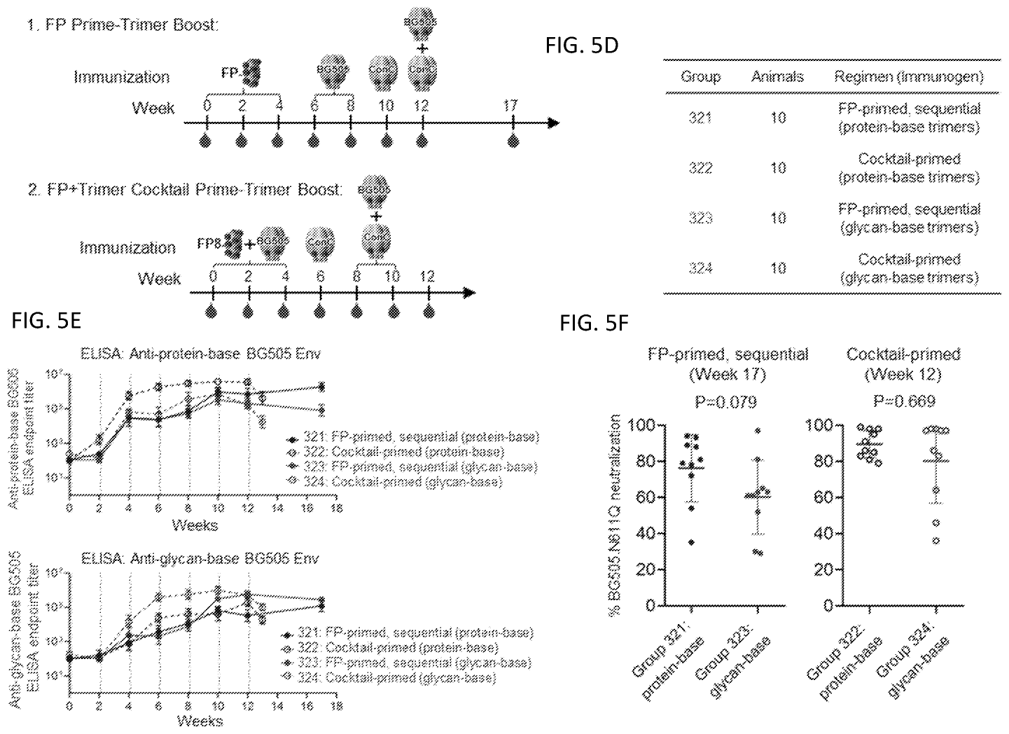

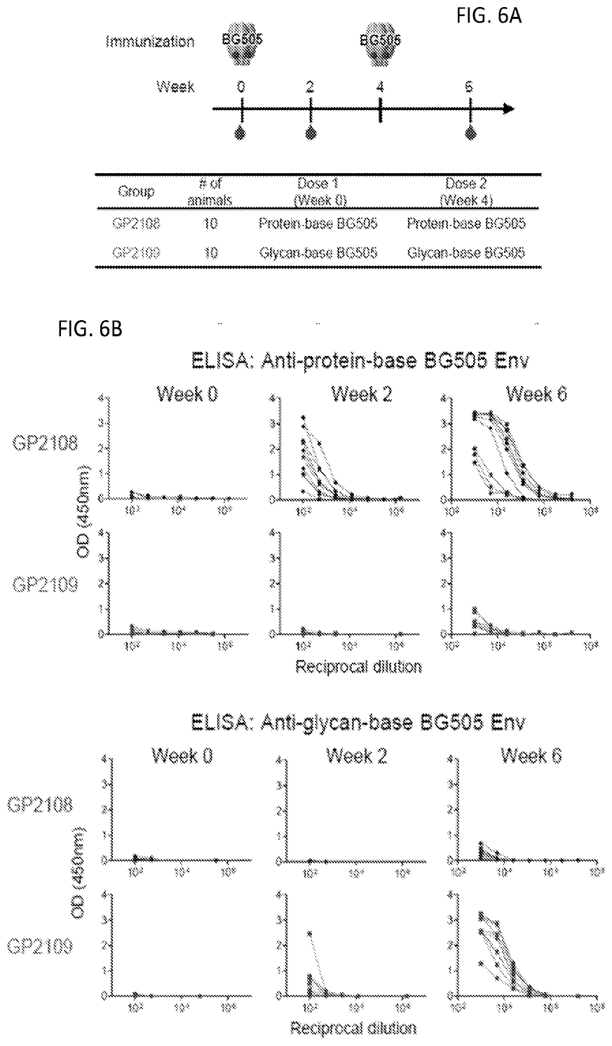

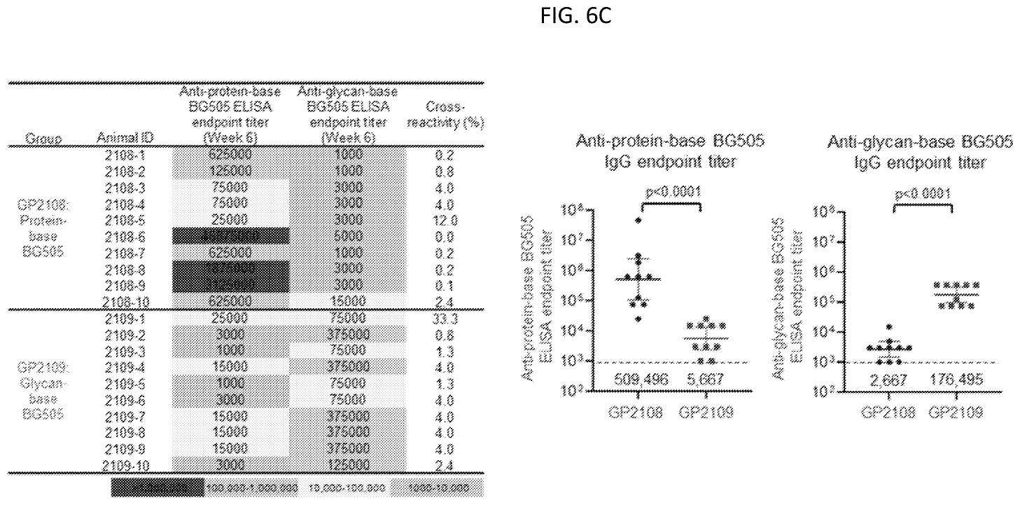

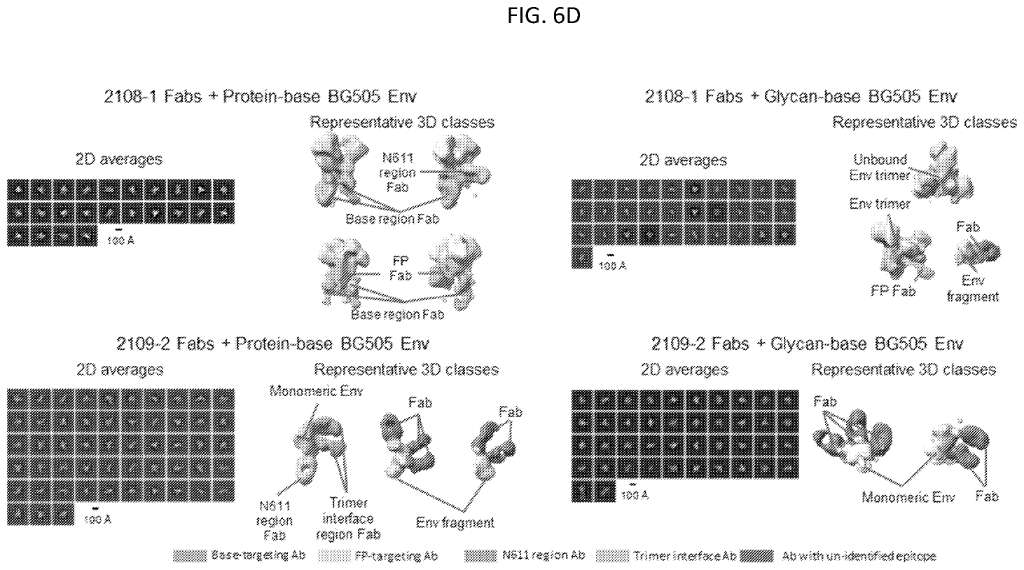

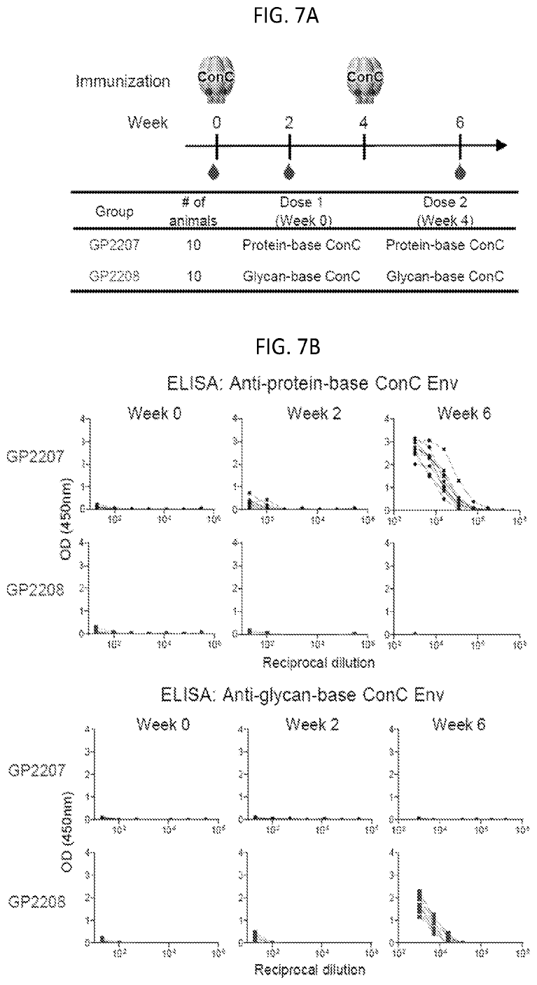

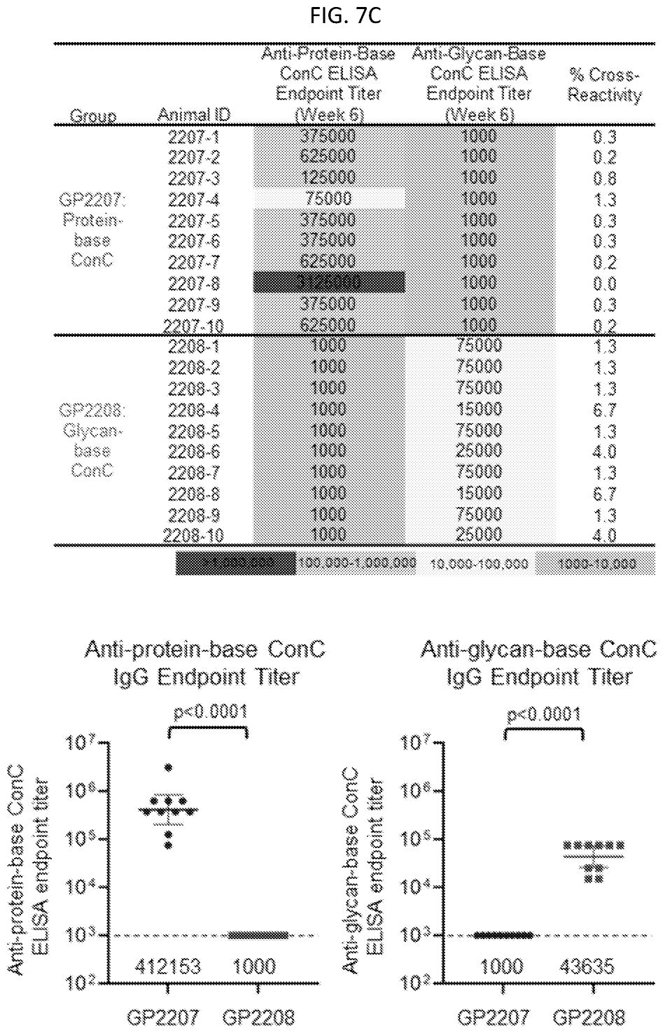

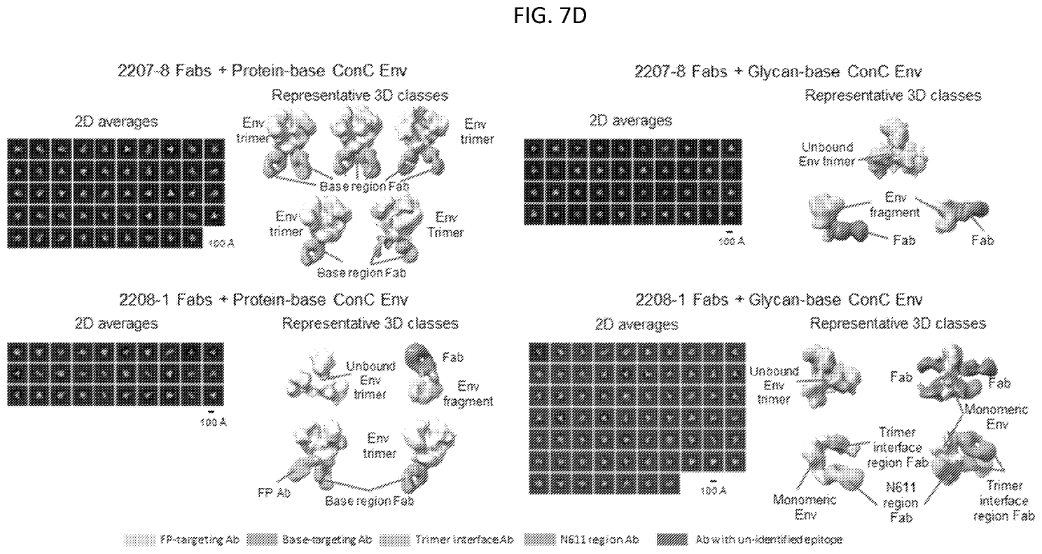

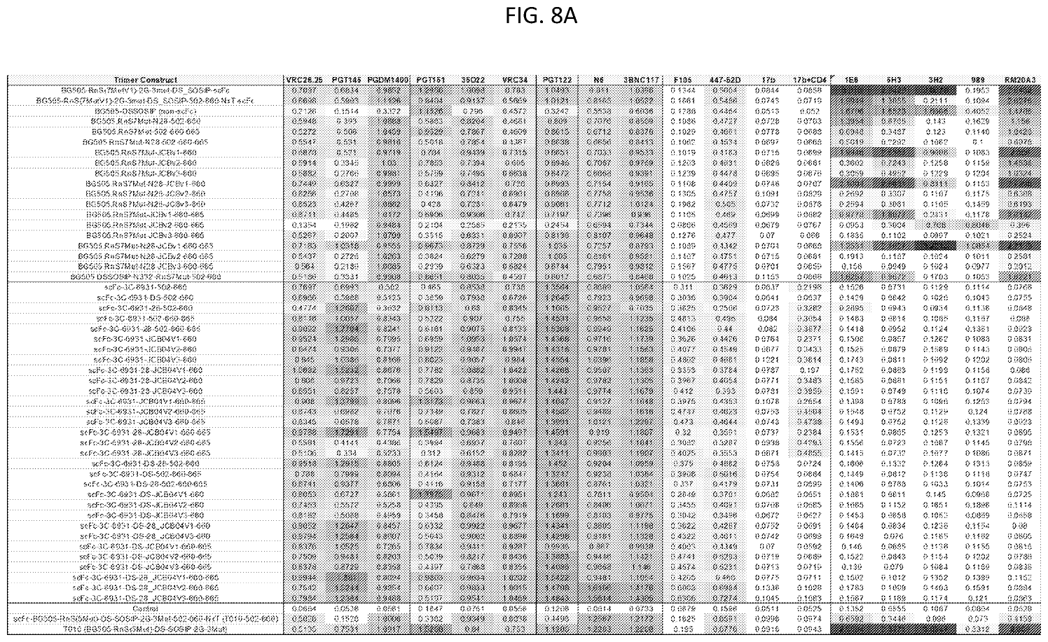

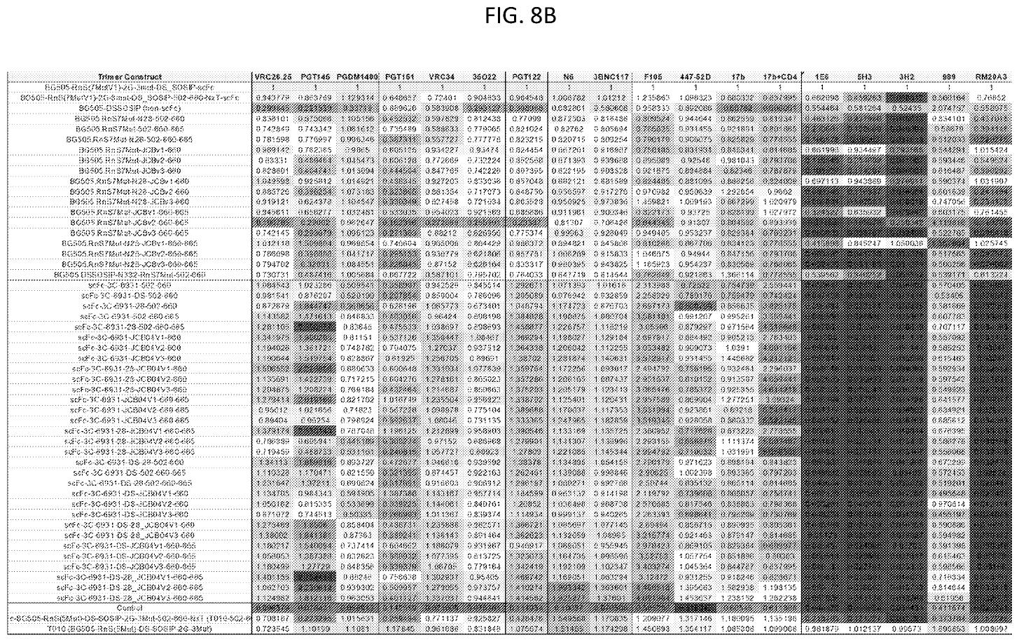

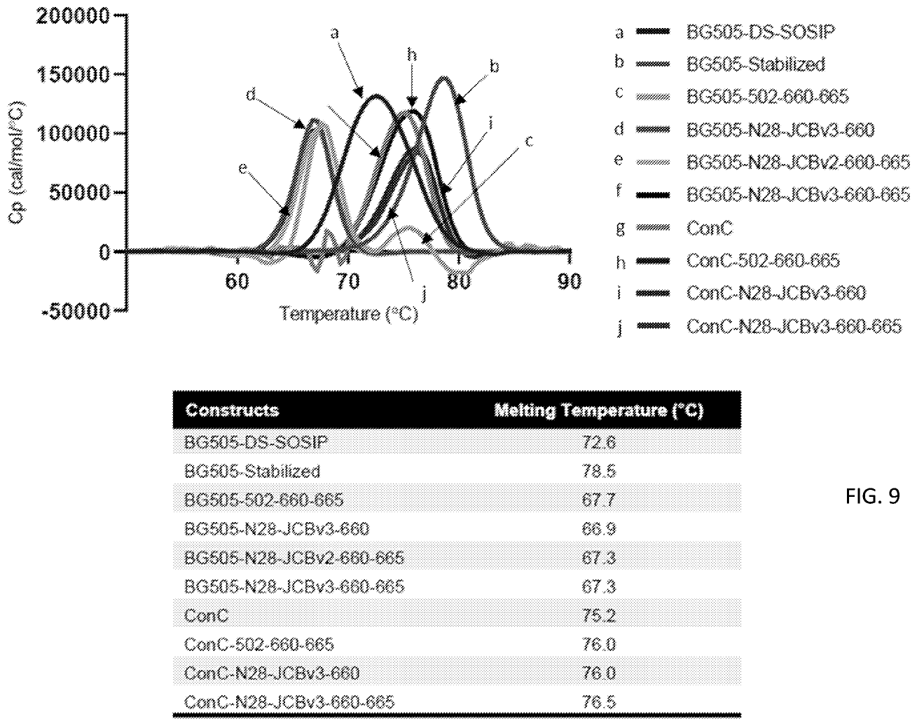

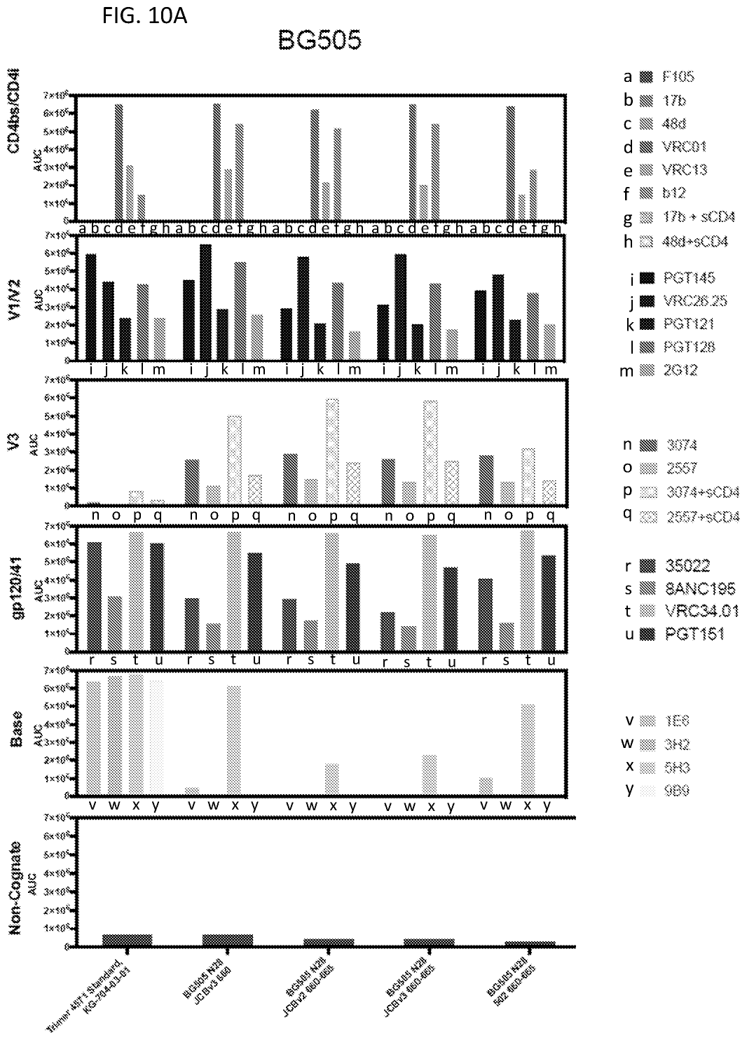

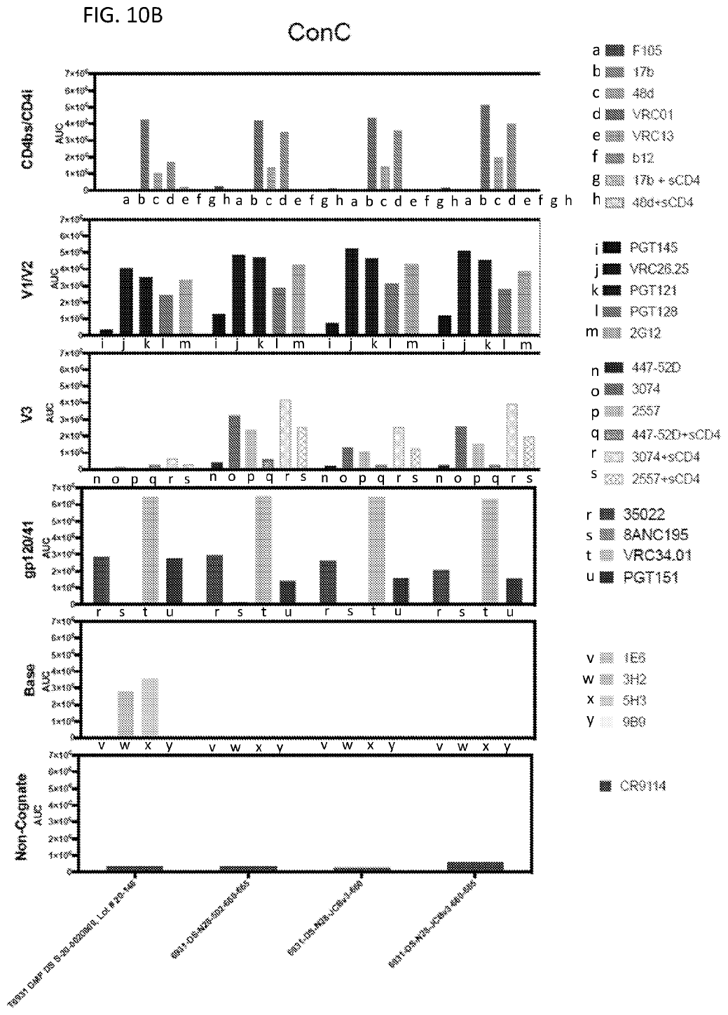

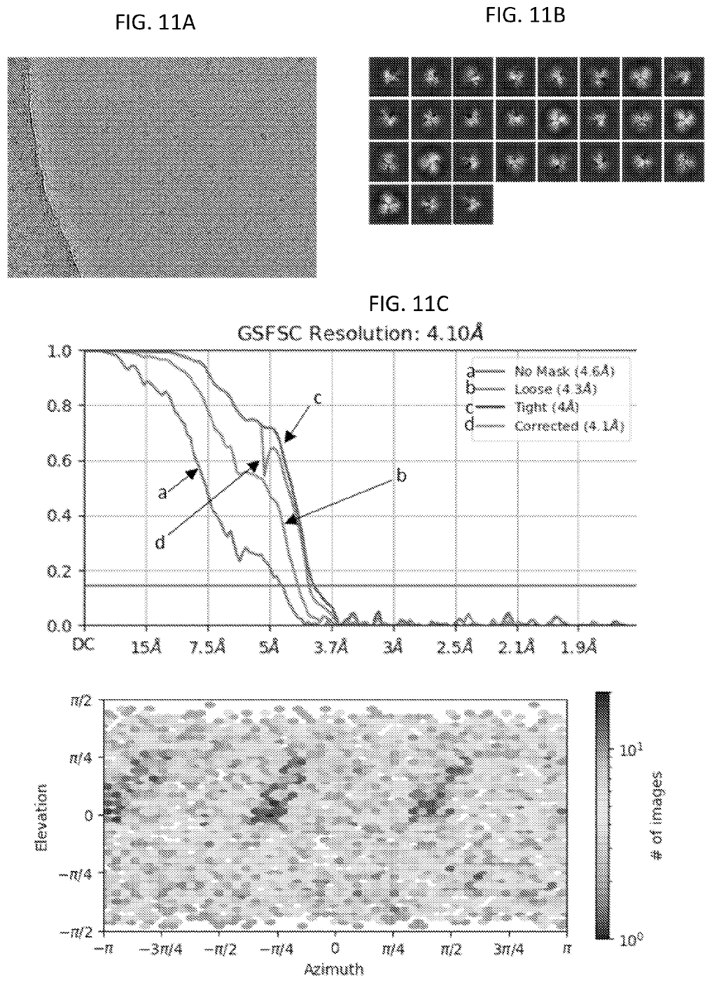

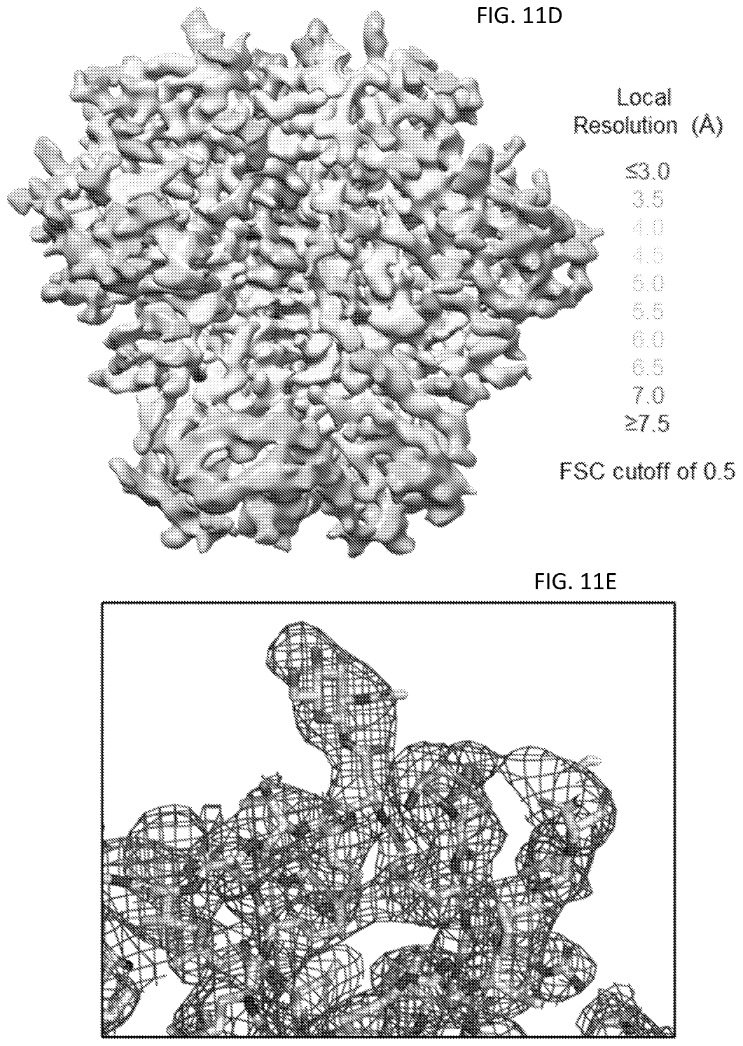

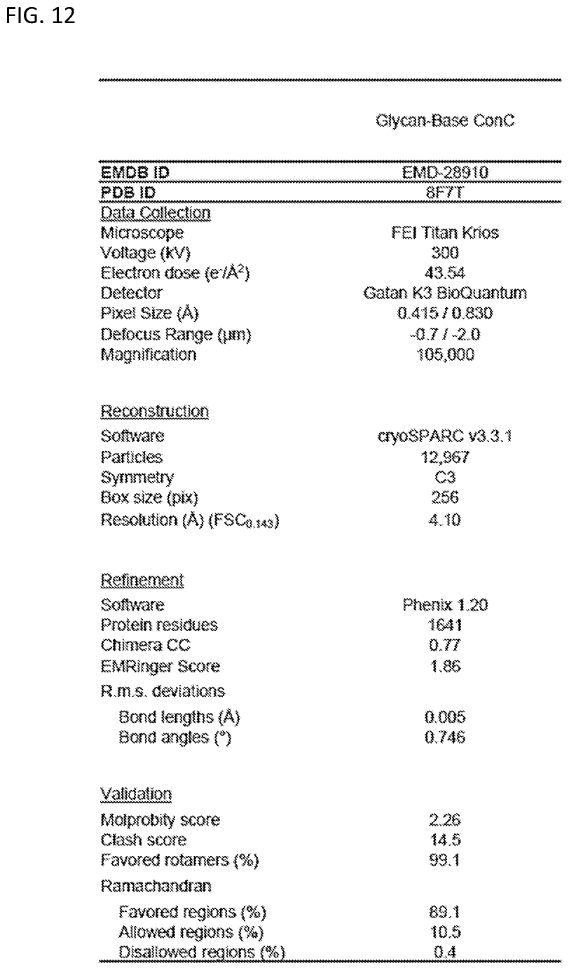

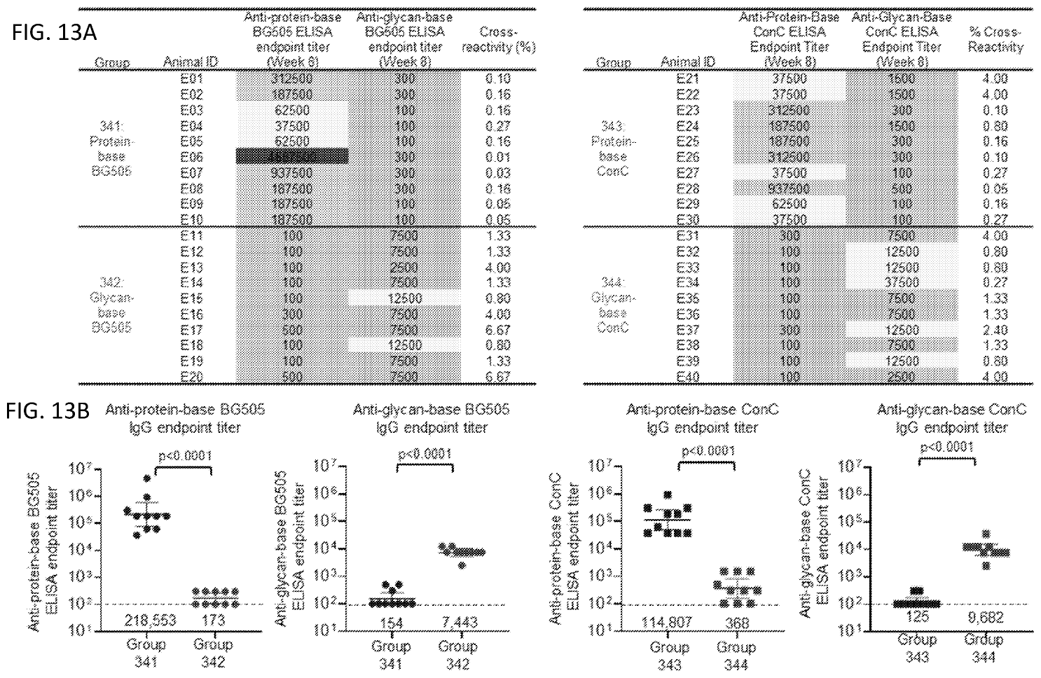

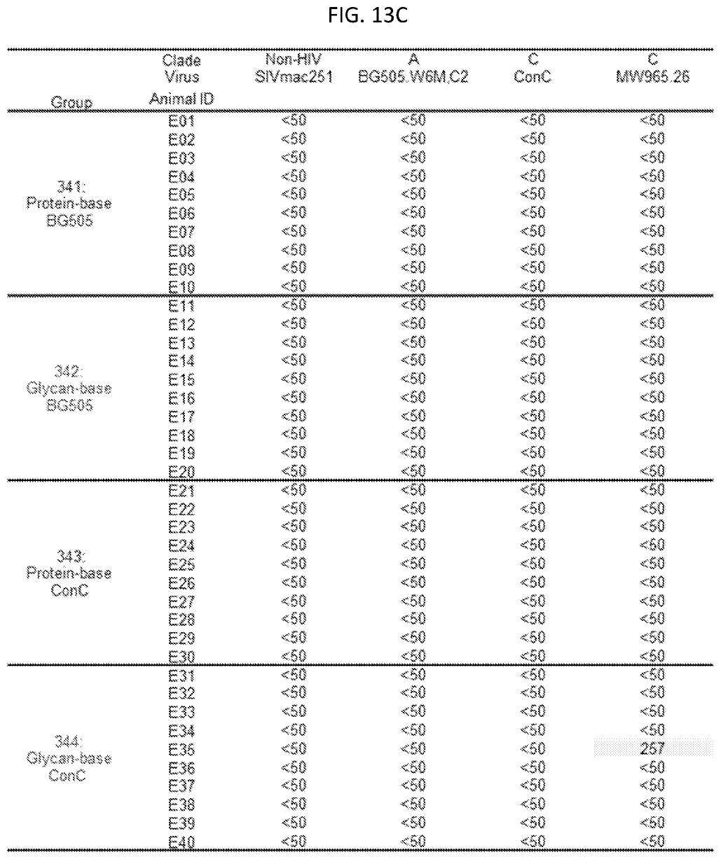

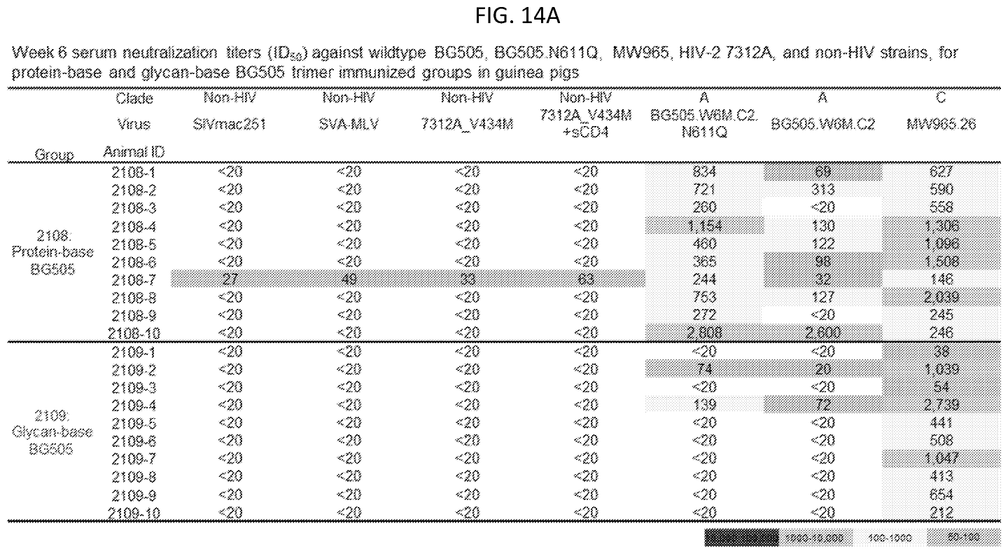

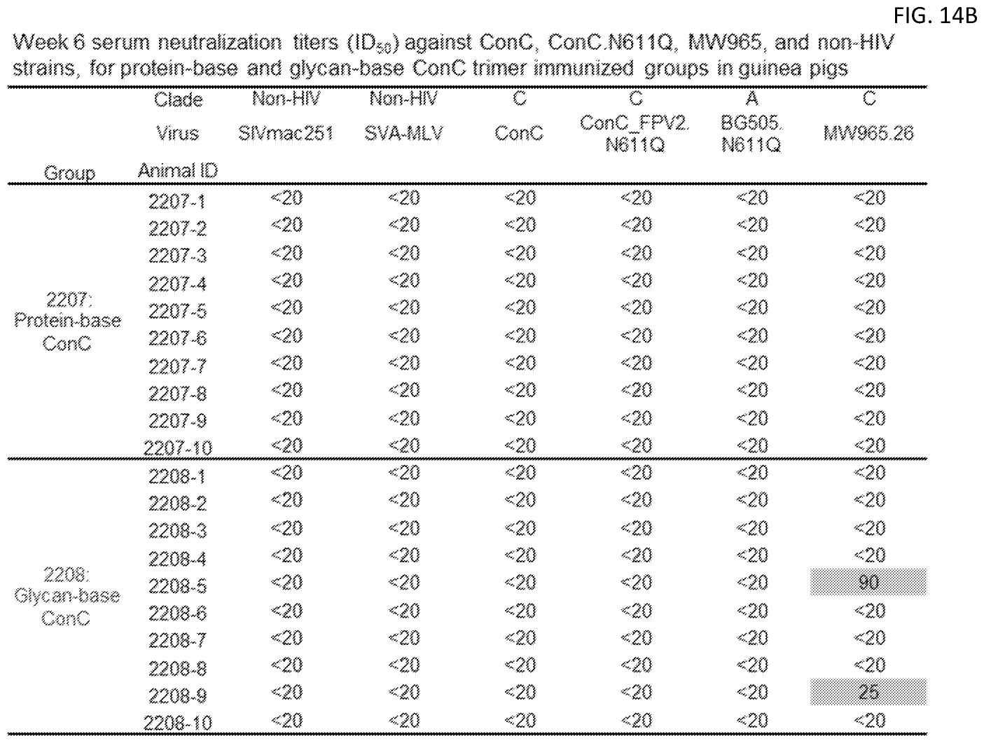

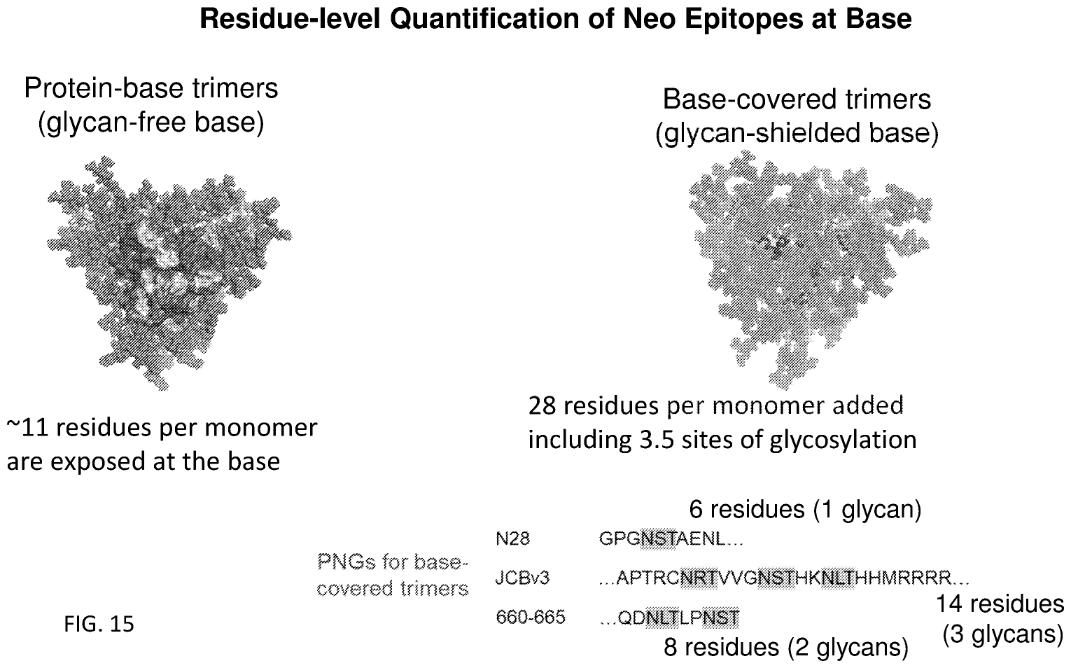

FIGS. 8A and 8B. Antigenic screening of base-covered trimer designs, related to FIG. 1. (8A) Original high-throughput ELISA data. (8B) ELISA raw data values normalized by the reference strain BG505-RnS(7MutVl)-2G-3mut-DS_SOSIP. We consider a trimer to be in the prefusion-closed conformation if either VRC26.25, PGT145, or PGDM1400 binding is greater 0.75.

FIG. 9. Differential Scanning Calorimetry for top BG505 and ConC base covered variants, related to FIG. 2. DSC scans of glycan-base candidates for BG505 and ConC as compared to the protein-base templates, with calculated melting temperature (Tm) for each construct.

FIGS. 10A and 10B. MSD for top BG505 and ConC base-covered variants, related to FIG. 3. MSD antigenicity analysis of glycan-base candidates for BG505 and ConC. In both cases, glycan-base constructs show similar binding to antibody panel as the protein-base counterpart, with the exception of slightly high V3 reactivity in most cases.

FIGS. 11A-11E. Cryo-EM details of Glycan-base ConC, related to FIG. 4. (A) Representative micrograph. (B) Representative 2D class averages. (C) The gold-standard Fourier shell correlation resulted in a resolution of 3.39 A for the overall map using non-uniform refinement with C3 symmetry (upper panel); the orientations of all particles used in the final refinement are shown as a heatmap (lower panel). (D) Local resolution of the final reconstruction. (E) Representative map after sharping and improvement in Resolve. Map is contoured at 2a.

FIG. 12. Cryo-EM details of Glycan-base ConC, related to FIG. 4. Data collection, processing and model refinement statistics for the Glycan-base ConC structure.

FIGS. 13A-13C. ELISA endpoint titers and serum neutralization (ID50) titers elicited at Week 8 for BG505 or ConC trimer immunized mice, related to FIG. 5. (A) Week 8 ELISA endpoint titers using a starting dilution of 1:100 and cross-reactivity (%) of responses are listed for each animal (B) Group comparison shown for week 8 ELISA endpoint titers against protein-base and glycan-base variants of BG505 trimer (left) and ConC trimer (right) (C) Week 8 neutralization (ID50) titers against wildtype BG505, ConC, MW965.26, and SIVmac251 strains.

FIGS. 14A-14B. Week 6 neutralization titers (ID50) for BG505 and ConC trimer immunized groups in guinea pigs, related to FIGS. 6, 7. (A) (B).

FIG. 15 illustrates the possibility that the VRC7873 and VRC7678 trimers may contain neoepitopes in the base region of the trimer. Sequences shown are: GPGNSTAENL (SEQ ID NO: 159), APTRCNRTVVGVSTHKNLTHHMRRRR (SEQ ID NO: 160), AND QDNLTPLNST (SEQ ID NO: 161).

### SEQUENCES

The nucleic and amino acid sequences listed in the accompanying sequence listing are shown using standard letter abbreviations for nucleotide bases, and single letter code for amino acids, as defined in 37 C.F.R. 1.822. Only one strand of each nucleic acid sequence is shown, but the complementary strand is understood as included by any reference to the displayed strand. The Sequence Listing is submitted as an XML file in the form of the file named “4239-108060-02 Sequence Listing” (−291,563 bytes), which was created on Mar. 27, 2023, which is incorporated by reference herein.

### DETAILED DESCRIPTION

The HIV-1 Env trimer is a target for vaccine design as well as a conformational machine that facilitates virus entry by transitioning between prefusion-closed, CD4-bound, and co-receptor-bound conformations before rearranging into a postfusion state. Vaccine designers have successfully restricted the conformation of the HIV-1-Env trimer to its prefusion-closed state, as this state is recognized by most broadly neutralizing—but not by non-neutralizing—antibodies. However, when soluble HIV-1 Env trimers restricted to the prefusion closed conformation are used to immunize animals, the immunodominance of the base of soluble HIV-1 Env trimers reduces the immune response to the remainder of the trimer, potentially reducing their development as an effective HIV-1 immunogens. Provided herein are soluble HIV-1 Env ectodomain trimers that are stabilized in the prefusion conformation and which also contain modification to reduce the immunodominance of the base of soluble HIV-1 Env trimer. The modifications are shown to be effective in multiple HIV-1 Env trimers and can be incorporated into other strains of HIV-1 Env to provide improved immunogenic and antigenic characteristics.

### I. Summary of Terms

Unless otherwise noted, technical terms are used according to conventional usage. Definitions of common terms in molecular biology may be found in Krebs et al. (eds.), Lewin's genes XII, published by Jones & Bartlett Learning, 2017; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particularly suitable methods and materials are described herein. In case of conflict, the present specification, including explanations of terms, will control. In addition, the materials, methods, and examples are illustrative only and not intended to be limiting. To facilitate review of the various implementations, the following explanations of terms are provided:

Adjuvant: A component of an immunogenic composition used to enhance antigenicity. In some implementations, an adjuvant can include a suspension of minerals (alum, aluminum hydroxide, or phosphate) on which antigen is adsorbed; or water-in-oil emulsion, for example, in which antigen solution is emulsified in mineral oil (Freund incomplete adjuvant), sometimes with the inclusion of killed mycobacteria (Freund's complete adjuvant) to further enhance antigenicity (inhibits degradation of antigen and/or causes influx of macrophages). In some implementations, the adjuvant used in a disclosed immunogenic composition is a combination of lecithin and carbomer homopolymer (such as the ADJUPLEX™ adjuvant available from Advanced BioAdjuvants, LLC, see also Wegmann, Clin Vaccine Immunol, 22(9): 1004-1012, 2015). Additional adjuvants for use in the disclosed immunogenic compositions include the QS21 purified plant extract, Matrix M, AS01, MF59, and ALFQ adjuvants. Immunostimulatory oligonucleotides (such as those including a CpG motif) can also be used as adjuvants. Adjuvants include biological molecules (a “biological adjuvant”), such as costimulatory molecules. Exemplary adjuvants include IL-2, RANTES, GM-CSF, TNF-α, IFN-γ, G-CSF, LFA-3, CD72, B7-1, B7-2, OX-40L, 4-1BBL and toll-like receptor (TLR) agonists, such as TLR-9 agonists. The person of ordinary skill in the art is familiar with adjuvants (see, e.g., Singh (ed.) Vaccine Adjuvants and Delivery Systems. Wiley-Interscience, 2007). Adjuvants can be used in combination with the disclosed immunogens.

Administration: The introduction of a composition into a subject by a chosen route. Administration can be local or systemic. For example, if the chosen route is intravenous, the composition (such as a composition including a disclosed immunogen) is administered by introducing the composition into a vein of the subject. Exemplary routes of administration include, but are not limited to, oral, injection (such as subcutaneous, intramuscular, intradermal, intraperitoneal, and intravenous), sublingual, rectal, transdermal (for example, topical), intranasal, vaginal, and inhalation routes.

Amino acid substitution: The replacement of one amino acid in a polypeptide with a different amino acid. In some examples, an amino acid in a polypeptide is substituted with an amino acid from a homologous polypeptide, for example, an amino acid in a recombinant Clade A HIV-1 Env polypeptide can be substituted with the corresponding amino acid from a Clade B HIV-1 Env polypeptide.

Antibody: An immunoglobulin, antigen-binding fragment, or derivative thereof, that specifically binds and recognizes an analyte (antigen), such as HIV-1 Env. The term “antibody” is used herein in the broadest sense and encompasses various antibody structures, including but not limited to monoclonal antibodies, polyclonal antibodies, multispecific antibodies (e.g., bispecific antibodies), and antibody fragments, so long as they exhibit the desired antigen-binding activity. Non-limiting examples of antibodies include, for example, intact immunoglobulins and variants and fragments thereof that retain binding affinity for the antigen. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

Antibody fragments include antigen binding fragments either produced by the modification of whole antibodies or those synthesized de novo using recombinant DNA methodologies (see, e.g., Kontermann and Dubel (Ed), Antibody Engineering, Vols. 1-2, 2^{nd} Ed., Springer Press, 2010). Light and heavy chain variable regions contain a “framework” region interrupted by three hypervariable regions, also called “complementarity-determining regions” or “CDRs” (see, e.g., Kabat et al., Sequences of Proteins of Immunological Interest, U.S. Department of Health and Human Services, 1991). The framework region of an antibody, that is the combined framework regions of the constituent light and heavy chains, serves to position and align the CDRs in three-dimensional space. The CDRs are primarily responsible for binding to an epitope of an antigen.

Carrier: An immunogenic molecule to which an antigen (such as an HIV-1 Env ectodomain trimer) can be linked. When linked to a carrier, the antigen may become more immunogenic. Carriers are chosen to increase the immunogenicity of the antigen and/or to elicit antibodies against the carrier which are diagnostically, analytically, and/or therapeutically beneficial. Useful carriers include polymeric carriers, which can be natural (for example, proteins from bacteria or viruses), semi-synthetic or synthetic materials containing one or more functional groups to which a reactant moiety can be attached.

CD4: Cluster of differentiation factor 4 polypeptide; a T-cell surface protein that mediates interaction with the MHC class II molecule. CD4 also serves as the primary receptor site for HIV-1 on T-cells during HIV-1 infection. CD4 is known to bind to gp120 from HIV-1. The known sequence of the CD4 precursor has a hydrophobic signal peptide, an extracellular region of approximately 370 amino acids, a highly hydrophobic stretch with significant identity to the membrane-spanning domain of the class II MHC beta chain, and a highly charged intracellular sequence of 40 resides (Maddon, Cell 42:93, 1985).

Conservative variants: “Conservative” amino acid substitutions are those substitutions that do not substantially affect or decrease a function of a protein, such as the ability of the protein to elicit an immune response when administered to a subject. The term conservative variation also includes the use of a substituted amino acid in place of an unsubstituted parent amino acid. Furthermore, individual substitutions, deletions or additions which alter, add or delete a single amino acid or a small percentage of amino acids (for instance less than 5%, in some implementations less than 1%) in an encoded sequence are conservative variations where the alterations result in the substitution of an amino acid with a chemically similar amino acid.

The following six groups are examples of amino acids that are considered to be conservative substitutions for one another:
1) Alanine (A), Serine (S), Threonine (T);
2) Aspartic acid (D), Glutanic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Non-conservative substitutions are those that reduce an activity or function of the soluble Env protein, such as the ability to elicit an immune response when administered to a subject. For instance, if an amino acid residue is essential for a function of the protein, even an otherwise conservative substitution may disrupt that activity. Thus, a conservative substitution does not alter the basic function of a protein of interest.

Control: A reference standard. In some implementations, the control is a negative control sample obtained from a healthy patient. In other implementations, the control is a positive control sample obtained from a patient diagnosed with HIV-1 infection. In still other implementations, the control is a historical control or standard reference value or range of values (such as a previously tested control sample, such as a group of HIV-1 patients with known prognosis or outcome, or group of samples that represent baseline or normal values).

A difference between a test sample and a control can be an increase or conversely a decrease. The difference can be a qualitative difference or a quantitative difference, for example, a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Covalent bond: An interatomic bond between two atoms, characterized by the sharing of one or more pairs of electrons by the atoms. The terms “covalently bound” or “covalently linked” refer to making two separate molecules into one contiguous molecule. The terms include reference to joining an antigen (such as an HIV-1 Env ectodomain trimer) either directly or indirectly to a carrier molecule, for example indirectly with an intervening linker molecule, such as a peptide or non-peptide linker.

Degenerate variant: In the context of the present disclosure, a “degenerate variant” refers to a polynucleotide encoding a polypeptide (such as a disclosed immunogen) that includes a sequence that is degenerate as a result of the genetic code. There are 20 natural amino acids, most of which are specified by more than one codon. Therefore, all degenerate nucleotide sequences encoding a peptide are included as long as the amino acid sequence of the peptide encoded by the nucleotide sequence is unchanged.

Detecting: To identify the existence, presence, or fact of something. General methods of detecting may be supplemented with the protocols and reagents disclosed herein. For example, included herein are methods of detecting the level of a protein in a sample or a subject.

Effective amount: An amount of agent, such as an immunogen, that is sufficient to elicit a desired response, such as an immune response in a subject. It is understood that to obtain a protective immune response against an antigen of interest can require multiple administrations of a disclosed immunogen, and/or administration of a disclosed immunogen as the “prime” in a prime boost protocol wherein the boost immunogen can be different from the prime immunogen. Accordingly, an effective amount of a disclosed immunogen can be the amount of the immunogen sufficient to elicit a priming immune response in a subject that can be subsequently boosted with the same or a different immunogen to elicit a protective immune response.

In one example, a desired response is to elicit an immune response that inhibits or prevents HIV-1 infection. HIV-1 infection does not need to be completely eliminated or prevented for the composition to be effective.

Expression: Transcription or translation of a nucleic acid sequence. For example, a gene is expressed when its DNA is transcribed into an RNA or RNA fragment, which in some examples is processed to become mRNA. A gene may also be expressed when its mRNA is translated into an amino acid sequence, such as a protein or a protein fragment. In a particular example, a heterologous gene is expressed when it is transcribed into an RNA. In another example, a heterologous gene is expressed when its RNA is translated into an amino acid sequence. The term “expression” is used herein to denote either transcription or translation. Regulation of expression can include controls on transcription, translation, RNA transport and processing, degradation of intermediary molecules such as mRNA, or through activation, inactivation, compartmentalization or degradation of specific protein molecules after they are produced.

Expression control sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, a start codon (ATG) in front of a protein-encoding gene, splicing signals for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like may be used. In one implementation, when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences.

A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

Expression vector: A vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Non-limiting examples of expression vectors include cosmids, plasmids (e.g., naked or contained in liposomes) and viruses (e.g., lentiviruses, retroviruses, adenoviruses, and adeno-associated viruses) that incorporate the recombinant polynucleotide.

Heterologous: A heterologous polypeptide or polynucleotide refers to a polypeptide or polynucleotide derived from a different source or species.

Host cells: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Human Immunodeficiency Virus Type 1 (HIV-1): A retrovirus that causes immunosuppression in humans (HIV-1 disease), and leads to a disease complex known as the acquired immunodeficiency syndrome (AIDS). “HIV-1 disease” refers to a well-recognized constellation of signs and symptoms (including the development of opportunistic infections) in persons who are infected by an HIV-1 virus, as determined by antibody or western blot studies. Laboratory findings associated with this disease include a progressive decline in T cells. Related viruses that are used as animal models include simian immunodeficiency virus (SIV), and feline immunodeficiency virus (FIV). Treatment of HIV-1 with HAART has been effective in reducing the viral burden and ameliorating the effects of HIV-1 infection in infected individuals.

HIV-1 envelope protein (Env): The HIV-1 Env protein is initially synthesized as a precursor protein of 845-870 amino acids in size. Individual precursor polypeptides form a homotrimer and undergo glycosylation within the Golgi apparatus as well as processing to remove the signal peptide, and cleavage by a cellular protease between approximately positions 511/512 to generate separate gp120 and gp41 polypeptide chains, which remain associated as gp120-gp41 protomers within the homotrimer. The ectodomain (that is, the extracellular portion) of the HIV-1 Env trimer undergoes several structural rearrangements from a prefusion closed conformation that evades antibody recognition, through intermediate conformations that bind to receptors CD4 and co-receptor (either CCR5 or CXCR4), to a postfusion conformation. The HIV-1 Env ectodomain comprises the gp120 protein (approximately HIV-1 Env positions 31-511) and the gp41 ectodomain (approximately HIV-1 Env positions 512-664). An HIV-1 Env ectodomain trimer comprises a protein complex of three HIV-1 Env ectodomains. As used herein “HIV-1 Env ectodomain trimer” includes both soluble trimers (that is, trimers without gp41 transmembrane domain or cytoplasmic tail) and membrane anchored trimers (for example, trimers including a full-length gp41).

Mature gp120 includes approximately HIV-1 Env residues 31-511, contains most of the external, surface-exposed, domains of the HIV-1 Env trimer, and it is gp120 which binds both to cellular CD4 receptors and to cellular chemokine receptors (such as CCR5). The mature gp120 wild-type polypeptide is heavily N-glycosylated, giving rise to an apparent molecular weight of 120 kD. Native gp120 includes five conserved regions (C1-C5) and five regions of high variability (V1-V5).

Mature gp41 includes approximately HIV-1 Env residues 512-860, and includes cytosolic-, transmembrane-, and ecto-domains. The gp41 ectodomain (including approximately HIV-1 Env residues 512-644) can interact with gp120 to form an HIV-1 Env protomer that trimerizes to form the HIV-1 Env trimer.

The prefusion closed conformation of the HIV-1 Env ectodomain trimer is a structural conformation adopted by HIV-1 Env ectodomain trimer after cellular processing to a mature prefusion state with distinct gp120 and gp41 polypeptide chains, and before specific binding to the CD4 receptor. The three-dimensional structure of an exemplary HIV-1 Env ectodomain trimer in the prefusion closed conformation is known (see, e.g., Pancera et al., Nature, 514:455-461, 2014).

In the prefusion closed conformation, the HIV-1 Env ectodomain trimer includes a V1V2 domain “cap” at its membrane distal apex, with the V1V2 domain of each Env protomer in the trimer coming together at the membrane distal apex. At the membrane proximal aspect, the prefusion closed conformation of the HIV-1 Env ectodomain trimer includes distinct α6 and α7 helices. CD4 binding causes changes in the conformation of the HIV-1 Env ectodomain trimer, including disruption of the V1V1 domain cap, which “opens” as each V1V2 domain moves outward from the longitudinal axis of the Env trimer, and formation of the HR1 helix, which includes both the α6 and α7 helices (which are no longer distinct). These conformational changes bring the N-terminus of the fusion peptide within close proximity of the target cell membrane, and expose “CD4-induced” epitopes (such as the 17b epitope) that are present in the CD4-bound open conformation, but not the prefusion closed conformation, of the HIV-1 Env ectodomain trimer.

A standardized numbering scheme for HIV-1 Env proteins (the HXB2 numbering scheme) is set forth in Numbering Positions in HIV Relative to HXB2CG Bette Korber et al., Human Retroviruses and AIDS 1998: A Compilation and Analysis of Nucleic Acid and Amino Acid Sequences. Korber et al., Eds. Theoretical Biology and Biophysics Group, Los Alamos National Laboratory, Los Alamos, NM, which is incorporated by reference herein in its entirety. For reference, the amino acid sequence of HIV-1 Env of HXB2 is set forth as SEQ ID NO: 1 (GENBANK® GI:1906382, incorporated by reference herein).

HXB2 (Clade B, SEQ ID NO: 1):
MRVKEKYQHLWRWGWRWGTMLLGMLMICSATEKLWVTVYYGVPVWKEATT
TLFCASDAKAYDTEVHNVWATHACVPTDPNPQEVVLVNVTENFNMWKNDM
VEQMHEDIISLWDQSLKPCVKLTPLCVSLKCTDLKNDTNTNSSSGRMIME
KGEIKNCSFNISTSIRGKVQKEYAFFYKLDIIPIDNDTTSYKLISCNTSV
ITQACPKVSFEPIPIHYCAPAGFAILKCNNKTFNGTGPCTNVSTVQCTHG
IRPVVSTQLLLNGSLAEEEVVIRSVNFTDNAKTIIVQLNTSVEINCTRPN
NNTRKRIRIQRGPGRAFVTIGKIGNMRQAHCNISRAKWNNTLKQIASKLR
EQFGNNKTIIFKQSSGGDPEIVTHSFNCGGEFFYCNSTQLFNSTWFNSTW
STEGSNNTEGSDTITLPCRIKQIINMWQKVGKAMYAPPISGQIRCSSNIT
GLLLTRDGGNSNNESEIFRPGGGDMRDNWRSELYKYKVVKIEPLGVAPTK
AKRRVVQREKRAVGIGALFLGFLGAAGSTMGAASMTLTVQARQLLSGIVQ
QQNNLLRAIEAQQHLLQLTVWGIKQLQARILAVERYLKDQQLLGIWGCSG
KLICTTAVPWNASWSNKSLEQIWNHTTWMEWDREINNYTSLIHSLIEESQ
NQQEKNEQELLELDKWASLWNWFNITNWLWYIKLFIMIVGGLVGLRIVFA
VLSIVNRVRQGYSPLSFQTHLPTPRGPDRPEGIEEEGGERDRDRSIRLVN
GSLALIWDDLRSLCLFSYHRLRDLLLIVTRIVELLGRRGWEALKYWWNLL
QYWSQELKNSAVSLLNATAIAVAEGTDRVIEVVQGACRAIRHIPRRIRQG
LERILL

HIV-1 Env ectodomain trimer stabilized in a prefusion closed conformation: A HIV-1 Env ectodomain trimer having one or more amino acid substitutions, additions, deletions, or insertions compared to a native HIV-1 Env sequence that provide for increased retention of the prefusion closed conformation upon CD4 binding compared to a corresponding native HIV-1 Env sequence. In some implementations, the HIV-1 Env ectodomain trimer can include one or more cysteine substitutions that allow formation of a non-natural disulfide bond that stabilizes the HIV-1 Env ectodomain trimer in its prefusion closed conformation.

An HIV-1 Env ectodomain trimer stabilized in the prefusion closed conformation has at least 90% (such as at least 95% or at least 99%) reduced transition to the CD4-bound open conformation upon CD4 binding compared to a corresponding native HIV-1 Env sequence. The “stabilization” of the prefusion closed conformation by the one or more amino acid substitutions, additions, deletions, or insertions can be, for example, energetic stabilization (for example, reducing the energy of the prefusion closed conformation relative to the CD4-bound open conformation) and/or kinetic stabilization (for example, reducing the rate of transition from the prefusion closed conformation to the prefusion closed conformation). Additionally, stabilization of the HIV-1 Env ectodomain trimer in the prefusion closed conformation can include an increase in resistance to denaturation compared to a corresponding native HIV-1 Env sequence.

Methods of determining if a HIV-1 Env ectodomain trimer is in the prefusion closed conformation are provided herein, and include (but are not limited to) negative stain electron microscopy and antibody binding assays using a prefusion closed conformation specific antibody, such as VRC26 or PGT145. Methods of determining if a HIV-1 Env ectodomain trimer is in the CD4-bound open conformation are also provided herein, and include (but are not limited to) negative stain electron microscopy and antibody binding assays using a CD4-bound open conformation specific antibody, such as 17b, which binds to a CD4-induced epitope. Transition from the prefusion closed conformation upon CD4 binding can be assayed, for example, by incubating a HIV-1 Env ectodomain trimer of interest that is in the prefusion closed conformation with a molar excess of CD4, and determining if the HIV-1 Env ectodomain trimer retains the prefusion closed conformation (or transitions to the CD4-bound open conformation) by negative stain electron microscopy analysis, or antigenic analysis.

HIV-1 gp140: A HIV Env polypeptide including gp120 and the gp41 ectodomain, but not the gp41 transmembrane or cytosolic domains. HIV-1 gp140 polypeptides can trimerize to form a soluble HIV-1 Env ectodomain trimer.

HIV-1 gp145: A HIV Env polypeptide including gp120, the gp41 ectodomain, and the gp41 transmembrane domain. HIV-1 gp145 polypeptides can trimerize to form a membrane-anchored HIV-1 Env ectodomain trimers.

HIV-1 gp160: A HIV Env polypeptide including gp120 and the entire gp41 protein (ectodomain, transmembrane domain, and cytosolic tail).

HIV-1 neutralizing antibody: An antibody that reduces the infectious titer of HIV-1 by binding to HIV-1 Env protein and inhibiting HIV-1 function. In some implementations, neutralizing antibodies to HIV-1 can inhibit the infectivity of multiple strains of HIV-1, Teir-2 strain from multiple clades of HIV-1. In some implementations, a disclosed immunogen can be administered to a subject to elicit an immune response that includes production of antibodies that specifically bind to the HIV-1 Env trimer and neutralize Teir-2 strains of HIV-1 from multiple HIV-1 clades.

Immunogenic conjugate: A composition composed of at least two heterologous molecules (such as an HIV-1 Env trimer and a carrier, such as a protein carrier) linked together that stimulates or elicits an immune response to a molecule in the conjugate in a vertebrate. In some implementations where the conjugate include a viral antigen, the immune response is protective in that it enables the vertebrate animal to better resist infection from the virus from which the antigen is derived.

Immune response: A response of a cell of the immune system, such as a B cell, T cell, or monocyte, to a stimulus. In one implementation, the response is specific for a particular antigen (an “antigen-specific response”). In one implementation, an immune response is a T cell response, such as a CD4+ response or a CD8+ response. In another implementation, the response is a B cell response, and results in the production of specific antibodies. “Priming an immune response” refers to treatment of a subject with a “prime” immunogen to induce an immune response that is subsequently “boosted” with a boost immunogen. Together, the prime and boost immunizations produce the desired immune response in the subject. “Enhancing an immune response” refers to co-administration of an adjuvant and an immunogenic agent, wherein the adjuvant increases the desired immune response to the immunogenic agent compared to administration of the immunogenic agent to the subject in the absence of the adjuvant.

Immunogen: A protein or a portion thereof that is capable of inducing an immune response in a mammal, such as a mammal infected or at risk of infection with a pathogen.

Immunogenic composition: A composition comprising a disclosed immunogen, or a nucleic acid molecule or vector encoding a disclosed immunogen, that elicits a measurable CTL response against the immunogen, or elicits a measurable B cell response (such as production of antibodies) against the immunogen, when administered to a subject. It further refers to isolated nucleic acids encoding an immunogen, such as a nucleic acid that can be used to express the immunogen (and thus be used to elicit an immune response against this immunogen). For in vivo use, the immunogenic composition will typically include the protein or nucleic acid molecule in a pharmaceutically acceptable carrier and may also include other agents, such as an adjuvant.

Inhibiting or treating a disease: Inhibiting the full development of a disease or condition, for example, in a subject who is at risk for a disease such as acquired immunodeficiency syndrome (AIDS). “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. Inhibiting a disease can include preventing or reducing the risk of the disease, such as preventing or reducing the risk of viral infection. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology.

Isolated: An “isolated” biological component has been substantially separated or purified away from other biological components, such as other biological components in which the component naturally occurs, such as other chromosomal and extrachromosomal DNA, RNA, and proteins. Proteins, peptides, nucleic acids, and viruses that have been “isolated” include those purified by standard purification methods. Isolated does not require absolute purity, and can include protein, peptide, nucleic acid, or virus molecules that are at least 50% isolated, such as at least 75%, 80%, 90%, 95%, 98%, 99%, or even 99.9% isolated.

Linked: The term “linked” means joined together, either directly or indirectly. For example, a first moiety may be covalently or noncovalently (e.g., electrostatically) linked to a second moiety. This includes, but is not limited to, covalently bonding one molecule to another molecule, noncovalently bonding one molecule to another (e.g. electrostatically bonding), non-covalently bonding one molecule to another molecule by hydrogen bonding, non-covalently bonding one molecule to another molecule by van der Waals forces, and any and all combinations of such couplings. Indirect attachment is possible, such as by using a “linker”. In several implementations, linked components are associated in a chemical or physical manner so that the components are not freely dispersible from one another, at least until contacting a cell, such as an immune cell.

Linker: One or more molecules or groups of atoms positioned between two moieties. Typically, linkers are bifunctional, i.e., the linker includes a functional group at each end, wherein the functional groups are used to couple the linker to the two moieties. The two functional groups may be the same, i.e., a homobifunctional linker, or different, i.e., a heterobifunctional linker. In several implementations, a peptide linker can be used to link the C-terminus of a first protein to the N-tenninus of a second protein. Non-limiting examples of peptide linkers include glycine-serine peptide linkers, which are typically not more than 10 amino acids in length. Typically, such linkage is accomplished using molecular biology techniques to genetically manipulate DNA encoding the first polypeptide linked to the second polypeptide by the peptide linker.

Native protein, sequence, or disulfide bond: A polypeptide, sequence or disulfide bond that has not been modified, for example, by selective mutation. For example, selective mutation to focus the antigenicity of the antigen to a target epitope, or to introduce a disulfide bond into a protein that does not occur in the native protein. Native protein or native sequence are also referred to as wild-type protein or wild-type sequence. A non-native disulfide bond is a disulfide bond that is not present in a native protein, for example, a disulfide bond that forms in a protein due to introduction of one or more cysteine residues into the protein by genetic engineering.

Nucleic acid molecule: A polymeric form of nucleotides, which may include both sense and anti-sense strands of RNA, cDNA, genomic DNA, and synthetic forms and mixed polymers of the above. A nucleotide refers to a ribonucleotide, deoxynucleotide or a modified form of either type of nucleotide. The term “nucleic acid molecule” as used herein is synonymous with “nucleic acid” and “polynucleotide.” A nucleic acid molecule is usually at least 10 bases in length, unless otherwise specified. The term includes single- and double-stranded forms of DNA. A polynucleotide may include either or both naturally occurring and modified nucleotides linked together by naturally occurring and/or non-naturally occurring nucleotide linkages. “cDNA” refers to a DNA that is complementary or identical to an mRNA, in either single stranded or double stranded form. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked nucleic acid sequences are contiguous and, where necessary to join two protein-coding regions, in the same reading frame.

Pharmaceutically acceptable carriers: The pharmaceutically acceptable carriers of use are conventional. Remington: The Science and Practice of Pharmacy, 22^{nd} ed., London, UK: Pharmaceutical Press, 2013, describes compositions and formulations suitable for pharmaceutical delivery of the disclosed agents.

In general, the nature of the carrier will depend on the particular mode of administration being employed. For instance, parenteral formulations usually include injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, added preservatives (such as non-natural preservatives), and pH buffering agents and the like, for example sodium acetate or sorbitan monolaurate. In particular examples, the pharmaceutically acceptable carrier is sterile and suitable for parenteral administration to a subject for example, by injection. In some implementations, the active agent and pharmaceutically acceptable carrier are provided in a unit dosage form such as a pill or in a selected quantity in a vial. Unit dosage forms can include one dosage or multiple dosages (for example, in a vial from which metered dosages of the agents can selectively be dispensed).

Polypeptide: Any chain of amino acids, regardless of length or post-translational modification (e.g., glycosylation or phosphorylation). “Polypeptide” applies to amino acid polymers including naturally occurring amino acid polymers and non-naturally occurring amino acid polymer as well as in which one or more amino acid residue is a non-natural amino acid, for example, an artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used herein to refer to a polymer of amino acid residues.

Prime-boost immunization: An immunotherapy including administration of multiple immunogens over a period of time to elicit the desired immune response.

Recombinant: A recombinant nucleic acid is one that has a sequence that is not naturally occurring or has a sequence that is made by an artificial combination of two otherwise separated segments of sequence. This artificial combination can be accomplished, for example, the artificial manipulation of isolated segments of nucleic acids, for example, using genetic engineering techniques. A recombinant protein is one that has a sequence that is not naturally occurring or has a sequence that is made by an artificial combination of two otherwise separated segments of sequence. In several implementations, a recombinant protein is encoded by a heterologous (for example, recombinant) nucleic acid that has been introduced into a host cell, such as a bacterial or eukaryotic cell. The nucleic acid can be introduced, for example, on an expression vector having signals capable of expressing the protein encoded by the introduced nucleic acid or the nucleic acid can be integrated into the host cell chromosome.

Sequence identity: The similarity between amino acid sequences is expressed in terms of the similarity between the sequences, otherwise referred to as sequence identity. Sequence identity is frequently measured in terms of percentage identity; the higher the percentage, the more similar the two sequences are. Homologs, orthologs, or variants of a polypeptide will possess a relatively high degree of sequence identity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. In the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

Variants of a polypeptide are typically characterized by possession of at least about 75%, for example, at least about 80%, 85%, 90%, 91%, 92%, 93%, 94%, 95%, 96%, 97%, 98% or 99% sequence identity counted over the full length alignment with the amino acid sequence of interest. Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet.

As used herein, reference to “at least 90% identity” (or similar language) refers to “at least 90%, at least 91%, at least 92%, at least 93%, at least 94%, at least 95%, at least 96%, at least 97%, at least 98%, at least 99%, or even 100% identity” to a specified reference sequence.

Signal Peptide: A short amino acid sequence (e.g., approximately 18-30 amino acids in length) that directs newly synthesized secretory or membrane proteins to and through membranes (for example, the endoplasmic reticulum membrane). Signal peptides are typically located at the N-terminus of a polypeptide and are removed by signal peptidases after the polypeptide has crossed the membrane. Signal peptide sequences typically contain three common structural features: an N-terminal polar basic region (n-region), a hydrophobic core, and a hydrophilic c-region). An exemplary signal peptide sequence is set forth as residues 1-29 of SEQ ID NO: 1.

Specifically bind: When referring to the formation of an antibody:antigen protein complex, or a protein:protein complex, refers to a binding reaction which determines the presence of a target protein, peptide, or polysaccharide (for example, a glycoprotein), in the presence of a heterogeneous population of proteins and other biologics. Thus, under designated conditions, a particular antibody or protein binds preferentially to a particular target protein, peptide or polysaccharide (such as an antigen present on the surface of a pathogen, for example, gp120) and does not bind in a significant amount to other proteins or polysaccharides present in the sample or subject. Specific binding can be determined by standard methods. A first protein or antibody specifically binds to a target protein when the interaction has a K_{D} of less than 10⁻⁷ Molar, such as less than 10⁻⁸ Molar, less than 10⁻⁹, or even less than 10⁻¹⁰ Molar.

Subject: Living multicellular vertebrate organisms, a category that includes human and non-human mammals. In an example, a subject is a human. In an additional example, a subject is selected that is in need of inhibiting of an HIV-1 infection. For example, the subject is either uninfected and at risk of HIV-1 infection or is infected in need of treatment.

Transmembrane domain: An amino acid sequence that inserts into a lipid bilayer, such as the lipid bilayer of a cell or virus or virus-like particle. A transmembrane domain can be used to anchor an antigen to a membrane.

Under conditions sufficient for: A phrase that is used to describe any environment that permits a desired activity.

Vaccine: A pharmaceutical composition that elicits a prophylactic or therapeutic immune response in a subject. In some cases, the immune response is a protective immune response. Typically, a vaccine elicits an antigen-specific immune response to an antigen of a pathogen, for example a viral pathogen, or to a cellular constituent correlated with a pathological condition. A vaccine may include a polynucleotide (such as a nucleic acid encoding a disclosed antigen), a peptide or polypeptide (such as a disclosed antigen), a virus, a cell or one or more cellular constituents. In one specific, non-limiting example, a vaccine reduces the severity of the symptoms associated with HIV-1 infection and/or decreases the viral load compared to a control. In another non-limiting example, a vaccine reduces HIV-1 infection compared to a control.

Vector: An entity containing a DNA or RNA molecule bearing a promoter(s) that is operationally linked to the coding sequence of an immunogenic protein of interest and can express the coding sequence. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. A vector can include nucleic acid sequences that permit it to replicate in a host cell, such as an origin of replication. A vector can also include one or more selectable marker genes and other genetic elements. Viral vectors are recombinant nucleic acid vectors having at least some nucleic acid sequences derived from one or more viruses.

A non-limiting example of a DNA-based expression vector is pCDNA3.1, which can include includes a mammalian expression enhancer and promoter (such as a CMV promoter). Non-limiting examples of viral vectors include adeno-associated virus (AAV) vectors as well as Poxvirus vector (e.g., Vaccinia, MVA, avian Pox, or Adenovirus).

### II. Immunogens

Implementations of immunogens comprising a soluble HIV-1 Env ectodomain trimer that is stabilized in a prefusion closed conformation and includes N-linked glycan sequons that are glycosylated during production to occlude the “base” region of the trimer are provided below. The immunogens can be used to generate a neutralizing immune response to HIV-1 in a subject, for example, to treat or prevent an HIV-1 infection in the subject.

### Soluble HIV-1 Env Ectodomain Trimers

Provided herein are soluble HIV-1 Env ectodomain trimers comprising protomers (each comprising a gp120 protein and a gp41 ectodomain) that are modified from a native form (e.g., by introduction of one or more amino acid substitutions, additions, and insertions) to be stabilized in a prefusion closed conformation and to include N-linked glycan sequons that occlude the “base” region of the trimer. The soluble HIV-1 Env ectodomain trimers have reduced binding to CD4 compared to native HIV-1 Env ectodomain trimers, but retain binding affinity for broadly neutralizing antibodies, such as PG9, PG16, VRC26, PGT145, VRCO1, VRC07, N6, 35022, 8ANC195, PGT151, and/or PGT121, and elicit a reduce immune response to the ‘base’ region of the trimer compared to trimers lacking N-linked glycan sequons. Glycan occlusion of the base region of the trimer can be measured, for example, using antibodies that bind to protein epitopes on the base region, such as 1E6, 3H2, 5H3, and 9B9. Administration of an effective amount of a disclosed soluble HIV-1 Env ectodomain trimer to a subject elicits a neutralizing immune response to HIV-1 in the subject.

The protomers of the soluble HIV-1 Env ectodomain trimer comprise or consist of HIV-1 Env positions 31 to one of 653-664 modified with amino acid substitutions, additions, and/or insertions as described herein.

The protomers of the disclosed soluble HIV-1 Env ectodomain trimer include several amino acid substitutions that stabilize the trimer in the prefusion closed conformation. These substitutions comprise:
(i) the “SOS” substitutions, which are cysteine substitutions at HIV-1 Env positions 501 and 605 (for example, by A501C and T605C substitutions) to form a non-natural intra-protomer disulfide bond,
(ii) the “DS” substitutions, which are cysteine substitutions at HIV-1 Env positions 201 and 433 (e.g., by introduction of 1201C and A433C substitutions) to form a non-natural intra-protomer disulfide bond; and
(iii) the “IP” substitution, which is a proline substitution at HIV-1 Env position 559 (for example, by an 1559P substitution);
(iv) the “3mut” substitutions, which are methionine, leucine, and proline substitutions at HIV-1 Env positions 302, 320, and 329, respectively (for example, N302M, T320L, and A329P substitutions).
   The presence of the SOS, IP, DS, and 3mut substitutions contributes to the stabilization of the HIV-1 Env ectodomain in the prefusion closed conformation.

Additionally, the protomers of the disclosed soluble HTV-1 Env ectodomain trimer include several amino acid substitutions, additions, and/or insertions to introduce N-linked glycan sequons (N-X-[S/T]consensus) at the ‘base’ region of the trimer. These substitutions comprise:
(i) optionally an addition of three amino acids immediately N-terminal to HIV-1 Env position 31 to introduce an N-linked glycan sequon;
(ii) amino acid substitutions and/or insertions to introduce one, two, or three N-linked glycan sequons between HIV-1 Env positions 502 and 509, wherein no more than 15 amino acids are inserted; and
(iii) amino acid substitutions between HIV-1 Env positions 650-664 to introduce one or two N-linked glycan sequons, and/or addition of up to 60 amino acids at the C-terminus of the protomers to introduce one to five N-linked glycan sequons.
   Glycosylation of the N-linked glycan sequons during production of the soluble HIV-1 Env trimer in cells effectively covers the ‘base’ of the soluble trimer to reduce any base-directed immune response.

In some implementations, the protomers of the disclosed soluble HIV-1 Env ectodomain trimer include the addition of three amino acids immediately N-terminal to HIV-1 Env position 31 to introduce an N-linked glycan sequon.

In some implementations, the amino acid substitutions and/or insertions to introduce one, two, or three N-linked glycan sequons between HIV-1 Env positions 502 and 509, wherein no more than 15 amino acids are inserted is an amino acid substitution to introduce an N-linked glycan sequon at HIV-1 Env position 502. In some implementations, the amino acid substitutions and/or insertions to introduce one, two, or three N-linked glycan sequons between HIV-1 Env positions 502 and 509, wherein no more than 15 amino acids are inserted is an amino acid substitution to introduce an N-linked glycan sequon at HIV-1 Env position 502 and/or insertion of up to 15 amino acids with at least two N-linked glycan sequons between HIV-1 Env positions 507/508 (such as insertion of amino acids NSTHKNLTHHM, SEQ ID NO: 11). In some implementations, the amino acid substitutions and/or insertions to introduce one, two, or three N-linked glycan sequons between HIV-1 Env positions 502 and 509, wherein no more than 15 amino acids are inserted is insertion of up to 15 amino acids with at least two N-linked glycan sequons between HIV-1 Env positions 507/508 (such as insertion of amino acids NSTHKNLTHHM, SEQ ID NO: 11).

In some implementations, the amino acid substitutions between HIV-1 Env positions 650-664 to introduce one or two N-linked glycan sequons are amino acid substitutions to introduce an N-linked glycan sequon at one or two of positions 650, 656, or 660. In some implementations, the amino acid substitutions between HIV-t Env positions 650-664 to introduce one or two N-linked glycan sequons is amino acid substitutions to introduce a single N-linked glycan sequon at positions 650. In some implementations, the amino acid substitutions between HIV-1 Env positions 650-664 to introduce one or two N-linked glycan sequons is amino acid substitutions to introduce a single N-linked glycan sequon at positions 656. In some implementations, the amino acid substitutions between HIV-1 Env positions 650-664 to introduce one or two N-linked glycan sequons is amino acid substitutions to introduce a single N-linked glycan sequon at positions 660. In several such implementations, the soluble HIV-1 Env trimer comprises an addition of three amino acids to introduce an N-linked glycan sequon at the C-terminus of protomers in the trimer.

In some implementations, the addition of up to 60 amino acids at the C-terminus of the protomers to introduce one to five N-linked glycan sequons is an insertion of three amino acids at the C-terminus of the protomers to introduce an N-linked glycan sequon. In some implementations, the addition of up to 60 amino acids at the C-terminus of the protomers to introduce one to five N-linked glycan sequons is an addition of one of the following sequences at the C-terminus of the protomers, in the trimer:

SEQ ID NO: 12
GGYnVtYnVtGGYnVtYnVtIGGSGG,
SEQ ID NO: 13
GGGSSGVRLWnTtKIMLGQVHnVtKGWLIFVAEQEELYVRVQNGFRKVQL
EARTPLPR,

Native HIV-1 Env sequences include a furin cleavage site between positions 508 and 512 (HXB2 numbering), that separates gp120 and gp41. The protomers of the disclosed soluble HIV-1 Env ectodomain trimer optionally include an enhanced cleavage site between gp120 and gp41 proteins. In some implementations, the enhanced cleavage site can include substitution of any one of RRRRRR (SEQ ID NO: 21), GRRRRRR (SEQ ID NO: 14), GGSGRRRRRR (SEQ ID NO: 15), GRRRRRRRRR (SEQ ID NO: 16), or GNSTHKQLTHHMRRRRRR (SEQ ID NO: 17) for the amino acids of a gp120/gp41 furin cleavage site. In an example, the enhanced cleavage site is substitution of six arginine resides for the four residues of the native cleavage site. In some implementations, the protomers of the disclosed soluble HIV-1 Env ectodomain trimer include the enhanced cleavage site between gp120 and gp41 proteins, such as substitution of six arginine resides for the four residues of the native cleavage site.

In some implementations, the soluble HIV-1 Env ectodomain trimer comprises protomers comprising or consisting of HIV-1 Env positions 31 to one of 653-664 further modified with amino acid substitutions to introduce the SOS, IP, DS, and 3mut modifications, substitution of RRRRRR (SEQ ID NO: 21) for the amino acids of a gp120/gp41 furin cleavage site, and wherein protomers of the trimer further comprise the following to introduce N-linked glycan sequons:
(i) optionally the addition of three amino acids immediately N-terminal to HIV-1 Env position 31 to introduce an N-linked glycan sequon; and
(ii) amino acid substitutions to introduce an N-linked glycan sequon at HIV-1 Env position 502, insertion of up to 15 amino acids with at least two N-linked glycan sequons between HIV-1 Env positions 507/508, or both;
(iii) amino acid substitutions to introduce N-linked glycan sequons at one two or three of HIV-1 Env positions 650, 656, and 660; and optionally the amino acid addition at the C-terminus of the protomers to introduce up to five N-linked glycan sequons.

In some implementations, the soluble HIV-1 Env ectodomain trimer comprises protomers comprising or consisting of HIV-1 Env positions 31 to one of 653-664 further modified with amino acid substitutions to introduce the SOS, IP, DS, and 3mut modifications, substitution of RRRRRR (SEQ ID NO: 21) for the amino acids of a gp120/gp41 furin cleavage site, and wherein protomers of the trimer further comprise the following to introduce N-linked glycan sequons:
(i) addition of three amino acids immediately N-terminal to HIV-1 Env position 31 to introduce an N-linked glycan sequon;
(ii) amino acid substitution to introduce an N-linked glycan sequon at HIV-1 Env position 502;
(iii) insertion of amino acids NSTHKNLTHHM (SEQ ID NO: 11) between HIV-1 Env positions 507/508 to introduce two N-linked glycan sequons;
(iv) amino acid substitution to introduce an N-linked glycan sequon at HIV-1 Env position 660; and
(v) addition of three amino acids at the C-terminus of the protomers to introduce an N-linked glycan sequon.

In some implementations, the soluble HIV-1 Env ectodomain trimer comprises protomers comprising or consisting of HIV-1 Env positions 31 to one of 653-664 further modified with amino acid substitutions to introduce the SOS, IP, DS, and 3mut modifications, substitution of RRRRRR (SEQ ID NO: 21) for the amino acids of a gp120/gp41 furin cleavage site, and wherein protomers of the trimer further comprise the following to introduce N-linked glycan sequons:
(i) amino acid substitution to introduce an N-linked glycan sequon at HIV-1 Env position 502;
(ii) amino acid substitutions to introduce an N-linked glycan sequon at one of HIV-1 Env positions 650, 656, or 660;
(iii) optionally addition of three amino acids at the C-terminus of the protomers to introduce an N-linked glycan sequon, or addition of up to 60 amino acids at the C-terminus of the protomers to introduce up to four N-linked glycan sequons.

In some implementations, the protomers of the soluble HIV-1 Env ectodomain trimer can further include an N-linked glycosylation site at HIV-1 Env position 332 (if not already present on the ectodomain). For example, by T332N substitution in the case of BG505-based immunogens. The presence of the glycosylation site at N332 allows for binding by 2G12 antibody.

In some implementations, the protomers of the soluble HIV-1 Env ectodomain trimer can further include a lysine residue at HIV-1 Env position 168 (if not already present on the ectodomain). For example, the lysine residue can be added by amino acid substitution (such as an E168K substitution in the case of the JR-FL based immunogens). The presence of the lysine residue at position 168 allows for binding of particular broadly neutralizing antibodies to the VIV2 loop of gp120.

In some implementations, the protomers of the soluble HIV-1 Env ectodomain trimer can further include one or more (such as all) of 2041, 535N, 573F, 588E, 589V, 651F, and 6551 amino acids (HXB2 numbering) if not already present in the protomer. For example, by A2041, M535N, I573F, K588E, D589V, N651F, and K655I substitutions in the case of BG505-based immunogens. In some implementations, the protomers of the soluble HIV-1 Env ectodomain trimer can further include one or more amino acid substitutions to introduce the sequence AENL for positions 31-35 of the protomer, if not already present.

The prefusion closed conformation of the HIV-1 Env trimer has been disclosed, for example, in Pancera et al., Nature, 514, 455-461, 2014 and PCT App. No. PCT/US2015/048729, each of which is incorporated by reference herein in its entirety. In some implementations, the protomers of the HIV-1 Env ectodomain trimers disclosed herein can further include one of more modifications as disclosed in PCT App. No. PCT/US2015/048729 to stabilize the soluble HIV-1 Env ectodomain trimer in the prefusion closed conformation. For example, the HIV-1 Env ectodomain trimer can include a prefusion closed conformation wherein the V1V2 domain of each Env ectodomain protomer in the trimer comes together at the membrane distal apex. At the membrane proximal aspect, the HIV-1 Env ectodomain trimer in the prefusion closed conformation includes distinct α6 and α7 helices; the α7 helix does not start until after residue 570. For example, in the prefusion closed conformation, the interprotomer distance between residues 200 and 313 can be less than 5 Angstroms.

HIV-1 can be classified into four groups: the “major” group M, the “outlier” group O, group N, and group P. Within group M, there are several genetically distinct clades (or subtypes) of HIV-1. The disclosed soluble HIV-1 Env proteins can be derived from any type of HIV, such as groups M, N, O, or P, or clade, such as clade A, B, C, D, F, G, H, J, or K, and the like. HIV-1 Env proteins from the different HIV-1 clades, as well as nucleic acid sequences encoding such proteins and methods for the manipulation and insertion of such nucleic acid sequences into vectors, are known (see, e.g., HIV Sequence Compendium, Division of AIDS, National Institute of Allergy and Infectious Diseases (2013); HIV Sequence Database (hiv-web.lanl.gov/content/hiv-db/mainpage.html); see, e.g., Sambrook et al. (Molecular Cloning: A Laboratory Manual, 4^{th} ed, Cold Spring Harbor, New York, 2012) and Ausubel et al. (In Current Protocols in Molecular Biology, John Wiley & Sons, New York, through supplement 104, 2013). Exemplary native HIV-1 Env protein sequences are available in the HIV Sequence Database (hiv-web.lanl.gov/content/hiv-db/mainpage.html).

In some implementations, the protomers of the soluble HIV-1 Env ectodomain trimer include an amino acid sequence of a native HIV-1 Env protein, for example, from genetic subtype A-F as available in the HIV Sequence Database (hiv-web.lanl.gov/content/hiv-db/mainpage.html) or an amino acid sequence at least 95% (such as at least 96%, at least 97%, at least 98% or at least 99%) identical thereto that has been modified by one or more amino acid substitutions, additions, deletions, and/or insertions as discussed herein, for example, to stabilize the soluble HIV-1 Env ectodomain trimer in the prefusion closed conformation, and to introduce N-linked glycan sequons to cover the membrane-proximal base of the trimer.

In some implementations, the protomers of the soluble HIV-1 Env ectodomain trimer comprise or consist of an amino acid sequence that is at least 95% (such as at least 96%, at least 97%, at least 98%, at least 99%, or 100%) identical to HIV-1 Env positions 31 to one of 653-664 of a native HIV-1 Env sequence, for example, from genetic subtype A-F as available in the HIV Sequence Database (hiv-web.lanl.gov/content/hiv-db/mainpage.html) that has been modified by one or more amino acid substitutions, additions, deletions, and/or insertions as discussed herein, for example, to stabilize the soluble HIV-1 Env ectodomain trimer in the prefusion closed conformation, and to introduce N-linked glycan sequons and to introduce N-linked glycan sequons to cover the membrane-proximal base of the trimer.

In some implementations, the protomers of the soluble HIV-1 Env ectodomain trimer comprise or consist of an amino acid sequence that is at least 95% (such as at least 96%, at least 97%, at least 98%, at least 99%, or 100%) identical to HIV-1 Env positions 31 to one of 653-664 of a native HIV-1 Env sequence, for example, selected from the native HIV-1 Env sequences in the following table, that has been modified by one or more amino acid substitutions, additions, deletions, and/or insertions as discussed herein, for example, to stabilize the soluble HIV-1 Env ectodomain trimer in the prefusion closed conformation, and to introduce N-linked glycan sequons to cover the membrane-proximal base of the trimer. The table below include consensus HIV-1 Env sequences for HIV-t Env from different HIV-I clades, which are considered “native” HIV-1 Env sequences herein.

CONSENSUS_A1 (Clade A, SEQ ID NO: 22):
MRVMGIQRNCQHLLRWGTMILGMIIICSAAENLWVTVYYGVPVWKDAETTLFCASDAKAYETEMHNVWATHACVPTDPN
PQEIHLENVTEEFNMWKNNMVEQMHTDIISLWDQSLKPCVKLTPLCVTLNCSNVNVTNNTTNTHEEEIKNCSFNMTTEL
RDKKQKVYSLFYRLQVVQINENNSNSSYRLINCNTSAITQACPKVSFEPIPIHYCAPAGFAILKCKDKEFNGTGPCKNV
STVQCTHGIKPVVSTQLLLNGSLAEEEVIIRSENITNNAKTIIVQLTKPVKINCTRPNNNTRKSIRIGPGQAFYATGDI
IGDIRQAHCNVSRSEWNKTLQKVAKQLRKYFKNKTIIFTNSSGGDLEITTHSFNCGGEFFYCNTSGLFNSTWNNGTMKN
TITLPCRIKQIINMWQRAGQAMYAPPIQGVIRCESNITGLLLTRDGGNNNTNETFRPGGGDMRDNWRSELYKYKVVKIE
PLGVAPTRAKRRVVEREKRAVGIGAVFLGFLGAAGSTMGAASITLTVQARQLLSGIVQQQSNLLRAIEAQQHLLKLTVW
GIKQLQARVLAVERYLKDQQLLGIWGCSGKLICTTNVPWNSSWSNKSQNEIWDNMTWLQWDKEISNYTHIIYNLIEESQ
NQQEKNEQDLLALDKWANLWNWFDISNWLWYIKIFIMIVGGLIGLRIVFAVLSVINRVRQGYSPLSFQTHTPNPRGLDR
PGRIEEEGGEQGRDRSIRLVSGFLALAWDDLRSLCLFSYHRLRDFILIAARTVELLGHSSLKGLRLGWEGLKYLWNLLL
YWGRELKISAINLVDTIAIAVAGWTDRVIEIGQRIGRAILHIPRRIRQGLERALL
CONSENSUS_A2 (Clade A, SEQ ID NO: 23):
MRVMGTQRNYQHLWRWGILILGMLIMCKATDLWVTVYYGVPVWKDADTTLFCASDAKAYDTEVHNVWATHACVPTDPNP
QEVNLENVTEDFNMWKNNMVEQMHEDIISLWDQSLKPCVKLTPLCVTLNCSNANTTNNSTMEEIKNCSYNITTELRDKT
QKVYSLFYKLDVVQLDESNKSEYYYRLINCNTSAITQACPKVSFEPIPIHYCAPAGFAILKCKDPRFNGTGSCNNVSSV
QCTHGIKPVASTQLLLNGSLAEGKVMIRSENITNNAKNIIVQFNKPVPITCIRPNNNTRKSIRFGPGQAFYTNDIIGDI
RQAHCNINKTKWNATLQKVAEQLREHFPNKTIIFTNSSGGDLEITTHSFNCGGEFFYCNTTGLFNSTWKNGTTNNTEQM
ITLPCRIKQIINMWQRVGRAMYAPPIAGVIKCISNITGIILTRDGGNNETETFRPGGGDMRDNWRSELYKYKVVKIEPL
GVAPTRAKRRVVEREKRAVGMGAVFLGFLGAAGSTMGAASITLTVQARQLLSGIVQQQSNLLKAIEAQQHLLKLTVWGI
KQLQARVLALERYLQDQQLLGIWGCSGKLICATTVPWNSSWSNKTQEEIWNNMTWLQWDKEISNYTNIIYKLLEESQNQ
QEKNEQDLLALDKWANLWNWFNITNWLWYIRIFIMIVGGLIGLRIVIAIISVVNRVRQGYSPLSFQIPTPNPEGLDRPG
RIEEGGGEQGRDRSIRLVSGFLALAWDDLRSLCLFSYHRLRDCILIAARTVELLGHSSLXGLRLGWEGLKYLWNLLLYW
GRELKNSAISLLDTIAVAVAEWTDRVIEIGQRACRAILNIPRRIRQGFERALL
CONSENSUS_B (Clade B, SEQ ID NO: 24):
MRVKGIRKNYQHLWRWGTMLLGMLMICSAAEKLWVTVYYGVPVWKEATTTLFCASDAKAYDTEVHNVWATHACVPTDPN
PQEVVLENVTENFNMWKNNMVEQMHEDIISLWDQSLKPCVKLIPLCVTLNCTDLMNATNTNTTIIYRWRGEIKNCSFNI
TTSIRDKVQKEYALFYKLDVVPIDNDNTSYRLISCNTSVITQACPKVSFEPIPIHYCAPAGFAILKCNDKKFNGTGPCT
NVSTVQCTHGIRPVVSTQLLLNGSLAEEEVVIRSENFTDNAKTIIVQLNESVEINCTRPNNNTRKSIHIGPGRAFYTTG
EIIGDIRQAHCNISRAKWNNTLKQIVKKLREQFGNKTIVFNQSSGGDPEIVMHSFNCGGEFFYCNTTQLFNSTWNGTWN
NTEGNITLPCRIKQIINMWQEVGKAMYAPPIRGQIRCSSNITGLLLTRDGGNNETEIFRPGGGDMRDNWRSELYKYKVV
KIEPLGVAPTKAKRRVVQREKRAVGIGAMFLGFLGAAGSTMGAASMTLTVQARQLLSGIVQQQNNLLRAIEAQQHLLQL
TVWGIKQLQARVLAVERYLKDQQLLGIWGCSGKLICTTAVPWNASWSNKSLDEIWDNMTWMEWEREIDNYTSLIYTLIE
ESQNQQEKNEQELLELDKWASLWNWFDITNWLWYIKIFIMIVGGLVGLRIVFAVLSIVNRVRQGYSPLSFQTRLPAPRG
PDRPEGIEEEGGERDRDRSGRLVDGFLALIWDDLRSLCLFSYHRLRDLLLIVTRIVELLGRRGWEVLKYWWNLLQYWSQ
ELKNSAVSLLNATAIAVAEGTDRVIEVVQRACRAILHIPRRIRQGLERALL
CONSENSUS_C (Clade C, SEQ ID NO: 25):
MRVRGILRNCQQWWIWGILGFWMLMICNVVGNLWVTVYYGVPVWKEAKTTLFCASDAKAYEKEVHNVWATHACVPTDPN
PQEIVLENVTENFNMWKNDMVDQMHEDIISLWDQSLKPCVKLIPLCVTLNCTNATNATNTMGEIKNCSFNITTELRDKK
QKVYALFYRLDIVPLNENNSYRLINCNTSAITQACPKVSFDPIPIHYCAPAGYAILKCNNKTFNGTGPCNNVSTVQCTH
GIKPVVSTQLLLNGSLAEEEIIIRSENLTNNAKTIIVHLNESVEIVCTRPNNNTRKSIRIGPGQTFYATGDIIGDIRQA
HCNISEDKWNKTLQKVSKKLKEHFPNKTIKFEPSSGGDLEITTHSFNCRGEFFYCNTSKLFNSTYNSINSTITLPCRIK
QIINMWQEVGRAMYAPPIAGNITCKSNITGLLLTRDGGKNNTETFRPGGGDMRDNWRSELYKYKVVEIKPLGIAPTKAK
RRVVEREKRAVGIGAVFLGFLGAAGSTMGAASITLTVQARQLLSGIVQQQSNLLRAIEAQQHMLQLTVWGIKQLQTRVL
AIERYLKDQQLLGIWGCSGKLICTTAVPWNSSWSNKSQEDIWDNMTWMQWDREISNYTDTIYRLLEDSQNQQEKNEKDL
LALDSWKNLWNWFDITNWLWYIKIFIMIVGGLIGLRIIFAVLSIVNRVRQGYSPLSFQTLTPNPRGPDRLGRIEEEGGE
QDRDRSIRLVSGFLALAWDDLRSLCLFSYHRLRDFILIAARAVELLGRSSLRGLQRGWEALKYLGSLVQYWGLELKKSA
ISLLDTIAIAVAEGTDRIIELIQRICRAIRNIPRRIRQGFEAALQ
CONSENSUS_D (Clade D, SEQ ID NO: 26):
MRVRGIQRNYQHLWRWGIMLLGMLMICSVAENLWVTVYYGVPVWKEATTTLFCASDAKSYKTEAHNIWATHACVPTDPN
PQEIELENVTENFNMWKNNMVEQMHEDIISLWDQSLKPCVKLIPLCVTLNCTDVKRNNTSNDTNEGEMKNCSFNITTEI
RDKKKQVHALFYKLDVVPIDDNNSNTSYRLINCNTSAITQACPKVTFEPIPIHYCAPAGFAILKCKDKKFNGTGPCKNV
STVQCTHGIRPVVSTQLLLNGSLAEEEIIIRSENLTNNAKIIIVQLNESVTINCTRPYNNTRQRTPIGPGQALYTTRIK
GDIRQAHCNISRAEWNKTLQQVAKKLGDLLNKITIIFKPSSGGDPEITTHSFNCGGEFFYCNTSRLFNSTWNNTKWNST
GKITLPCRIKQIINMWQGVGKAMYAPPIEGLIKCSSNITGLLLTRDGGANNSHNETFRPGGGDMRDNWRSELYKYKVVK
IEPLGVAPTRAKRRVVEREKRAIGLGAMFLGFLGAAGSTMGAASMTLTVQARQLLSGIVQQQNNLLRAIEAQQHLLQLT
VWGIKQLQARILAVERYLKDQQLLGIWGCSGKHICTTTVPWNSSWSNKSLDEIWNNMTWMEWEREIDNYTGLIYSLIEE
SQNQQEKNEQELLELDKWASLWNWFSITQWLWYIKIFIMIVGGLIGLRIVFAVLSLVNRVRQGYSPLSFQTLLPAPRGP
DRPEGIEEEGGEQGRGRSIRLVNGFSALIWDDLRNLCLFSYHRLRDLILIAARIVELLGRRGWEALKYLWNLLQYWIQE
LKNSAISLFDTTAIAVAEGTDRVIEIVQRACRAILNIPTRIRQGLERALL
CONSENSUS_F1 (Clade F, SEQ ID NO: 27):
MRVRGMQRNWQHLGKWGLLFLGILIICNAAENLWVTVYYGVPVWKEATTTLFCASDAKSYEKEVHNVWATHACVPTDPN
PQEVVLENVTENFDMWKNNMVEQMHTDIISLWDQSLKPCVKLTPLCVTLNCTDVNATNNDTNDNKTGAIQNCSFNMTTE
VRDKKLKVHALFYKLDIVPISNNNSKYRLINCNTSTITQACPKVSWDPIPIHYCAPAGYAILKCNDKRFNGTGPCKNVS
TVQCTHGIKPVVSTQLLLNGSLAEEDIIIRSQNISDNAKTIIVHLNESVQINCTRPNNNTRKSIHLGPGQAFYATGEII
GDIRKAHCNISGTQWNKTLEQVKAKLKSHFPNKTIKFNSSSGGDLEITMHSFNCRGEFFYCNTSGLFNDTGSNGTITLP
CRIKQIVNMWQEVGRAMYAAPIAGNITCNSNITGLLLTRDGGQNNTETFRPGGGNMKDNWRSELYKYKVVEIEPLGVAP
TKAKRQVVKRERRAVGIGAVFLGFLGAAGSTMGAASITLTVQARQLLSGIVQQQNNLLRAIEAQQHLLQLTVWGIKQLQ
ARVLAVERYLKDQQLLGLWGCSGKLICTTNVPWNSSWSNKSQDEIWNNMTWMEWEKEISNYSNIIYRLIEESQNQQEKN
EQELLALDKWASLWNWFDISNWLWYIKIFIMIVGGLIGLRIVFAVLSIVNRVRKGYSPLSLQTLIPSPREPDRPEGIEE
GGGEQGKDRSVRLVNGFLALVWDDLRNLCLFSYRHLRDFILIAARIVDRGLRRGWEALKYLGNLTQYWSQELKNSAISL
LNTTAIVVAEGTDRVIEALQRAGRAVLNIPRRIRQGLERALL
CONSENSUS_F2 (Clade F, SEQ ID NO: 28):
MRVREMQRNWQHLGKWGLLFLGILIICNAADNLWVTVYYGVPVWKEATTTLFCASDAKAYEREVHNVWATYACVPTDPS
PQELVLGNVTENFNMWKNNMVDQMHEDIISLWDQSLKPCVKLTPLCVTLNCTDVNVTINTTNVTLGEIKNCSFNITTEI
KDKKKKEYALFYRLDVVPINNSIVYRLISCNTSTVTQACPKVSFEPIPIHYCAPAGFAILKCNDKKFNGTGLCRNVSTV
QCTHGIRPVVSTQLLLNGSLAEEDIIIRSENISDNTKTIIVQFNRSVEINCTRPNNNTRKSIRIGPGRAFYATGDIIGD
IRKAYCNINRTLWNETLKKVAEEFKNHFNITVTFNPSSGGDLEITTHSFNCRGEFFYCNTSDLFNNTEVNNTKTITLPC
RIRQFVNMWQRVGRAMYAPPIAGQIQCNSNITGLLLTRDGGKNGSETLRPGGGDMRDNWRSELYKYKVVKIEPLGVAPT
KAKRQVVQREKRAVGIGAVLLGFLGAAGSTMGAASITLTVQARQLLSGIVQQQSNLLKAIEAQCHLLQLTVWGIKQLQA
RILAVERYLKDQQLLGIWGCSGKLICTTNVPWNSSWSNKSQDEIWDNMTWMQWEKEISNYTDTIYRLIEDAQNQQEKNE
QDLLALDKWDNLWSWFTITNWLWYIKIFIMIVGGLIGLRIVFAVLSVVNRVRQGYSPLSLQTLIPNPRGPERPGGIEEE
GGEQDRDRSIRLVSGFLALAWDDLRSLCLFSYRHLRDFILIAARTVDMGLKRGWEALKYLWNLPQYWGQELKNSAISLL
DTTAIAVAEGTDRIIEVLQRAGRAVLHIPRRIRQGFERALL
JRFL (Clade B, SEQ ID NO: 29):
MRVKGIRKSYQYLWKGGTLLLGILMICSAVEKLWVTVYYGVPVWKEATTTLFCASDAKAYDTEVHNVWATHACVPTDPN
PQEVVLENVTEHFNMWKNNMVEQMQEDIISLWDQSLKPCVKLIPLCVTLNCKDVNATNTTNDSEGTMERGEIKNCSFNI
TTSIRDEVQKEYALFYKLDVVPIDNNNTSYRLISCDTSVITQACPKISFEPIPIHYCAPAGFAILKCNDKTFNGKGPCK
NVSTVQCTHGIRPVVSTQLLLNGSLAEEEVVIRSDNFTNNAKTIIVQLKESVEINCTRPNNNTRKSIHIGPGRAFYTTG
EIIGDIRQAHCNISRAKWNDTLKQIVIKLREQFENKTIVFNHSSGGDPEIVMHSFNCGGEFFYCNSTQLFNSTWNNNTE
GSNNTEGNTITLPCRIKQIINMWQEVGKAMYAPPIRGQIRCSSNITGLLLTRDGGINENGTEIFRPGGGDMRDNWRSEL
YKYKVVKIEPLGVAPTKAKRRVVQREKRAVGIGAVFLGFLGAAGSTMGAASMTLTVQARLLLSGIVQQQNNLLRAIEAQ
QRMLQLTVWGIKQLQARVLAVERYLGDQQLLGIWGCSGKLICTTAVPWNASWSNKSLDRIWNNMTWMEWEREIDNYTSE
IYTLIEESQNQQEKNEQELLELDKWASLWNWFDITKWLWYIKIFIMIVGGLVGLRLVFTVLSIVNRVRQGYSPLSFQTL
LPAPRGPDRPEGIEEEGGERDRDRSGRLVNGFLALIWVDLRSLCLFSYHRLRDLLLTVTRIVELLGRRGWEVLKYWWNL
LQYWSQELKNSAVSLLNATAIAVAEGTDRIIEALQRTYRAILHIPTRIRQGLERALL
BaL (Clade B, SEQ ID NO: 30):
MRVKEKYQHLWRWGWRWGTMLLGMLMICSATEKLWVTVYYGVPVWKEATTTLFCASDAKAYDTEVHNVWATHACVPTDP
NPQEVELENVTENFNMWKNNMVEQMHEDIISLWDQSLKPCVKLTPLCVTLNCTDLRNATNGNDTNTTSSSREMMGGGEM
KNCSFKITTNIRGKVQKEYALFYKLDIVPIDNNSNNRYRLISCNTSVITQACPKISFEPIPIHYCAPAGFAILKCKDKK
FNGKGPCSNVSTVQCTHGIRPVVSTQLLLNGSLAEEEVVIRSENFADNAKTIIVQLNESVEINCTRPNNNTRKSIHIGP
GRALYTTGKIIGDIRQAHCNLSRAKWNDTLNKIVIKLREQFGNKTIVFKHSSGGDPEIVTHSFNCGGEFFYCNSTQLFN
STWNVTEESNNTVENNTITLPCRIKQIINMWQKVGRAMYAPPIRGQIRCSSNITGLLLTRDGGPEDNKTEVFRPGGGDM
RDNWRSELYKYKVVKIEPLGVAPTKAKRRVVQREKRAVGIGAVFLGFLGAAGSTMGAASMTLTVQARLLLSGIVQQQNN
LLRAIEAQQHLLQLTVWGIKQLQARVLAVERYLRDQQLLGIWGCSGKLICTTAVPWNASWSNKSLNKIWDNMTWMEWDR
EINNYTSIIYSLIEESQNQQEKNEQELLELDKWASLWNWFEITEWLWYIKIFIMIIGGLIGLRIVFSVLSIMNRVRQGY
SPLSFQTHLPASRGPDRPGGIEEEGGERDRDRSGRLVNGSLALIWDDLRSLCLFSYHRLRDLLLIVTRIVELLGRRGWE
ALKYWWNLLQYWSQELKNSAVSLLNATAIAVAEGTDRVIEVVQGACRAIRHIPRRIRQGLERILL
BG505 (Clade A, SEQ ID NO: 31)
MRVMGIQRNCQHLFRWGTMILGMIIICSAAENLWVTVYYGVPVWKDAETTLFCASDAKAYETEKHNVWATHACVPTQPN
PQEIHLENVTEEFNMWKNNMVEQMHTDIISLWDQSLKPCVKLTPLCVTLQCTNVTNNITQDMRGELKNCSFNMTTELRD
KKQKVYSLFYRLDVVQINENQGNRSNNSNKEYRLINCNTSAITQACPKVSFEPIPIHYCAPAGFAILKCKDKKFNGTGP
CPSVSTVQCTHGIKPVVSTQLLLNGSLAEEEVMIRSENITNNAKNILVQFNTPVQINCTRPNNNTRKSIRIGPGQAFYA
TGDIIGDIRQAHCTVSKATWNETLGKVVKQLRKHFGNNTIIRFANSSGGDLEVTTHSFNCGGEFFYCNTSGLFNSTWIS
NTSVQGSNSTGSNDSITLPCRIKQIINMWQRIGQAMYAPPIQGVIRCVSNITGLILTRDGGSINSTTETFRPGGGDMRD
NWRSELYKYKVVKIEPLGVAPTRAKRRVVGREKRAVGIGAVFLGFLGAAGSTMGAASMTLTVQARNLLSGIVQQQSNLL
RAIEAQQHLLKLTVWGIKQLQARVLAVERYLRDQQLLGIWGCSGKLICTTNVPWNSSWSNRNLSEIWDNMTWLQWDKEI
SNYTQIIYGLLEESQNQQEKNEQDLLALDKWASLWNWFDISNWLWYIKIFIMIVGGLIGLRIVFAVLSVIHRVRQGYSP
LSFQTHTPNPRGLDRPERIEEEDGEQDRGRSTRLVSGFLALAWDDLRSLCLFCYHRLRDFILIAARIVELLGHSSLKGL
RLGWEGLKYLWNLLAYWGRELKISAINLFDTIAIAVAEWTDRVIEIGQRLCRAFLHIPRRIRQGLERALL
HXB2 (Clade B, SEQ ID NO: 1)
MRVKEKYQHLWRWGWRWGTMLLGMLMICSATEKLWVTVYYGVPVWKEATTTLFCASDAKAYDTEVHNVWATHACVPTDP
NPQEVVLVNVTENFNMWKNDMVEQMHEDIISLWDQSLKPCVKLTPLCVSLKCTDLKNDTNTNSSSGRMIMEKGEIKNCS
FNISTSIRGKVQKEYAFFYKLDIIPIDNDTTSYKLTSCNTSVITQACPKVSFEPIPIHYCAPAGFAILKCNNKTFNGTG
PCTNVSTVQCTHGIRPVVSTQLLLNGSLAEEEVVIRSVNFTDNAKTIIVQLNTSVEINCTRPNNNTRKRIRIQRGPGRA
FVTIGKIGNMRQAHCNISRAKWNNTLKQIASKLREQFGNNKTIIFKQSSGGDPEIVTHSFNCGGEFFYCNSTQLFNSTW
FNSTWSTEGSNNTEGSDTITLPCRIKQIINMWQKVGKAMYAPPISGQIRCSSNITGLLLTRDGGNSNNESEIFRPGGGD
MRDNWRSELYKYKVVKIEPLGVAPTKAKRRVVQREKRAVGIGALFLGFLGAAGSTMGAASMTLTVQARQLLSGIVQQQN
NLLRAIEAQQHLLQLTVWGIKQLQARILAVERYLKDQQLLGIWGCSGKLICTTAVPWNASWSNKSLEQIWNHTTWMEWD
REINNYTSLIHSLIEESQNQQEKNEQELLELDKWASLWNWFNITNWLWYIKLFIMIVGGLVGLRIVFAVLSIVNRVRQG
YSPLSFQTHLPTPRGPDRPEGIEEEGGERDRDRSIRLVNGSLALIWDDLRSLCLFSYHRLRDLLLIVTRIVELLGRRGW
EALKYWWNLLQYWSQELKNSAVSLLNATAIAVAEGTDRVIEVVQGACRAIRHIPRRIRQGLERILL
WITO.33 (Clade B, SEQ ID NO: 32)
MKVMGTKKNYQHLWRWGIMLLGMLMMSSAAEQLWVTVYYGVPVWREANTTLFCASDAKAYDTEVHNVWATHACVPTDPN
PQEVVMGNVTEDFNMWKNNMVEQMHEDIISLWDQSLKPCVKLTPLCVTLHCTNVTISSTNGSTANVTMREEMKNCSFNT
TTVIRDKIQKEYALFYKLDIVPIEGKNTNTGYRLINCNTSVITQACPKVSFEPIPIHYCAPAGFAILKCNNKTFNGKGP
CRNVSTVQCTHGIKPVVSTQLLLNGSLAEEDIIIRSENFTNNGKNIIVQLKEPVKINCTRPGNNTRRSINIGPGRAFYA
TGAIIGDIRKAHCNISTEQWNNTLTQIVDKLREQFGNKTIIFNQSSGGDPEVVMHTFNCGGEFFYCNSTQLFNSTWFNN
GTSTWNSTADNITLPCRIKQVINMWQEVGKAMYAPPIRGQIDCSSNITGLILTRDGGSNSSQNETFRPGGGNMKDNWRS
ELYKYKVVKIEPLGIAPTRAKRRVVQREKRAVTLGAVFLGFLGAAGSTMGAASLTLTVQARLLLSGIVQQQSNLLRAIE
AQQHMLQLTVWGIKQLQARVLAIERYLKDQQLLGIWGCSGKLICTTTVPWNTSWSNKSYDYIWNNMTWMQWEREIDNYT
GFIYTLIEESQNQQEKNELELLELDKWASLWNWFNITNWLWYIKLFIMIIGGLVGLRIVCAVLSIVNRVRQGYSPLSFQ
TRLPNPRGPDRPEETEGEGGERDRDRSARLVNGFLAIIWDDLRSLCLFSYHRLRDLLLIVARVVEILGRRGWEILKYWW
NLLKYWSQELKNSAVSLLNVTAIAVAEGTDRVIEIVQRAVRAILHIPTRIRQGFERALL
DU422 (Clade C, SEQ ID NO: 33):
MRVRGIPRNWPQWWIWGILGFWMIIICRVVGNLDLWVTVYYGVPVWKEAKTTLFCASDAKAYDKEVHNVWATHACVPTD
PNPQEIVLENVTENFNMWKNDMVDQMHEDIISLWDQSLKPCVKLTPLCVTLNCKNVNISANANATATLNSSMNGEIKNC
SFNTTTELRDKKQKVYALFYKPDVVPLNGGEHNETGEYILINCNSSTITQACPKVSFDPIPIHYCAPAGYAILKCNNKT
FNGTGPCNNVSTVQCTHGIKPVVSTQLLLNGSLAEEEIIVRSENLTNNIKTIIVHLNKSVEIKCTRPNNNTRKSVRIGP
GQTFYATGEIIGDIREAHCNISRETWNSTLIQVKEKLREHYNKTIKFEPSSGGDLEVTTHSFNCRGEFFYCDTTKLFNE
TKLFNESEYVDNKTIILPCRIKQIINMWQEVGRAMYAPPIEGNITCKSNITGLLLTWDGGENSTEGVFRPGGGNMKDNW
RSELYKYKVVEIKPLGVAPTKSKRKVVGREKRAVGLGAVLLGFLGAAGSTMGAASITLTVQARQLLSGIVQQQSNLLRA
IEAQQHLLQLTVWGIKQLQTRVLAIERYLKDQQLLGLWGCSGKLICATAVPWNSSWSNKSLGDIWDNMTWMQWDREISN
YTNTIFRLLEDSQNQQEKNEKDLLALDSWKNLWNWFDITNWLWYIKIFIMIVGGLIGLRIIFGVLAIVKRVRQGYSPLS
FQTLIPNPRGPDRLGRIEEEGGEQDKDRSIRLVSGFLALAWDDLRSLCLFSYHQLRDFILTAARAAELLGRSSLRGLQR
GWEVLKYLGNLVQYWGLELKRSAINLFDTIAIAVAEGTDRIIEVIQRICRAIRYIPTRIRQGFEAALL
CAP256.SU (Clade C, SEQ ID NO: 34)
MTVTGTWRNYQQWWIWGILGFWMLMICNGLWVTVYYGVPVWREAKTTLFCASDAKSYEKEVHNVWATHACVPTDPNPQE
LVLKNVTENFNMWKNDMVDQMHEDIISLWDQSLKPCVKLTPLCVTLNCSDAKVNINATYNGTREEIKNCSFNATTELRD
KKKKEYALFYRLDIVPLNKEGNNNSEYRLINCNTSVITQACPKVTFDPIPIHYCAPAGYAILKCNNKTFNGTGPCNNVS
TVQCTHGIKPVVSTQLLLNGSLAEEEIIIRSENLTDNVKTIIVHLNESVEINCTRPNNNTRKSIRIGPGQTFYATGDII
GDIRQAHCNISEIKWEKTLQRVSEKLREHFNKTIIFNQSSGGDLEITTHSFNCGGEFFYCNTSDLFFNKTFDETYSTGS
NSTNSTITLPCRIKQIINMWQEVGRAMYASPIAGEITCKSNITGLLLTRDGGGNNSTEETFRPGGGNMRDNWRSELYKY
KVVEVKPLGIAPTEARRRVVQKEKRAVVGLGAVFLGFLGTAGSTMGAASITLTVQARQLLSGIVQQQSNLLRAIEAQQH
MLQLTVWGIKQLQARVLAIERYLKDQQLLGMWGCSGKLICTTNVYWNSSWSNKTYNEIWDNMTWMQWDREIDNYTDTIY
KLLEVSQKQQESNEKDLLALDSWNNLWNWFDISKWLWYIKIFIMIVGGLIGLRIIFAVLSLVNRVRQGYSPLSFQTLTP
NPRELDRLGGIEEEGGEQDRDRSIRLVSGFFSLAWNDLRSLCLFCYHRLRDFILIAGRAVELLGRSSLQGLQRGWEILK
YLGSLVQYWGLELKKSAINLFDTIAIAVAEGTDRIIEFLQRIVRAILHIPRRIRQGFEAALQ

In some implementations, the protomers of the soluble HIV-1 Env ectodomain trimer comprise or consist of an amino acid sequence at least 95% (such as at least 96%, at least 97%, at least 98%, or at least 99%) identical to any one of SEQ ID NOs: 2-10, or any one of SEQ ID NOs: 35-150. In some implementations, the protomers of the soluble HIV-1 Env ectodomain trimer comprise or consist of SEQ ID NOs: 2-10, or any one of SEQ ID NOs: 35-150.

- BG505.W6M.C2_RnS7Mutv1-DS-SOSIP-2G-3Mut
SEQ ID NO: 2
AENLWVTVYYGVPVWKDAETTLFCASDAKAYETEKHNVWATHACVPTDPN
PQEIHLENVTEEFNMWKNNMVEQMHTDIISLWDQSLKPCVKLTPLCVTLQ
CTNVTNNITDDMRGELKNCSFNMTTELRDKKQKVYSLFYRLDVVQINENQ
GNRSNNSNKEYRLINCNTSAcTQiCPKVSFEPIPIHYCAPAGFAILKCKD
KKFNGTGPCknVSTVQCTHGIKPVVSTQLLLNGSLAEEEVMIRSENITNN
AKNIiVQFNTPVQINCTRPNNmTRKSIRGPGQAFYAlGDIIGDIRQpHCT
VSKATWNETLGKVVKQLRKHFGNNTIIfFANSSGGDLEVTTHSFNCGGEF
FYCNTSGLFNSTWISNTSVQGSNSTGSNDSITLPCRIKQIINMWQRvGQc
MYAPPIQGVIRCVSNITGLILTRDGGSTNSTTETFRPGGGDMRDNWRSEL
YKYKVVKIEPLGVAPTRcKRRVVGrrrrrrAVGIGAVFLGFLGAAGSTMG
AASnTLTVQARqLLSGIVQQQSNLLRApEAQQHLLKLgVWGfKQLQARVL
AVERYLevQQLLGIWGCSGKLICcTNVPWNSSWSNRNLSEIWDNMTWLQW
DKEIgNYTQIIYGLLEESQfQQEiNEQDLLALD
- BG505.W6M.C2_RnS5Mut-DS-SOSIP-2G-3Mut_K502N-
R504T-L660N-A662T
SEQ ID NO: 3
AENLWVTVYYGVPVWKDAETTLFCASDAKAYETEKHNVWATHACVPTDPN
PQEIHLENVTEEFNMWKNNMVEQMHTDIISLWDQSLKPCVKLTPLCVTLQ
CTNVTNNITDDMRGELKNCSFNMTTELRDKKQKVYSLFYRLDVVQINENQ
GNRSNNSNKEYRLINCNTSAcTQACPKVSFEPIPIHYCAPAGFAILKCKD
KKFNGTGPCkNVSTVQCTHGIKPVVSTQLLLNGSLAEEEVMIRSENITNN
AKNIiVQFNTPVQINCTRPNNmTRKSIRIGPGQAFYAlGDIIGDIRQpHC
TVSKATWNETLGKVVKQLRKHFGNNTIIfFANSSGGDLEVTTHSFNCGGE
FFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSE
LYKYKVVKIEPLGVAPTRcnRtVVGrrrrrrAVGIGAVFLGFLGAAGSTM
GAASnTLTVQARqLLSGIVQQQSNLLRApEAQQHLLKLgVWGIKQLQARV
LAVERYLevQQLLGIWGCSGKLICcTNVPWNSSWSNRNLSEIWDNMTWLQ
WDKEIgNYTQIIYGLLEESQfQQEiNEQDnLtLD
- JCB-7mutv1_668-772-778-782
SEQ ID NO: 4
AENLWVTVYYGVPVWKDAETTLFCASDAKAYETEKHNVWATHACVPTDPN
PQEIHLENVTEEFNMWKNNMVEQMHTDIISLWDQSLKPCVKLTPLCVTLQ
CTNVTNNITDDMRGELKNCSFNMTTELRDKKQKVYSLFYRLDVVQINENQ
GNRSNNSNKEYRLINCNTSAcTQiCPKVSFEPIPIHYCAPAGFAILKCKD
KKFNGTGPCkSVSTVQCTHGIKPVVSTQLLLNGSLAEEEVMIRSENITNN
AKNIiVQFNTPVQINCTRPNNmTRKSIRIGPGQAFYAlGDIIGDIRQpHC
NVSKATWNETLGKVVKQLRKHFGNNTIIfFANSSGGDLEVTTHSFNCGGE
FFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSE
LYKYKVVKIEPLGVAPTRcnRtVVGrrrrrrAVGIGAVFLGFLGAAGSTM
GAASnTLTVQARqLLSGIVQQQSNLLRApEAQQHLLKLgVWGfKQLQARV
LAVERYLevQQLLGIWGCSGKLICcTNVPWNSSWSNRNLSEIWDNMTWLQ
WDKEIgNYTQIIYGLLEESQfQQEiNEQDnLtLDGGYnVtYnVtGGYnVt
YnVtIGGSGG
- JG2_7mut3_N656.659
SEQ ID NO: 5
AENLWVTVYYGVPVWKDAETTLFCASDAKAYETEKHNVWATHACVPTDPN
PQEIHLENVTEEFNMWKNNMVEQMHTDIISLWDQSLKPCVKLTPLCVTLQ
CTNVTNNITDDMRGELKNCSFNMTTELRDKKQKVYSLFYRLDVVQINENQ
GNRSNNSNKEYRLINCNTSAcTQiCPKVSFEPIPIHYCAPAGFAILKCKD
KKFNGTGPCkSVSTVQCTHGIKPVVSTQLLLNGSLAEEEVMIRSENITNN
AKNIiVQFNTPVQINCTRPNNmTRKSIRIGPGQAFYAlGDIIGDIRQpHC
NVSKATWNETLGKVVKQLRKHFGNNTIIfFANSSGGDLEVTTHSFNCGGE
FFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSE
LYKYKVVKIEPLGVAPTRcnRtVVGrrrrrrAVGIGAVFLGFLGAAGSTM
GAASnTLTVQARqLLSGIVQQQSNLLRApEAQQHLLKLgVWGfKQLQARV
LAVERYLevQQLLGIWGCSGKLICcTNVPWNSSWSNRNLSEIWDNMTWLQ
WDKEIgNYTQIIYGLLEESQfQQEiNEtD
- JCB-Mut7v1-3hsh01-G7-18
SEQ ID NO: 6
AENLWVTVYYGVPVWKDAETTLFCASDAKAYETEKHNVWATHACVPTDPN
PQEIHLENVTEEFNMWKNNMVEQMHTDIISLWDQSLKPCVKLTPLCVTLQ
CTNVTNNITDDMRGELKNCSFNMTTELRDKKQKVYSLFYRLDVVQINENQ
GNRSNNSNKEYRLINCNTSAcTQiCPKVSFEPIPIHYCAPAGFAILKCKD
KKFNGTGPCkSVSTVQCTHGIKPVVSTQLLLNGSLAEEEVMIRSENITNN
AKNIiVQFNTPVQINCTRPNNmTRKSIRIGPGQAFYAlGDIIGDIRQpHC
NVSKATWNETLGKVVKQLRKHFGNNTIIfFANSSGGDLEVTTHSFNCGGE
FFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSE
LYKYKVVKIEPLGVAPTRcnRtVVGrrrrrrAVGIGAVFLGFLGAAGSTM
GAASnTLTVQARqLLSGIVQQQSNLLRApEAQQHLLKLgVWGfKQLQARV
LAVERYLevQQLLGIWGCSGKLICcTNVPWNSSWSNRNLSEIWDNMTWLQ
WDKEIgNYTQIIYGLLEESQfQQEiNEQDnLtLDGGGSSGVRLWnTtKIM
LGQVHnVtKGWLIFVAEQEELYVRVQNGFRKVQLEARTPLPR
- 7Mutv1-Gly332-dGly241-base_glycan-Q617N_Q619T_
L504T
SEQ ID NO: 7
AENLWVTVYYGVPVWKDAETTLFCASDAKAYETEKHNVWATHACVPTDPN
PQEIHLENVTEEFNMWKNNMVEQMHTDIISLWDQSLKPCVKLTPLCVTLQ
CTNVTNNITDDMRGELKNCSFNMTTELRDKKQKVYSLFYRLDVVQINENQ
GNRSNNSNKEYRLINCNTSAcTQiCPKVSFEPIPIHYCAPAGFAILKCKD
KKFNGTGPCkSVSTVQCTHGIKPVVSTQLLLNGSLAEEEVMIRSENITNN
AKNIiVQFNTPVQINCTRPNNmTRKSIRIGPGQAFYAlGDIIGDIRQpHC
NVSKATWNETLGKVVKQLRKHFGNNTIIfFANSSGGDLEVTTHSFNCGGE
FFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSE
LYKYKVVKIEPLGVAPTRcnRtVVGrrrrrrAVGIGAVFLGFLGAAGSTM
GAASnTtTVQARqLLSGIVQQQSNLLRApEAQQHLLKLgVWGfKQLQARV
LAVERYLevQQLLGIWGCSGKLICcTNVPWNSSWSNRNLSEIWDNMTWLQ
WDKEIgNYTQIIYGLLEESnftQEiNEQDnLtLD
- VRC 7678 (6931_DS-N28-JCBv3-N660-N665.667)
(ConC based)
SEQ ID NO: 8
nStAENLWVTVYYGVPVWKEAKTTLFCASDAKAYEKEVHNVWATHACVPT
DPNPQEMVLENVTENFNMWKNDMVDQMHEDIISLWDQSLKPCVKLTPLCV
TLNCTNVNVTNTNNNNMKEEMKNCSFNTTTEIRDKKQKEYALFYRLDIVP
LNENSSEYRLINCNTSTcTQICPKVSFDPIPIHYCAPAGYAILKCNNKTF
NGTGPCNNVSTVQCTHGIKPVVSTQLLINGSLAEEEIIIRSENLTDNAKT
IIVHLNESVEINCTRPNNmTRKSIRIGPGQTFYAIGDIIGDIRQpHCNIS
EAKWNKTLQRVKKKLKEHFPNKTIKFAPSSGGDLEITTHSFNCRGEFFYC
NTSKLFNSTYNNTTSNSTITLPCRIKQIINMWQEVGRCMYAPPIAGnITC
KSNITGLLLTRDGGNNNNNTETFRPGGGDMRDNWRSELYKYKVVEIKPLG
IAPTKcnRtVVEnsthknlthhmrrrrrrAVGLGAVFLGFLGAAGSTMGA
ASNTLTVQARQLLSGIVQQQSNLLRAPEAQQHMLQLGVWGFKQLQARVLA
IERYLEVQQLLGIWGCSGKLICCTAVPWNSTWSNKTQEDIWDNMTWMQWD
REIGNYTDTIYRLLEESQFQQEINEKDNLTLPNST
- VRC 7873 (4571_RnS7Mutv1-2G-3Mut-N28-JCBv3-N660-
N665.667_613S_620S) (BG505-based)
SEQ ID NO: 9
NSTAENLWVTVYYGVPVWKDAETTLFCASDAKAYETEKHNVWATHACVPT
DPNPQEIHLENVTEEFNMWKNNMVEQMHTDIISLWDQSLKPCVKLTPLCV
TLQCTNVTNNITDDMRGELKNCSFNMTTELRDKKQKVYSLFYRLDVVQIN
ENQGNRSNNSNKEYRLINCNTSACTQICPKVSFEPIPIHYCAPAGFAILK
CKDKKFNGTGPCKSVSTVQCTHGIKPVVSTQLLLNGSLAEEEVMIRSENI
TNNAKNIIVQFNTPVQINCTRPNNMTRKSIRIGPGQAFYALGDIIGDIRQ
PHCNVSKATWNETLGKVVKQLRKHFGNNTIIFFANSSGGDLEVTTHSFNC
GGEFFYCNTSGLFNSTWISNTSVQGSNSTGSNDSITLPCRIKQIINMWQR
VGQCMYAPPIQGVIRCVSNITGLILTRDGGSTNSTTETFRPGGGDMRDNW
RSELYKYKVVKIEPLGVAPTRCNRTVVGNSTHKNLTHHMRRRRRRAVGIG
AVFLGFLGAAGSTMGAASNTLTVQARQLLSGIVQQQSNLLRAPEAQQHLL
KLGVWGFKQLQARVLAVERYLEVQQLLGIWGCSGKLICCTNVPWNSSWSN
RNLSEIWDNMTWLQWDKEIGNYTQIIYGLLEESQFQQEINEQDNLTLPNS
T
- VRC 7677 (BG505-based)
SEQ ID NO: 10
nStAENLWVTVYYGVPVWKDAETTLFCASDAKAYETEKHNVWATHACVPT
DPNPQEIHLENVTEEFNMWKNNMVEQMHTDIISLWDQSLKPCVKLTPLCV
TLQCTNVTNNITDDMRGELKNCSFNMTTELRDKKQKVYSLFYRLDVVQIN
ENQGNRSNNSNKEYRLINCNTSAcTQICPKVSFEPIPIHYCAPAGFAILK
CKDKKFNGTGPCKSVSTVQCTHGIKPVVSTQLLLNGSLAEEEVMIRSENI
TNNAKNIIVQFNTPVQINCTRPNNmTRKSIRIGPGQAFYAlGDIIGDIRQ
pHCNVSKATWNETLGKVVKQLRKHFGNNTIIFFANSSGGDLEVTTHSFNC
GGEFFYCNTSGLFNSTWISNTSVQGSNSTGSNDSITLPCRIKQIINMWQR
VGQCMYAPPIQGVIRCVSNITGLILTRDGGSTNSTTETFRPGGGDMRDNW
RSELYKYKVVKIEPLGVAPTRcnRtVVGnsthknlthhmrrrrrrAVGIG
AVFLGFLGAAGSTMGAASNTLTVQARQLLSGIVQQQSNLLRAPEAQQHLL
KLGVWGFKQLQARVLAVERYLEVQQLLGIWGCSGKLICCTNVPWNSTWSN
RNLTEIWDNMTWLQWDKEIGNYTQIIYGLLEESQFQQEINEQDNLTLPNS
T

VRC7873 is identical to VRC7677 except for T613S and T620S substitutions.

In the protomers of the purified trimer, the Env protein typically does not include a signal peptide, as the signal peptide is proteolytically cleaved during cellular processing. In implementations including a soluble HIV-1 Env ectodomain trimer, the gp41 ectodomain is not linked to a transmembrane domain or other membrane anchor.

Stabilization of the soluble HIV-1 Env ectodomain trimer in the prefusion closed conformation reduces transition of the HIV-1 Env ectodomain to the CD4-bound open conformation. Thus, soluble HIV-1 Env ectodomain trimers that are stabilized in this conformation can be specifically bound by an antibody that is specific for the prefusion closed conformation of HIV-1 Env (e.g., VRC26, PGT151, PGT122, or PGT145), but are not specifically bound by an antibody specific for the CD4-bound open conformation, of HIV-1 Env (e.g., 17b mAb in the presence of sCD4). Methods of determining if a soluble HIV-1 Env ectodomain trimer includes a CD4-induced epitope are described, for example, in PCT App. No. PCT/US2015/048729. For example, the antibody binding assay can be conducted in the presence of a molar excess of soluble CD4 as described in Sanders et al. (Plos Pathogens, 9, e1003618, 2013).

In several implementations, the soluble HIV-1 Env ectodomain trimers can be specifically bound by an antibody that specifically binds to the V1V2 domain on a HIV-1 Env trimer, but not an Env monomer. Exemplary antibodies with such antigen binding characteristics include the PGT141, PGT142, PGT143, PGT144, PGT145, and VRC26 antibodies. Additional examples include the PG9, PG16, and CH01-CH04 antibodies. Accordingly, in some implementations the soluble HIV-1 Env ectodomain trimer specifically binds to an antibody (such as a PGT141, PGT142, PGT143, PGT144, PGT145, and VRC26 antibody) that specifically binds to the V1V2 domain of a HIV-1 Env in its trimeric, but not monomeric, form with a dissociation constant of less than 10⁻⁶ Molar, such as less than 10⁻⁷ Molar, less than 10⁻⁸ Molar, or less than 10⁻⁹ Molar. Specific binding can be determined by methods known in the art. The determination of specific binding may readily be made by using or adapting routine procedures, such as ELISA, immunocompetition, surface plasmon resonance, or other immunosorbant assays (described in many standard texts, including Greenfield, Antibodies, A Laboratory Manual, 2nd ed. Cold Spring Harbor Laboratory Press, New York (2014).

Several implementations include a multimer of the soluble HIV-t Env ectodomain trimer, for example, a multimer including 2, 3, 4, 5, 6, 7, 8, 9, or 10, or more of the soluble HIV-1 Env ectodomain trimers or immunogenic fragment thereof.

In view of the conservation and breadth of knowledge of HIV-1 Env sequences, corresponding HIV-1 Env amino acid positions between different HIV-1 Env strains and subtypes can be readily identified. The HXB2 numbering system has been developed to assist comparison between different HIV-1 amino acid and nucleic acid sequences (see, e.g., Korber et al., Human Retroviruses and AIDS 1998: A Compilation and Analysis of Nucleic Acid and Amino Acid Sequences. Korber B, Kuiken C L, Foley B, Hahn B, McCutchan F, Mellors J W, and Sodroski J, Eds. Theoretical Biology and Biophysics Group, Los Alamos National Laboratory, Los Alamos, NM, which is incorporated by reference herein in its entirety). The numbering of amino acid substitutions disclosed herein is made according to the HXB2 numbering system, unless context indicates otherwise.

It is understood that some variations can be made in the amino acid sequence of a protein without affecting the activity of the protein. Such variations include insertion of amino acid residues, deletions of amino acid residues, and substitutions of amino acid residues. These variations in sequence can be naturally occurring variations or they can be engineered through the use of genetic engineering techniques. Examples of such techniques are found in see, e.g., Sambrook et al. (Molecular Cloning: A Laboratory Manual, 4^{th} ed, Cold Spring Harbor, New York, 2012) and Ausubel et al. (In Current Protocols in Molecular Biology, John Wiley & Sons, New York, through supplement 104, 2013, both of which are incorporated herein by reference in their entirety.

The HIV-1 Env ectodomain trimer provided herein is soluble in aqueous solution. In some implementations, the HIV-1 Env ectodomain trimer dissolves to a concentration of at least 0.5 mg/ml (such as at least 1.0 mg/ml, 1.5 mg/ml, 2.0 mg/ml, 3.0 mg/ml, 4.0 mg/ml or at least 5.0 mg/ml) in phosphate buffered saline (pH 7.4) at room temperature (e.g., 20-22 degrees Celsius) and remains dissolved for at least for at least 12 hours (such as at least 24 hours, at least 48 hours, at least one week, at least two weeks, or more time). In one implementation, the phosphate buffered saline includes NaCl (137 mM), KCl (2.7 mM), Na₂HPO₄ (10 mM), KH₂PO₄ (1.8 mM) at pH 7.4. In some implementations, the phosphate buffered saline further includes CaCl₂ (1 mM) and MgCl2 (0.5 mM). Determining if a protein remains in solution over time can be accomplished using appropriate techniques. For example, the concentration of the protein dissolved in an aqueous solution can be tested over time using standard methods.

The soluble HIV-1 Env ectodomain trimer can be derivatized or linked to another molecule (such as another peptide or protein). In general, the soluble HIV-1 Env ectodomain trimer is derivatized such that the binding to broadly neutralizing antibodies to the trimer is not affected adversely by the derivatization or labeling. For example, the soluble HIV-1 Env ectodomain trimer can be functionally linked (by chemical coupling, genetic fusion, noncovalent association or otherwise) to one or more other molecular entities, such as an antibody or protein or detection tag.

### Carrier Molecules

In some implementations, a disclosed HIV-1 Env ectodomain trimer can be linked to a carrier protein by a linker (such as a peptide linker) or can be directly linked to the carrier protein (for example, by conjugation, or synthesis as a fusion protein) too form an immunogenic conjugate.

Suitable linkers are well known to those of skill in the art and include, but are not limited to, straight or branched-chain carbon linkers, heterocyclic carbon linkers or peptide linkers. One skilled in the art will recognize, for an immunogenic conjugate from two or more constituents, each of the constituents will contain the necessary reactive groups. Representative combinations of such groups are amino with carboxyl to form amide linkages or carboxy with hydroxyl to form ester linkages or amino with alkyl halides to form alkylamino linkages or thiols with thiols to form disulfides or thiols with maleimides or alkylhalides to form thioethers. Hydroxyl, carboxyl, amino and other functionalities, where not present may be introduced by known methods. Likewise, as those skilled in the art will recognize, a wide variety of linking groups may be employed. In some cases, the linking group can be designed to be either hydrophilic or hydrophobic in order to enhance the desired binding characteristics of the HIV-1 Env ectodomain trimer and the carrier. The covalent linkages should be stable relative to the solution conditions under which the conjugate is subjected.

In some implementations, the linkers may be joined to the constituent amino acids through their side groups (such as through a disulfide linkage to cysteine) or to the alpha carbon amino and carboxyl groups of the terminal amino acids. In some implementations, the HIV-1 Env ectodomain protomer, the linker, and the carrier can be encoded as a single fusion polypeptide such that the HIV-1 Env ectodomain protomer and the carrier are joined by peptide bonds.

The procedure for attaching a molecule to a polypeptide varies according to the chemical structure of the molecule. Polypeptides typically contain a variety of functional groups; for example, carboxylic acid (COOH), free amine (—NH₂) or sulfhydryl (—SH) groups, which are available for reaction with a suitable functional group on a polypeptide. Alternatively, the polypeptide is derivatized to expose or attach additional reactive functional groups. The derivatization may involve attachment of any of a number of linker molecules such as those available from Pierce Chemical Company, Rockford, IL.

It can be advantageous to produce conjugates in which more than one HIV-1 Env ectodomain trimer is conjugated to a single carrier protein. In several implementations, the conjugation of multiple HIV-1 Env ectodomain trimers to a single carrier protein is possible because the carrier protein has multiple lysine or cysteine side-chains that can serve as sites of attachment. The amount of HIV-1 Env ectodomain trimer reacted with the amount of carrier may vary depending upon the specific HIV-1 Env ectodomain trimer and the carrier protein. However, the respective amounts should be sufficient to introduce from 1-30 chains of HIV-1 Env ectodomain trimer onto the carrier protein. The resulting number of HIV-1 Env ectodomain trimer linked to a single carrier molecule may vary depending upon the specific HIV-1 Env ectodomain trimer and the carrier protein. In some implementations, from 1 to 30, such as about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 HIV-1 Env ectodomain trimer can be linked to each carrier protein molecule. “About” in this context refers to plus or minus 5% when measuring an average number of HIV-1 Env ectodomain trimer per carrier molecule in the conjugate. Thus, in some implementations, the average ratio of HIV-1 Env ectodomain trimer to carrier protein molecules is between about 1:1 and about 30:1, such as about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, or about 20:1, about 21:1, about 22:1, about 23:1, about 24:1, about 25:1, about 26:1, about 27:1, about 28:1, about 29:1, or about 30:1, for example, between about 1:1 and about 15:1, between about 5:1 and about 20:1, or between about 10:1 and about 30:1.

In some implementations (such as when KLH is used as a carrier), from 1 to 1000, such as about 50, about 100, about 200, about 300, about 400, about 500, about 700, about 1000, about 9, about 10, about 11, about 12, about 13, about 14, about 15, about 16, about 17, about 18, or about 19 HIV-1 Env ectodomain trimer molecules can be linked to each carrier protein. “About” in this context refers to plus or minus 5% when measuring an average number of HIV-1 Env ectodomain trimer molecules per carrier molecule in the conjugate. Thus, in some implementations, the average ratio of HIV-1 Env ectodomain trimer molecule to carrier protein is between about 1:1 and about 1000:1, such as between about 100:1 and about 500:1, between about 500:1 and about 10000:1, or between about 250:1 and about 750:1.

Examples of suitable carriers are those that can increase the immunogenicity of the conjugate and/or elicit antibodies against the carrier which are diagnostically, analytically, and/or therapeutically beneficial. Useful carriers include polymeric carriers, which can be natural, recombinantly produced, semi-synthetic or synthetic materials containing one or more amino groups, such as those present in a lysine amino acid residue present in the carrier, to which a reactant moiety can be attached. Carriers that fulfill these criteria are generally known in the art (see, for example, Fattom et al., Infect. Immun. 58:2309-12, 1990; Devi et al., PNAS 88:7175-79, 1991; Szu et al., Infect. Immun. 59:4555-61, 1991; Szu et al., J. Exp. Med. 166:1510-24, 1987; and Pavliakova et al., Infect. Immun. 68:2161-66, 2000). A carrier can be useful even if the antibody that it elicits is not of benefit by itself.

Specific, non-limiting examples of suitable polypeptide carriers include, but are not limited to, natural, semi-synthetic or synthetic polypeptides or proteins from bacteria or viruses. In one implementation, bacterial products for use as carriers include bacterial toxins. Bacterial toxins include bacterial products that mediate toxic effects, inflammatory responses, stress, shock, chronic sequelae, or mortality in a susceptible host. Specific, non-limiting examples of bacterial toxins include, but are not limited to: B. anthracis PA (for example, as encoded by bases 143779 to 146073 of GENBANK® Accession No. NC 007322); B. anthracis LF (for example, as encoded by the complement of bases 149357 to 151786 of GENBANK® Accession No. NC 007322); bacterial toxins and toxoids, such as tetanus toxin/toxoid (for example, as described in U.S. Pat. Nos. 5,601,826 and 6,696,065); diphtheria toxin/toxoid (for example, as described in U.S. Pat. Nos. 4,709,017 and 6,696,065), such as tetanus toxin heavy chain C fragment; P. aeruginosa exotoxin/toxoid (for example, as described in U.S. Pat. Nos. 4,428,931, 4,488,991 and 5,602,095); pertussis toxin/toxoid (for example, as described in U.S. Pat. Nos. 4,997,915, 6,399,076 and 6,696,065); and C. perfringens exotoxin/toxoid (for example, as described in U.S. Pat. Nos. 5,817,317 and 6,403,094) C. difficile toxin B or A, or analogs or mimetics of and combinations of two or more thereof. Viral proteins, such as hepatitis B surface antigen (for example, as described in U.S. Pat. Nos. 5,151,023 and 6,013,264) and core antigen (for example, as described in U.S. Pat. Nos. 4,547,367 and 4,547,368) can also be used as carriers, as well as proteins from higher organisms such as keyhole limpet hemocyanin (KLH), horseshoe crab hemocyanin, Concholepas Concholepas Hemocyanin (CCH), Ovalbumin (OVA), edestin, mammalian serum albumins (such as bovine serum albumin), and mammalian immunoglobulins. In some examples, the carrier is bovine serum albumin.

In some implementations, the carrier is selected from one of: Keyhole Limpet Hemocyanin (KLH), tetanus toxoid, tetanus toxin heavy chain C fragment, diphtheria toxoid, diphtheria toxin variant CRM197, or H influenza protein D (HiD). CRM197 is a genetically detoxified form of diphtheria toxin; a single mutation at position 52, substituting glutamic acid for glycine, causes the ADP-ribosyltransferase activity of the native diphtheria toxin to be lost. For description of protein carriers for vaccines, see Pichichero, Protein carriers of conjugate vaccines: characteristics, development, and clinical trials, Hum Vaccin Immunother., 9: 2505-2523,2013, which is incorporated by reference herein in its entirety).

Following conjugation of the HIV-1 Env ectodomain trimer to the carrier protein, the conjugate can be purified by a variety of techniques well known to one of skill in the art. One goal of the purification step is to separate the unconjugated HIV-1 Env ectodomain trimer or carrier from the conjugate. The conjugates can be purified away from unconjugated HIV-1 Env ectodomain trimer or carrier by any number of standard techniques including, for example, size exclusion chromatography, density gradient centrifugation, hydrophobic interaction chromatography, or ammonium sulfate fractionation. See, for example, Anderson et al., J. Immunol. 137:1181-86, 1986 and Jennings & Lugowski, J. Immunol. 127:1011-18, 1981. The compositions and purity of the conjugates can be determined by GLC-MS and MALDI-TOF spectrometry, for example.

In several implementations, the disclosed immunogenic conjugates can be formulated into immunogenic composition (such as vaccines), for example by the addition of a pharmaceutically acceptable carrier and/or adjuvant.

### III. Polynucleotides and Expression

Polynucleotides encoding a disclosed immunogen are also provided. These polynucleotides include DNA, cDNA and RNA sequences which encode the antigen. One of skill in the art can readily use the genetic code to construct a variety of functionally equivalent nucleic acids, such as nucleic acids which differ in sequence but which encode the same protein sequence, or encode a conjugate or fusion protein including the nucleic acid sequence.

In several implementations, the nucleic acid molecule encodes a precursor of a protomer of a disclosed HIV-1 Env trimer, that, when expressed in cells under appropriate conditions, forms HIV-1 Env trimers and is processed into the mature form of the HIV-1 Env protein.

In additional implementations, a nucleic acid molecule encoding a disclosed immunogen can be included in a viral vector, for example, for expression of the immunogen in a host cell, or for immunization of a subject. In some implementations, the viral vectors are administered to a subject as part of a prime-boost vaccination. In several implementations, the viral vectors are included in a vaccine, such as a primer vaccine or a booster vaccine for use in a prime-boost vaccination.

In several examples, the viral vector can be replication-competent. For example, the viral vector can have a mutation in the viral genome that does not inhibit viral replication in host cells. The viral vector also can be conditionally replication-competent. In other examples, the viral vector is replication-deficient in host cells.

A number of viral vectors have been constructed, that can be used to express the disclosed antigens, including polyoma, i.e., SV40 (Madzak et al., 1992, J. Gen. Virol., 73:15331536), adenovirus (Berkner, 1992, Cur. Top. Microbiol. Immunol., 158:39-6; Berliner et al., 1988, Bio Techniques, 6:616-629; Gorziglia et al., 1992, J. Virol., 66:4407-4412; Quantin et al., 1992, Proc. Natl. Acad. Sci. USA, 89:2581-2584; Rosenfeld et al., 1992, Cell, 68:143-155; Wilkinson et al., 1992, Nucl. Acids Res., 20:2233-2239; Stratford-Perricaudet et al., 1990, Hum. Gene Ther., 1:241-256), vaccinia virus (Mackett et al., 1992, Biotechnology, 24:495-499), adeno-associated virus (Muzyczka, 1992, Curr. Top. Microbiol. Immunol., 158:91-123; On et al., 1990, Gene, 89:279-282), herpes viruses including HSV and EBV (Margolskee, 1992, Curr. Top. Microbiol. Immunol., 158:67-90; Johnson et al., 1992, J. Virol., 66:29522965; Fink et al., 1992, Hum. Gene Ther. 3:11-19; Breakfield et al., 1987, Mol. Neurobiol., 1:337-371; Fresse et al., 1990, Biochem. Pharmacol., 40:2189-2199), Sindbis viruses (H. Herweijer et al., 1995, Human Gene Therapy 6:1161-1167; U.S. Pat. Nos. 5,091,309 and 5,2217,879), alphaviruses (S. Schlesinger, 1993, Trends Biotechnol. 11:18-22; I. Frolov et al., 1996, Proc. Natl. Acad. Sci. USA 93:11371-11377) and retroviruses of avian (Brandyopadhyay et al., 1984, Mol. Cell Biol., 4:749-754; Petropouplos et al., 1992, J. Virol., 66:3291-3297), murine (Miller, 1992, Curr. Top. Microbiol. Immunol., 158:1-24; Miller et al., 1985, Mol. Cell Biol., 5:431-437; Sorge et al., 1984, Mol. Cell Biol., 4:1730-1737; Mann et al., 1985, J. Virol., 54:401-407), and human origin (Page et al., 1990, J. Virol., 64:5370-5276; Buchschalcher et al., 1992, J. Virol., 66:2731-2739). Baculovirus (Autographa californica multinuclear polyhedrosis virus; AcMNPV) vectors are also known in the art, and may be obtained from commercial sources (such as PharMingen, San Diego, Calif.; Protein Sciences Corp., Meriden, Conn.; Stratagene, La Jolla, Calif.).

In several implementations, the viral vector can include an adenoviral vector that expresses a disclosed immunogen. Adenovirus from various origins, subtypes, or mixture of subtypes can be used as the source of the viral genome for the adenoviral vector. Non-human adenovirus (e.g., simian, chimpanzee, gorilla, avian, canine, ovine, or bovine adenoviruses) can be used to generate the adenoviral vector. For example, a simian adenovirus can be used as the source of the viral genome of the adenoviral vector. A simian adenovirus can be of serotype 1, 3, 7, 11, 16, 18, 19, 20, 27, 33, 38, 39, 48, 49, 50, or any other simian adenoviral serotype. A simian adenovirus can be referred to by using any suitable abbreviation known in the art, such as, for example, SV, SAdV, SAV or sAV. In some examples, a simian adenoviral vector is a simian adenoviral vector of serotype 3, 7, 11, 16, 18, 19, 20, 27, 33, 38, or 39. In one example, a chimpanzee serotype C Ad3 vector is used (see, e.g., Peruzzi et al., Vaccine, 27:1293-1300, 2009). Human adenovirus can be used as the source of the viral genome for the adenoviral vector. Human adenovirus can be of various subgroups or serotypes. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22, 23, 24, 25, 26, 27, 28, 29, 30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serotype. The person of ordinary skill in the art is familiar with replication competent and deficient adenoviral vectors (including singly and multiply replication deficient adenoviral vectors). Examples of replication-deficient adenoviral vectors, including multiply replication-deficient adenoviral vectors, are disclosed in U.S. Pat. Nos. 5,837,511; 5,851,806; 5,994,106; 6,127,175; 6,482,616; and 7,195,896, and International Patent Application Nos. WO 94/28152, WO 95/02697, WO 95/16772, WO 95/34671, WO 96/22378, WO 97/12986, WO 97/21826, and WO 03/02231 1.

### V. Immunogenic Compositions

Immunogenic compositions comprising a disclosed immunogen and a pharmaceutically acceptable carrier are also provided. Such compositions can be administered to subjects by a variety of administration modes, for example, intramuscular, subcutaneous, intravenous, intra-arterial, intra-articular, intraperitoneal, or parenteral routes. Methods for preparing administrable compositions are described in more detail in such publications as Remington: The Science and Practice of Pharmacy, 22^{nd} ed., London, UK: Pharmaceutical Press, 2013.

Thus, an immunogen described herein can be formulated with pharmaceutically acceptable carriers to help retain biological activity while also promoting increased stability during storage within an acceptable temperature range. Potential carriers include, but are not limited to, physiologically balanced culture medium, phosphate buffer saline solution, water, emulsions (e.g., oil/water or water/oil emulsions), various types of wetting agents, cryoprotective additives or stabilizers such as proteins, peptides or hydrolysates (e.g., albumin, gelatin), sugars (e.g., sucrose, lactose, sorbitol), amino acids (e.g., sodium glutamate), or other protective agents. The resulting aqueous solutions may be packaged for use as is or lyophilized. Lyophilized preparations are combined with a sterile solution prior to administration for either single or multiple dosing.

Formulated compositions, especially liquid formulations, may contain a bacteriostat to prevent or minimize degradation during storage, including but not limited to effective concentrations (usually ≤1% w/v) of benzyl alcohol, phenol, m-cresol, chlorobutanol, methylparaben, and/or propylparaben. A bacteriostat may be contraindicated for some patients; therefore, a lyophilized formulation may be reconstituted in a solution either containing or not containing such a component.

The pharmaceutical compositions of the disclosure can contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate.

The pharmaceutical composition may optionally include an adjuvant to enhance an immune response of the host. Suitable adjuvants are, for example, toll-like receptor agonists, alum, AlPO4, alhydrogel, Lipid-A and derivatives or variants thereof, oil-emulsions, saponins, neutral liposomes, liposomes containing the vaccine and cytokines, non-ionic block copolymers, and chemokines.

Non-ionic block polymers containing polyoxyethylene (POE) and polyxylpropylene (POP), such as POE-POP-POE block copolymers, MPL™ (3-O-deacylated monophosphoryl lipid A; Corixa, Hamilton, IN) and IL-12 (Genetics Institute, Cambridge, MA), may be used as an adjuvant (Newman et al., 1998, Critical Reviews in Therapeutic Drug Carrier Systems 15:89-142). These adjuvants have the advantage in that they help to stimulate the immune system in a non-specific way, thus enhancing the immune response to a pharmaceutical product.

In some implementations, the composition can be provided as a sterile composition. The pharmaceutical composition typically contains an effective amount of a disclosed imnunogen and can be prepared by conventional techniques. Typically, the amount of immunogen in each dose of the immunogenic composition is selected as an amount which elicits an immune response without significant, adverse side effects. In some implementations, the composition can be provided in unit dosage form for use to elicit an immune response in a subject, for example, to prevent HIV-1 infection in the subject. A unit dosage form contains a suitable single preselected dosage for administration to a subject, or suitable marked or measured multiples of two or more preselected unit dosages, and/or a metering mechanism for administering the unit dose or multiples thereof. In other implementations, the composition further includes an adjuvant.

### VI. Methods of Inducing an Immune Response

The disclosed immunogens, polynucleotides and vectors encoding the disclosed immunogens, and compositions including same, can be used in methods of inducing an immune response to HIV-1 to prevent, inhibit, and/or treat an HIV-1 infection.

When inhibiting, treating, or preventing HIV-1 infection, the methods can be used either to avoid infection in an HIV-1 seronegative subject (e.g., by inducing an immune response that protects against HIV-1 infection), or to treat existing infection in an HIV-1 seropositive subject. The HIV-1 seropositive subject may or may not carry a diagnosis of AIDS. Hence in some implementations the methods involve selecting a subject at risk for contracting HIV-1 infection, or a subject at risk of developing AIDS (such as a subject with HIV-1 infection), and administering a disclosed immunogen to the subject to elicit an immune response to HIV-1 in the subject.

To identify subjects for prophylaxis or treatment according to the methods of the disclosure, accepted screening methods are employed to determine risk factors associated with a targeted or suspected disease or condition, or to determine the status of an existing disease or condition in a subject. These screening methods include, for example, conventional work-ups to determine environmental, familial, occupational, and other such risk factors that may be associated with the targeted or suspected disease or condition, as well as diagnostic methods, such as various ELISA and other immunoassay methods to detect and/or characterize HIV-1 infection. These and other routine methods allow the clinician to select patients in need of therapy using the methods and pharmaceutical compositions of the disclosure. In accordance with these methods and principles, a composition can be administered according to the teachings herein, or other conventional methods, as an independent prophylaxis or treatment program, or as a follow-up, adjunct or coordinate treatment regimen to other treatments.

The disclosed immunogens can be used in coordinate (or prime-boost) immunization protocols or combinatorial formulations. In certain implementations, novel combinatorial immunogenic compositions and coordinate immunization protocols employ separate immunogens or formulations, each directed toward eliciting an anti-HIV-1 immune response, such as an immune response to HIV-1 Env protein. Separate immunogenic compositions that elicit the anti-HIV-1 immune response can be combined in a polyvalent immunogenic composition administered to a subject in a single immunization step, or they can be administered separately (in monovalent immunogenic compositions) in a coordinate immunization protocol.

In one implementation, a suitable immunization regimen includes at least two separate inoculations with one or more immunogenic compositions including a disclosed immunogen, with a second inoculation being administered more than about two, about three to eight, or about four, weeks following the first inoculation. A third inoculation can be administered several months after the second inoculation, and in specific implementations, more than about five months after the first inoculation, more than about six months to about two years after the first inoculation, or about eight months to about one year after the first inoculation. Periodic inoculations beyond the third are also desirable to enhance the subject's “immune memory.” The adequacy of the vaccination parameters chosen, e.g., formulation, dose, regimen and the like, can be determined by taking aliquots of serum from the subject and assaying antibody titers during the course of the immunization program. Alternatively, the T cell populations can be monitored by conventional methods. In addition, the clinical condition of the subject can be monitored for the desired effect, e.g., prevention of HIV-1 infection or progression to AIDS, improvement in disease state (e.g., reduction in viral load), or reduction in transmission frequency to an uninfected partner. If such monitoring indicates that vaccination is sub-optimal, the subject can be boosted with an additional dose of immunogenic composition, and the vaccination parameters can be modified in a fashion expected to potentiate the immune response. Thus, for example, a dose of a disclosed immunogen can be increased or the route of administration can be changed.

It is contemplated that there can be several boosts, and that each boost can be a different immunogen. It is also contemplated in some examples that the boost may be the same immunogen as another boost, or the prime.

The prime and the boost can be administered as a single dose or multiple doses, for example, two doses, three doses, four doses, five doses, six doses or more can be administered to a subject over days, weeks or months. Multiple boosts can also be given, such one to five, or more.

Different dosages can be used in a series of sequential inoculations. For example, a relatively large dose in a primary inoculation and then a boost with relatively smaller doses. The immune response against the selected antigenic surface can be elicited by one or more inoculations of a subject.

In several implementations, a disclosed immunogen can be administered to the subject simultaneously with the administration of an adjuvant. In other implementations, the immunogen can be administered to the subject after the administration of an adjuvant and within a sufficient amount of time to elicit the immune response.

Determination of effective dosages in this context is typically based on animal model studies followed up by human clinical trials and is guided by administration protocols that significantly reduce the occurrence or severity of targeted disease symptoms or conditions in the subject, or that elicit a desired response in the subject (such as a neutralizing immune response). Suitable models in this regard include, for example, murine, rat, porcine, feline, ferret, non-human primate, and other accepted animal model subjects known in the art. Alternatively, effective dosages can be determined using in vitro models (for example, immunologic and histopathologic assays). Using such models, only ordinary calculations and adjustments are required to determine an appropriate concentration and dose to administer an effective amount of the composition (for example, amounts that are effective to elicit a desired immune response or alleviate one or more symptoms of a targeted disease). In alternative implementations, an effective amount or effective dose of the composition may simply inhibit or enhance one or more selected biological activities correlated with a disease or condition, as set forth herein, for either therapeutic or diagnostic purposes.

Dosage can be varied by the attending clinician to maintain a desired concentration at a target site (for example, systemic circulation). Higher or lower concentrations can be selected based on the mode of delivery, for example, trans-epidermal, rectal, oral, pulmonary, or intranasal delivery versus intravenous or subcutaneous delivery. The actual dosage of disclosed immunogen will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the composition for eliciting the desired activity or biological response in the subject. Dosage regimens can be adjusted to provide an optimum prophylactic or therapeutic response.

A non-limiting range for an effective amount of the disclosed immunogen within the methods and immunogenic compositions of the disclosure is about 0.0001 mg/kg body weight to about 10 mg/kg body weight, such as about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.5 mg/kg, about 2 mg/kg, about 2.5 mg/kg, about 3 mg/kg, about 4 mg/kg, about 5 mg/kg, or about 10 mg/kg, for example, 0.01 mg/kg to about 1 mg/kg body weight, about 0.05 mg/kg to about 5 mg/kg body weight, about 0.2 mg/kg to about 2 mg/kg body weight, or about 1.0 mg/kg to about 10 mg/kg body weight. In some implementations, the dosage includes a set amount of a disclosed immunogen such as from about 1-300 μg, for example, a dosage of about 1, 2, 3, 4, 5, 6, 7, 8, 9, 10, 15, 20, 30, 40, 50, 60, 70, 80, 90, 100, 150, 200, 250, or about 300 μg.

The dosage and number of doses will depend on the setting, for example, in an adult or anyone primed by prior HIV-1 infection or immunization, a single dose may be a sufficient booster. In naïve subjects, in some examples, at least two doses would be given, for example, at least three doses. In some implementations, an annual boost is given, for example, along with an annual influenza vaccination.

HIV-1 infection does not need to be completely inhibited for the methods to be effective. For example, elicitation of an immune response to HIV-1 with one or more of the disclosed immunogens can reduce or inhibit HIV-1 infection by a desired amount, for example, by at least 10%, at least 20%, at least 50%, at least 60%, at least 70%, at least 80%, at least 90%, at least 95%, at least 98%, or even at least 100% (elimination or prevention of detectable HIV-1 infected cells), as compared to HIV-1 infection in the absence of the therapeutic agent. In additional examples, HIV-1 replication can be reduced or inhibited by the disclosed methods. HIV-1 replication does not need to be completely eliminated for the method to be effective. For example, the immune response elicited using one or more of the disclosed immunogens can reduce HIV-1 replication by a desired amount, for example, by at least 10%, at least 20%, at least 50%, at least 60%, at least 70%, at least 80%, at least 90%, at least 95%, at least 98%, or even at least 100% (elimination or prevention of detectable HIV-1 replication), as compared to HIV-1 replication in the absence of the immune response.

To successfully reproduce itself, HIV-1 must convert its RNA genome to DNA, which is then imported into the host cell's nucleus and inserted into the host genome through the action of HIV-1 integrase. Because HIV-1's primary cellular target, CD4+ T-Cells, can function as the memory cells of the immune system, integrated HIV-1 can remain dormant for the duration of these cells' lifetime. Memory T-Cells may survive for many years and possibly for decades. This latent HIV-1 reservoir can be measured by co-culturing CD4+ T-Cells from infected patients with CD4+ T-Cells from uninfected donors and measuring HIV-1 protein or RNA (See, e.g., Archin et al., AIDS, 22:1131-1135, 2008). In some implementations, the provided methods of treating or inhibiting HIV-1 infection include reduction or elimination of the latent reservoir of HIV-1 infected cells in a subject. For example, a reduction of at least 10%, at least 20%, at least 50%, at least 60%, at least 70%, at least 80%, at least 90%, at least 95%, at least 98%, or even at least 100% (elimination of detectable HIV-1) of the latent reservoir of HIV-1 infected cells in a subject, as compared to the latent reservoir of HIV-1 infected cells in a subject in the absence of the treatment with one or more of the provided immunogens.

Following immunization of a subject, serum can be collected from the subject at appropriate time points, frozen, and stored for neutralization testing. Methods to assay for neutralization activity, and include, but are not limited to, plaque reduction neutralization (PRNT) assays, microneutralization assays, flow cytometry based assays, single-cycle infection assays (e.g., as described in Martin et al. (2003) Nature Biotechnology 21:71-76), and pseudovirus neutralization assays (e.g., as described in Georgiev et al. (Science, 340, 751-756, 2013), Seaman et al. (J. Virol., 84, 1439-1452, 2005), and Mascola et al. (J. Virol., 79, 10103-10107, 2005), each of which is incorporated by reference herein in its entirety. In some implementations, the serum neutralization activity can be assayed using a panel of HIV-1 pseudoviruses as described in Georgiev et al., Science, 340, 751-756, 2013 or Seaman et al. J. Virol., 84, 1439-1452, 2005. Briefly, pseudovirus stocks are prepared by co-transfection of 293T cells with an HIV-1 Env-deficient backbone and an expression plasmid encoding the Env gene of interest. The serum to be assayed is diluted in Dulbecco's modified Eagle medium-10% FCS (Gibco) and mixed with pseudovirus. After 30 min, 10,000 TZM-bl cells are added, and the plates are incubated for 48 hours. Assays are developed with a luciferase assay system (Promega, Madison, WI), and the relative light units (RLU) are read on a luminometer (Perkin-Elmer, Waltham, MA). To account for background, a cutoff of ID₅₀≥40 can be used as a criterion for the presence of serum neutralization activity against a given pseudovirus.

In some implementations, administration of an effective amount of one or more of the disclosed immunogens to a subject (e.g., by a prime-boost administration of a DNA vector encoding a disclosed immunogen (prime) followed by a protein nanoparticle including a disclosed immunogen (boost)) elicits a neutralizing immune response in the subject, wherein serum from the subject neutralizes, with an ID₅₀≥40, at least 10% (such as at least 15%, at least 20%, at least 30%, at least 40%, at least 50%, or at least 70%) of pseudoviruses is a panel of pseudoviruses including the HIV-1 Env proteins listed in Table S5 or Table S6 of Georgiev et al. (Science, 340, 751-756, 2013), or Table 1 of Seaman et al. (J. Virol., 84, 1439-1452, 2005).

One approach to administration of nucleic acids is direct immunization with plasmid DNA, such as with a mammalian expression plasmid. Immunization by nucleic acid constructs is taught, for example, in U.S. Pat. No. 5,643,578 (which describes methods of immunizing vertebrates by introducing DNA encoding a desired antigen to elicit a cell-mediated or a humoral response), and U.S. Pat. Nos. 5,593,972 and 5,817,637 (which describe operably linking a nucleic acid sequence encoding an antigen to regulatory sequences enabling expression). U.S. Pat. No. 5,880,103 describes several methods of delivery of nucleic acids encoding immunogenic peptides or other antigens to an organism. The methods include liposomal delivery of the nucleic acids (or of the synthetic peptides themselves), and immune-stimulating constructs, or ISCOMS™ negatively charged cage-like structures of 30-40 nm in size formed spontaneously on mixing cholesterol and Quil ATM (saponin). Protective immunity has been generated in a variety of experimental models of infection, including toxoplasmosis and Epstein-Barr virus-induced tumors, using ISCOMS™ as the delivery vehicle for antigens (Mowat and Donachie, Immunol. Today 12:383, 1991). Doses of antigen as low as 1 μg encapsulated in ISCOMS™ have been found to produce Class I mediated CTL responses (Takahashi et al., Nature 344:873, 1990).

In some implementations, a plasmid DNA vaccine is used to express a disclosed immunogen in a subject. For example, a nucleic acid molecule encoding a disclosed immunogen can be administered to a subject to elicit an immune response to HIV-1 gp120. In some implementations, the nucleic acid molecule can be included on a plasmid vector for DNA immunization, such as the pVRC8400 vector (described in Barouch et al., J. Virol, 79, 8828-8834, 2005, which is incorporated by reference herein).

In another approach to using nucleic acids for immunization, a disclosed immunogen (such as a protomer of a HIV-1 Env ectodomain trimer) can be expressed by attenuated viral hosts or vectors or bacterial vectors. Recombinant vaccinia virus, adeno-associated virus (AAV), herpes virus, retrovirus, cytogmeglo virus or other viral vectors can be used to express the peptide or protein, thereby eliciting a CTL response. For example, vaccinia vectors and methods useful in immunization protocols are described in U.S. Pat. No. 4,722,848. BCG (Bacillus Calmette Guerin) provides another vector for expression of the peptides (see Stover, Nature 351:456-460, 1991).

In one implementation, a nucleic acid encoding a disclosed immunogen (such as a protomer of a HIV-1 Env ectodomain trimer) is introduced directly into cells. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter. Typically, the DNA is injected into muscle, although it can also be injected directly into other sites. Dosages for injection are usually around 0.5 g/kg to about 50 mg/kg, and typically are about 0.005 mg/kg to about 5 mg/kg (see, e.g., U.S. Pat. No. 5,589,466).

### EXAMPLES

The following examples are provided to illustrate particular features of certain implementations, but the scope of the claims should not be limited to those features exemplified.

### Example 1

### Materials and Methods

The following provides a description of materials and methods used in the examiner provided herein.

### Cell Lines

The EXPI 293 and FreeStyle 293F cell lines were obtained from Thermo Fisher.

### High-Throughput ELISA Screening

2.5×10⁴ log-phase HEK 293T cells in 100 μL of RealFect Expression medium (ABI Scientific, VA) per well were inoculated in a 96-well cell culture microplate (Corning Scientific, NY) and allowed to grow for 24 hours at 37° C., 5% C02. Immediately before transfection, 40 μL of spent medium per well was removed. 250 ng of plasmid DNA encoding a HIV-1 trimer variant (GenScript synthesized) in 10 μL of Opti-MEM Reduced Serum medium (Thermo Fisher Scientific, CA) was mixed with 0.75 μL of TrueFect Max transfection reagent (United Biosystems, MD) in 10 μL of Opti-MEM Reduced Serum medium at room temperature (RT) for 15 minutes, then mixed with growing cells per well in 96-well cell culture microplate. Transfected cells were incubated at 37° C. and 5% C02 for overnight (about 15 hours), and then fed with 25 μL per well of CelBooster medium (Cell Growth Enhancer for adherent cells, ABI Scientific) with additional 3× Streptomycin-Penicillin and 10% FBS. Four days post transfection, the supernatant in the cell well was harvested, and analyzed in a 96-well plate formatted ELISA. Briefly, 96-well ELISA plates (Nunc Maxisorp, Thermo Fisher Scientific) were coated with 100 μl per well of Lectin at a concentration of 5 μg/ml (Galanthus Nivalis, SIGMA) in PBS overnight at 4° C., followed by blocking with a standard block solution (1% BSA and 0.05% Tween in PBS). 30 μL of expression supernatant and 70 μL of PBS per well were incubated in Lectin-coated plate at RT for 2 hrs. Captured trimer proteins were characterized by incubating with various primary antibodies at a concentration of 10 μg/ml at RT for 60 minutes, followed by detecting bound primary antibodies with anti-human IgG Fc HRP-conjugate (Jackson ImmunoResearch Labs, PA) at RT for 30 minutes. After final washing, the reaction signal was detected by addition of 100 μl per well of BioFX-TMB (SurModics, MN) at RT for 10 minutes. The reaction was stopped by addition of 100 μl per well of 0.5N H2SO4. The signal was measured at 450 nm wavelength on a microplate reader (SpectraMax Plus, Molecular Devices, CA).

### HIV Env Expression and Purification

HIV Env trimers were purified in a manner as previously described for SARS-CoV2-S2P probes (Zhou et al., 2020). The Env trimers, genetically fused to a single-chain Fc domain, were transiently transfected into 293Freestyle cells and allowed to grow for 5 days at 37° C. The protein was purified from the supernatant using Protein A Sepharose Fast Flow resin (Cytivia), and the tag cleaved using HRV-3C protease. The collected trimer was applied to a Superdex S-200 gel filtration column equilibrated in PBS, pH 7.4. After gel filtration, the peak containing the HIV trimer was concentrated and supplemented with 10% glycerol, flash frozen in liquid nitrogen, and stored at −80° C. until use.

### Fusion Peptide-Conjugate Immunogens

The FP8v1-rTTHC conjugate was produced by coupling the FP8v1 peptide sequence appended with a C-terminal cysteine (AVGIGAVF-C) to recombinant tetanus toxoid heavy chain fragment (rTTHC) using a sulfosuccinimidyl(4-iodoacetyl)aminobenzoate (sulfo-SIAB) heterobifunctional crosslinker (VRC Production Program) (Ou et al., 2020). The conjugation ratio of peptide to carrier protein for the FP8v1-rTTHC conjugate was 5.4, as determined by amino acid analysis (Yang et al., 2020).

### Differential Scanning Calorimetry

DSC scans were performed on the HIV trimers using a Microcal VP-DSC instrument (GE Healthcare/MicroCal). Protein samples at 0.25 mg/ml were loaded and heated from 20° C. to 95° C. at a rate of 1° C. per minute. Melting temperatures (Tm) were calculated using the included Origin software.

### Site-Specific N-Glycan Profiling and Glycan Occupancy by LC-MS/MS

An LC-MS/MS^{E} method was used to determine the glycosylation site occupancy and glycan profiles of the individual glycosites (Ivleva et al., 2019). Briefly, the purified samples were buffer-exchanged to 50 mM ammonium bicarbonate (J. T. Baker, Phillipsburg, NJ) denatured with RapiGest (Waters, Milford, MA), reduced with DTT (ThermoFisher Life Technologies, Grand Island, NY). Four complementary proteolytic digests were applied using trypsin, chymotrypsin, LysC, and a mixture of trypsin with chymotrypsin (New England Biolab, Ipswich, MA). The portion of the resulting digests was deglycosylated with a mixture of PNGase F, Endo H (Promega, Madison, WI), and alpha 1-2,3 mannosidase (New England Biolab, Ipswich, MA). The digests were a subject for RPLC separation on an Acquity H-Class chromatography system with MS/MS^{E}analysis of glycan structures on an SYNAPT G2 QTof mass spectrometer, both from Waters (Milford, MA). The data processing was performed using BiopharmaLynx followed by manual inspection of the MS/MS spectra. Glycan occupancy was estimated based on the relative amounts of the non-modified and deamidated components resulting from the deglycosylation.

### Surface Plasmon Resonance

Surface Plasmon Resonance analysis was performed to determine the affinity of the purified HIV trimers to soluble CD4 (sCD4) using a Biacore T-200 instrument (GE Healthcare). 2G12 antibody was immobilized onto a CM5 chip to approximately 2,000 RU, after which the HIV trimers were loaded onto the immobilized antibody. sCD4 was injected at five concentrations (30-500 nM) incrementally in a single cycle configuration at 50 μl/min every 60 seconds, followed by a dissociation phase of 30 minutes. All loading and binding phases were performed in HBP-EP+ buffer (10 mM HEPES pH 7.4, 150 mM NaCl, 3 mM EDTA, and 0.05% P-20) Binding curves of the injection series were corrected with corresponding blank channels and fit using the Biacore T-200 Evaluation software. Related to CD4 affinity, we note that current data indicates that alteration in CD4 affinity inversely relates to the stability of the pre-fusion closed conformation of the Env trimer. This effect has been previously reported with fewer stabilizations than reported here, decreasing affinity from 1 nM to 400 nM with increasing modifications (Chuang et al., 2020).

### Antigenic Analysis of Env Trimers by MSD-ECLIA

Standard 96-well bare Multi-Array plates (catalog no. L15XA-3; MSD) were coated with all or a subset of a panel of HIV-neutralizing (VRC01, b12, VRC13, PGT121, PGT128, 2G12, PGT145, CAP256-VRC26.25, 35022, 8ANC195, PGT151, and VRC34.01 and non-neutralizing or weakly neutralizing monoclonal (F105, 17b [±soluble CD4 {sCD4}], 48D [±sCD4], 447-52D [±sCD4], 3074 [±sCD4], 2557 [±sCD4]) and base binding antibodies (1E6, 989, 5H3, 3H2) noncognate (anti-influenza antibody Mota IgG) antibodies in duplicate (30 l/well) at a concentration of 4 μg/ml diluted in 1×PBS by incubating overnight at 4° C. The following day, the plates were washed (wash buffer, 0.05% Tween 20 μlus 1×PBS) and blocked with 150 μl of blocking buffer (5% [wt/vol]blocker A [catalog no. R93BA-4; MSD]) by incubating for 1 h on a vibrational shaker (Heidolph Titramax 100, catalog no. 544-11200-00) at 650 rpm. All incubations were performed at room temperature except for the coating step. During the incubation, trimers were titrated in serial 2× dilutions starting at a concentration of 5 μg/ml of the trimer in the assay diluent (1% [wt/vol] MSD blocker A plus 0.05% Tween 20). For sCD4 induction, the trimer was combined with sCD4 at a constant molar concentration of 1 μM before being added to the MSD plate. After the incubation with blocking buffer was complete, the plates were washed, and the diluted trimer was transferred (25 μl/well) to the MSD plates and incubated for 2 h on the vibrational shaker at 650 rpm. After the 2-h incubation with trimer, the plates were washed again and 2G12 antibody labeled with Sulfo-Tag (catalog no. R91AO-1; MSD) at a conjugation ratio of 1:15 (2G12: Sulfo-Tag), which was diluted in assay diluent at 4 μg/ml, added to the plates (25 μl/well), and incubated for 1 h on the vibrational shaker at 650 rpm. The plates were washed and read using read buffer (Read Buffer T, catalog no. R92TC-1; MSD) on the MSD Sector Imager 600 or equivalent instrument.

### Cryo-EM Single Particle Analysis of Base-Glycan ConC

Base-glycan ConC trimer at a concentration of 5 mg/ml was supplemented with 0.01% DDM and frozen onto Quantifoil R 2/2 grids using a FEI Vitrobot Mark IV plunger at 4° C. and 95% relative humidity. Datasets were collected at NICE cryo-EM facility on an FEI Titan Krios electron microscope equipped with a Gatan K3 summit DED operated in the super-resolution mode (pixel size before binning: 0.415 Å). Cryosparc 3.3 (Punjani et al., 2017) was used for CTF, 2D classifications, ab initio 3D reconstructions, homogenous and non-uniform refinements. Initial reconstructions were performed with C1 symmetry, before moving to C3 symmetry for the final maps. The structure of the protein-base ConC (PDB 6CK9) was docked into the map, and refined by alternating rounds of manual building in WinCoot (Emsley and Cowtan, 2004) and automated refinement in Phenix (Adams et al., 2004). Figures were generated using Pymol and Chimera (Pettersen et al., 2004).

### Animal Protocols and Immunizations

All animal experiments were reviewed and approved by the Animal Care and Use Committee of the Vaccine Research Center (VRC), NIAID, NIH. Animals were housed and cared for in accordance with local, state, federal, and institute policies in an American Association for Accreditation of Laboratory Animal Care-accredited facility at the VRC.

For mouse studies, female C57BL/6 mice around 8 weeks old (Jackson Laboratory, Wilmington, MA) were immunized in two-week intervals for FP-primed regimens or in three-week intervals for trimer-only immunization regimens. For each immunization, 25 μg HIV-1 Env trimer or 25 μg FP8v1-rTTHC conjugate immunogens were formulated with 20% (v/v) Adjuplex adjuvant (research-grade Adjuplex®, Advanced BioAdjuvants LLC of Omaha (ABA)) in a final injection volume of 100 μl. Cocktails of FP8v1-rTTHC and BG505 Env trimer were prepared by mixing 25 μg of each immunogen and cocktails of BG505 and ConC trimers were prepared by mixing 12.5 g of each trimer prior to diluting in PBS with 20% (v/v) Adjuplex. Immunizations were administered intramuscularly as two separate injections of 50 μL each to the caudal thigh muscle of the two hind legs. Sera samples were collected two weeks after each immunization for serological analyses.

For guinea pig studies, two-month-old female Hartley guinea pigs with body weights of 300 g (Charles River Laboratories, Wilmington, MA) were immunized every four weeks. For each immunization, 25 ptg of HIV-1 Env trimer was diluted in PBS with 20% (v/v) Adjuplex (80 μL of research-grade Adjuplex®, Advanced BioAdjuvants LLC of Omaha (ABA)) in a final volume of 400 μL. Immunizations were given intramuscularly as two separate injections of 200 μL into each quadriceps muscle. Sera samples were collected for serological analyses two weeks following each immunization.

### Neutralization Assays

Neutralization assays were performed using single round of infection HIV-1 Env-pseudoviruses and TZM-bl target cells, as previously described (Cheng et al., 2019; Sarzotti-Kelsoe et al., 2014). The Δ611 mutant of BG505 is especially sensitive to FP-directed neutralization and was used to assess FP-directed responses. Neutralization curves were fit by nonlinear regression using a 5-parameter hill slope equation. The 50% and 80% inhibitory dilutions (ID50 and ID80) were reported as the reciprocal of the dilutions required to inhibit infection by 50% and 80%, respectively. Single-point assays were performed in duplicate at a dilution of 1:50, and data reported as percent neutralization.

### Enzyme-Linked Immunosorbent Assay (ELISA)

Anti-trimer ELISA were performed using lectin-captured HIV-1 trimers, as previously described (Cheng et al., 2019). Ninety-six-well plates (Costar® High Binding Half-Area; Corning, Kennebunk, ME) were coated overnight at 4° C. with 50 l/well snowdrop lectin from Galanthus nivalis (Sigma-Aldrich, St. Louis, MO) in PBS. Plates were washed five times with PBS-T (PBS plus 0.05% Tween) between each subsequent step. After being coated, plates were blocked with 100 l/well of blocking buffer (5% skim milk in PBS) and incubated at room temperature for 60 min, followed by trimer capture with 2 μg/ml HIV-1 trimers in 10% FBS-PBS for 2 hours at room temperature. Next, 50 μl/well serially diluted sera (5-fold; starting dilution of 1:100 or 1:1000) in 0.2% Tween-PBS buffer was added and incubated for 1 hour at room temperature. Following incubation, goat anti-guinea pig IgG conjugated to horseradish peroxidase (Invitrogen, Waltham, MA) diluted 1:5,000 in 5% skim milk (BD Life Sciences, Sparks, MD) PBS buffer or goat anti-mouse IgG conjugated to horseradish peroxidase (Invitrogen, Waltham, MA) diluted 1:2,000 in 5% skim milk (BD Life Sciences, Sparks, MD) PBS buffer was added at 50 μl/well for 60 min at room temperature. Plates were washed five times with PBS-T and developed with 50 μl/well tetramethylbenzidine (TMB) substrate (SureBlue; KPL, Gaithersburg, MD) for 10 min at room temperature before the addition of 50 μl/well 1 N sulfuric acid (Fisher Chemical, Fair Lawn, NJ), without washing, to stop the reaction. Plates were read at 450 nm (SpectraMax using SoftMax Pro, version 5, software; Molecular Devices, Sunnyvale, CA), and optical densities (OD) were analyzed following subtraction of the nonspecific horseradish peroxidase background activity. The endpoint titer was defined as the reciprocal of the greatest dilution with an OD value above 0.1 (2 times average raw plate background).

### Preparation of HIV-1 Env-Fab Complexes for EMPEM

1 mg of total Fab from immunized guinea pigs was incubated overnight with 10-20 μg HIV-1 Env trimers at room temperature. Env-Fab complexes were purified by size exclusion chromatography using a Superose 6 Increase 10/300 column (Cytiva) in a buffer containing 150 mM NaCl and 5 mM HEPES, pH 7.4. The fractions containing the Env-Fab complexes were pooled and concentrated to ˜1 mg/ml.

### Negative-Stain EM

The HIV-1 Env or purified Env-Fab complexes were diluted to achieve a trimer concentration of approximately 0.02 mg/ml, 4.8 μl of the diluted protein solution was adsorbed to freshly glow-discharged carbon-coated grids for ˜15 seconds, then dried with wick-paper and rinsed 3 times with 4.8 μl of buffer containing 10 mM HEPES, pH 7.0, and 150 mM NaCl before stained with 0.75% uranyl formate for 30 seconds. Datasets were collected using a Thermo Scientific Talos F200C transmission electron microscope operated at 200 kV and equipped with a Ceta camera. The nominal magnification was 57,000×, corresponding to a pixel size of 2.53 Å, and the defocus was set at ˜1.2 μm. For EMPEM datasets, micrographs were collected to ensure more than 100,000 single particles could be picked. Particle-picking, 2D and 3D classification were carried out using CryoSparc 3.3 (Punjani et al., 2017).

### Example 2

### Base-Covered Soluble HIV-1 Env Ectodomain Trimers

Soluble HIV-1-envelope (Env) trimers elicit immune responses that target their solvent-exposed protein bases, the result of removal of these trimers from their native membrane-bound context. To assess whether glycosylation could mask these responses, we introduced sequons encoding potential N-linked glycosylation sites (PNGSs) into base-proximal regions. Expression and antigenic analysis indicated trimers bearing six-introduced PNGSs to have reduced base recognition. Cryo-EM analysis revealed trimers with introduced PNGSs to be prone to disassembly and introduced PNGS to be disordered. Immunogenically, protein-base and glycan-base trimers induced reciprocally symmetric ELISA responses, in which only a small fraction of the response to glycan-base trimers could be recognized by protein-base trimers and vice versa. EM polyclonal epitope mapping (EMPEM) revealed glycan-base trimers—even those that were stable trimers biochemically—to elicit antibodies that recognized disassembled trimers. Introduced glycans can thus mask the protein-base, but may destabilize the trimer, yielding neo-epitopes that dominate the immune response.

### Introduction

The HIV-1 envelope (Env) trimer, comprising three gp120 subunits and three gp41-transmembrane subunits, uses many strategies to evade the elicitation of neutralizing antibodies. It changes shape from the closed conformations to open conformations (Kwong et al., 2002) and disassembles into highly immunogenic subunits (McKeating et al., 1991; Moore et al., 1990), which elicit only antibodies incapable of neutralizing functional virus. Thus, HIV-1 infected individuals are rapidly antibody positive, but elicited antibodies are generally non-neutralizing. Indeed, broadly neutralizing antibodies are elicited in only a minority of infected individuals and only after years of infection and high viremia (Hraber et al., 2014).

The introduction of soluble Env trimers, stabilized in a particular susceptible prefusion-closed conformation, by artificial disulfides, helix-killing prolines, and other often structure-based alterations (Kwon et al., 2015; Sanders et al., 2013), fixed both conformational and disassembly issues, and these prefusion-closed soluble trimers could elicit autologous neutralization against diverse Tier-2 neutralization strains (de Taeye et al., 2015). Analysis of the elicited response in mice and non-human primates, however, indicated a substantial portion of the response elicited by these soluble trimers to be directed to the exposed trimer base (Bianchi et al., 2018; Corrigan et al., 2021; Cottrell et al., 2020; Hu et al., 2015; Nogal et al., 2020). While the exposed base comprises less than 10% of the trimer surface, the VRC018 clinical study with BG505 DS-SOSIP revealed >90% of the immune response to be directed to the protein base (Houser et al., 2022).

Here, we test the impact of adding N-linked glycans to the exposed trimer base. To soluble trimers stabilized in the prefusion-closed conformation, we added up to three sequons encoding potential N-linked glycosylation sites (PNGSs) to each of the sequence segments comprising the exposed protein base. We tested 16 variants of each prefusion-stabilized trimer, selecting 4 for expression, and then a single lead for characterization of glycosylation, of antigenicity against a panel of antibodies, of structure by electron microscopy, and of immunogenicity in mice and guinea pigs.

### Results

### Design and Selection of Glycan-Base Env Trimers

We chose two prefusion stabilized trimers as initial templates, the aforementioned BG505 DS-SOSIP (Kwon et al., 2015) as well as a consensus clade C trimer stabilized by multiple substitutions (ConC) (Chuang et al., 2019), for which we have manufactured for clinical assessment (Gulla et al., 2021). Further, we added multiple prefusion stabilizing mutations to the BG505-DS-SOSIP template, repair- and stabilized-based stabilizing mutations (Rutten et al., 2018), 3Mut (Chuang et al., 2020), and 2G mutations (Guenaga et al., 2017), and to the ConC trimer (Rutten et al., 2018), we further stabilized by adding the DS substitution (Kwon et al., 2015) and altered the fusion peptide sequence by replacing an Isoleucine with a Leucine (Chuang et al., 2020). The matrix of modifications was introduced into template sequences based on BG505 and ConC Env sequences:

BG505-RnS7Mutv1-DS-SOSIP-2G-3Mut
(SEQ ID NO: 19)
AENLWVTVYYGVPVWKDAETTLFCASDAKAYETEKHNVWATHACVPTDPN
PQETHLENVTEEFNMWKNNMVEQMHTDIISLWDQSLKPCVKLTPLCVTLQ
CTNVTNNITDDMRGELKNCSFNMTTELRDKKQKVYSLFYRLDVVQINENQ
GNRSNNSNKEYRLINCNTSAcTQiCPKVSFEPIPIHYCAPAGFAILKCKD
KKFNGTGPCknVSTVQCTHGIKPVVSTQLLLNGSLAEEEVMIRSENITNN
AKNIiVQFNTPVQINCTRPNNmTRKSIRIGPGQAFYAlGDIIGDIRQpHC
TVSKATWNETLGKVVKQLRKHFGNNTIIfFANSSGGDLEVTTHSFNCGGE
FFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSE
LYKYKVVKIEPLGVAPTRcKRRVVGrrrrrrAVGIGAVFLGFLGAAGSTM
GAASnTLTVQARqLLSGIVQQQSNLLRApEAQQHLLKLgVWGfKQLQARV
LAVERYLevQQLLGIWGCSGKLICcTNVPWNSSWSNRNLSEIWDNMTWLQ
WDKEIgNYTQIIYGLLEESQfQQEiNEQDLLALD
VRC6931 (ConC-FP8v2 RnS-3mut-2G-SOSIP.664)
(SEQ ID NO: 20)
NLWVTVYYGVPVWKEAKTTLFCASDAKAYEKEVIINVWATIIACVPTDPN
PQEMVLENVTENFNMWKNDMVDQMIIEDIISLWDQSLKPCVKLTPLCVTL
NCTNVNVTNTNNNNMKEEMKNCSFNTTTEIRDKKQKEYALFYRLDIVPLN
ENSSEYRLINCNTSTITQICPKVSFDPIPIHYCAPAGYAILKCNNKTFNG
TGPCNNVSTVQCTHGIKPVVSTQLLLNGSLAEEEIIIRSENLTDNAKTII
VHLNESVEINCTRPNNMTRKSIRIGPGQTFYALGDIIGDIRQPHCNISEA
KWNKTLQRVKKKLKEHFPNKTIKFAPSSGGDLEITTHSFNCRGEFFYCNT
SKLFNSTYNNTTSNSTITLPCRIKQIINMWQEVGRAMYAPPIAGNITCKS
NITGLLLTRDGGNNNNNTETFRPGGGDMRDNWRSELYKYKVVEIKPLGIA
PTKCKRRVVERRRRRRAVGLGAVFLGFLGAAGSTMGAASNTLTVQARQLL
SGIVQQQSNLLRAPEAQQHMLQLGVWGFKQLQARVLAIERYLEVQQLLGI
WGCSGKLICCTAVPWNSSWSNKSQEDIWDNMTWMQWDREIGNYTDTIYRL
LEESQFQQEINEKDLLALD

The exposed Env-protein base on these trimers is made up of three different sequence segments: the N terminus of the gp120 subunit, the furin-cleavage site connecting the C-terminus of gp120 and the N-terminus of gp41, and the C terminus of gp41, where this subunit was genetically clipped from the membrane (FIG. 1A). We introduced a PNGS at residue 28 at the N terminus of gp120, one or two PNGSs to the C terminus of gp41, and one to three PNGSs in the linker preceding the furin cleavage site at the gp120-gp41 juncture. The PNGSs at the furin site were added as part of a sequence, which we termed “JCB-cleavage sequence”. This sequence derives from an influenza variant (Hamilton et al., 2012), and we found its insertion upstream of the “RRRRRR” cleavage loop resulted in more efficient furin cleavage, as well as enabling multiple PNGS additions. (FIG. 1B).

We synthesized constructs encoding each of the 16 glycan-base variants for both BG505 and ConC trimers. We used these to transfect 293-cells in 96-well format, producing supernatants with the glycan-variants, which we assessed antigenically for recognition by broadly neutralizing antibodies, for recognition by antibodies that bind open trimer conformations (F105, 447-52D, 17b, and 17b in the presence of CD4), and for recognition by five-protein base directed antibodies (1E6, 5H3, 3H2, 989, and RM20A3) (FIGS. 1C and 8A-8B). In general, the antigenic properties of the glycosylated trimers were generally similar, with the main difference being the number of antibodies against the base that lost binding. Overall, trimers with 4-6 introduced PNGSs appeared to show the greatest reduction in base-directed antibody recognition, and we modeled these (FIG. 1D) and selected four for further characterization.

### Physical Properties of Glycan-Base Env Trimers

The four selected constructs in both the BG505 and ConC background strains were expressed at a larger scale, to allow for more thorough antigenic and biophysical characterizations. As a first pass of screening, the efficiency of furin cleavage was analyzed by comparing the intensity of the gp120 band on Coomassie stained SDS-PAGE to that of the gp140 band (FIG. 2A). Reasonable cleavage, greater than 80%, was observed for all constructs; however, for both BG505 and ConC, the cleavage was more efficient in the constructs with the N28, JCBv2 or JCBv3, 600 and 665 modifications (FIG. 2C).

To test for the ability of the glycosylated bases to block immunoreactivity, the constructs for both BG505 and ConC were tested against sera from NHPs immunized with the either the respective protein-base trimer alone or primed with fusion peptide and then boosted with protein-base trimer. As the trimer alone sera primarily target the base of the HIV Env protein (Corrigan et al., 2021), whereas the fusion peptide primed sera are far less base-targeting, a reduction in the ratio of this reactivity would indicate successful blocking of this surface. For the BG505 constructs, all performed equally well, with an approximately 9-fold reduction between the two sera. In the case of the ConC constructs, the ratio was more variable, ranging from approximately 3- to 5-fold reduction (FIGS. 2B and 2C). As the constructs with the most added PNGSs, N28-JCBv3-660-665, for both BG505 and ConC were the best cleaved, and in the case of ConC provided the highest differential in sera binding, they were selected for further analysis. The sequence for these constructs is provided below:

VRC 7678 (6931_DS-N28-JCBv3-N660-N665.667)
(ConC based)
SEQ ID NO: 8
nStAENLWVTVYYGVPVWKEAKTTLFCASDAKAYEKEVHNVWATHACVPT
DPNPQEMVLENVTENFNMWKNDMVDQMHEDIISLWDQSLKPCVKLTPLCV
TLNCTNVNVTNTNNNNMKEEMKNCSFNTTTEIRDKKQKEYALFYRLDIVP
LNENSSEYRLINCNTSTcTQICPKVSFDPIPIHYCAPAGYAILKCNNKTF
NGTGPCNNVSTVQCTHGIKPVVSTQLLLNGSLAEEEIIIRSENLTDNAKT
IIVHLNESVEINCTRPNNmTRKSIRIGPGQTFYAlGDIIGDIRQpHCNIS
EAKWNKTLQRVKKKLKEHFPNKTIKFAPSSGGDLEITTHSFNCRGEFFYC
NTSKLFNSTYNNTTSNSTITLPCRIKQIINMWQEVGRCMYAPPIAGnITC
KSNITGLLLTRDGGNNNNNTETFRPGGGDMRDNWRSELYKYKVVEIKPLG
IAPTKcnRtVVEnsthknlthhmrrrrrrAVGLGAVFLGFLGAAGSTMGA
ASNTLTVQARQLLSGIVQQQSNLLRAPEAQQHMLQLGVWGFKQLQARVLA
IERYLEVQQLLGIWGCSGKLICCTAVPWNSTWSNKTQEDIWDNMTWMQWD
REIGNYTDTIYRLLEESQFQQEINEKDNLTLPNST
VRC 7873 (4571_RnS7Mutv1-2G-3Mut-N28-JCBv3-
N660-N665.667_613S_620S) (BG505-based)
SEQ ID NO: 9
NSTAENLWVTVYYGVPVWKDAETTLFCASDAKAYETEKHNVWATHACVPT
DPNPQEIHLENVTEEFNMWKNNMVEQMHTDIISLWDQSLKPCVKLTPLCV
TLQCTNVTNNITDDMRGELKNCSFNMTTELRDKKQKVYSLFYRLDVVQIN
ENQGNRSNNSNKEYRLINCNTSACTQICPKVSFEPIPIHYCAPAGFAILK
CKDKKFNGTGPCKSVSTVQCTHGIKPVVSTQLLINGSLAEEEVMIRSENI
TNNAKNIIVQFNTPVQINCTRPNNMTRKSIRIGPGQAFYALGDIIGDIRQ
PHCNVSKATWNETLGKVVKQLRKHFGNNTIIFFANSSGGDLEVTTHSFNC
GGEFFYCNTSGLFNSTWISNTSVQGSNSTGSNDSITLPCRIKQIINMWQR
VGQCMYAPPIQGVIRCVSNITGLILTRDGGSTNSTTETFRPGGGDMRDNW
RSELYKYKVVKIEPLGVAPTRCNRTVVGNSTHKNLTHHMRRRRRRAVGIG
AVFLGFLGAAGSTMGAASNTLTVQARQLLSGIVQQQSNLLRAPEAQQHLL
KLGVWGFKQLQARVLAVERYLEVQQLLGIWGCSGKLICCTNVPWNSSWSN
RNLSEIWDNMTWLQWDKEIGNYTQIIYGLLEESQFQQEINEQDNLTLPNS
T

Both constructs of N28-JCBv3-660-665 for BG505 and ConC (hereafter referred to as BG505 glycan-base trimer and ConC glycan-base trimer) migrated through a size exclusion column as expected with the primary peak representing the trimeric fraction (FIG. 2D). Moreover, differential scanning calorimetry (DSC) showed these two glycan-base trimers to be well-folded, stable proteins with Tm's of 67.3° C. and 76.5° C. for the BG505 and ConC variants, respectively (FIGS. 2E and 8A-8B). This stability is slightly reduced for BG505 compared to its protein-base counterpart, but the stability is actually increased for the ConC glycan-base constructs, likely owing to the additional DS stabilization added to the glycan variants. Finally, the trimeric nature of these proteins was confirmed by NS-EM, wherein both constructs appeared with the characteristic shape of HIV Env constructs, with a minimum of monomers or other mis-folded particles seen (FIG. 2F).

### Glycosylation and Antigenic Profiles of BG505 and ConC Glycan-Base Trimers

Glycosylation profiles and glycan occupancy of the six engineered PNGSs and other base glycans in gp41 were assessed for BG505 and ConC glycan-base trimer variants using LC-MS/MS peptide mapping methods (Ivleva et al., 2019) (FIG. 3A). The majority of base glycans were predominantly high-mannose type, except for the N28 glycosite which contained primarily complex-type glycan. Aside from the N508 glycan, all other engineered PNGSs within gp120 were nearly fully occupied (99-100%). In contrast, the engineered base glycans in gp41 displayed decreased occupancy, with the N660 glycan showing <45% occupancy and the C-terminal N665 glycan <6% occupied. Native base glycans at N611 and N616 (ConC) or N618 (BG505) were found to have increased glycan occupancy in the glycan-base trimers compared to their protein-base counterparts, likely a result from the use of NxT sequons to optimize glycan occupancy (Derking et al., 2021). Interestingly, the glycan type for the N61 t and N616/N618 sites switched from complex-type in the protein-base trimers to high-mannose type in the glycan-base trimers (FIG. 3A), likely a consequence of the increased glycosylation density around the based, as increased glycan density reduces glycan processing.

To characterize further the glycan-base trimers, we assayed their binding affinities to a soluble construct of CD4 containing the first two domains (sCD4) by surface plasmon resonance (FIG. 3B). While the binding affinity of the protein-base BG505 was slightly lower than that previously reported, the affinity of the glycan-base BG505 was comparable to that previously reported for stabilized BG505 (Chuang et al., 2020).

Antigenic profiles for the BG505 and ConC glycan-base trimers were evaluated in comparison to the protein-base counterparts using the Meso Scale Discovery (MSD) platform (FIGS. 3C and 9). Glycan-base and protein-base BG505 and ConC trimers exhibited comparable binding affinity to a panel of antibodies targeting the CD4-binding site (CD4bs), V1/V2-apex, V3-glycan, and gp120/gp41 interface; however, the glycan-base BG505 trimer (but not the ConC version) displayed increased binding to weakly neutralizing, V3-directed antibodies 3074 and 2557. Notably, the glycan-base trimers showed reduced or no recognition by several base-directed antibodies, indicating the ability of the engineered glycans to mask the trimer base.

Structure of ConC Env Trimer with Glycan-Base Trimers

To determine if there were any changes in the structure of the HIV Env trimers with the additions of the glycan motifs, we sought to determine the cryoEM structure of the glycan-base trimers. The BG505 glycan-base trimer protein appeared nearly completely trimeric by negative-stain EM (FIG. 2E). Further, when imaged on frozen grids, the glycan-base ConC trimer yielded well-formed trimers. (FIG. 4A).

We solved the cryoEM structure of the glycan-base ConC trimer to 4.1A resolution (FIGS. 4B, 10, and 12). Other than the additional DS mutation, the structure appeared indistinguishable from the published structure of the protein-base ConC (FIGS. 4C and 12) (Rutten et al., 2018). One difference observed in the structure was an increase in the mobility in the base of the trimer (FIGS. 4C and 11). The base as whole was shifted nearly 5A, and the C-terminal 10 residues were not resolved in the cryo-EM density. This included the region where PNGSs were added, so we were not able to visualize the structure of the glycan additions. Moreover, the N-terminal addition (N28) and the addition proximal to the fusion peptide (JCBv3) were also absent from the electron density map, demonstrating that in all cases, the addition of the glycosylation sites, or the glycans themselves, increased the mobility of these regions.

### Murine Immunogenicity Reveals Reciprocally Symmetric Responses

To evaluate immunogenicity of the glycan-base Env trimers, four groups of C57BL/6 mice (n=10/group) were immunized three times at 3-week intervals with either protein-base or glycan-base trimers of BG505 and ConC (FIG. 5A). Serum anti-trimer ELISA responses against either protein-base or glycan-base trimers were measured for each group at week 8, two weeks following the third trimer immunization (FIGS. 5B and 5C). ELISA titers elicited by each group were reciprocally symmetric, with only a small fraction of the response from immunization of the glycan-base BG505 or ConC trimer able to recognize the corresponding protein-base trimer, and vice versa (FIGS. 5B, 5C, 13). Similar to observations reported by others, no neutralizing activity (ID₅₀) was detected against the autologous BG505 and ConC pseudoviruses for any of the mice at week 8 after the third trimer immunization although sera from one animal immunized with the glycan-base ConC trimer (Group 344) did show neutralizing activity against the heterologous MW965.26 strain (FIG. 13).

Prior studies have shown that an epitope-focused vaccine approach based on priming with HIV-1 fusion peptide (FP)-carrier conjugates and boosting with prefusion-closed stabilized HIV-1 Env trimers can elicit cross-clade HIV-1 neutralizing responses in multiple animal models (Xu et al., 2018). To assess the utility of glycan-base trimers as boosting reagents, immunogenicity of the glycan-base trimers was evaluated in the context of two different FP-primed vaccine regimens (FIG. 5D). Two groups of C57BL/6 mice (n=10/group) were immunized following an FP-primed trimer-boosted sequential regimen whereby animals were primed three times every two weeks with FP8v1-rTTHC, a fusion the N-terminal eight residues of the most prevalent HIV fusion peptide sequence (FP8v1) conjugated to the recombinant tetanus toxoid heavy chain fragment C (rTTHC) carrier protein (Ou et al., 2020). After FP priming, animals were boosted sequentially with either the protein-base or glycan-base BG505 trimer two times, followed by a single immunization with the ConC trimer and finally once with a BG505+ConC trimer cocktail. Another two groups of mice followed a “cocktail-primed” regimen whereby animals were primed three times every two weeks with a cocktail of FP8v1-rTTHC combined with either the protein-base or glycan-base variant of BG505 trimer. Following the FP+trimer cocktail prime, animals were boosted once with the ConC trimer variant and twice with a BG505+ConC trimer cocktail.

Longitudinal development of serum antibody responses to trimer were measured by ELISA against either the protein-base or glycan-base BG505 trimer (FIG. 5E). In general, anti-trimer ELISA titers developed following the second FP or FP+trimer immunization and increased gradually throughout the trimer boosts for each group. For the FP+trimer cocktail primed groups, ELISA titers were overall higher against the matching trimer immunogen during the course of immunization whereas FP-primed, sequential groups induced more similar responses against each trimer.

After the final immunization, sera were assessed at a 1:50 dilution for neutralizing activity against the BG505.N611Q viral variant, which lacks the N611 glycan for enhanced sensitivity to FP-targeted neutralization (Xu et al., 2018). Neutralizing activity against the BG505.N611Q strain was detected for all groups after the last immunization while protein-base and glycan-base trimers induced overall similar neutralizing responses, with no statistically significant difference observed between groups for each vaccine regimen (FIG. 5F).

### Glycan-Base BG505 Trimer Elicits Immune Responses in Guinea Pigs

To obtain a more comprehensive understanding of the immune responses elicited by the glycan-base Env trimers, we also immunized guinea pigs with either glycan-base or protein-base BG505 trimers. In the first set of experiments, animals (n=10/group) were immunized twice, 4 weeks apart, with either the protein-base or glycan-base BG505 trimer, administered with Adjuplex adjuvant (FIG. 6A). Anti-trimer ELISA responses against either protein-base or glycan-base BG505 trimer were measured for each group at 0, 2, and 6 weeks and showed an increase in responses after each immunization with the corresponding trimer immunogen (FIG. 6B). Similar to the responses observed in mice, ELISA titers elicited by each group appeared reciprocally symmetric, with the response to either the glycan-base or protein-base BG505 trimer showing low cross-reactivity to the alternate trimer (FIGS. 6B and 6C).

Neutralization was assessed at week 6, two weeks following the second trimer immunization, against wild-type BG505, BG505.N611Q, and MW965.26 HIV-1 pseudoviruses and 7312A_V434M, an HIV-2 strain with enhanced sensitivity to CD4-induced antibodies (FIG. 14). Animals immunized with the protein-base BG505 trimer elicited consistent neutralizing responses against the autologous wildtype BG505 (8 of 10 animals) and BG505.N611Q (10 of 10 animals) viruses while only sporadic neutralizing activity against BG505 was observed for the glycan-base BG505 trimer-immunized group (2 of 10 animals). Neutralizing activity against the heterologous tier 1 MW965.26 strain was similar between the two groups and there was little response to the HIV-2 7312A_V434M virus.

To map the antibody responses elicited by protein-base and glycan-base BG505 trimers, we performed electron microscopy polyclonal epitope mapping (EMPEM) (Han et al., 2021) using sera collected at week 6, two weeks following the second trimer dose, from one animal in each group (FIG. 6D). Antibodies elicited from immunization with the protein-base BG505 trimer were observed to target the base, N611 and FP regions of the trimer, consistent with other studies with SOSIP-stabilized Env trimers (Bianchi et al., 2018; Cottrell et al., 2020; Dingens et al., 2021; Hu et al., 2015).

### Glycan-Base ConC Trimer Elicits Immune Responses in Guinea Pigs

Parallel immunogenicity studies were done in guinea pigs using protein-base and glycan-base ConC trimers. Two groups of animals (n=10/group) were immunized twice, 4 weeks apart, with either the protein-base or glycan-base ConC trimer (FIG. 7A). Anti-trimer ELISA responses followed similar trends to that observed with the BG505 trimers, showing reciprocal binding responses (FIG. 7B) and low cross-reactivity to the alternate trimer (FIG. 7C). No neutralizing activity (ID₅₀) was detected against the wildtype or N611Q variant of ConC pseudovirus for any of the animals from either group at week 6 after the second trimer immunization although sera from two animals immunized with the glycan-base ConC trimer showed neutralizing activity against the heterologous MW965.26 strain (FIG. 14). EMPEM analysis was carried out with week 6 sera collected after the second trimer dose from one animal in each group (FIG. 7D).

### Discussion

To evade immune recognition, viruses utilize glycosylation to cover exposed antigenic protein surfaces. HIV-1 Env is heavily glycosylated with N-linked glycans that shield its potential neutralizing epitopes. Soluble Env trimer immunogens that have been removed by genetic cleavage from their native membrane-bound form, however, have exposed protein bases that become immunogenically dominant (Corrigan et al., 2021). In this study, we introduced N-linked glycans to cover the artificial base of the soluble Env trimers, to focus immune responses on the neutralizing epitopes. Antigenically, we observed that trimers with six-introduced PNGSs to have reduced base responses, and immunogenically these trimers elicited reciprocally symmetry ELISA titers.

The glycan-base ConC trimer is shown to have utility as a boosting immunogen, as we show with mice primed using FP-rTTHC or cocktail of FP-rTTHC and trimer and boosted with glycan-base trimers elicited equivalent neutralizing responses to those boosted with protein-base trimers. Because the glycan-base trimers have reduced responses to base-targeting antibodies compared with the protein-base trimers, alternating boosts with glycan-base trimers and protein-base trimers may help to avoid the dominating base immunogenicity from the exposed protein base of the soluble trimers.

### REFERENCES

Adams et al. (2004). Recent developments in the PHENIX software for automated crystallographic structure determination. J Synchrotron Radiat 11, 53-5.
Bianchi et al. (2018). Electron-Microscopy-Based Epitope Mapping Defines Specificities of Polyclonal Antibodies Elicited during HIV-1 BG505 Envelope Trimer Immunization. Immunity 49, 288-300 e8.
Cheng et al. (2019). Consistent elicitation of cross-clade HIV-neutralizing responses achieved in guinea pigs after fusion peptide priming by repetitive envelope trimer boosting. PLoS One 14, e0215163.
Chuang et al. (2020). Development of a 3Mut-Apex-Stabilized Envelope Trimer That Expands HIV-1 Neutralization Breadth When Used To Boost Fusion Peptide-Directed Vaccine-Elicited Responses. J Virol 94.
Chuang et al. (2019). Structural Survey of Broadly Neutralizing Antibodies Targeting the HIV-1 Env Trimer Delineates Epitope Categories and Characteristics of Recognition. Structure 27, 196-206 e6.
Corrigan et al. (2021). Fusion peptide priming reduces immune responses to HIV-1 envelope trimer base. Cell Rep 35, 108937.
Cottrell et al. (2020). Mapping the immunogenic landscape of near-native HIV-1 envelope trimers in non-human primates. PLoS Pathog 16, e1008753. de Taeye et al. (2015). Immunogenicity of Stabilized HIV-1 Envelope Trimers with Reduced Exposure of Non-neutralizing Epitopes. Cell 163, 1702-15.
Derking et al. (2021). Enhancing glycan occupancy of soluble HIV-1 envelope trimers to mimic the native viral spike. Cell Rep 35, 108933.
Dingens et al. (2021). High-resolution mapping of the neutralizing and binding specificities of polyclonal sera post-HIV Env trimer vaccination. Elife 10.
Duan et al. (2018). Glycan Masking Focuses Imnnune Responses to the HIV-1 CD4-Binding Site and Enhances Elicitation of VRC01-Class Precursor Antibodies. Immunity 49, 301-311 e5.
Emsley, P., and Cowtan, K. (2004). Coot: model-building tools for molecular graphics. Acta Crystallogr D Biol Crystallogr 60, 2126-32.
Guenaga et al. (2017). Glycine Substitution at Helix-to-Coil Transitions Facilitates the Structural Determination of a Stabilized Subtype C HIV Envelope Glycoprotein. Immunity 46, 792-803 e3.
Gulla et al. (2021). A non-affinity purification process for GMP production of prefusion-closed HIV-1 envelope trimers from clades A and C for clinical evaluation. Vaccine 39, 3379-3387.
Hamilton et al. (2012). Acquisition of a novel eleven amino acid insertion directly N-terminal to a tetrabasic cleavage site confers intracellular cleavage of an H7N7 influenza virus hemagglutinin. Virology 434, 88-95.
Han et al. (2021). Polyclonal epitope mapping reveals temporal dynamics and diversity of human antibody responses to H5N1 vaccination. Cell Rep 34, 108682.
Houser et al. (2022). Safety and immunogenicity of an HIV-1 prefusion-stabilized envelope trimer (Trimer 4571) vaccine in healthy adults: A first-in-human open-label, randomized, dose-escalation, phase 1 clinical trial. EClinicalMedicine 48, 101477.
Hraber et al. (2014). Prevalence of broadly neutralizing antibody responses during chronic HIV-1 infection. AIDS 28, 163-9.
Hu et al. (2015). Murine Antibody Responses to Cleaved Soluble HIV-1 Envelope Trimers Are Highly Restricted in Specificity. J Virol 89, 10383-98.
Ivleva et al. (2019). Overcoming Challenges in Structural Characterization of HIV-1 Envelope Glycoprotein by LC-MS/MS. J Am Soc Mass Spectrom 30, 1663-1678.
Kraft et al. (2022). Antigen- and scaffold-specific antibody responses to protein nanoparticle immunogens. Cell Rep Med 3, 100780.
Kwon et al. (2015). Crystal structure, conformational fixation and entry-related interactions of mature ligand-free HIV-1 Env. Nat Struct Mol Biol 22, 522-31.
Kwong et al. (2002). HIV-1 evades antibody-mediated neutralization through conformational masking of receptor-binding sites. Nature 420, 678-82.
McKeating et al. (1991). Differential loss of envelope glycoprotein gp120 from virions of human immunodeficiency virus type 1 isolates: effects on infectivity and neutralization. J Virol 65, 852-60.
Moore et al. (1990). Dissociation of gp120 from HIV-1 virions induced by soluble CD4. Science 250, 1139-42.
Nogal et al. (2020). Mapping Polyclonal Antibody Responses in Non-human Primates Vaccinated with HIV Env Trimer Subunit Vaccines. Cell Rep 30, 3755-3765 e7.
Ou et al. (2020). Preclinical Development of a Fusion Peptide Conjugate as an HIV Vaccine Immunogen. Sci Rep 10, 3032.
Pantophlet, R., Wilson, I. A., and Burton, D. R. (2003). Hyperglycosylated mutants of human immunodeficiency virus (HIV) type 1 monomeric gp120 as novel antigens for HIV vaccine design. J Virol 77, 5889-901.
Pettersen et al. (2004). UCSF Chimera—a visualization system for exploratory research and analysis. J Comput Chem 25, 1605-12.
Punjani et al. (2017). cryoSPARC: algorithms for rapid unsupervised cryo-EM structure determination. Nat Methods 14, 290-296.
Rutten et al. (2018). A Universal Approach to Optimize the Folding and Stability of Prefusion-Closed HIV-1 Envelope Trimers. Cell Rep 23, 584-595.
Sanders et al. (2013). A next-generation cleaved, soluble HIV-1 Env trimer, BG505 SOSIP.664 gp140, expresses multiple epitopes for broadly neutralizing but not non-neutralizing antibodies. PLoS Pathog 9, e1003618.
Sarzotti-Kelsoe et al. (2014). Optimization and validation of the TZM-bl assay for standardized assessments of neutralizing antibodies against HIV-1. J Immunol Methods 409, 131-46.
Schiffner et al. (2016). Chemical Cross-Linking Stabilizes Native-Like HIV-1 Envelope Glycoprotein Trimer Antigens. J Virol 90, 813-28.
Tang et al. (2007). EMAN2: an extensible image processing suite for electron microscopy. Journal of structural biology 157, 38-46.
Turner et al. (2021). Disassembly of HIV envelope glycoprotein trimer immunogens is driven by antibodies elicited via immunization. Sci Adv 7, eabh2791.
Xu et al. (2018). Epitope-based vaccine design yields fusion peptide-directed antibodies that neutralize diverse strains of HIV-1. Nat Med 24, 857-867.
Yang et al. (2020). A unique algorithm for the determination of peptide-carrier protein conjugation ratio by amino acid analysis using intrinsic internal standard. Vaccine 38, 4507-4511.
Zhang et al. (2021). A multiclade env-gag VLP mRNA vaccine elicits tier-2 HIV-1-neutralizing antibodies and reduces the risk of heterologous SHIV infection in macaques. Nat Med 27, 2234-2245.
Zhou et al. (2020). Structure-Based Design with Tag-Based Purification and In-Process Biotinylation Enable Streamlined Development of SARS-CoV-2 Spike Molecular Probes. Cell Rep 33, 108322.

### Example 3

### Additional Base-Covered Soluble HIV-1 Env Ectodomain Trimers

Further analysis of the novel glycan-base trimers described above identified the potential for these constructs to have a neo-epitope at the base due to insertion of so many amino acids in the base region of the trimer, which might generate a distinct non-neutralizing response to the base region (FIG. 15). To address this possibility, structure-based design of additional glycan-base covered soluble HIV-1 Env constructs was performed. Sequences of these constructs are listed below. These were produced as described in Examples 1 and 2, and screened for high binding to HIV-1 neutralizing antibodies, in particular antibodies that recognized the prefusion closed state of the trimer. Additionally, the designs were ranked for decreased binding to base-binding antibodies as well as to V3 epitope which would indicate trimer opening. Based on this analysis, the top designs are the following:
SEQ ID NO: 2—BG505.W6M.C2_RnS7Mutv1-DS-SOSIP-2G-3Mut
SEQ ID NO: 3—BG505.W6M.C2_RnS5Mut-DS-SOSIP-2G-3Mut_K502N-R504T-L660N-A662T
SEQ ID NO: 4—JCB-7mutvl_668-772-778-782
SEQ ID NO: 5—JG2_7mut3_N656.659
SEQ ID NO: 6—JCB-Mut7v1-3hsh01-G7-18
SEQ ID NO: 7-7Mutv1-Gly332-dGly241-base_glycan-Q617N_Q619T_L504T The sequences for the additional glycan-base covered HIV-1 Env trimers are provided below:

- BG505.W6M.C2_RnS7Mutv1-DS-SOSIP-2G-3Mut
SEQ ID NO: 2
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCknVSTVQCTHGIKPVVSTQLLLNGSLAEEEV
MIRSENITNNAKNIiVQFNTPVQINCTRPNNmTRKSIRGPGQAFYAlGDIIGDIRQpHCTVSKATWNETLGKVVKQLRKH
FGNNTIIfFANSSGGDLEVITHSFNCGGEFFYCNTSGLFNSTWISNTSVQGSNSTGSNDSITLPCRIKQIINMWQRvGQc
MYAPPIQGVIRCVSNITGLILTRDGGSTNSTTETFRPGGGDMRDNWRSELYKYKVVKIEPLGVAPTRcKRRVVGrrrrrr
AVGIGAVFLGFLGAAGSTMGAASnTLTVQARqLLSGIVQQQSNLLRApEAQQHLLKLqVWGfKQLQARVLAVERYLevQQ
LLGIWGCSGKLICcTNVPWNSSWSNRNLSEIWDNMTWLQWDKEIgNYTQIIYGLLEESQfQQEiNEQDLLALD
- BG505.W6M.C2_RnS5Mut-DS-SOSIP-2G-3Mut_K502N-
R504T-L660N-A662T
SEQ ID NO: 3
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQACPKVSFEPIPIHYCAPAGFAILKCKDKKFNGTGPCkNVSTVQCTHGIKPVVSTQLLLNGSLAEEEV
MIRSENITNNAKNIiVQFNTPVQINCTRPNNmTRKSIRIGPGQAFYAlGDIIGDIRQpHCTVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGIGAVFLGFLGAAGSTMGAASnTLTVQARqLLSGIVQQQSNLLRApEAQQHLLKLgVWGIKQLQARVLAVERYLevQ
QLLGIWGCSCKLICcTNVPWNSSWSNRNLSEIWDNMTWLQWDKEIgNYTQIIYGLLEESQfQQEiNEQDnLtLD
>JCB-7mutv1_668-772-777-781
(SEQ ID NO: 35)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLINGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESQfQQEiNEQDnLtLDGGYNVT
YNVTGYNVTYNVTIGGSGG
>JCB-7mutv1_668-772-778-782
(SEQ ID NO: 4)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVINNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESQfQQEiNEQDnLtLDGGYNVT
YNVTGGYNVTYNVTIGGSGG
>JCB-7mutv1_668-772-779-783
(SEQ ID NO: 36)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVINNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESQfQQEiNEQDnLtLDGGYNVT
YNVTGGYNVTYNVTIGGSGG
>JCB-7mutv1_668-772-779
(SEQ ID NO: 37)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVINNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESQfQQEiNEQDnLtLDGGYNVT
YNVTGGYNVTYNVTIGGSGG
>JCB-7mutv1_668-772-781-785
(SEQ ID NO: 38)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVINNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESQfQQEiNEQDnLtLDGGYNVT
YNVTGGYNVTYNVTIGGSGG
>JCB-7mutv1_505
(SEQ ID NO: 39)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtNVTVVGrr
rrrrAVGIGAVFLGFLGAAGSTMGAASNTLTVQARqLLSGIVQQQSNLLRAPEAQQHLLKLgVWGfKQLQARVLAVERYL
evQQLLGIWGCSGKLICcTNVPWNSSWSNRNLSEIWDNMTWLQWDKEIgNYTQIIYGLLEESQfQQEiNEQDnLtLD
>JCB-7mutv1_506
(SEQ ID NO: 40)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtYNVTVVGr
rrrrrAVGIGAVFLGFLGAAGSTMGAASnTLTVQARqLLSGIVQQQSNLLRApEAQQHLLKLgVWGfKQLQARVLAVERY
LevQQLLGIWGCSGKLICcTNVPWNSSWSNRNLSEIWDNMTWLQWDKEIgNYTQIIYGLLEESQfQQEiNEQDnLtLD
>JCB-7mutv1_505-507b
(SEQ ID NO: 41)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtNVTYNVTV
VGrrrrrrAVGIGAVFLGFLGAAGSTMGAASnTLTVQARqLLSGIVQQQSNLLRApEAQQHLLKLqVWGfKQLQARVLAV
ERYLevQQLLGIWGCSGKLICcTNVPWNSSWSNRNLSEIWDNMTWLQWDKEIgNYTQIIYGLLEESQfQQEiNEQDnLtL
D
>JCB-7mutv1_505-507d
(SEQ ID NO: 42)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtNVTGGYNV
TVVGrrrrrrAVGIGAVFLGFLGAAGSTMGAASnTLTVQARqLLSGIVQQQSNLLRApEAQQHLLKLgVWGfKQLQARVL
AVERYLevQQLLGIWGCSGKLICcTNVPWNSSWSNRNLSEIWDNMTWLQWDKEIgNYTQIIYGLLEESQfQQEiNEQDnL
tLD
>JCB-7mutv1_505-507d2
(SEQ ID NO: 43)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtNVTGGYNV
TDDGrrrrrrAVGIGAVFLGFLGAAGSTMGAASnTLTVQARqLLSGIVQQQSNLLRApEAQQHLLKLgVWGfKQLQARVL
AVERYLevQQLLGIWGCSGKLICcTNVPWNSSWSNRNLSEIWDNMTWLQWDKEIgNYTQIIYGLLEESQfQQEiNEQDnL
tLD
>JCB-7mutv1_33
(SEQ ID NO: 44)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSINSTTETFRPGGGDMRDNWRSELYKYKVVKIEPLGVAPTRcnRtVVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESQfQQEiNEQDnLtLD
>JCB-7mutv1_32
(SEQ ID NO: 45)
ANNTWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSCKLICcTNVPWNSSWSNRNLSEIWDNMTWLQWDKEIgNYTQIIYGLLEESQfQQEiNEQDnLtLD
>JCB-7mutv1_31-33
(SEQ ID NO: 46)
NVTNLTVTVYYGVPVWKDAETTLFCASDAKAYETEKHNVWATHACVPTDPNPQEIHLENVTEEFNMWKNNMVEQMHTDII
SLWDQSLKPCVKLTPLCVTLQCTNVTNNITDDMRGELKNCSFNMTTELRDKKQKVYSLFYRLDVVQINENQGNRSNNSNK
EYRLINCNTSAcTQiCPKVSFEPIPIHYCAPAGFAILKCKDKKFNGTGPCkSVSTVQCTHGIKPVVSTQLLINGSLAEEE
VMIRSENITNNAKNIiVQFNTPVQINCTRPNNmTRKSIRIGPGQAFYAlGDIIGDIRQpHCnVSKATWNETLGKVVKQLR
KHFGNNTIIfFANSSGGDLEVTTHSFNCGGEFFYCNTSGLFNSTWISNTSVQGSNSTGSNDSITLPCRIKQIINMWQRvG
QcMYAPPIQGVIRCVSNITGLILTRDGGSTNSTTETFRPGGGDMRDNWRSELYKYKVVKIEPLGVAPTRcnRtVVGrrrr
rrAVGIGAVFLGFLGAAGSTMGAASnTLTVQARqLLSGIVQQQSNLLRApEAQQHLLKLgVWGfKQLQARVLAVERYLev
QQLLGIWGCSGKLICcTNVPWNSSWSNRNLSEIWDNMTWLQWDKEIgNYTQIIYGLLEESQfQQEiNEQDnLtLD
>JCB-7mutv1_31-33 + 1
(SEQ ID NO: 47)
NVTNNTWVTVYYGVPVWKDAETTLFCASDAKAYETEKHNVWATHACVPTDPNPQEIHLENVTEEFNMWKNNMVEQMHTDI
ISLWDQSLKPCVKLTPLCVILQCTNVTNNITDDMRGELKNCSFNMTTELRDKKQKVYSLFYRLDVVQINENQGNRSNNSN
KEYRLINCNTSAcTQiCPKVSFEPIPIHYCAPAGFAILKCKDKKFNGTGPCkSVSTVQCTHGIKPVVSTQLLLNGSLAEE
EVMIRSENITNNAKNIiVQFNTPVQINCTRPNNmTRKSIRIGPGQAFYAlGDIIGDIRQpHCnVSKATWNETLGKVVKQL
RKHFGNNTIIfFANSSGGDLEVTTHSFNCGGEFFYCNTSGLFNSTWISNTSVQGSNSTGSNDSITLPCRIKQIINMWQRv
GQcMYAPPIQGVIRCVSNITGLILTRDGGSTNSTTETFRPGGGDMRDNWRSELYKYKVVKIEPLGVAPTRcnRtVVGrrr
rrrAVGIGAVFLGFLGAAGSTMGAASnTLTVQARqLLSGIVQQQSNLLRApEAQQHLLKLgVWGfKQLQARVLAVERYLc
vQQLLGIWGCSGKLICcTNVPWNSSWSNRNLSEIWDNMTWLQWDKEIgNYTQIIYGLLEESQfQQEiNEQDnLtLD
>JCB-7mutv1_31-33a
(SEQ ID NO: 48)
NVTYNNTWVTVYYGVPVWKDAETTLFCASDAKAYETEKHNVWATHACVPTDPNPQEIHLENVTEEFNMWKNNMVEQMHTD
IISLWDQSLKPCVKLTPLCVTLQCTNVTNNITDDMRGELKNCSFNMTTELRDKKQKVYSLFYRLDVVQINENQGNRSNNS
NKEYRLINCNTSAcTQiCPKVSFEPIPIHYCAPAGFAILKCKDKKFNGTGPCkSVSTVQCTHGIKPVVSTQLLLNGSLAE
EEVMIRSENITNNAKNIiVQFNTPVQINCTRPNNmTRKSIRIGPGQAFYAlGDIIGDIRQpHCnVSKATWNETLGKVVKQ
LRKHFGNNTIIfFANSSGGDLEVTTHSFNCGGEFFYCNTSGLFNSTWISNTSVQGSNSTGSNDSITLPCRIKQIINMWQR
vGQcMYAPPIQGVIRCVSNITGLILTRDGGSTNSTTETFRPGGGDMRDNWRSELYKYKVVKIEPLGVAPTRcnRtVVGrr
rrrrAVGIGAVFLGFLGAAGSTMGAASnTLTVQARqLLSGIVQQQSNLLRApEAQQHLLKLgVWGfKQLQARVLAVERYL
evQQLLGIWGCSGKLICcTNVPWNSSWSNRNLSEIWQNMTWLQWDKEIgNYTQIIYGLLEESQfQQEiNEQDnLtLD
>JCB-7mutv1_505-668-772-779-783
(SEQ ID NO: 49)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELXNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtNVTVVGrr
rrrrAVGIGAVFLGFLGAAGSTMGAASnTLTVQARqLLSGIVQQQSNLLRApEAQQHLLKLgVWGfKQLQARVLAVERYL
evQQLLGIWGCSGKLICcTNVPWNSSWSNRNLSEIWDNMTWLQWDKEIgNYTQIIYGLLEESQfQQEiNEQDnLtLDGGY
NVTYNVTGSGYNVTYNVTIGGSGG
>JCB-7mutv1_505-507d-668-772-779-783
(SEQ ID NO: 50)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELXNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtNVTGGYNV
TVVGrrrrrrAVGIGAVFLGFLGAAGSTMGAASnTLTVQARqLLSGIVQQQSNLLRApEAQQHLLKLgVWGfKQLQARVL
AVERYLevQQLLGIWGCSGKLICcTNVPWNSSWSNRNLSEIWDNMTWLQWDKEIgNYTQIIYGLLEESQfQCEiNEQDnL
tLDGGYNVTYNVTGSGYNVTYNVTIGGSGG
>JCB-7mutv1_33-668-772-779
(SEQ ID NO: 51)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELXNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESQfQQEiNEQDnLtLDGGYNVT
YNVTGSGYNVTIGGSGG
>JCB-7mutv1_33-668-772-779-783
(SEQ ID NO: 52)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELXNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESQfQQEiNEQDnLtLDGGYNVT
YNVTGSGYNVTYNVTIGGSGG
>JCB-7mutv1_32-668-772-779
(SEQ ID NO: 53)
ANNTWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELXNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESQfQQEiNEQDnLtLDGGYNVT
YNVTGSGYNVTIGGSGG
>JCB-7mutv1_32-668-772-779-783
(SEQ ID NO: 54)
ANNTWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELXNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESQfQQEiNEQDnLtLDGGYNVT
YNVTGSGYNVTYNVTIGGSGG
>JCB-7mutv1_31-33a-668-772-779-783
(SEQ ID NO: 55)
NVTYNNTWVTVYYGVPVWKDAETTLFCASDAKAYETEKHNVWATHACVPTDPNPQEIHLENVTEEFNMWKNNMVEQMHTD
IISLWDQSLKPCVKLTPLCVTLQCTNVTNNITDDMRGELKNCSFNMTTELRDKKQKVYSLFYRLDVVQINENQGNRSNNS
NKEYRLINCNTSAcTQiCPKVSFEPIPIHYCAPAGFAILKCKDKKFNGTGPCkSVSTVQCTHGIKPVVSTQLLLNGSLAE
EEVMIRSENITNNAKNIiVQFNTPVQINCTRPNNmTRKSIRIGPGQAFYAlGDIIGDIRQpHCnVSKATWNETLGKVVKQ
LRKHFGNNTIIfFANSSGGDLEVTTHSFNCGGEFFYCNTSGLFNSTWISNTSVQGSNSTGSNDSITLPCRIKQIINMWQR
vGQcMYAPPIQGVIRCVSNITGLILTRDGGSINSTTETFRPGGGDMRDNWRSELYKYKVVKIEPLGVAPTRcnRtVVGrr
rrrrAVGIGAVFLGFLGAAGSTMGAASnTLTVQARqLLSGIVQQQSNLLRApEAQQHLLKLgVWGfKQLQARVLAVERYL
evQQLLGIWGCSGKLICcTNVPWNSSWSNRNLSEIWQNMTWLQWDKEIgNYTQIIYGLLEESQfQQEiNEQDnLtLDGGY
NVTYNVTGSGYNVTYNVTIGGSGG
>JCB-7mutv1_31-33-68-772-779-783
(SEQ ID NO: 56)
NVTNLTVTVYYGVPVWKDAETTLFCASDAKAYETEKHNVWATHACVPTDPNPQEIHLENVTEEFNMWKNNMVEQMHTDII
SLWDQSLKPCVKLTPLCVTLQCTNVTNNITDDMRGELKNCSFNMTTELRDKKQKVYSLFYRLDVVQINENQGNRSNNSNK
EYRLINCNTSAcTQiCPKVSFEPIPIHYCAPAGFAILKCKDKKFNGTGPCkSVSTVQCTHGIKPVVSTQLLLNGSLAEEE
VMIRSENITNNAKNIiVQFNTPVQINCTRPNNmTRKSIRIGPGQAFYAlGDIIGDIRQpHCnVSKATWNETLGKVVKQLR
KHFGNNTIIfFANSSGGDLEVTTHSFNCGGEFFYCNTSGLFNSTWISNTSVQGSNSTGSNDSITLPCRIKQIINMWQRvG
QcMYAPPIQGVIRCVSNITGLILTRDGGSTNSTTETFRPGGGDMRDNWRSELYKYKVVKIEPLGVAPTRcnRtVVGrrrr
rrAVGIGAVFLGFLGAAGSTMGAASnTLTVQARqLLSGIVQQQSNLLRApEAQQHLLKLgVWGfKQLQARVLAVERYLev
QQLLGIWGCSGKLICcTNVPWNSSWSNRNLSEIWDNMTWLQWDKEIgNYTQIIYGLLEESQfQQEiNEQDnLtLDGGYNV
TYNVTGSGYNVTYNVTIGGSGG
>JCB-7mutv1_32-505
(SEQ ID NO: 57)
ANNTWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtNVTVVGrr
rrrrAVGIGAVFLGFLGAAGSTMGAASnTLTVQARqLLSGIVQQQSNLLRApEAQQHLLKLgVWGfKQLQARVLAVERYL
evQQLLGIWGCSGKLICcTNVPWNSSWSNRNLSEIWDNMTWLQWDKEIgNYTQIIYGLLEESQfQQEiNEQDnLtLD
>JCB-7mutv1_31-33-505
(SEQ ID NO: 58)
NVTNLTVTVYYGVPVWKDAETTLFCASDAKAYETEKHNVWATHACVPTDPNPQEIHLENVTEEFNMWKNNMVEQMHTDII
SLWDQSLKPCVKLTPLCVTLQCTNVTNNITDDMRGELKNCSFNMTTELRDKKQKVYSLFYRLDVVQINENQGNRSNNSNK
EYRLINCNTSAcTQiCPKVSFEPIPIHYCAPAGFAILKCKDKKFNGTGPCkSVSTVQCTHGIKPVVSTQLLLNGSLAEEE
VMIRSENITNNAKNIiVQFNTPVQINCTRPNNmTRKSIRIGPGQAFYAlGDIIGDIRQpHCnVSKATWNETLGKVVKQLR
KHFGNNTIIfFANSSGGDLEVTTHSFNCGGEFFYCNTSGLFNSTWISNTSVQGSNSTGSNDSITLPCRIKQIINMWQRvG
QcMYAPPIQGVIRCVSNITGLILTRDGGSTNSTTETFRPGGGDMRDNWRSELYKYKVVKIEPLGVAPTRcnRtNVTVVGr
rrrrrAVGIGAVFLGFLGAAGSTMGAASnTLTVQARqLLSGIVQQQSNLLRApEAQQHLLKLgVWGfKQLQARVLAVERY
LevQQLLGIWGCSGKLICcTNVPWNSSWSNRNLSEIWDNMTWLQWDKEIgNYTQIIYGLLEESQfQQEiNEQDnLtLD
>JCB-7mutv1_31-33a-505
(SEQ ID NO: 59)
NVTYNNTWVTVYYGVPVWKDAETTLFCASDAKAYETEKHNVWATHACVPTDPNPQEIHLENVTEEFNMWKNNMVEQMHTD
IISLWDQSLKPCVKLTPLCVTLQCTNVTNNITDDMRGELKNCSFNMTTELRDKKQKVYSLFYRLDVVQINENQGNRSNNS
NKEYRLINCNTSAcTQiCPKVSFEPIPIHYCAPAGFAILKCKDKKFNGTGPCkSVSTVQCTHGIKPVVSTQLLLNGSLAE
EEVMIRSENITNNAKNIiVQFNTPVQINCTRPNNmTRKSIRIGPGQAFYAlGDIIGDIRQpHCnVSKATWNETLGKVVKQ
LRKHFGNNTIIfFANSSGGDLEVTTHSFNCGGEFFYCNTSGLFNSTWISNTSVQGSNSTGSNDSITLPCRIKQIINMWQR
vGQcMYAPPIQGVIRCVSNITGLILTRDGGSTNSTTETFRPGGGDMRDNWRSELYKYKVVKIEPLGVAPTRcnRtNVTVV
GrrrrrrAVGIGAVFLGFLGAAGSTMGAASnTLTVQARqLLSGIVQQQSNLLRApEAQQHLLKLgVWGfKQLQARVLAVE
RYLevQQLLGIWGCSGKLICcTNVPWNSSWSNRNLSEIWDNMTWLQWDKEIgNYTQIIYGLLEESQfQQEiNEQDnLtLD
>JCB-7mutv1_31-33-505-668-772-779-783
(SEQ ID NO: 60)
NVTNLTVTVYYGVPVWKDAETTLFCASDAKAYETEKHNVWATHACVPTDPNPQEIHLENVTEEFNMWKNNMVEQMHTDII
SLWDQSLKPCVKLTPLCVTLQCTNVTNNITDDMRGELKNCSFNMTTELRDKKQKVYSLFYRLDVVQINENQGNRSNNSNK
EYRLINCNTSAcTQiCPKVSFEPIPIHYCAPAGFAILKCKDKKFNGTGPCkSVSTVQCTHGIKPVVSTQLLLNGSLAEEE
VMIRSENITNNAKNIiVQFNTPVQINCTRPNNmTRKSIRIGPGQAFYAlGDIIGDIRQpHCnVSKATWNETLGKVVKQLR
KHFGNNTIIfFANSSGGDLEVTTHSFNCGGEFFYCNTSGLFNSTWISNTSVQGSNSTGSNDSITLPCRIKQIINMWQRvG
QcMYAPPIQGVIRCVSNITGLILTRDGGSTNSTTETFRPGGGDMRDNWRSELYKYKVVKIEPLGVAPTRcnRtNVTVVGr
rrrrrAVGIGAVFLGFLGAAGSTMGAASnTLTVQARqLLSGIVQQQSNLLRApEAQQHLLKLgVWGfKQLQARVLAVERY
LcvQQLLGIWGCSGKLICcTNVPWNSSWSNRNLSEIWDNMTWLQWDKEIgNYTQIIYGLLEESQfQQEiNEQDnLtLDGG
YNVTYNVTGSGYNVTYNVTIGGSGG
>JCB-7mutv1_31-33a-505-668-772-779-783
(SEQ ID NO: 61)
NVTYNNTWVTVYYGVPVWKDAETTLFCASDAKAYETEKHNVWATHACVPTDPNPQEIHLENVTEEFNMWKNNMVEQMHTD
IISLWDQSLKPCVKLTPLCVTLQCTNVTNNITDDMRGELKNCSFNMTTELRDKKQKVYSLFYRLDVVQINENQGNRSNNS
NKEYRLINCNTSAcTQiCPKVSFEPIPIHYCAPAGFAILKCKDKKFNGTGPCkSVSTVQCTHGIKPVVSTQLLLNGSLAE
EEVMIRSENITNNAKNIiVQFNTPVQINCTRPNNmTRKSIRIGPGQAFYAlGDIIGDIRQpHCnVSKATWNETLGKVVKQ
LRKHFGNNTIIfFANSSGGDLEVTTHSFNCGGEFFYCNTSGLFNSTWISNTSVQGSNSTGSNDSITLPCRIKQIINMWQR
vGQcMYAPPIQGVIRCVSNITGLILTRDGGSTNSTTETFRPGGGDMRDNWRSELYKYKVVKIEPLGVAPTRcnRtNVTVV
GrrrrrrAVGIGAVFLGFLGAAGSTMGAASnTLTVQARqLLSGIVQQQSNLLRApEAQQHLLKLgVWGfKQLQARVLAVE
RYLevQQLLGIWGCSGKLICcTNVPWNSSWSNRNLSEIWDNMTWLQWDKEIgNYTQIIYGLLEESQfQQEiNEQDnLtLD
GGYNVTYNVTGSGYNVTYNVTIGGSGG
>JCB-7mutv1_31-33a-505-507d-668-772-779-783
(SEQ ID NO: 62)
NVTYNNTWVTVYYGVPVWKDAETTLFCASDAKAYETEKHNVWATHACVPTDPNPQEIHLENVTEEFNMWKNNMVEQMHTD
IISLWDQSLKPCVKLTPLCVTLQCTNVTNNITDDMRGELKNCSFNMTTELRDKKQKVYSLFYRLDVVQINENQGNRSNNS
NKEYRLINCNTSAcTQiCPKVSFEPIPIHYCAPAGFAILKCKDKKFNGTGPCkSVSTVQCTHGIKPVVSTQLLLNGSLAE
EEVMIRSENITNNAKNIiVQFNTPVQINCTRPNNmTRKSIRIGPGQAFYAlGDIIGDIRQpHCnVSKATWNETLGKVVKQ
LRKHFGNNTIIfFANSSGGDLEVTTHSFNCGGEFFYCNTSGLFNSTWISNTSVQGSNSTGSNDSITLPCRIKQIINMWQR
vGQcMYAPPIQGVIRCVSNITGLILTRDGGSTNSTTETFRPGGGDMRDNWRSELYKYKVVKIEPLGVAPTRcnRtNVTGG
YNVTVVGrrrrrrAVGIGAVFLGFLGAAGSTMGAASnTLTVQARqLLSGIVQQQSNLLRApEAQQHLLKLgVWGfKQLQA
RVLAVERYLevQQLLGIWGCSGKLICcTNVPWNSSWSNRNLSEIWDNMTWLQWDKEIgNYTQIIYGLLEESQfQQEiNEQ
DnLtLDGGYNVTYNVTGSGYNVTYNVTIGGSGG
>JCB-7mutv1_31-33a-505-507d-668-772-779
(SEQ ID NO: 63)
NVTYNNTWVTVYYGVPVWKDAETTLFCASDAKAYETEKHNVWATHACVPTDPNPQEIHLENVTEEFNMWKNNMVEQMHTD
IISLWDQSLKPCVKLTPLCVTLQCTNVTNNITDDMRGELKNCSFNMTTELRDKKQKVYSLFYRLDVVQINENQGNRSNNS
NKEYRLINCNTSAcTQiCPKVSFEPIPIHYCAPAGFAILKCKDKKFNGTGPCkSVSTVQCTHGIKPVVSTQLLLNGSLAE
EEVMIRSENITNNAKNIiVQFNTPVQINCTRPNNmTRKSIRIGPGQAFYAlGDIIGDIRQpHCnVSKATWNETLGKVVKQ
LRKHFGNNTIIfFANSSGGDLEVTTHSFNCGGEFFYCNTSGLFNSTWISNTSVQGSNSTGSNDSITLPCRIKQIINMWQR
vGQcMYAPPIQGVIRCVSNITGLILTRDGGSTNSTTETFRPGGGDMRDNWRSELYKYKVVKIEPLGVAPTRcnRtNVTGG
YNVTVVGrrrrrrAVGIGAVFLGFLGAAGSTMGAASnTLTVQARqLLSGIVQQQSNLLRApEAQQHLLKLgVWGfKQLQA
RVLAVERYLevQQLLGIWGCSGKLICcTNVPWNSSWSNRNLSEIWDNMTWLQWDKEIgNYTQIIYGLLEESQfQQEiNEQ
DnLtLDGGYNVTYNVTGSGYNVTIGGSGG
>JCB-7mutv1_31-33a-505-507d-668-772
(SEQ ID NO: 64)
NVTYNNTWVTVYYGVPVWKDAETTLFCASDAKAYETEKHNVWATHACVPTDPNPQEIHLENVTEEFNMWKNNMVEQMHTD
IISLWDQSLKPCVKLTPLCVTLQCTNVTNNITDDMRGELKNCSFNMTTELRDKKQKVYSLFYRLDVVQINENQGNRSNNS
NKEYRLINCNTSAcTQiCPKVSFEPIPIHYCAPAGFAILKCKDKKFNGTGPCkSVSTVQCTHGIKPVVSTQLLLNGSLAE
EEVMIRSENITNNAKNIiVQFNTPVQINCTRPNNmTRKSIRIGPGQAFYAlGDIIGDIRQpHCnVSKATWNETLGKVVKQ
LRKHFGNNTIIfFANSSGGDLEVTTHSFNCGGEFFYCNTSGLFNSTWISNTSVQGSNSTGSNDSITLPCRIKQIINMWQR
vGQcMYAPPIQGVIRCVSNITGLILTRDGGSTNSTTETFRPGGGDMRDNWRSELYKYKVVKIEPLGVAPTRcnRtNVTGG
YNVTVVGrrrrrrAVGIGAVFLGFLGAAGSTMGAASnTLTVQARqLLSGIVQQQSNLLRApEAQQHLLKLgVWGfKQLQA
RVLAVERYLevQQLLGIWGCSGKLICcTNVPWNSSWSNRNLSEIWDNMTWLQWDKEIgNYTQIIYGLLEESQfQQEiNEQ
DnLtLDGGYNVTYNVTIGGSGG
>JCB-7mutv1_31-33-505-668-772
(SEQ ID NO: 65)
NVTNLTVTVYYGVPVWKDAETTLFCASDAKAYETEKHNVWATHACVPTDPNPQEIHLENVTEEFNMWINNMVEQMHTDII
SLWDQSLKPCVKLTPLCVTLQCTNVTNNITDDMRGELKNCSFNMTTELRDKKQKVYSLFYRLDVVQINENQGNRSNNSNK
EYRLINCNTSAcTQiCPKVSFEPIPIHYCAPAGFAILKCKDKKFNGTGPCkSVSTVQCTHGIKPVVSTQLLLNGSLAEEE
VMIRSENITNNAKNIiVQFNTPVQINCTRPNNmTRKSIRIGPGQAFYAlGDIIGDIRQpHCnVSKATWNETLGKVVKQLR
KHFGNNTIIfFANSSGGDLEVTTHSFNCGGEFFYCNTSGLFNSTWISNTSVQGSNSTGSNDSITLPCRIKQIINMWQRvG
QcMYAPPIQGVIRCVSNITGLILTRDGGSTNSTTETFRPGGGDMRDNWRSELYKYKVVKIEPLGVAPTRcnRtNVTVVGr
rrrrrAVGIGAVFLGFLGAAGSTMGAASnTLTVQARqLLSGIVQQQSNLLRApEAQQHLLKLgVWGfKQLQARVLAVERY
LevQQLLGIWGCSGKLICcTNVPWNSSWSNRNLSEIWDNMTWLQWDKEIgNYTQIIYGLLEESQfQQEiNEQDnLtLDGG
YNVTYNVTIGGSGG
>JCB-7mutv1_31-33-505-668-772-779
(SEQ ID NO: 66)
NVTNLTVTVYYGVPVWKDAETTLFCASDAKAYETEKHNVWATHACVPTDPNPQEIHLENVTEEFNMWKNNMVEQMHTDII
SLWDQSLKPCVKLTPLCVTLQCTNVTNNITDDMRGELKNCSFNMTTELRDKKQKVYSLFYRLDVVQINENQGNRSNNSNK
EYRLINCNTSAcTQiCPKVSFEPIPIHYCAPAGFAILKCKDKKFNGTGPCkSVSTVQCTHGIKPVVSTQLLLNGSLAEEE
VMIRSENITNNAKNIiVQFNTPVQINCTRPNNmTRKSIRIGPGQAFYAlGDIIGDIRQpHCnVSKATWNETLGKVVKQLR
KHFGNNTIIfFANSSGGDLEVTTHSFNCGGEFFYCNTSGLFNSTWISNTSVQGSNSTGSNDSITLPCRIKQIINMWQRvG
QcMYAPPIQGVIRCVSNITGLILTRDGGSTNSTTETFRPGGGDMRDNWRSELYKYKVVKIEPLGVAPTRcnRtNVTVVGr
rrrrrAVGIGAVFLGFLGAAGSTMGAASnTLTVQARqLLSGIVQQQSNLLRApEAQQHLLKLgVWGfKQLQARVLAVERY
LevQQLLGIWGCSGKLICcTNVPWNSSWSNRNLSEIWDNMTWLQWDKEIgNYTQIIYGLLEESQfQQEiNEQDnLtLDGG
YNVTYNVTGSGYNVTIGGSGG
>JCB-7mutv1_33-505-668-772
(SEQ ID NO: 67)
AENLTVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtNVTVVGrr
rrrrAVGIGAVFLGFLGAAGSTMGAASnTLTVQARqLLSGIVQQQSNLLRApEAQQHLLKLgVWGfKQLQARVLAVERYL
evQQLLGIWGCSGKLICcTNVPWNSSWSNRNLSEIWDNMTWLQWDKEIgNYTQIIYGLLEESQfQQEiNEQDnLtLDGGY
NVTYNVTIGGSGG
>JCB-7mutv1_32-505-668-772
(SEQ ID NO: 68)
ANNTWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtNVTVVGrr
rrrrAVGIGAVFLGFLGAAGSTMGAASnTLTVQARqLLSGIVQQQSNLLRApEAQQHLLKLgVWGfKQLQARVLAVERYL
evQQLLGIWGCSGKLICcTNVPWNSSWSNRNLSEIWDNMTWLQWDKEIgNYTQIIYGLLEESQfQQEiNEQDnLtLDGGY
NVTYNVTIGGSGG
>JCB-7mutv1_32-505-668-772-779
(SEQ ID NO: 69)
ANNTWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtNVTVVGrr
rrrrAVGIGAVFLGFLGAAGSTMGAASnTLTVQARqLLSGIVQQQSNLLRApEAQQHLLKLgVWGfKQLQARVLAVERYL
evQQLLGIWGCSGKLICcTNVPWNSSWSNRNLSEIWDNMTWLQWDKEIgNYTQIIYGLLEESQfQQEiNEQDnLtLDGGY
NVTYNVTGSGYNVTIGGSGG
>JCB-Mut7v1-3hsh01
(SEQ ID NO: 70)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSACTQICPKVSFEPIPIHYCAPAGFAILKCKDKKFNGTGPCKSVSTVQCTHGIKPVVSTQLLLNGSLAEEEV
MIRSENITNNAKNIIVQFNTPVQINCTRPNNMTRKSIRIGPGQAFYALGDIIGDIRQPHCNVSKATWNETLGKVVKQLRK
HFGNNTIIFFANSSGGDLEVTTHSFNCGGEFFYCNTSGLFNSTWISNTSVQGSNSTGSNDSITLPCRIKQIINMWQRVGQ
CMYAPPIQGVIRCVSNITGLILTRDGGSINSTTETFRPGGGDMRDNWRSELYKYKVVKIEPLGVAPTRCNRTVVGRRRRR
RAVGIGAVFLGFLGAAGSTMGAASNTLTVQARQLLSGIVQQQSNLLRAPEAQQHLLKLGVWGFKQLQARVLAVERYLEVQ
QLLGIWGCSGKLICCTNVPWNSSWSNRNLSEIWDNMTWLQWDKEIGNYTQIIYGLLEESQFQQEINEQDNLILDGGGSSG
VRLWATRQAMLGQVHEVPEGWLIFVAEQEELYVRVQNGFRKVQLEARTPLPR
>JCB-Mut7v1-31-33 + 1_3hsh01
(SEQ ID NO: 71)
NVTNNTWVTVYYGVPVWKDAETTLFCASDAKAYETEKHNVWATHACVPTDPNPQEIHLENVTEEFNMWKNNMVEQMHTDI
ISLWDQSLKPCVKLTPLCVTLQCTNVTNNITDDMRGELKNCSFNMTTELRDKKQKVYSLFYRLDVVQINENQGNRSNNSN
KEYRLINCNTSACTQICPKVSFEPIPIHYCAPAGFAILKCKDKKFNGTGPCKSVSTVQCTHGIKPVVSTQLLLNGSLAEE
EVMIRSENITNNAKNIIVQFNTPVQINCTRPNNMTRKSIRIGPGQAFYALGDIIGDIRQPHCNVSKATWNETLGKVVKQL
RKHFGNNTIIFFANSSGGDLEVTTHSFNCGGEFFYCNTSGLFNSTWISNTSVQGSNSTGSNDSIILPCRIKQIINMWQRV
GQCMYAPPIQGVIRCVSNITGLILTRDGGSTNSTTETFRPGGGDMRDNWRSELYKYKVVKIEPLGVAPTRCNRTVVGRRR
RRRAVGIGAVFLGFLGAAGSTMGAASNTLTVQARQLLSGIVQQQSNLLRAPEAQQHLLKLGVWGFKQLQARVLAVERYLE
VQQLLGIWGCSGKLICCTNVPWNSSWSNRNLSEIWDNMTWLQWDKEIGNYTQIIYGLLEESQFQQEINEQDNLTLDGGGS
SGVRLWATRQAMLGQVHEVPEGWLIFVAEQEELYVRVQNGFRKVQLEARTPLPR
>JCB-Mut7v1-31-33a_3hsh01
(SEQ ID NO: 72)
NVTYNNTWVTVYYGVPVWKDAETTLFCASDAKAYETEKHNVWATHACVPTDPNPQEIHLENVTEEFNMWKNNMVEQMHTD
IISLWDQSLKPCVKLTPLCVTLQCTNVTNNITDDMRGELKNCSFNMTTELRDKKQKVYSLFYRLDVVQINENQGNRSNNS
NKEYRLINCNTSACTQICPKVSFEPIPIHYCAPAGFAILKCKDKKFNGTGPCKSVSTVQCTHGIKPVVSTQLLLNGSLAE
EEVMIRSENITNNAKNIIVQFNTPVQINCTRPNNMTRKSIRIGPGQAFYALGDIIGDIRQPHCNVSKATWNETLGKVVKQ
LRKHFGNNTIIFFANSSGGDLEVTTHSFNCGGEFFYCNTSGLFNSTWISNTSVQGSNSTGSNDSITLPCRIKQIINMWQR
VGQCMYAPPIQGVIRCVSNITGLILTRDGGSTNSTTETFRPGGGDMRDNWRSELYKYKVVKIEPLGVAPTRCNRTVVGRR
RRRRAVGIGAVFLGFLGAAGSTMGAASNTLTVQARQLLSGIVQQQSNLLRAPEAQQHLLKLGVWGFKQLQARVLAVERYL
EVQQLLGIWGCSGKLICCTNVPWNSSWSNRNLSEIWDNMTWLQWDKEIGNYTQIIYGLLEESQFQQEINEQDNLTLDGGG
SSGVRLWATRQAMLGQVHEVPEGWLIFVAEQEELYVRVQNGFRKVQLEARTPLPR
>JCB-Mut7v1-31-33a-505_3hsh01
(SEQ ID NO: 73)
NVTYNNTWVTVYYGVPVWKDAETTLFCASDAKAYETEKHNVWATHACVPTDPNPQEIHLENVTEEFNMWKNNMVEQMHTD
IISLWDQSLKPCVKLTPLCVTLQCTNVTNNITDDMRGELKNCSFNMTTELRDKKQKVYSLFYRLDVVQINENQGNRSNNS
NKEYRLINCNTSACTQICPKVSFEPIPIHYCAPAGFAILKCKDKKFNGTGPCKSVSTVQCTHGIKPVVSTQLLLNGSLAE
EEVMIRSENITNNAKNIIVQFNTPVQINCTRPNNMTRKSIRIGPGQAFYALGDIIGDIRQPHCNVSKATWNETLGKVVKQ
LRKHFGNNTIIFFANSSGGDLEVTTHSFNCGGEFFYCNTSGLFNSTWISNTSVQGSNSTGSNDSITLPCRIKQIINMWQR
VGQCMYAPPIQGVIRCVSNITGLILTRDGGSTNSTTETFRPGGGDMRDNWRSELYKYKVVKIEPLGVAPTRCNRTNVTVV
GRRRRRRAVGIGAVFLGFLGAAGSTMGAASNTLTVQARQLLSGIVQQQSNLLRAPEAQQHLLKLGVWGFKQLQARVLAVE
RYLEVQQLLGIWGCSGKLICCTNVPWNSSWSNRNLSEIWDNMTWLQWDKEIGNYTQIIYGLLEESQFQQEINEQDNLTLD
GGGSSGVRLWATRQAMLGQVHEVPEGWLFVAEQEELYVRVQNGFRKVQLEARTPLPR
>JCB-Mut7v1-3hsh01b
(SEQ ID NO: 74)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSACTQICPKVSFEPIPIHYCAPAGFAILKCKDKKFNGTGPCKSVSTVQCTHGIKPVVSTQLLLNGSLAEEEV
MIRSENITNNAKNIIVQFNTPVQINCTRPNNMTRKSIRIGPGQAFYALGDIIGDIRQPHCNVSKATWNETLGKVVKQLRK
HFGNNTIIFFANSSGGDLEVTTHSFNCGGEFFYCNTSGLFNSTWISNTSVQGSNSTGSNDSITLPCRIKQIINMWQRVGQ
CMYAPPIQGVIRCVSNITGLILTRDGGSTNSTTETFRPGGGDMRDNWRSELYKYKVVKIEPLGVAPTRCNRTVVGRRRRR
RAVGIGAVFLGFLGAAGSTMGAASNTLTVQARQLLSGIVQQQSNLLRAPEAQQHLLKLGVWGFKQLQARVLAVERYLEVQ
QLLGIWGCSGKLICCTNVPWNSSWSNRNLSEIWDNMTWLQWDKEIGNYTQIIYGLLEESQFQQEINEQDNLTLDGGNVTG
VRLWATRQAMLGQVHEVPEGWLIFVAEQEELYVRVQNGFRKVQLEARTPLPR
>JCB-Mut7v1-3hsh01-G18
(SEQ ID NO: 75)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSACTQICPKVSFEPIPIHYCAPAGFAILKCKDKKFNGTGPCKSVSTVQCTHGIKPVVSTQLLLNGSLAEEEV
MIRSENITNNAKNIIVQFNTPVQINCTRPNNMTRKSIRIGPGQAFYALGDIIGDIRQPHCNVSKATWNETLGKVVKQLRK
HFGNNTIIFFANSSGGDLEVTTHSFNCGGEFFYCNTSGLFNSTWISNTSVQGSNSTGSNDSITLPCRIKQIINMWQRVGQ
CMYAPPIQGVIRCVSNITGLILTRDGGSTNSTTETFRPGGGDMRDNWRSELYKYKVVKIEPLGVAPTRCNRTVVGRRRRR
RAVGIGAVFLGFLGAAGSTMGAASNTLTVQARQLLSGIVQQQSNLLRAPEAQQHLLKLGVWGFKQLQARVLAVERYLEVQ
QLLGIWGCSCKLICCTNVPWNSSWSNRNLSEIWDNMTWLQWDKEIGNYTQIIYGLLEESQFQQEINEQDNLTLDGGGSSG
VRLWATRQAMLGQVHNVTKGWLIFVAEQEELYVRVQNGFRKVQLEARTPLPR
>JCB-Mut7v1-3hsh01-G7
(SEQ ID NO: 76)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSACTQICPKVSFEPIPIHYCAPAGFAILKCKDKKFNGTGPCKSVSTVQCTHGIKPVVSTQLLLNGSLAEEEV
MIRSENITNNAKNIIVQFNTPVQINCTRPNNMTRKSIRIGPGQAFYALGDIIGDIRQPHCNVSKATWNETLGKVVKQLRK
HFGNNTIIFFANSSGGDLEVTTHSFNCGGEFFYCNTSGLFNSTWISNTSVQGSNSTGSNDSITLPCRIKQIINMWQRVGQ
CMYAPPIQGVIRCVSNITGLILTRDGGSTNSTTETFRPGGGDMRDNWRSELYKYKVVKIEPLGVAPTRCNRTVVGRRRRR
RAVGIGAVFLGFLGAAGSTMGAASNTLTVQARQLLSGIVQQQSNLLRAPEAQQHLLKLGVWGFKQLQARVLAVERYLEVQ
QLLGIWGCSCKLICCTNVPWNSSWSNRNLSEIWDNMTWLQWDKEIGNYTQIIYGLLEESQFQQEINEQDNLTLDGGGSSG
VRLWNTTKIMLGQVHEVPEGWLIFVAEQEELYVRVQNGFRKVQLEARTPLPR
>JCB-Mut7v1-3hsh01-G7-18
(SEQ ID NO: 6)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSACTQICPKVSFEPIPIHYCAPAGFAILKCKDKKFNGTGPCKSVSTVQCTHGIKPVVSTQLLLNGSLAEEEV
MIRSENITNNAKNIIVQFNTPVQINCTRPNNMTRKSIRIGPGQAFYALGDIIGDIRQPHCNVSKATWNETLGKVVKQLRK
HFGNNTIIFFANSSGGDLEVTTHSFNCGGEFFYCNTSGLFNSTWISNTSVQGSNSTGSNDSITLPCRIKQIINMWQRVGQ
CMYAPPIQGVIRCVSNITGLILTRDGGSTNSTTETFRPGGGDMRDNWRSELYKYKVVKIEPLGVAPTRCNRTVVGRRRRR
RAVGIGAVFLGFLGAAGSTMGAASNTLTVQARQLLSGIVQQQSNLLRAPEAQQHLLKLGVWGFKQLQARVLAVERYLEVQ
QLLGIWGCSCKLICCTNVPWNSSWSNRNLSEIWDNMTWLQWDKEIGNYTQIIYGLLEESQFQQEINEQDNLTLDGGGSSG
VRLWNTTKIMLGQVHNVTKGWLIFVAEQEELYVRVQNGFRKVQLEARTPLPR
>JCB-Mut7v1-3hsh01-G7-18-30-47
(SEQ ID NO: 77)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSACTQICPKVSFEPIPIHYCAPAGFAILKCKDKKFNGTGPCKSVSTVQCTHGIKPVVSTQLLLNGSLAEEEV
MIRSENITNNAKNIIVQFNTPVQINCTRPNNMTRKSIRIGPGQAFYALGDIIGDIRQPHCNVSKATWNETLGKVVKQLRK
HFGNNTIIFFANSSGGDLEVTTHSFNCGGEFFYCNTSGLFNSTWISNTSVQGSNSTGSNDSITLPCRIKQIINMWQRVGQ
CMYAPPIQGVIRCVSNITGLILTRDGGSTNSTTETFRPGGGDMRDNWRSELYKYKVVKIEPLGVAPTRCNRTVVGRRRRR
RAVGIGAVFLGFLGAAGSTMGAASNTLTVQARQLLSGIVQQQSNLLRAPEAQQHLLKLGVWGFKQLQARVLAVERYLEVQ
QLLGIWGCSCKLICCTNVPWNSSWSNRNLSEIWDNMTWLQWDKEIGNYTQIIYGLLEESQFQQEINEQDNLTLDGGGSSG
VRLWNTTKIMLGQVHNVTKGWLIFVAENETLYVRVQNGFRKVQLNVTTPLPR
>JCB-Mut7v1-3hsh02
(SEQ ID NO: 78)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSACTQICPKVSFEPIPIHYCAPAGFAILKCKDKKFNGTGPCKSVSTVQCTHGIKPVVSTQLLLNGSLAEEEV
MIRSENITNNAKNIIVQFNTPVQINCTRPNNMTRKSIRIGPGQAFYALGDIIGDIRQPHCNVSKATWNETLGKVVKQLRK
HFGNNTIIFFANSSGGDLEVTTHSFNCGGEFFYCNTSGLFNSTWISNTSVQGSNSTGSNDSITLPCRIKQIINMWQRVGQ
CMYAPPIQGVIRCVSNITGLILTRDGGSTNSTTETFRPGGGDMRDNWRSELYKYKVVKIEPLGVAPTRCNRTVVGRRRRR
RAVGIGAVFLGFLGAAGSTMGAASNTLTVQARQLLSGIVQQQSNLLRAPEAQQHLLKLGVWGFKQLQARVLAVERYLEVQ
QLLGIWGCSGKLICCTNVPWNSSWSNRNLSEIWDNMTWLQWDKEIGNYTQIIYGLLEESQFQQEINEQDNLTLDGGSGGS
SGVRLWATRQAMLGQVHEVPEGWLIFVAEQEELYVRVQNGFRKVQLEARTPLPR
>JCB-Mut7v1-31-33 + 1_3hsh02
(SEQ ID NO: 79)
NVTNNTWVTVYYGVPVWKDAETTLFCASDAKAYETEKHNVWATHACVPTDPNPQEIHLENVTEEFNMWKNNMVEQMHTDI
ISLWDQSLKPCVKLTPLCVTLQCTNVTNNITDDMRGELKNCSFNMTTELRDKKQKVYSLFYRLDVVQINENQGNRSNNSN
KEYRLINCNTSACTQICPKVSFEPIPIHYCAPAGFAILKCKDKKFNGTGPCKSVSTVQCTHGIKPVVSTQLLLNGSLAEE
EVMIRSENITNNAKNIIVQFNTPVQINCTRPNNMTRKSIRIGPGQAFYALGDIIGDIRQPHCNVSKATWNETLGKVVKQL
RKHFGNNTIIFFANSSGGDLEVTTHSFNCGGEFFYCNTSGLFNSTWISNTSVQGSNSTGSNDSITLPCRIKQIINMWQRV
GQCMYAPPIQGVIRCVSNITGLILTRDGGSTNSTTETFRPGGGDMRDNWRSELYKYKVVKIEPLGVAPTRCNRTVVGRRR
RRRAVGIGAVFLGFLGAAGSTMGAASNTLTVQARQLLSGIVQQQSNLLRAPEAQQHLLKLGVWGFKQLQARVLAVERYLE
VQQLLGIWGCSGKLICCTNVPWNSSWSNRNLSEIWDNMTWLQWDKEIGNYTQIIYGLLEESQFQQEINEQDNLTLDGGSG
GSSGVRLWATRQAMLGQVHEVPEGWLIFVAEQEELYVRVQNGFRKVQLEARTPLPR
>JCB-Mut7v1-31-33a_3hsh02
(SEQ ID NO: 80)
NVTYNNTWVTVYYGVPVWKDAETTLFCASDAKAYETEKHNVWATHACVPTDPNPQEIHLENVTEEFNMWKNNMVEQMHTD
IISLWDQSLKPCVKLTPLCVTLQCTNVTNNITDDMRGELKNCSFNMTTELRDKKQKVYSLFYRLDVVQINENQGNRSNNS
NKEYRLINCNTSACTQICPKVSFEPIPIHYCAPAGFAILKCKDKKFNGTGPCKSVSTVQCTHGIKPVVSTQLLLNGSLAE
EEVMIRSENITNNAKNIIVQFNTPVQINCTRPNNMTRKSIRIGPGQAFYALGDIIGDIRQPHCNVSKATWNETLGKVVKQ
LRKHFGNNTIIFFANSSGGDLEVTTHSFNCGGEFFYCNTSGLFNSTWISNTSVQGSNSTGSNDSITLPCRIKQIINMWQR
VGQCMYAPPIQGVIRCVSNITGLILTRDGGSTNSTTETFRPGGGDMRDNWRSELYKYKVVKIEPLGVAPTRCNRTVVGRR
RRRRAVGIGAVFLGFLGAAGSTMGAASNTLTVQARQLLSGIVQQQSNLLRAPEAQQHLLKLGVWGFKQLQARVLAVERYL
EVQQLLGIWGCSGKLICCTNVPWNSSWSNRNLSEIWDNMTWLQWDKEIGNYTQIIYGLLEESQFQQEINEQDNLTLDGGS
GGSSGVRLWATRQAMLGQVHEVPEGWLIFVAEQEELYVRVQNGFRKVQLEARTPLPR
>JCB-Mut7v1-31-33a-505_3hsh02
(SEQ ID NO: 81)
NVTYNNTWVTVYYGVPVWKDAETTLFCASDAKAYETEKHNVWATHACVPTDPNPQEIHLENVTEEFNMWKNNMVEQMHTD
IISLWDQSLKPCVKLTPLCVTLQCTNVTNNITDDMRGELKNCSFNMTTELRDKKQKVYSLFYRLDVVQINENQGNRSNNS
NKEYRLINCNTSACTQICPKVSFEPIPIHYCAPAGFAILKCKDKKFNGTGPCKSVSTVQCTHGIKPVVSTQLLLNGSLAE
EEVMIRSENITNNAKNIIVQFNTPVQINCTRPNNMTRKSIRIGPGQAFYALGDIIGDIRQPHCNVSKATWNETLGKVVKQ
LRKHFGNNTIIFFANSSGGDLEVTTHSFNCGGEFFYCNTSGLFNSTWISNTSVQGSNSTGSNDSITLPCRIKQIINMWQR
VGQCMYAPPIQGVIRCVSNITGLILTRDGGSTNSTTETFRPGGGDMRDNWRSELYKYKVVKIEPLGVAPTRCNRTNVTVV
GRRRRRRAVGIGAVFLGFLGAAGSTMGAASNTLTVQARQLLSGIVQQQSNLLRAPEAQQHLLKLGVWGFKQLQARVLAVE
RYLEVQQLLGIWGCSGKLICCTNVPWNSSWSNRNLSEIWDNMTWLQWDKEIGNYTQIIYGLLEESQFQQEINEQDNLTLD
GGSGGSSGVRLWATRQAMLGQVHEVPEGWLIFVAEQEELYVRVQNGFRKVQLEARTPLPR
>JCB-Mut7v1-31-33a-505-507d_3hsh02
(SEQ ID NO: 82)
NVTYNNTWVTVYYGVPVWKDAETTLFCASDAKAYETEKHNVWATHACVPTDPNPQEIHLENVTEEFNMWKNNMVEQMHTD
IISLWDQSLKPCVKLTPLCVTLQCTNVTNNITDDMRGELKNCSFNMTTELRDKKQKVYSLFYRLDVVQINENQGNRSNNS
NKEYRLINCNTSACTQICPKVSFEPIPIHYCAPAGFAILKCKDKKFNGTGPCKSVSTVQCTHGIKPVVSTQLLLNGSLAE
EEVMIRSENITNNAKNIIVQFNTPVQINCTRPNNMTRKSIRIGPGQAFYALGDIIGDIRQPHCNVSKATWNETLGKVVKQ
LRKHFGNNTIIFFANSSGGDLEVTTHSFNCGGEFFYCNTSGLFNSTWISNTSVQGSNSTGSNDSITLPCRIKQIINMWQR
VGQCMYAPPIQGVIRCVSNITGLILTRDGGSTNSTTETFRPGGGDMRDNWRSELYKYKVVKIEPLGVAPTRCNRTNVTGG
YNVTVVGRRRRRRAVGIGAVFLGFLGAAGSTMGAASNTLTVQARQLLSGIVQQQSNLLRAPEAQQHLLKLGVWGFKQLQA
RVLAVERYLEVQQLLGIWGCSGKLICCTNVPWNSSWSNRNLSEIWDNMTWLQWDKEIGNYTQIIYGLLEESQFQQEINEQ
DNLTLDGGSGGSSGVRLWATRQAMLGQVHEVPEGWLIFVAEQEELYVRVQNGFRKVQLEARTPLPR
>JCB-Mut7v1-3hsh02b
(SEQ ID NO: 83)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSACTQICPKVSFEPIPIHYCAPAGFAILKCKDKKFNGTGPCKSVSTVQCTHGIKPVVSTQLLLNGSLAEEEV
MIRSENITNNAKNIIVQFNTPVQINCTRPNNMTRKSIRIGPGQAFYALGDIIGDIRQPHCNVSKATWNETLGKVVKQLRK
HFGNNTIIFFANSSGGDLEVTTHSFNCGGEFFYCNTSGLFNSTWISNTSVQGSNSTGSNDSITLPCRIKQIINMWQRVGQ
CMYAPPIQGVIRCVSNITGLILTRDGGSTNSTTETFRPGGGDMRDNWRSELYKYKVVKIEPLGVAPTRCNRTVVGRRRRR
RAVGIGAVFLGFLGAAGSTMGAASNTLTVQARQLLSGIVQQQSNLLRAPEAQQHLLKLGVWGFKQLQARVLAVERYLEVQ
QLLGIWGCSGKLICCTNVPWNSSWSNRNLSEIWDNMTWLQWDKEIGNYTQIIYGLLEESQFQQEINEQDNLTLDGGSGNV
TGVRLWATRQAMLGQVHEVPEGWLIFVAEQEELYVRVQNGFRKVQLEARTPLPR
>JCB-Mut7v1-3hsh02-G7-18
(SEQ ID NO: 84)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSACTQICPKVSFEPIPIHYCAPAGFAILKCKDKKFNGTGPCKSVSTVQCTHGIKPVVSTQLLLNGSLAEEEV
MIRSENITNNAKNIIVQFNTPVQINCTRPNNMTRKSIRIGPGQAFYALGDIIGDIRQPHCNVSKATWNETLGKVVKQLRK
HFGNNTIIFFANSSGGDLEVTTHSFNCGGEFFYCNTSGLFNSTWISNTSVQGSNSTGSNDSITLPCRIKQIINMWQRVGQ
CMYAPPIQGVIRCVSNITGLILTRDGGSTNSTTETFRPGGGDMRDNWRSELYKYKVVKIEPLGVAPTRCNRTVVGRRRRR
RAVGIGAVFLGFLGAAGSTMGAASNTLTVQARQLLSGIVQQQSNLLRAPEAQQHLLKLGVWGFKQLQARVLAVERYLEVQ
QLLGIWGCSCKLICCTNVPWNSSWSNRNLSEIWDNMTWLQWDKEIGNYTQIIYGLLEESQFQQEINEQDNLTLDGGSGGS
SGVRLWNTTKIMLGQVHNVTKGWLIFVAEQEELYVRVQNGERKVQLEARTPLPR
>JCB-Mut7v1-31-33a-3hsh02-G7-18
(SEQ ID NO: 85)
NVTYNNTWVTVYYGVPVWKDAETTLFCASDAKAYETEKHNVWATHACVPTDPNPQEIHLENVTEEFNMWKNNMVEQMHTD
IISLWDQSLKPCVKLTPLCVTLQCTNVTNNITDDMRGELKNCSFNMTTELRDKKQKVYSLFYRLDVVQINENQGNRSNNS
NKEYRLINCNTSACTQICPKVSFEPIPIHYCAPAGFAILKCKDKKFNGTGPCKSVSTVQCTHGIKPVVSTQLLLNGSLAE
EEVMIRSENITNNAKNIIVQFNTPVQINCTRPNNMTRKSIRIGPGQAFYALGDIIGDIRQPHCNVSKATWNETLGKVVKQ
LRKHFGNNTIIFFANSSGGDLEVTTHSFNCGGEFFYCNTSGLFNSTWISNTSVQGSNSTGSNDSITLPCRIKQIINMWQR
VGQCMYAPPIQGVIRCVSNITGLILTRDGGSINSTTETFRPGGGDMRDNWRSELYKYKVVKIEPLGVAPTRCNRTVVGRR
RRRRAVGIGAVFLGFLGAAGSTMGAASNTLTVQARQLLSGIVQQQSNLLRAPEAQQHLLKLGVWGFKQLQARVLAVERYL
EVQQLLGIWGCSGKLICCTNVPWNSSWSNRNLSEIWQNMTWLQWDKEIGNYTQIIYGLLEESQFQQEINEQDNLTLDGGS
GGSSGVRLWNTTKIMLGQVHNVTKGWLIFVAEQEELYVRVQNGFRKVQLEARTPLPR
>JCB-Mut7v1-3hsh02-G7-18-30-47
(SEQ ID NO: 86)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSACTQICPKVSFEPIPIHYCAPAGFAILKCKDKKFNGTGPCKSVSTVQCTHGIKPVVSTQLLLNGSLAEEEV
MIRSENITNNAKNIIVQFNTPVQINCTRPNNMTRKSIRIGPGQAFYALGDIIGDIRQPHCNVSKATWNETLGKVVKQLRK
HFGNNTIIFFANSSGGDLEVTTHSFNCGGEFFYCNTSGLFNSTWISNTSVQGSNSTGSNDSITLPCRIKQIINMWQRVGQ
CMYAPPIQGVIRCVSNITGLILTRDGGSTNSTTETFRPGGGDMRDNWRSELYKYKVVKIEPLGVAPTRCNRTVVGRRRRR
RAVGIGAVFLGFLGAAGSTMGAASNTLTVQARQLLSGIVQQQSNLLRAPEAQQHLLKLGVWGFKQLQARVLAVERYLEVQ
QLLGIWGCSCKLICCTNVPWNSSWSNRNLSEIWDNMTWLQWDKEIGNYTQIIYGLLEESQFQQEINEQDNLTLDGGSGGS
SGVRLWNTTKIMLGQVHNVTKGWLIFVAENETLYVRVQNGFRKVQLNVTTPLPR
>JCB-Mut7v1-3hsh03
(SEQ ID NO: 87)
SSGVRLWATRQAMLGQVHEVPEGWLIFVAEQEELYVRVQNGFRKVQLEARTPLPRGSGGGAENLWVTVYYGVPVWKDAET
TLFCASDAKAYETEKHNVWATHACVPTDPNPQEIHLENVTEEFNMWKNNMVEQMHTDIISLWDQSLKPCVKLTPLCVTLQ
CTNVTNNITDDMRGELKNCSFNMTTELRDKKQKVYSLFYRLDVVQINENQGNRSNNSNKEYRLINCNTSACTQICPKVSF
EPIPIHYCAPAGFAILKCKDKKFNGTGPCKSVSTVQCTHGIKPVVSTQLLLNGSLAEEEVMIRSENITNNAKNIIVQFNT
PVQINCTRPNNMTRKSIRIGPGQAFYALGDIIGDIRQPHCNVSKATWNETLGKVVKQLRKHFGNNTIIFFANSSGGDLEV
TTHSFNCGGEFFYCNTSGLFNSTWISNTSVQGSNSTGSNDSITLPCRIKQIINMWQRVGQCMYAPPIQGVIRCVSNITGL
ILTRDGGSTNSTTETFRPGGGDMRDNWRSELYKYKVVKIEPLGVAPTRCNRTVVGRRRRRRAVGIGAVFLGFLGAAGSTM
GAASNTLTVQARQLLSGIVQQQSNLLRAPEAQQHLLKLGVWGFKQLQARVLAVERYLEVQQLLGIWGCSGKLICCTNVPW
NSSWSNRNLSEIWDNMTWLQWDKEIGNYTQIIYGLLEESQFQQEINEQDNLTLD
>JCB-Mut7v1-668-772-3hsh03
(SEQ ID NO: 88)
SSGVRLWATRQAMLGQVHEVPEGWLIFVAEQEELYVRVQNGFRKVQLEARTPLPRGSGGGAENLWVTVYYGVPVWKDAET
TLFCASDAKAYETEKHNVWATHACVPTDPNPQEIHLENVTEEFNMWKNNMVEQMHTDIISLWDQSLKPCVKLTPLCVTLQ
CTNVTNNITDDMRGELKNCSFNMTTELRDKKQKVYSLFYRLDVVQINENQGNRSNNSNKEYRLINCNTSACTQICPKVSF
EPIPIHYCAPAGFAILKCKDKKFNGTGPCKSVSTVQCTHGIKPVVSTQLLLNGSLAEEEVMIRSENITNNAKNIIVQFNT
PVQINCTRPNNMTRKSIRIGPGQAFYALGDIIGDIRQPHCNVSKATWNETLGKVVKQLRKHFGNNTIIFFANSSGGDLEV
TTHSFNCGGEFFYCNTSGLFNSTWISNTSVQGSNSTGSNDSITLPCRIKQIINMWQRVGQCMYAPPIQGVIRCVSNITGL
ILTRDGGSTNSTTETFRPGGGDMRDNWRSELYKYKVVKIEPLGVAPTRCNRTVVGRRRRRRAVGIGAVFLGFLGAAGSTM
GAASNTLTVQARQLLSGIVQQQSNLLRAPEAQQHLLKLGVWGFKQLQARVLAVERYLEVQQLLGIWGCSGKLICCTNVPW
NSSWSNRNLSEIWDNMTWLQWDKEIGNYTQIIYGLLEESQFQQEINEQDNLTLDGGYNVTYNVTIGGSGG
JCBMut7v13133a3hsh03
(SEQ ID NO: 89)
SSGVRLWATRQAMLGQVHEVPEGWLIFVAEQEELYVRVQNGFRKVQLEARTPLPRGSGGGNVTYNNTWVTVYYGVPVWKD
AETTLFCASDAKAYETEKHNVWATHACVPTDPNPQEIHLENVTEEFNMWKNNMVEQMHTDIISLWDQSLKPCVKLTPLCV
TLQCTNVTNNITDDMRGELKNCSFNMTTELRDKKQKVYSLFYRLDVVQINENQGNRSNNSNKEYRLINCNTSACTQICPK
VSFEPIPIHYCAPAGFAILKCKDKKFNGTGPCKSVSTVQCTHGIKPVVSTQLLLNGSLAEEEVMIRSENITNNAKNIIVQ
FNTPVQINCTRPNNMTRKSIRIGPGQAFYALGDIIGDIRQPHCNVSKATWNETLGKVVKQLRKHFGNNTIIFFANSSGGD
LEVTTHSFNCGGEFFYCNTSGLFNSTWISNTSVQGSNSTGSNDSITLPCRIKQIINMWQRVGQCMYAPPIQGVIRCVSNI
TGLILTRDGGSTNSTTETFRPGGGDMRDNWRSELYKYKVVKIEPLGVAPTRCNRTVVGRRRRRRAVGIGAVFLGFLGAAG
STMGAASNTLTVQARQLLSGIVQQQSNLLRAPEAQQHLLKLGVWGFKQLQARVLAVERYLEVQQLLGIWGCSGKLICCTN
VPWNSSWSNRNLSEIWDNMTWLQWDKEIGNYTQIIYGLLEESQFQQEINEQDNLTLD
>JCB-Mut7v1-3hsh04
(SEQ ID NO: 90)
SSGVRLWATRQAMLGQVHEVPEGWLIFVAEQEELYVRVQNGFRKVQLEGSGAENLWVTVYYGVPVWKDAETTLFCASDAK
AYETEKHNVWATHACVPTDPNPQEIHLENVTEEFNMWKNNMVEQMHTDIISLWDQSLKPCVKLTPLCVTLQCTNVTNNIT
DDMRGELKNCSFNMTTELRDKKQKVYSLFYRLDVVQINENQGNRSNNSNKEYRLINCNTSACTQICPKVSFEPIPIHYCA
PAGFAILKCKDKKFNGTGPCKSVSTVQCTHGIKPVVSTQLLLNGSLAEEEVMIRSENIINNAKNIIVQFNTPVQINCTRP
NNMTRKSIRIGPGQAFYALGDIIGDIRQPHCNVSKATWNETLGKVVKQLRKHFGNNTIIFFANSSGGDLEVTTHSFNCGG
EFFYCNTSGLFNSTWISNTSVQGSNSTGSNDSITLPCRIKCIINMWQRVGQCMYAPPIQGVIRCVSNITGLILTRDGGST
NSTTETFRPGGGDMRDNWRSELYKYKVVKIEPLGVAPTRCNRTVVGRRRRRRAVGIGAVFLGFLGAAGSTMGAASNTLTV
QARQLLSGIVQQQSNLLRAPEAQQHLLKLGVWGFKQLQARVLAVERYLEVQQLLGIWGCSGKLICCTNVPWNSSWSNRNL
SEIWDNMTWLQWDKEIGNYTQIIYGLLEESQFQQEINEQDNLTLD
>JCB-Mut7v1-31-33a-3hsh04
(SEQ ID NO: 91)
SSGVRLWATRQAMLGQVHEVPEGWLIFVAEQEELYVRVQNGFRKVQLEGSGNVTYNNTWVTVYYGVPVWKDAETTLFCAS
DAKAYETEKHNVWATHACVPTDPNPQEIHLENVTEEENMWKNNMVEQMHTDIISLWDQSLKPCVKLTPLCVTLQCTNVTN
NITDDMRGELKNCSFNMTTELRDKKQKVYSLFYRLDVVQINENQGNRSNNSNKEYRLINCNTSACTQICPKVSFEPIPIH
YCAPAGFAILKCKDKKFNGTGPCKSVSTVQCTHGIKPVVSTQLLLNGSLAEEEVMIRSENITNNAKNIIVQFNTPVQINC
TRPNNMTRKSIRIGPGQAFYALGDIIGDIRQPHCNVSKATWNETLGKVVKQLRKHFGNNTIIFFANSSGGDLEVTTHSFN
CGGEFFYCNTSGLFNSTWISNTSVQGSNSTGSNDSITLPCRIKQIINMWQRVGQCMYAPPIQGVIRCVSNITGLILTRDG
GSTNSTTETFRPGGGDMRDNWRSELYKYKVVKIEPLGVAPTRCNRTVVGRRRRRRAVGIGAVFLGFLGAAGSTMGAASNT
LTVQARQLLSGIVQQQSNLLRAPEAQQHLLKLGVWGFKQLQARVLAVERYLEVQQLLGIWGCSGKLICCTNVPWNSSWSN
RNLSEIWDNMTWLQWDKEIGNYTQIIYGLLEESQFQQEINEQDNLTLD
>JCB-Mut7v1-3hsh05
(SEQ ID NO: 92)
SSGVRLWATRQAMLGQVHEVPEGWLIFVAEQEELYVRVQNGFRKVQLEGSGGGAENLWVTVYYGVPVWKDAETTLFCASD
AKAYETEKHNVWATHACVPTDPNPQEIHLENVTEEFNMWKNNMVEQMHTDIISLWDQSLKPCVKLTPLCVTLQCTNVTNN
ITDDMRGELKNCSFNMTTELRDKKQKVYSLFYRLDVVQINENQGNRSNNSNKEYRLINCNTSACTQICPKVSFEPIPIHY
CAPAGFAILKCKDKKFNGTGPCKSVSTVQCTHGIKPVVSTQLLLNGSLAEEEVMIRSENITNNAKNIIVQFNTPVQINCT
RPNNMTRKSIRIGPGQAFYALGDIIGDIRQPHCNVSKATWNETLGKVVKQLRKHFGNNTIIFFANSSGGDLEVTTHSFNC
GGEFFYCNTSGLFNSTWISNTSVQGSNSTGSNDSITLPCRIKQIINMWQRVGQCMYAPPIQGVIRCVSNITGLILTRDGG
STNSTTETFRPGGGDMRDNWRSELYKYKVVKIEPLGVAPTRCNRTVVGRRRRRRAVGIGAVFLGFLGAAGSTMGAASNTL
TVQARQLLSGIVQQQSNLLRAPEAQQHLLKLGVWGFKQLQARVLAVERYLEVQQLLGIWGCSGKLICCTNVPWNSSWSNR
NLSEIWDNMTWLQWDKEIGNYTQIIYGLLEESQFQQEINEQDNLTLD
>JCB-Mut7v1-31-33a-3hsh05
(SEQ ID NO: 93)
SSGVRLWATRQAMLGQVHEVPEGWLIFVAEQEELYVRVQNGFRKVQLEGSGGGNVTYNNTWVTVYYGVPVWKDAETTLFC
ASDAKAYETEKHNVWATHACVPTDPNPQEIHLENVTEEFNMWKNNMVEQMHTDIISLWDQSLKPCVKLTPLCVTLQCTNV
TNNITDDMRGELKNCSFNMTTELRDKKQKVYSLFYRLDVVQINENQGNRSNNSNKEYRLINCNTSACTQICPKVSFEPIP
IHYCAPAGFAILKCKDKKFNGTGPCKSVSTVQCTHGIKPVVSTQLLLNGSLAEEEVMIRSENITNNAKNIIVQFNTPVQI
NCTRPNNMTRKSIRIGPGQAFYALGDIIGDIRQPHCNVSKATWNETLGKVVKQLRKHFGNNTIIFFANSSGGDLEVTTHS
FNCGGEFFYCNTSGLFNSTWISNTSVQGSNSTGSNDSITLPCRIKQIINMWQRVGQCMYAPPIQGVIRCVSNITGLILTR
DGGSTNSTTETFRPGGGDMRDNWRSELYKYKVVKIEPLGVAPTRCNRTVVGRRRRRRAVGIGAVFLGFLGAAGSTMGAAS
NTLTVQARQLLSGIVQQQSNLLRAPEAQQHLLKLGVWGFKQLQARVLAVERYLEVQQLLGIWGCSGKLICCTNVPWNSSW
SNRNLSEIWDNMTWLQWDKEIGNYTQIIYGLLEESQFQQEINEQDNLTLD
>JCB-Mut7v1-31-33a_3hsh05-G-1-7-18-30
(SEQ ID NO: 94)
NVTGVRLWNTTKIMLGQVHNVTKGWLIFVAENETLYVRVQNGFRKVQLEGSGGGNVTYNNTWVTVYYGVPVWKDAETTLF
CASDAKAYETEKHNVWATHACVPTDPNPQEIHLENVTEEFNMWKNNMVEQMHTDIISLWDQSLKPCVKLTPLCVTLQCTN
VTNNITDDMRGELKNCSFNMTTELRDKKQKVYSLFYRLDVVQINENQGNRSNNSNKEYRLINCNTSACTQICPKVSFEPI
PIHYCAPAGFAILKCKDKKFNGTGPCKSVSTVQCTHGIKPVVSTQLLLNGSLAEEEVMIRSENITNNAKNIIVQFNTPVQ
INCTRPNNMTRKSIRIGPGQAFYALGDIIGDIRQPHCNVSKATWNETLGKVVKQLRKHFGNNTIIFFANSSGGDLEVTTH
SFNCGGEFFYCNTSGLFNSTWISNTSVQGSNSTGSNDSITLPCRIKQIINMWQRVGQCMYAPPIQGVIRCVSNITGLILT
RDGGSTNSTTETFRPGGGDMRDNWRSELYKYKVVKIEPLGVAPTRCNRTVVGRRRRRRAVGIGAVFLGFLGAAGSTMGAA
SNTLTVQARQLLSGIVQQQSNLLRAPEAQQHLLKLGVWGFKQLQARVLAVERYLEVQQLLGIWGCSGKLICCTNVPWNSS
WSNRNLSEIWDNMTWLQWDKEIGNYTQIIYGLLEESQFQQEINEQDNLTLD
>JG1_7mut3_N656.664
(SEQ ID NO: 95)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESQfQQEiNEtDnLtLD
>JG2_7mut3_N656.659
(SEQ ID NO: 5)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESQfQQEiNEtD
>JG3_7mut3_N656.659g
(SEQ ID NO: 96)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSCKLICcTNVPWNSSWSNRNLSEIWDNMTWLQWDKEIgNYTQIIYGLLEESQfQQEiNEtDg
>JG4_7mut3_N652.664
(SEQ ID NO: 97)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSCKLICcTNVPWNSSWSNRNLSEIWDNMTWLQWDKEIgNYTQIIYGLLEESQfnQLiNEQDnLLLD
>JG5_7mut3_N652.657
(SEQ ID NO: 98)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESQfnQtiNE
>JG6_7mut3_N649.664
(SEQ ID NO: 99)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSGKLICCTNVPWNSSWSNRNLSEIWDNMTWLQWDKEIgNYTQIIYGLLEEnQtQQEiNEQDnLtLD
>JG7_7mut3_N649.657
(SEQ ID NO: 100)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSCKLICCTNVPWNSSWSNRNLSEIWDNMTWLQWDKEIgNYTQIIYGLLEEnQtQQEiNE
>JG8_7mut3_N649.653
(SEQ ID NO: 101)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEEnQtQQ
>JG9_7mut 3_N645.664
(SEQ ID NO: 102)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGnLtESQfQQEiNEQDnLtLD
>JG10_7mut3_N645.657
(SEQ ID NO: 103)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGnLtESQfQQEiNE
>JG11_7mut3_N645.653
(SEQ ID NO: 104)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSCKLICcTNVPWNSSWSNRNLSEIWDNMTWLQWDKEIgNYTQIIYGnLtESQfQQ
>JG12_7mut3_N535.664
(SEQ ID NO: 105)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGIGAVFLGFLGAAGSTMGAASnTtTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESQfQQEiNEQDnLtLD
>JG13_7mut3_N535.653
(SEQ ID NO: 106)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGIGAVFLGFLGAAGSTMGAASnTtTVQARqLLSGIVQQQSNLLRApEAQQHLLKLgVWGfKQLQARVLAVERYLevQ
QLLGIWGCSCKLICcTNVPWNSSWSNRNLSEIWDNMTWLQWDKEIgNYTQIIYGLLEESQfQQ
>JG14_7mut3_N538.664
(SEQ ID NO: 107)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGIGAVFLGFLGAAGSTMGAASnTLnVtARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESQfQQEINEQDnLtLD
>JG15_7mut3_N538.653
(SEQ ID NO: 108)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGIGAVFLGFLGAAGSTMGAASnTLnVtARqLLSGIVQQQSNLLRApEAQQHLLKLgVWGfKQLQARVLAVERYLevQ
QLLGIWGCSEKLICcTNVPWNSSWSNRNLSEIWDNMTWLQWDKEIgNYTQIIYGLLEESQfQQ
>JG16_7mut3_N542.664
(SEQ ID NO: 109)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGIGAVFLGFLGAAGSTMGAASnTLTVQAnqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESQfQQEiNEQDnLtLD
>JG17_7mut3_N542.653
(SEQ ID NO: 110)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGIGAVFLGFLGAAGSTMGAASnTLTVQAngtLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESQfQQ
>JG18_7mut3_N535_N652.657
(SEQ ID NO: 111)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGIGAVFLGFLGAAGSTMGAASnTtTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESQfnQtiNE
>5Mut-R503N_V505T
(SEQ ID NO: 112)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQACPKVSFEPIPIHYCAPAGFAILKCKDKKFNGTGPCknVSTVQCTHGIKPVVSTQLLLNGSLAEEEV
MIRSENITNNAKNIiVQFNTPVQINCTRPNNmTRKSIRIGPGQAFYAlGDIIGDIRQpHCTVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKnRtVGrrrrr
rAVGIGAVFLGFLGAAGSTMGAASnTLTVQARqLLSGIVQQQSNLLRApEAQQHLLKLgVWGIKQLQARVLAVERYLevQ
QLLGIWGCSGKLICcTNVPWNSSWSNRNLSEIWDNMTWLQWDKEIgNYTQIIYGLLEESQfQQEiNEQDLLALD
>5Mut-R503N_V505T_G483N_V485T_Q617N_Q619T
(SEQ ID NO: 113)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQACPKVSFEPIPIHYCAPAGFAILKCKDKKFNGTGPCknVSTVQCTHGIKPVVSTQLLLNGSLAEEEV
MIRSENITNNAKNIiVQFNTPVQINCTRPNNmTRKSIRIGPGQAFYAlGDIIGDIRQpHCTVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKnRtVGrrrrr
rAVGInAtFLGFLGAAGSTMGAASNTLTVQARqLLSGIVQQQSNLLRApEAQQHLLKLgVWGIKQLQARVLAVERYLevQ
QLLGIWGCSGKLICcTNVPWNSSWSNRNLSEIWDNMTWLQWDKEIgNYTQIIYGLLEESnftQEiNEQDLLALD
>5Mut-R504N_V506T
(SEQ ID NO: 114)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQACPKVSFEPIPIHYCAPAGFAILKCKDKKFNGTGPCknVSTVQCTHGIKPVVSTQLLLNGSLAEEEV
MIRSENITNNAKNIiVQFNTPVQINCTRPNNmTRKSIRIGPGQAFYAlGDIIGDIRQpHCTVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKRnVtGrrrrr
rAVGIGAVFLGFLGAAGSTMGAASnTLTVQARqLLSGIVQQQSNLLRApEAQQHLLKLgVWGIKQLQARVLAVERYLevQ
QLLGIWGCSGKLICcTNVPWNSSWSNRNLSEIWDNMTWLQWDKEIgNYTQIIYGLLEESQfQQEiNEQDLLALD
>5Mut-R504N_V506T_G483N_V485T_Q617N_Q619T
(SEQ ID NO: 115)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQACPKVSFEPIPIHYCAPAGFAILKCKDKKFNGTGPCknVSTVQCTHGIKPVVSTQLLLNGSLAEEEV
MIRSENITNNAKNIiVQFNTPVQINCTRPNNmTRKSIRIGPGQAFYAlGDIIGDIRQpHCTVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKRnVtGrrrrr
rAVGInAtFLGFLGAAGSTMGAASnTLTVQARqLLSGIVQQQSNLLRApEAQQHLLKLgVWGIKQLQARVLAVERYLevQ
QLLGIWGCSCKLICcTNVPWNSSWSNRNLSEIWDNMTWLQWDKEIgNYTQIIYGLLEESnftQEiNEQDLLALD
>5Mut-L661N_L663T
(SEQ ID NO: 116)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQACPKVSFEPIPIHYCAPAGFAILKCKDKKFNGTGPCknVSTVQCTHGIKPVVSTQLLLNGSLAEEEV
MIRSENITNNAKNIiVQFNTPVQINCTRPNNmTRKSIRIGPGQAFYAlGDIIGDIRQpHCTVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKRRVVGrrrrr
rAVGIGAVFLGFLGAAGSTMGAASnTLTVQARqLLSGIVQQQSNLLRApEAQQHLLKLgVWGIKQLQARVLAVERYLevQ
QLLGIWGCSCKLICcTNVPWNSSWSNRNLSEIWDNMTWLQWDKEIgNYTQIIYGLLEESQfQQEiNEQDLnAtD
>5Mut-L661N_L663T_G483N_V485T_Q617N_Q619T
(SEQ ID NO: 117)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQACPKVSFEPIPIHYCAPAGFAILKCKDKKFNGTGPCknVSTVQCTHGIKPVVSTQLLLNGSLAEEEV
MIRSENITNNAKNIiVQFNTPVQINCTRPNNmTRKSIRIGPGQAFYAlGDIIGDIRQpHCTVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKRRVVGrrrrr
rAVGInAtFLGFLGAAGSTMGAASnTLTVQARqLLSGIVQQQSNLLRApEAQQHLLKLgVWGIKQLQARVLAVERYLevQ
QLLGIWGCSGKLICcTNVPWNSSWSNRNLSEIWDNMTWLQWDKEIgNYTQIIYGLLEESnftQEiNEQDLnAtD
>5Mut-R503N_V505T_A662N_D664T
(SEQ ID NO: 118)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQACPKVSFEPIPIHYCAPAGFAILKCKDKKFNGTGPCknVSTVQCTHGIKPVVSTQLLLNGSLAEEEV
MIRSENITNNAKNIiVQFNTPVQINCTRPNNmTRKSIRIGPGQAFYAlGDIIGDIRQpHCTVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKnRtVGrrrrr
rAVGIGAVFLGFLGAAGSTMGAASnTLTVQARqLLSGIVQQQSNLLRApEAQQHLLKLgVWGIKQLQARVLAVERYLevQ
QLLGIWGCSCKLICcTNVPWNSSWSNRNLSEIWDNMTWLQWDKEIgNYTQIIYGLLEESQfQQEiNEQDLLnLt
>5Mut-R503N_V505T_A662N_D664T_G483N_V485T_Q617N_Q619T
(SEQ ID NO: 119)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQACPKVSFEPIPIHYCAPAGFAILKCKDKKFNGTGPCknVSTVQCTHGIKPVVSTQLLLNGSLAEEEV
MIRSENITNNAKNIiVQFNTPVQINCTRPNNmTRKSIRIGPGQAFYAlGDIIGDIRQpHCTVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKnRtVGrrrrr
rAVGInAtFLGFLGAAGSTMGAASnTLTVQARqLLSGIVQQQSNLLRApEAQQHLLKLgVWGIKQLQARVLAVERYLevQ
QLLGIWGCSGKLICcTNVPWNSSWSNRNLSEIWDNMTWLQWDKEIgNYTQIIYGLLEESnftQEiNEQDLLnLt
>5Mut-R503N_V505T_A662N_D664T_A665_L666_D667
(SEQ ID NO: 120)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQACPKVSFEPIPIHYCAPAGFAILKCKDKKFNGTGPCknVSTVQCTHGIKPVVSTQLLLNGSLAEEEV
MIRSENITNNAKNIiVQFNTPVQINCTRPNNmTRKSIRIGPGQAFYAlGDIIGDIRQpHCTVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKnRtVGrrrrr
rAVGIGAVFLGFLGAAGSTMGAASnTLTVQARqLLSGIVQQQSNLLRApEAQQHLLKLgVWGIKQLQARVLAVERYLevQ
QLLGIWGCSGKLICcTNVPWNSSWSNRNLSEIWDNMTWLQWDKEIgNYTQIIYGLLEESQfQQEiNEQDLLnLtald
>5Mut-R503N_V505T_A662N_D664T_A665_L666_D667_G483N_V485T_Q617N_Q619T
(SEQ ID NO: 121)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQACPKVSFEPIPIHYCAPAGFAILKCKDKKFNGTGPCknVSTVQCTHGIKPVVSTQLLLNGSLAEEEV
MIRSENITNNAKNIiVQFNTPVQINCTRPNNmTRKSIRIGPGQAFYAlGDIIGDIRQpHCTVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKnRtVGrrrrr
rAVGInAtFLGFLGAAGSTMGAASnTLTVQARqLLSGIVQQQSNLLRApEAQQHLLKLgVWGIKQLQARVLAVERYLevQ
QLLGIWGCSCKLICcTNVPWNSSWSNRNLSEIWDNMTWLQWDKEIgNYTQIIYGLLEESnftQEiNEQDLLnLtald
>5Mut-base-glycan-G483N_V485T_Q617N_Q619T
(SEQ ID NO: 122)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQACPKVSFEPIPIHYCAPAGFAILKCKDKKFNGTGPCknVSTVQCTHGIKPVVSTQLLLNGSLAEEEV
MIRSENITNNAKNIiVQFNTPVQINCTRPNNmTRKSIRIGPGQAFYAlGDIIGDIRQpHCTVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGInAtFLGFLGAAGSTMGAASnTLTVQARqLLSGIVQQQSNLLRApEAQQHLLKLgVWGIKQLQARVLAVERYLevQ
QLLGIWGCSGKLICcTNVPWNSSWSNRNLSEIWDNMTWLQWDKEIgNYTQIIYGLLEESnftQEiNEQDnLtLD
>5Mut-base-glycan-
(SEQ ID NO: 123)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQACPKVSFEPIPIHYCAPAGFAILKCKDKKFNGTGPCknVSTVQCTHGIKPVVSTQLLLNGSLAEEEV
MIRSENITNNAKNIiVQFNTPVQINCTRPNNmTRKSIRIGPGQAFYAlGDIIGDIRQpHCTVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGInAtFLGFLGAAGSTMGAASnTtTVQARqLLSGIVQQQSNLLRApEAQQHLLKLgVWGIKQLQARVLAVERYLevQ
QLLGIWGCSGKLICcTNVPWNSSWSNRNLSEIWDNMTWLQWDKEIgNYTQIIYGLLEESnftQEiNEQDnLtLD
>5Mut-base-glycan-G483N_V485T_L504T
(SEQ ID NO: 124)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQACPKVSFEPIPIHYCAPAGFAILKCKDKKFNGTGPCknVSTVQCTHGIKPVVSTQLLLNGSLAEEEV
MIRSENITNNAKNIiVQFNTPVQINCTRPNNmTRKSIRIGPGQAFYAlGDIIGDIRQpHCTVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGInAtFLGFLGAAGSTMGAASnTtTVQARqLLSGIVQQQSNLLRApEAQQHLLKLgVWGIKQLQARVLAVERYLevQ
QLLGIWGCSCKLICcTNVPWNSSWSNRNLSEIWDNMTWLQWDKEIgNYTQIIYGLLEESQQQEiNEQDnLtLD
>5Mut-base-glycan-
(SEQ ID NO: 125)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQACPKVSFEPIPIHYCAPAGFAILKCKDKKFNGTGPCknVSTVQCTHGIKPVVSTQLLLNGSLAEEEV
MIRSENITNNAKNIiVQFNTPVQINCTRPNNmTRKSIRIGPGQAFYAlGDIIGDIRQpHCTVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcnRtVVGrrrrr
rAVGIGAVFLGFLGAAGSTMGAASnTtTVQARqLLSGIVQQQSNLLRApEAQQHLLKLgVWGIKQLQARVLAVERYLevQ
QLLGIWGCSCKLICcTNVPWNSSWSNRNLSEIWDNMTWLQWDKEIgNYTQIIYGLLEESnftQEiNEQDnLtLD
>7mutv1-R503N_V505T
(SEQ ID NO: 126)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCknVSTVQCTHGIKPVVSTQLLINGSLAEEEV
MIRSENITNNAKNIiVQFNTPVQINCTRPNNmTRKSIRIGPGQAFYAlGDIIGDIRDpHCTVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKnRtVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESQfQQEiNEQDLLALD
>7mutv1-R503N_V505T_G483N_V485T_Q617N_Q619T
(SEQ ID NO: 127)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCknVSTVQCTHGIKPVVSTQLLINGSLAEEEV
MIRSENITNNAKNIiVQFNTPVQINCTRPNNmTRKSIRIGPGQAFYAlGDIIGDIRDpHCTVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKnRtVGrrrrr
rAVGInAtFLGFLGAAGSTMGAASnTLTVQARqLLSGIVQQQSNLLRApEAQQHLLKLgVWGfKQLQARVLAVERYLevQ
QLLGIWGCSCKLICcTNVPWNSSWSNRNLSEIWDNMTWLQWDKEIgNYTQIIYGLLEESnftQEiNEQDLLALD
>7mutv1-R504N_V506T
(SEQ ID NO: 128)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCknVSTVQCTHGIKPVVSTQLLINGSLAEEEV
MIRSENITNNAKNIiVQFNTPVQINCTRPNNmTRKSIRIGPGQAFYAlGDIIGDIRDpHCTVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKRnVtGrrrrr
rAVGIGAVFLGFLGAAGSTMGAASNTLTVQARqLLSGIVQQQSNLLRAPEAQQHLLKLgVWGfKQLQARVLAVERYLevQ
QLLGIWGCSCKLICcTNVPWNSSWSNRNLSEIWDNMTWLQWDKEIgNYTQIIYGLLEESQfQQEiNEQDLLALD
>7mutv1-R504N_V506T_G483N_V485T_Q617N_Q619T
(SEQ ID NO: 129)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCknVSTVQCTHGIKPVVSTQLLINGSLAEEEV
MIRSENITNNAKNIiVQFNTPVQINCTRPNNmTRKSIRIGPGQAFYAlGDIIGDIRDpHCTVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKRnVtGrrrrr
rAVGInAtFLGFLGAAGSTMGAASnTLTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESnftQEiNEQDLLALD
>7mutv1-L661N_L663T
(SEQ ID NO: 130)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCknVSTVQCTHGIKPVVSTQLLINGSLAEEEV
MIRSENITNNAKNIiVQFNTPVQINCTRPNNmTRKSIRIGPGQAFYAlGDIIGDIRDpHCTVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKRRVVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESQfQQEiNEQDLnAtD
>7mutv1-L661N_L663T_G483N_V485T_Q617N_Q619T_R504N_V506T
(SEQ ID NO: 131)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCknVSTVQCTHGIKPVVSTQLLINGSLAEEEV
MIRSENITNNAKNIiVQFNTPVQINCTRPNNmTRKSIRIGPGQAFYAlGDIIGDIRDpHCTVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKRnVtGrrrrr
rAVGInAtFLGFLGAAGSTMGAASnTLTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESnftQEiNEQDLnAtD
>7mutv1-R503N_V505T_A662N_D664T
(SEQ ID NO: 132)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCknVSTVQCTHGIKPVVSTQLLINGSLAEEEV
MIRSENITNNAKNIiVQFNTPVQINCTRPNNmTRKSIRIGPGQAFYAlGDIIGDIRDpHCTVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKnRtVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESQfQQEiNEQDLLnLt
>7mutv1-R503N_V505T_A662N_D664T_G483N_V485T_Q617N_Q619T
(SEQ ID NO: 133)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCknVSTVQCTHGIKPVVSTQLLINGSLAEEEV
MIRSENITNNAKNIiVQFNTPVQINCTRPNNmTRKSIRIGPGQAFYAlGDIIGDIRDpHCTVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKnRtVGrrrrr
rAVGInAtFLGFLGAAGSTMGAASnTLTVQARqLLSGIVQQQSNLLRApEAQQHLLKLgVWGfKQLQARVLAVERYLevQ
QLLGIWGCSCKLICcTNVPWNSSWSNRNLSEIWDNMTWLQWDKEIgNYTQIIYGLLEESnftQEiNEQDLLnLt
>7mutv1-R503N_V505T_A662N_D664T_A665_L666_D667
(SEQ ID NO: 134)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCknVSTVQCTHGIKPVVSTQLLINGSLAEEEV
MIRSENITNNAKNIiVQFNTPVQINCTRPNNmTRKSIRIGPGQAFYAlGDIIGDIRDpHCTVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKnRtVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESQfQQEiNEQDLLnLtald
>7mutv1-R503N_V505T_A662N_D664T_A665_L666_D667_G483N_V485T_Q617N_Q619T
(SEQ ID NO: 135)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCknVSTVQCTHGIKPVVSTQLLINGSLAEEEV
MIRSENITNNAKNIiVQFNTPVQINCTRPNNmTRKSIRIGPGQAFYAlGDIIGDIRDpHCTVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKnRtVGrrrrr
rAVGInAtFLGFLGAAGSTMGAASnTLTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESnftQEiNEQDLLnLtald
>7Mutv1-Gly332-dGly241-R503N_V505T
(SEQ ID NO: 136)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKnRtVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESQfQQEiNEQDLLALD
>7Mutv1-Gly332-dGly241-R503N_V505T_G483N_V485T_Q617N_Q619T
(SEQ ID NO: 137)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKnRtVGrrrrr
rAVGInAtFLGFLGAAGSTMGAASnTLTVQARqLLSGIVQQQSNLLRApEAQQHLLKLgVWGfKQLQARVLAVERYLevQ
QLLGIWGCSCKLICcTNVPWNSSWSNRNLSEIWDNMTWLQWDKEIgNYTQIIYGLLEESnftQEiNEQDLLALD
>7Mutv1-Gly332-dGly241-R504N_V506T
(SEQ ID NO: 138)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKRnVtGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSCKLICcTNVPWNSSWSNRNLSEIWDNMTWLQWDKEIgNYTQIIYGLLEESQfQQEiNEQDLLALD
>7Mutv1-Gly332-dGly241-R504N_V506T_G483N_V485T_Q617N_Q619T
(SEQ ID NO: 139)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKRnVtGrrrrr
rAVGInAtFLGFLGAAGSTMGAASnTLTVQARqLLSGIVQQQSNLLRApEAQQHLLKLgVWGfKQLQARVLAVERYLevQ
QLLGIWGCSCKLICcTNVPWNSSWSNRNLSEIWDNMTWLQWDKEIgNYTQIIYGLLEESnftQEiNEQDLLALD
>7Mutv1-Gly332-dGly241-L661N_L663T
(SEQ ID NO: 140)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKRRVVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSCKLICcTNVPWNSSWSNRNLSEIWDNMTWLQWDKEIgNYTQIIYGLLEESQfQQEiNEQDLnAtD
>7Mutv1-Gly332-dGly241-L661N_L663T_G483N_V485T_Q617N_Q619T
(SEQ ID NO: 141)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKRRVVGrrrrr
rAVGInAtFLGFLGAAGSTMGAASnTLTVQARqLLSGIVQQQSNLLRApEAQQHLLKLgVWGfKQLQARVLAVERYLevQ
QLLGIWGCSCKLICcTNVPWNSSWSNRNLSEIWDNMTWLQWDKEIgNYTQIIYGLLEESnftQEiNEQDLnAtD
>7Mutv1-Gly332-dGly241-
(SEQ ID NO: 142)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKnRtVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESQfQQEiNEQDLLnLt
>7Mutv1-Gly332-dGly241-
(SEQ ID NO: 143)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKnRtVGrrrrr
rAVGInAtFLGFLGAAGSTMGAASnTLTVQARqLLSGIVQQQSNLLRApEAQQHLLKLgVWGfKQLQARVLAVERYLevQ
QLLGIWGCSCKLICcTNVPWNSSWSNRNLSEIWDNMTWLQWDKEIgNYTQIIYGLLEESnftQEiNEQDLLnLt
>7Mutv1-Gly332-dGly241-
(SEQ ID NO: 144)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKnRtVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESQfQQEiNEQDLLnLtald
>7Mutv1-Gly332-dGly241-
(SEQ ID NO: 145)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKnRtVGrrrrr
rAVGInAtFLGFLGAAGSTMGAASnTLTVQARqLLSGIVQQQSNLLRApEAQQHLLKLgVWGfKQLQARVLAVERYLevQ
QLLGIWGCSCKLICcTNVPWNSSWSNRNLSEIWDNMTWLQWDKEIgNYTQIIYGLLEESnftQEiNEQDLLnLtald
>7Mutv1-Gly332-dGly241-base_glycan-G483N_V485T_Q617N_Q619T
(SEQ ID NO: 146)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKnRtVGrrrrr
rAVGInAtFLGFLGAAGSTMGAASnTLTVQARqLLSGIVQQQSNLLRApEAQQHLLKLgVWGfKQLQARVLAVERYLcvQ
QLLGIWGCSCKLICcTNVPWNSSWSNRNLSEIWDNMTWLQWDKEIgNYTQIIYGLLEESnftQEiNEQDnLtLD
>7Mutv1-Gly332-dGly241-base_glycan-
(SEQ ID NO: 147)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKnRtVGrrrrr
rAVGInAtFLGFLGAAGSTMGAASnTtTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESnftQEiNEQDnLtLD
>7Mutv1-Gly332-dGly241-base_glycan-Q617N_Q619T
(SEQ ID NO: 148)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKnRtVGrrrrr
rAVGIGAVFLGFLGAAGSTMGAASnTLTVQARqLLSGIVQQQSNLLRApEAQQHLLKLgVWGfKQLQARVLAVERYLevQ
QLLGIWGCSCKLICcTNVPWNSSWSNRNLSEIWDNMTWLQWDKEIgNYTQIIYGLLEESnftQEiNEQDnLtLD
>7Mutv1-Gly332-dGly241-base_glycan-Q617N_Q619T_L504T
(SEQ ID NO: 7)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKnRtVGrrrrr
rAVGIGAVFLGFLGAAGSTMGAASnTtTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESnftQEiNEQDnLtLD
>7Mutv1-Gly332-dGly241-base_glycan-
(SEQ ID NO: 149)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKnRtVGrrrrr
rAVGInALFLGFLGAAGSTMGAASnTLTVQARqLLSGIVQQQSNLLRApEAQQHLLKLgVWGfKQLQARVLAVERYLevQ
QLLGIWGCSGKLICcTNVPWNSSWSNRNLSEIWDNMTWLQWDKEIgNYTQIIYGLLEESQfQQEiNEQDnLtLD
>7Mutv1-Gly332-dGly241-base_glycan-
(SEQ ID NO: 150)
AENLWVTVYYGVPVWKDAETTLFCASDAKAYETEKHNVWATHACVPTDPNPQEIHLENVTEEFNMWKNNMVEQMHTDIIS
LWDQSLKPCVKLTPLCVTLQCTNVTNNITDDMRGELKNCSFNMTTELRDKKQKVYSLFYRLDVVQINENQGNRSNNSNKE
YRLINCNTSAcTQiCPKVSFEPIPIHYCAPAGFAILKCKDKKFNGTGPCkSVSTVQCTHGIKPVVSTQLLLNGSLAEEEV
MIRSENITNNAKNIiVQFNTPVQINCTRPNNmTRKSIRIGPGQAFYAlGDIIGDIRQpHCnVSKATWNETLGKVVKQLRK
HFGNNTIIfFANSSGGDLEVTTHSFNCGGEFFYCNTSGLFNSTWISNTSVQGSNSTGSNDSITLPCRIKQIINMWQRvGQ
cMYAPPIQGVIRCVSNITGLILTRDGGSTNSTTETFRPGGGDMRDNWRSELYKYKVVKIEPLGVAPTRcKnRtVGrrrrr
rAVGInAtFLGFLGAAGSTMGAASnTtTVQARqLLSGIVQQQSNLLRApEAQQHLLKLgVWGfKQLQARVLAVERYLevQ
QLLGIWGCSCKLICcTNVPWNSSWSNRNLSEIWDNMTWLQWDKEIqNYTQIIYGLLEESQfQQEiNEQDnLtLD

It will be apparent that the precise details of the methods or compositions described may be varied or modified without departing from the spirit of the described implementations. We claim all such modifications and variations that fall within the scope and spirit of the claims below.

1. A soluble HIV-1 envelope (Env) ectodomain trimer, comprising:
protomers comprising or consisting of HIV-1 Env positions 31 to one of 653-664 and modified with amino acid substitutions, additions, and/or insertions as follows:
(A) amino acid substitutions for stabilization of the trimer in a prefusion closed conformation, the amino acid substitutions comprising:
(i) cysteine substitutions at HIV-1 Env positions 501 and 605 that form a non-natural intra-protomer disulfide bond;
(ii) cysteine substitutions at HIV-1 Env positions 201 and 433 that form a non-natural intra-protomer disulfide bond;
(iii) a proline substitution at HIV-1 Env position 559; and
(iv) methionine, leucine, and proline substitutions at HIV-1 Env positions 302, 320, and 329, respectively;
(B) amino acid substitutions, additions, and/or insertions to introduce N-linked glycan sequons as follows:
(i) optionally an addition of three amino acids immediately N-terminal to HIV-1 Env position 31 to introduce an N-linked glycan sequon;
(ii) amino acid substitutions and/or insertions to introduce one, two, or three N-linked glycan sequons between HIV-1 Env positions 502 and 509, wherein no more than 15 amino acids are inserted; and
(iii) amino acid substitutions between HIV-1 Env positions 650-664 to introduce one or two N-linked glycan sequons, and/or addition of up to 60 amino acids at the C-terminus of the protomers to introduce one to five N-linked glycan sequons;
(C) optionally a substitution of RRRRRR (SEQ ID NO: 21) for the amino acids of a gp120/gp41 furin cleavage site; and
wherein the HIV-1 Env positions are according to HXB2 numbering and the soluble HIV-1 Env ectodomain trimer elicits an immune response to HIV-1.

2. The soluble HIV-1 Env ectodomain trimer of claim 1, wherein the protomers comprise (B)(i) the addition of three amino acids immediately N-terminal to HIV-1 Env position 31 to introduce an N-linked glycan sequon.

3. The soluble HIV-1 Env ectodomain trimer of claim 1, wherein part (B)(ii) is amino acid substitution to introduce an N-linked glycan sequon at HIV-1 Env position 502, and/or insertion of up to 15 amino acids with at least two N-linked glycan sequons between HIV-1 Env positions 507/508.

4. The soluble HIV-1 Env ectodomain trimer of claim 3, wherein the insertion of up to 15 amino acids comprises or consists of insertion of amino acids NSTHKNLTHHM (SEQ ID NO: 11).

5. The soluble HIV-1 Env ectodomain trimer of claim 1, wherein part (B)(iii) is amino acid substitutions to introduce an N-linked glycan sequon at one or two of positions 650, 656, and 660, and/or addition of three amino acids at the C-terminus of the protomers to introduce an N-linked glycan sequon.

6. The soluble HIV-1 Env ectodomain trimer of claim 1, wherein part (B)(iii) is amino acid substitutions to introduce an N-linked glycan sequon at one or two of positions 650, 656, and 660, and/or addition of up to 60 amino acids at the C-terminus of the protomers to introduce up to four N-linked glycan sequons.

7. The soluble HIV-1 Env ectodomain trimer of claim 1, wherein the protomers of the trimer comprise the substitution of RRRRRR (SEQ ID NO: 21) for the amino acids of a gp120/gp41 furin cleavage site.

8. The soluble HIV-1 Env ectodomain trimer of claim 1, wherein (B) the amino acid substitutions, additions, and/or insertions to protomers of the trimer to introduce N-linked glycan sequons comprise:
(i) optionally the addition of three amino acids immediately N-terminal to HIV-1 Env position 31 to introduce an N-linked glycan sequon; and
(ii) amino acid substitutions to introduce an N-linked glycan sequon at HIV-1 Env position 502, insertion of up to 15 amino acids with at least two N-linked glycan sequons between HIV-1 Env positions 507/508, or both;
(iii) amino acid substitutions to introduce N-linked glycan sequons at one, two, or three of HIV-1 Env positions 650, 656, and 660; and optionally the amino acid addition at the C-terminus of the protomers to introduce up to five N-linked glycan sequons;
wherein the protomers of the trimer comprise the substitution of RRRRRR (SEQ ID NO: 21) for the amino acids of a gp120/gp41 furin cleavage site.

9. The soluble HIV-1 Env ectodomain trimer of claim 1,
wherein (B) the amino acid substitutions, additions, and/or insertions to protomers of the trimer to introduce N-linked glycan sequons comprise:
(i)addition of three amino acids immediately N-terminal to HIV-1 Env position 31 to introduce an N-linked glycan sequon;
(ii) amino acid substitution to introduce an N-linked glycan sequon at HIV-1 Env position 502;
insertion of amino acids NSTHKNLTHHM (SEQ ID NO: 11) between HIV-1 Env positions 507/508 to introduce two N-linked glycan sequons;
amino acid substitution to introduce an N-linked glycan sequon at HIV-1 Env position 660; and
(iii) addition of three amino acids at the C-terminus of the protomers to introduce an N-linked glycan sequon; and
(C) the protomers of the trimer comprise the substitution of RRRRRR (SEQ ID NO: 21) for the amino acids of a gp120/gp41 furin cleavage site.

10. The soluble HIV-1 Env ectodomain trimer of claim 1, wherein (B) the amino acid substitutions and/or insertions to protomers of the trimer to introduce N-linked glycan sequons comprise:
(ii) amino acid substitution to introduce an N-linked glycan sequon at HIV-1 Env position 502;
(iii) amino acid substitutions to introduce an N-linked glycan sequon at one of HIV-1 Env positions 650, 656, or 660;
optionally addition of three amino acids at the C-terminus of the protomers to introduce an N-linked glycan sequon, or addition of up to 60 amino acids at the C-terminus of the protomers to introduce up to four N-linked glycan sequons; and
(C) the protomers of the trimer comprise the substitution of RRRRRR (SEQ ID NO: 21) for the amino acids of a gp120/gp41 furin cleavage site.

11. The soluble HIV-1 Env ectodomain trimer of claim 1, further comprising one or more amino acid substitutions to introduce one or more of 2041, 535N, 573F, 588E, 589V, 651F, and 6551 amino acids (HXB2 numbering) if not already present in the protomer.

12. The soluble HIV-1 Env ectodomain trimer of claim 1, further comprising one or more amino acid substitutions to introduce the sequence AENL for positions 31-35 of the protomer, if not already present.

13. The soluble HIV-1 Env ectodomain trimer of claim 1, wherein the protomers do not comprise N-linked glycan sequons at HIV-1 Env positions 504 and 661.

14. The soluble HIV-1 Env ectodomain trimer of claim 1, wherein:
the cysteine substitutions at HIV-1 Env positions 201 and 433 are I201C and A433C substitutions;
the cysteine substitutions at HIV-1 Env positions 501 and 605 are A501C and T605C substitutions;
the proline substitution at HIV-1 Env position 559 is a I559P substitution;
the methionine substitution at HIV-1 Env position 302 is a N302M substitution;
the leucine substitution at HIV-1 Env position 320 is a T320L substitution; and/or
the proline substitution at HIV-1 Env position 329 is a A329P substitution.

15. The soluble HIV-1 Env ectodomain trimer of claim 1, wherein the protomers of the soluble HIV-1 Env ectodomain trimer further comprise one or more amino acid substitutions to introduce an N-linked glycosylation site at position 332 if the N-linked glycosylation site is not already present.

16. The soluble HIV-1 Env ectodomain trimer of claim 1, selected from one of: a soluble Clade A HIV-1 Env ectodomain trimer, a soluble Clade B HIV-1 Env ectodomain trimer, a soluble Clade C HIV-1 Env ectodomain trimer, a soluble Clade D HIV-1 Env ectodomain trimer, and a soluble Clade F HIV-1 Env ectodomain trimer, that comprises the amino acid substitutions.

17. The soluble HIV-1 Env ectodomain trimer of claim 1, wherein the protomers of the trimer comprise or consist of an amino acid sequence at least 95% identical to HXB2 positions 31 to one of 653-664 of a native HIV-1 Env sequence further modified with the amino acid substitutions, additions, and/or insertions.

18. The soluble HIV-1 Env ectodomain trimer of claim 17, wherein the native HIV-1 Env protein sequence is set forth as any one of SEQ ID NOs: 22-34.

19. The soluble HIV-1 Env ectodomain trimer of claim 1, wherein the protomers of the soluble HIV-1 Env ectodomain trimer further comprises one or more additional amino acid substitutions.

20. The soluble HIV-1 Env ectodomain trimer of claim 1, wherein the soluble HIV-1 Env ectodomain trimer binds to sCD4 with an affinity of tighter than 350 nM.

21. The soluble HIV-1 Env ectodomain trimer of claim 1, wherein the protomers comprise or consist of an amino acid sequence at least 95% identical to any one of SEQ ID NOs: 2-10.

22. The soluble HIV-1 Env ectodomain trimer of claim 1, wherein the protomers comprise or consists of the amino acid sequence set forth as any one of SEQ ID NOs: 2-10.

23. The soluble HIV-1 Env ectodomain trimer of claim 1, conjugated to a heterologous carrier.

24. The soluble HIV-1 Env ectodomain trimer of claim 1, wherein the N-linked glycan sequons are glycosylated.

25. A protein nanoparticle comprising the soluble HIV-1 Env ectodomain trimer of claim 1.

26. An isolated nucleic acid molecule encoding a protomer of the soluble HIV-1 Env ectodomain trimer of claim 1.

27. The isolated nucleic acid molecule of claim 26, encoding a precursor of the protomer of the soluble HIV-1 Env ectodomain trimer.

28. The isolated nucleic acid molecule of claim 26, operably linked to a promoter.

29. The isolated nucleic acid molecule of claim 26, wherein the nucleic acid molecule is an RNA molecule.

30. A vector comprising the isolated nucleic acid molecule of claim 26.

31. An immunogenic composition for use to elicit an immune response to HIV-1 in a subject, comprising the soluble HIV-1 Env ectodomain trimer of claim 1 or a nucleic acid encoding the soluble HIV-1 Env ectodomain trimer of claim 1, and a pharmaceutically acceptable carrier.

32. The immunogenic composition of claim 31, further comprising an adjuvant.

33. A method of producing a soluble HIV-1 Env ectodomain trimer, comprising:
expressing the nucleic acid molecule of claim 26 in a host cell; and
purifying the soluble HIV-1 Env ectodomain trimer.

34. A method for eliciting an immune response to HIV-1 in a subject, comprising administering to the subject an effective amount of the soluble HIV-1 Env ectodomain trimer of claim 1 or a nucleic acid encoding the soluble HIV-1 Env ectodomain trimer of claim 1, to elicit the immune response.

35. The method of claim 34, wherein the soluble HIV-1 Env ectodomain trimer or nucleic acid encoding the soluble HIV-1 Env ectodomain trimer, is administered as a boost in a prime-boost immunization protocol to induce the immune response to HIV-1 Env in the subject.

36. The method of claim 34, wherein the immune response treats or inhibits HIV-1 infection in the subject.

37. (canceled)
